(19) United States
(12) Patent Application Publication
(10) Pub. No.: US 2024/0287573 A1
(43) Pub. Date: Aug. 29, 2024

(54) E3 LIGASE FUSION PROTEINS FOR PROXIMITY DETECTION

(71) Applicant: MONTE ROSA THERAPEUTICS, INC., Boston, MA (US)

(72) Inventors: Amine SADOK, Boston, MA (US); Debora BONENFANT, Sierentz (FR)

(21) Appl. No.: 18/565,163

(22) PCT Filed: Jun. 3, 2022
(86) PCT No.: PCT/US22/32117

§ 371 Date: Nov. 29, 2023

Related U.S. Application Data
(60) Provisional application No. 63/197,195, filed on Jun. 4, 2021.

(51) Int. Cl. C12Q 1/48; C07K 14/705; G01N 33/68

(52) U.S. Cl. CPC C12Q 1/48; C07K 14/705; G01N 33/6848

(57) ABSTRACT

Described herein are fusion protein(s), e.g., fusion protein(s) comprising: i) a cereblon protein, e.g., a human cereblon protein, and ii) a Proximity Labeling Enzyme. Also described are polynucleotide sequence(s) encoding the fusion protein(s), vector(s) comprising the polynucleotide sequence(s), and cells transformed with the vector(s). Also described herein are methods of using the fusion protein(s), polynucleotide sequence(s), vector(s), and cell(s).

### CLAIM OF PRIORITY

This application claims the benefit of U.S. Provisional Application Ser. No. 63/197,195, filed on 4 Jun. 2021. The entire contents of the foregoing are incorporated herein by reference.

### TECHNICAL FIELD

Described herein are fusion protein(s), e.g., fusion protein(s) comprising: i) an E3 ligase substrate receptor, e.g., a cereblon protein, e.g., a human cereblon protein, and ii) a Proximity Labeling Enzyme, e.g., a promiscuous biotinylation enzyme. Also described are polynucleotide sequence(s) encoding the fusion protein(s), vector(s) comprising the polynucleotide sequence(s), and cells transformed with the vector(s). Also described herein are methods of using the fusion protein(s), polynucleotide sequence(s), vector(s), and cell(s).

### BACKGROUND

The ubiquitin proteasome system can be manipulated, e.g., with various small molecules to trigger interaction, e.g., targeted degradation of specific proteins of interest. Promoting the targeted degradation of, e.g., pathogenic proteins using small molecule degraders is emerging as a new modality in the treatment of diseases. Therefore, there is a need for methods for identifying proximity dependent interaction of E3 ligases and target proteins.

### SUMMARY

Provided herein are fusion protein(s), e.g., fusion protein(s) comprising: i) an E3 ligase substrate receptor, e.g., a cereblon protein, e.g., a human cereblon protein, and ii) a Proximity Labeling Enzyme, e.g., a promiscuous biotinylation enzyme. Also described are polynucleotide sequence(s) encoding the fusion protein(s), vector(s) comprising the polynucleotide sequence(s), and cells transformed with the vector(s). Also described herein are methods of using the fusion protein(s), polynucleotide sequence(s), vector(s), and cell(s).

Provided herein are systems for detecting modulator-dependent proximity-based interactions between an E3 ligase and a target protein comprising: a) cell(s) expressing one or more fusion proteins, each fusion protein comprising an E3 ligase substrate receptor and a proximity labeling enzyme; and b) an E3 ligase binding modulator.

In some embodiments, the system further comprises c) second cell(s) expressing one or more fusion protein, each fusion protein comprising a mutant of the E3 ligase substrate receptor that is unable to bind the modulator at a canonical binding site.

Also provided herein are methods for detecting the interaction of an E3 ligase and a target comprising: a) providing (i) cell(s) expressing a fusion protein comprising an E3 ligase substrate receptor and a proximity labeling enzyme; and (ii) optionally, an E3 ligase binding modulator; b) incubating the cell(s) and, optionally, the modulator under conditions effective for the proximity labeling enzyme to label protein(s) in the proximity of the fusion protein; and c) determining the presence and/or amount of labeled protein(s), thereby detecting the interaction of an E3 ligase and a target.

Also provided herein are methods for detecting modulator-dependent interaction(s) between an E3 ligase and one or more target(s) comprising: I) a) providing i) first cell(s) expressing a fusion protein comprising an E3 ligase substrate receptor and a proximity labeling enzyme; and ii) an E3 ligase binding modulator; b) incubating the first cell(s) and modulator under conditions effective for the proximity labeling enzyme to label protein(s) in the proximity of the fusion protein; and c) detecting the presence and/or amount of labeled protein(s); II) a) providing i) second cell(s) expressing the fusion protein; and ii) a negative control for the modulator; b) incubating the second cell(s) and negative control under conditions effective for the proximity labeling enzyme to label protein(s) in the proximity of the fusion protein; and c) detecting the presence and/or amount of labeled protein(s); III) comparing the presence and/or amount of the protein(s) detected in step I to the presence and/or amount of those detected in step II; and IV) determining, based on the comparing in step III, whether the protein(s) are target(s) that interact with the E3 ligase in a modulator-dependent manner.

Also provided herein are methods for detecting modulator-dependent interaction(s) between an E3 ligase and one or more target(s) comprising: I) a) providing i) first cell(s) expressing i) a fusion protein comprising an E3 ligase substrate receptor and a proximity labeling enzyme; and ii) an E3 ligase modulator; b) incubating the first cell(s) and modulator under conditions effective for the proximity labeling enzyme to label protein(s) in the proximity of the fusion protein; and c) detecting the presence and/or amount of labeled protein(s); II) a) providing second cell(s) expressing i) a fusion comprising a proximity labeling enzyme and an E3 ligase substrate receptor that is unable to bind the modulator at a canonical binding site; and ii) a modulator; b) incubating the second cell(s) and modulator under conditions effective for the proximity labeling enzyme to label protein(s) in the proximity of the fusion protein; and c) detecting the presence and/or amount of labeled protein(s); III) comparing the presence and/or amount of the protein(s) detected in step I to the presence and/or amount of those detected in step II; and IV) determining, based on the comparing in step III, whether the protein(s) are target(s) that interact with the E3 ligase in a modulator-dependent manner.

Also provided herein are methods for detecting modulator-dependent interaction between an E3 ligase and one or more target(s) comprising: I) a) providing first cell(s) expressing i) a first fusion protein comprising an E3 ligase substrate receptor and a proximity labeling enzyme; and ii) an E3 ligase binding modulator; b) incubating the first cell(s) and modulator under conditions effective for the proximity labeling enzyme to label protein(s) in the proximity of the fusion protein; and c) detecting the presence and/or amount of labeled protein(s); II) a) providing second cell(s) expressing i) the first fusion protein; and ii) a negative control for the modulator; b) incubating the second cell(s) and negative control under conditions effective for the proximity labeling enzyme to label protein(s) in the proximity of the fusion protein; and c) detecting the presence and/or amount of labeled protein(s); III) a) providing third cell(s) expressing i) a second fusion protein comprising a proximity labeling enzyme and an E3 ligase substrate receptor that is unable to bind the modulator at a canonical binding site; and ii) the modulator; b) incubating the third cell(s) and modulator under conditions effective for the proximity labeling enzyme to label protein(s) in the proximity of the fusion protein; c) detecting the presence and or amount of labeled protein(s); IV) comparing the presence and/or amount of the protein(s) detected in step I to the presence and/or amount of those detected in step II and/or step III; and V) determining, based on the comparing in step IV, whether the protein(s) are target(s) that interact with the E3 ligase in a modulator-dependent manner.

Also provided herein are methods for validating a predicted modulator-dependent interaction between an E3 ligase and target(s) comprising: I) a) providing i) first cell(s) expressing the target(s) and a fusion protein comprising an E3 ligase substrate receptor and a proximity labeling enzyme; and ii) an E3 ligase binding modulator; b) incubating the first cell(s) and modulator under conditions effective for the proximity labeling enzyme to label protein(s) in the proximity of the fusion protein; and c) detecting the presence and/or amount of labeled protein(s); II) a) providing i) second cell(s) expressing the target(s) and the fusion protein; and ii) a negative control for the modulator; b) incubating the second cell(s) and negative control under conditions effective for the proximity labeling enzyme to label protein(s) in the proximity of the fusion protein; and c) detecting the presence and/or amount of labeled protein(s); III) comparing the presence and/or amount of labeled target(s), from step I with those from step II; and IV) validating the predicted modulator-dependent interaction between the E3 ligase and target(s) or not based on the comparing of step III.

Also provided herein are methods for validating a predicted modulator-dependent interaction between an E3 ligase and target(s) comprising: I) a) providing i) first cell(s) expressing the target(s) and a fusion protein comprising an E3 ligase substrate receptor and a proximity labeling enzyme; and ii) an E3 ligase binding modulator; b) incubating the first cell(s) and modulator under conditions effective for the proximity labeling enzyme to label protein(s) in the proximity of the fusion protein; and c) detecting the presence and/or amount of labeled protein(s); II) a) providing i) second cell(s) expressing the target(s) and a fusion protein comprising a proximity labeling enzyme and an E3 ligase substrate receptor that is unable to bind the modulator at a canonical binding site; and ii) the modulator; b) incubating the second cell(s) and modulator under conditions effective for the proximity labeling enzyme to label protein(s) when in the proximity of the fusion protein; and c) detecting the presence and/or amount of labeled protein(s); III) comparing the presence and/or amount of labeled target(s). from step I with those from step II; and IV) validating the predicted modulator-dependent interaction between the E3 ligase and target(s) or not based on the comparing of step III.

Also provided herein are methods for validating a predicted modulator-dependent interaction between an E3 ligase and target(s) comprising: I) a) providing i) first cell(s) expressing the target(s) and a fusion protein comprising an E3 ligase substrate receptor and a proximity labeling enzyme; and ii) an E3 ligase binding modulator; b) incubating the first cell(s) and modulator under conditions effective for the proximity labeling enzyme to label protein(s) in the proximity of the fusion protein; and c) detecting the presence and/or amount of labeled target(s); II) a) providing i) second cell(s) expressing the target(s) and the fusion protein; and ii) a negative control for the modulator; b) incubating the second cell(s) and negative control under conditions effective for the proximity labeling enzyme to label protein(s) in the proximity of the fusion protein; and c) detecting the presence and/or amount of labeled target(s); III) a) providing i) third cell(s) expressing the target(s) and a fusion protein comprising a proximity labeling enzyme and an E3 ligase substrate receptor that is unable to bind the modulator at a canonical binding site; and ii) a modulator; b) incubating the third cell(s) and modulator under conditions effective for the proximity labeling enzyme to label protein(s) in the proximity of the fusion protein; and c) detecting the presence and/or amount of labeled target(s); IV) comparing the presence and/or amount of labeled target(s) from step I to those from step II and/or III; and V) validating a predicted modulator-dependent interaction between an E3 ligase and target(s) or not based on the comparing of step IV.

Also provided herein are methods for identification of E3 ligase(s) that interact with target(s) in a modulator-dependent manner or not comprising: I) a) providing i) first cell(s) expressing the target(s) and one or more fusion protein(s) each comprising an E3 ligase substrate receptor and a proximity labeling enzyme; and ii) an E3 ligase binding modulator; b) incubating the cell(s) and modulator under conditions effective for the proximity labeling enzyme to label protein(s) in the proximity of the fusion protein(s); and c) detecting the presence and/or amount of labeled protein(s); II) a) providing i) second cell(s) expressing the fusion protein(s); and ii) a negative control for the modulator; b) incubating the second cell(s) and negative control under conditions effective for the proximity labeling enzyme to label protein(s) in the proximity of the fusion protein(s); and c) detecting the presence and/or amount of labeled protein(s); III) comparing the presence and/or amount of labeled target(s), from step I to those in step II; and IV) identifying E3 ligase(s) that interact with target(s) in a modulator-dependent manner not based on the comparing of step III.

Also provided herein are methods for identification of E3 ligase(s) that interact with target(s) in a modulator-dependent manner or not comprising: I) a) providing i) first cell(s) expressing the target(s) and one or more fusion proteins each comprising an E3 ligase substrate receptor and a proximity labeling enzyme; and ii) an E3 ligase binding modulator; b) incubating the first cell(s) and modulator under conditions effective for the proximity labeling enzyme to label protein(s) in the proximity of the fusion protein; and c) detecting labeled protein(s); II) a) providing i) second cell(s) expressing the target(s) and one or more a fusion protein(s) corresponding to the fusion proteins of (I)(a), each comprising a proximity labeling enzyme and a mutant(s) of the E3 ligases substrate receptor(s) of the fusion proteins of (I)(a) that is unable to bind the modulator at a canonical binding site; and ii) the modulator; b) incubating the second cell(s) and modulator under conditions effective for the proximity labeling enzyme to label protein(s) in the proximity of the fusion protein; and c) detecting labeled protein(s); III) comparing the presence and/or amount of labeled target(s) from step I to those from step II; and IV) identifying, based on the comparing in step III, E3 ligase(s) that interact with target(s) in a modulator-dependent manner or not.

Also described herein are methods for identification of E3 ligase(s) that interact with target(s) in a modulator-dependent manner or not comprising: I) a) providing i) first cell(s) expressing the target(s) and a fusion protein comprising an E3 ligase substrate receptor and a proximity labeling enzyme; and ii) an E3 ligase binding modulator; b) incubating the first cell(s) and modulator under conditions effective for the proximity labeling enzyme to label protein(s) in the proximity of the fusion protein; and c) detecting the presence and/or amount of labeled protein(s); II) a) providing i) second cell(s) expressing the target(s) and the fusion protein; and ii) a negative control for the modulator; b) incubating the second cell(s) and negative control under conditions effective for the proximity labeling enzyme to label protein(s) in the proximity of the fusion protein; and c) detecting the presence and/or amount of labeled protein(s); III) a) providing i) third cell(s) expressing the target(s) and a fusion protein comprising a proximity labeling enzyme and an E3 ligases substrate receptor that is unable to bind the modulator at a canonical binding site; and ii) an E3 ligase binding modulator; b) incubating the third cell(s) and modulator under conditions effective for the proximity labeling enzyme to label protein(s) in the proximity of the fusion protein; and c) detecting the presence and/or amount of labeled protein(s); IV) comparing the presence and/or amount of labeled target(s) from step I to those from step II and/or III; and V) determining, based on the comparing in step IV, whether the E3 ligase(s) interact with the target(s) in a modulator-dependent manner or not.

Also described herein are methods for identifying non-canonical E3 ligase substrate receptor binding sites comprising: I) a) providing i) first cell(s) expressing the target(s) and a fusion protein comprising an E3 ligase substrate receptor and a proximity labeling enzyme; and ii) an E3 ligase binding modulator, wherein the E3 ligase substrate receptor is unable to bind the modulator at a canonical binding site; b) incubating the first cell(s) and modulator under conditions effective for the proximity labeling enzyme to label protein(s) in the proximity of the fusion protein(s); and c) detecting the presence and/or amount of labeled protein(s); II) a) providing i) second cell(s) expressing the target(s) and a fusion protein comprising a proximity labeling enzyme and an E3 ligase substrate receptor that is unable to bind the modulator at a canonical binding site; and ii) a negative control for the modulator; b) incubating the second cell(s) and negative control under conditions effective for the proximity labeling enzyme to label protein(s) in the proximity of the fusion protein(s); and c) detecting the presence and/or amount of labeled protein(s); III) comparing the presence and/or amount of labeled target(s), from step I to those in step II; and IV) identifying non-canonical E3 binding sites that interact with a modulator and/or target based on the comparing of step III.

In some embodiments of the methods described herein, the negative control for the modulator is DMSO.

In some embodiments, of the methods described herein, conditions effective for the proximity labeling enzyme to label protein(s) in the proximity of the fusion protein comprise incubating in a composition comprising a substrate for the proximity labeling enzyme. In some embodiments, the substrate for the proximity labeling enzyme is biotin.

In some embodiments, of the methods described herein, incubation is carried out in the presence of a 26S proteasome inhibitor. In some embodiments, the 26S proteasome inhibitor is selected from the group consisting of bortezomib, ixazomib, carfilzomib, MG-132, MG-115, oprozomib, marizomib, MLN9708, and combinations thereof.

In some embodiments, of the methods described herein, detecting the presence and/or amount of labeled protein(s) comprises quantitative mass spectrometry and/or Western Blot analysis.

In some embodiments, of the methods described herein, the target is identified as having a modulator-dependent interaction with an E3 ligase, or vice-versa, when the amount of the target protein that is labeled after incubation with the modulator is greater than the amount of the target protein that is labeled after incubation under the same conditions with a negative control for the modulator.

In some embodiments, of the methods described herein, the target is identified as having a modulator-dependent interaction with an E3 ligase when the amount of the target protein that is labeled after incubation with a modulator is greater than the amount of the target protein that is labeled after incubation under the same conditions except where the E3 ligase is a mutant that is unable to bind the modulator at a canonical binding site. In some embodiments, the log2 fold change of the target protein when incubated with the modulator versus the control or mutant is at least 0.5, at least 1, at least 1.5, at least 2, or at least 3.

In some embodiments, of the systems or methods described herein, the E3 ligase substrate receptor is selected from the group consisting of CRBN (SEQ ID NO: 4), VHL (SEQ ID NO: 31), BIRC1 (SEQ ID NO: 32), BIRC2 (SEQ ID NO: 33), BIRC3 (SEQ ID NO: 34), BIRC4 (SEQ ID NO: 35), BIRC5 (SEQ ID NO: 36), BIRC6 (SEQ ID NO: 37), BIRC7 (SEQ ID NO: 38), BIRC8 (SEQ ID NO: 39), KEAP1 (SEQ ID NO: 40), DCAF15 (SEQ ID NO: 41), RNF4 (SEQ ID NO: 42) RNF4 isoform 2 (SEQ ID NO: 43), RNF114 (SEQ ID NO: 44), RNF114 isoform 2 (SEQ ID NO: 45), DCAF16 (SEQ ID NO: 46) AHR (SEQ ID NO: 47), MDM2 (SEQ ID NO: 48), UBR2 (SEQ ID NO: 49), SPOP (SEQ ID NO: 50), KLHL3 (SEQ ID NO: 51), KLHL12 (SEQ ID NO: 52), KLHL20 (SEQ ID NO: 53), KLHDC2 (SEQ ID NO: 54), SPSB1 (SEQ ID NO: 55), SPSB2 (SEQ ID NO: 56), SBSB4 (SEQ ID NO: 57), SOCS2 (SEQ ID NO: 58), SOCS6 (SEQ ID NO: 59), FBXO4 (SEQ ID NO: 60), FBXO31 (SEQ ID NO: 61), BTRC (SEQ ID NO: 62), FBW7 (SEQ ID NO: 63), CDC20 (SEQ ID NO: 64), ITCH (SEQ ID NO: 65), PML (SEQ ID NO: 66), TRIM21 (SEQ ID NO: 67), TRIM24 (SEQ ID NO: 68), TRIM33 (SEQ ID NO: 69), GID4 (SEQ ID NO: 70), DCAF11 (SEQ ID NO: 71), and an enzymatically active portion or variant of any one of the foregoing E3 ligase substrate receptors.

In some embodiments, the E3 ligase has an amino acid sequence of at least 95% identity to CRBN (SEQ ID NO: 4).

In some embodiments, the E3 ligase that does not bind the modulator at a canonical binding site has an amino acid sequence of at least 95% identity to CRBN (SEQ ID NO: 4). In some embodiments, the E3 ligase comprises mutations Y384A and W386A.

In some embodiments, the proximity labeling enzyme is a promiscuous biotinylation enzyme. In some embodiments, the promiscuous biotinylation enzyme is selected from the group consisting of SEQ ID NO: 15, SEQ ID NO: 17, SEQ ID NO: 18, SEQ ID NO: 20, SEQ ID NO: 22, SEQ ID NO: 23, SEQ ID NO: 25, SEQ ID NO: 27, SEQ ID NO: 29, SEQ ID NO: 23 with the mutation corresponding to R118S of SEQ ID NO: 14, SEQ ID NO: 23 with the mutation corresponding to R118G of SEQ ID NO: 14, SEQ ID NO: 25 with the mutation corresponding to R118S of SEQ ID NO: 14, SEQ ID NO: 25 with the mutation corresponding to R118G of SEQ ID NO: 14, SEQ ID NO: 27 with the mutation corresponding to R118S of SEQ ID NO: 14, SEQ ID NO: 27 with the mutation corresponding to R118G of SEQ ID NO: 14, SEQ ID NO: 29 with the mutation corresponding to R118S of SEQ ID NO: 14, and SEQ ID NO: 29 with the mutation corresponding to R118G of SEQ ID NO: 14.

In some embodiments, one or more of the fusion protein(s) further comprises a linker between the E3 ligase and the proximity labeling enzyme. In some embodiments, the linker(s) are each independently 1, 2, 3, 4, 5, 6, 7, 8, 9, 10, 11, 12, 13, 14, 15, 16, 17, 18, 19, or 20 amino acids long.

In some embodiments, the fusion protein comprises SEQ ID NO: 1.

In some embodiments, one or more of the fusion protein(s) further comprises a self-cleaving peptide, optionally T2A and/or a detection label, optionally a fluorescent protein, optionally green fluorescent protein (GFP), optionally eGFP.

In some embodiments, a fusion protein comprises SEQ ID NO: 2.

In some embodiments, the E3 ligase binding modulator is a compound selected from those in Table 4 and Table 5.

In some embodiments, the cell is selected from the group consisting of HEK293T cells, CAL51 cells, HCT116 cells, MCF7 cells, SKMEL28 cells, THP1 cells, U937 cells, and combinations thereof.

Exemplary modulator compounds, cells, and target compounds suitable for the systems and methods are set forth herein.

Also provided herein are cell(s), fusion protein(s), and vector(s) of the systems or methods described herein as well as cell(s) comprising the vector(s). Also provided herein are protein complex(es) comprising a fusion protein described herein and a target protein as well as cell(s) comprising the protein complex(es).

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a workflow showing a chemocentric approach to identify novel neosubstrates.

FIGS. 2A and 2B show an overview of an exemplary method for target detection and/or identification with an E3 ligase/Proximity Labeling Enzyme fusion protein in the presence of an E3 ligase binding modulator.

FIG. 3 shows the map of an exemplary TurboID-CRBN fusion protein containing expression vector.

FIG. 4 shows quantitative mass spectrometry detection of modulator-dependent cereblon-target interactions. The targets in the upper right quadrant are predicted to interact with cereblon in a modulator-dependent manner. Certain of them are labelled.

### DETAILED DESCRIPTION

Described herein are fusion protein(s), e.g., fusion protein(s) comprising: i) an E3 ligase substrate receptor, e.g., a cereblon protein, e.g., a human cereblon protein, and ii) a Proximity Labeling Enzyme, e.g., a promiscuous biotinylation enzyme. Also described are polynucleotide sequence(s) encoding the fusion protein(s), vector(s) comprising the polynucleotide sequence(s), cell(s) transformed with the vector(s), and cell(s) expressing the fusion protein(s). Also described herein are methods of using the fusion protein(s), polynucleotide sequence(s), vector(s), and cell(s).

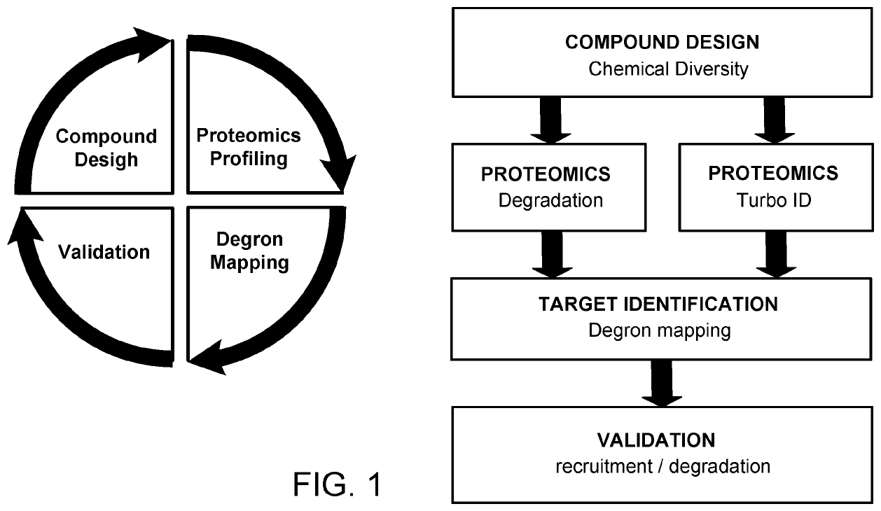

In some cases, he methods of using the fusion protein(s) is integrated into a chemocentric approach to identify neosubstrates, e.g., as shown in FIG. 1.

### Fusion Proteins

The fusion proteins described herein comprise an E3 ligase substrate receptor described herein, e.g., a cereblon protein, e.g., a human cereblon protein, a variant thereof, or an enzymatically active portion thereof, genetically fused to a Proximity Labeling Enzyme described herein, e.g., a Proximity Labeling Enzyme.

As used herein, an “enzymatically active portion” of an E3 ligase is one that retains the ability to ubiquitinate protein(s), e.g., to form an E3 ubiquitin ligase complex able to ubiquitinate protein(s).

In some embodiments, the fusion protein comprises, from C-terminal to N-terminal: (a) a Proximity Labeling Enzyme, e.g., a Proximity Labeling Enzyme described herein; and (b) an E3 ligase substrate receptor, e.g., an E3 ligase substrate receptor described herein. In some embodiments, the E3 ligase substrate receptor does not comprise a leading methionine (M).

In some embodiments, the fusion protein comprises, from C-terminal to N-terminal: (a) an E3 ligase substrate receptor, e.g., an E3 ligase substrate receptor described herein; and (b) a Proximity Labeling Enzyme, e.g., a Proximity Labeling Enzyme described herein. In some embodiments, the Proximity Labeling Enzyme does not comprise a leading methionine (M).

In some embodiments, the fusion protein comprises a linker between the E3 ligase substrate receptor and the Proximity Labeling Enzyme. In some embodiments, the linker is from 1 to 20 amino acids long, e.g., in some embodiments the linker is 1, 2, 3, 4, 5, 6, 7, 8, 9, 10, 11, 12, 13, 14, 15, 16, 17, 18, 19, or 20 amino acids long. In some embodiments, the linker is GSG.

In some embodiments, the fusion protein comprises or consists of SEQ ID NO: 1 or SEQ ID NO: 2. In some embodiments, the fusion protein comprises or consists of an amino acid sequence at least 80%, e.g., at least 90%, at least 95%, or at least 99% identical to SEQ ID NO: 1 or SEQ ID NO: 2.

### E3 Ligases and E3 Ligase Substrate Receptors

The fusion proteins described herein comprise an E3 ligase substrate receptor. E3 ligases are known and described in the art. See, e.g., Ishida et al., “E3 Ligase Ligands for PROTACs: How They Were Found and How to Discover New Ones,” SLAS Discovery 26(4):484-502 (2021).

In some embodiments, the E3 ligase substrate receptor is an E3 ligase substrate receptor selected from the group consisting of CRBN (SEQ ID NO: 4), VHL (SEQ ID NO: 31), BIRC1 (SEQ ID NO: 32), BIRC2 (SEQ ID NO: 33), BIRC3 (SEQ ID NO: 34), BIRC4 (SEQ ID NO: 35), BIRC5 (SEQ ID NO: 36), BIRC6 (SEQ ID NO: 37), BIRC7 (SEQ ID NO: 38), BIRC8 (SEQ ID NO: 39), KEAP1 (SEQ ID NO: 40), DCAF15 (SEQ ID NO: 41), RNF4 (SEQ ID NO: 42) RNF4 isoform 2 (SEQ ID NO: 43), RNF114 (SEQ ID NO: 44), RNF114 isoform 2 (SEQ ID NO: 45), DCAF16 (SEQ ID NO: 46) AHR (SEQ ID NO: 47), MDM2 (SEQ ID NO: 48), UBR2 (SEQ ID NO: 49), SPOP (SEQ ID NO: 50), KLHL3 (SEQ ID NO: 51), KLHL12 (SEQ ID NO: 52), KLHL20 (SEQ ID NO: 53), KLHDC2 (SEQ ID NO: 54), SPSB1 (SEQ ID NO: 55), SPSB2 (SEQ ID NO: 56), SBSB4 (SEQ ID NO: 57), SOCS2 (SEQ ID NO: 58), SOCS6 (SEQ ID NO: 59), FBXO4 (SEQ ID NO: 60), FBXO31 (SEQ ID NO: 61), BTRC (SEQ ID NO: 62), FBW7 (SEQ ID NO: 63), CDC20 (SEQ ID NO: 64), ITCH (SEQ ID NO: 65), PML (SEQ ID NO: 66), TRIM21 (SEQ ID NO: 67), TRIM24 (SEQ ID NO: 68), TRIM33 (SEQ ID NO: 69), GID4 (SEQ ID NO: 70), and DCAF11 (SEQ ID NO: 71).

In some embodiments, the E3 ligase substrate receptor is at least 80%, e.g., at least 90%, at least 95%, or at least 99% identical to an E3 ligase selected from the group consisting of CRBN (SEQ ID NO: 4), VHL (SEQ ID NO: 31), BIRC1 (SEQ ID NO: 32), BIRC2 (SEQ ID NO: 33), BIRC3 (SEQ ID NO: 34), BIRC4 (SEQ ID NO: 35), BIRC5 (SEQ ID NO: 36), BIRC6 (SEQ ID NO: 37), BIRC7 (SEQ ID NO: 38), BIRC8 (SEQ ID NO: 39), KEAP1 (SEQ ID NO: 40), DCAF15 (SEQ ID NO: 41), RNF4 (SEQ ID NO: 42) RNF4 isoform 2 (SEQ ID NO: 43), RNF114 (SEQ ID NO: 44), RNF114 isoform 2 (SEQ ID NO: 45), DCAF16 (SEQ ID NO: 46) AHR (SEQ ID NO: 47), MDM2 (SEQ ID NO: 48), UBR2 (SEQ ID NO: 49), SPOP (SEQ ID NO: 50), KLHL3 (SEQ ID NO: 51), KLHL12 (SEQ ID NO: 52), KLHL20 (SEQ ID NO: 53), KLHDC2 (SEQ ID NO: 54), SPSB1 (SEQ ID NO: 55), SPSB2 (SEQ ID NO: 56), SBSB4 (SEQ ID NO: 57), SOCS2 (SEQ ID NO: 58), SOCS6 (SEQ ID NO: 59), FBXO4 (SEQ ID NO: 60), FBXO31 (SEQ ID NO: 61), BTRC (SEQ ID NO: 62), FBW7 (SEQ ID NO: 63), CDC20 (SEQ ID NO: 64), ITCH (SEQ ID NO: 65), PML (SEQ ID NO: 66), TRIM21 (SEQ ID NO: 67), TRIM24 (SEQ ID NO: 68), TRIM33 (SEQ ID NO: 69), GID4 (SEQ ID NO: 70), and DCAF11 (SEQ ID NO: 71).

In some embodiments, the E3 ligase substrate receptor is an enzymatically active portion of an E3 ligase selected from the group consisting of CRBN (SEQ ID NO: 4), VHL (SEQ ID NO: 31), BIRC1 (SEQ ID NO: 32), BIRC2 (SEQ ID NO: 33), BIRC3 (SEQ ID NO: 34), BIRC4 (SEQ ID NO: 35), BIRC5 (SEQ ID NO: 36), BIRC6 (SEQ ID NO: 37), BIRC7 (SEQ ID NO: 38), BIRC8 (SEQ ID NO: 39), KEAP1 (SEQ ID NO: 40), DCAF15 (SEQ ID NO: 41), RNF4 (SEQ ID NO: 42) RNF4 isoform 2 (SEQ ID NO: 43), RNF114 (SEQ ID NO: 44), RNF114 isoform 2 (SEQ ID NO: 45), DCAF16 (SEQ ID NO: 46) AHR (SEQ ID NO: 47), MDM2 (SEQ ID NO: 48), UBR2 (SEQ ID NO: 49), SPOP (SEQ ID NO: 50), KLHL3 (SEQ ID NO: 51), KLHL12 (SEQ ID NO: 52), KLHL20 (SEQ ID NO: 53), KLHDC2 (SEQ ID NO: 54), SPSB1 (SEQ ID NO: 55), SPSB2 (SEQ ID NO: 56), SBSB4 (SEQ ID NO: 57), SOCS2 (SEQ ID NO: 58), SOCS6 (SEQ ID NO: 59), FBXO4 (SEQ ID NO: 60), FBX031 (SEQ ID NO: 61), BTRC (SEQ ID NO: 62), FBW7 (SEQ ID NO: 63), CDC20 (SEQ ID NO: 64), ITCH (SEQ ID NO: 65), PML (SEQ ID NO: 66), TRIM21 (SEQ ID NO: 67), TRIM24 (SEQ ID NO: 68), TRIM33 (SEQ ID NO: 69), GID4 (SEQ ID NO: 70), and DCAF11 (SEQ ID NO: 71).

In some embodiments, the E3 ligase substrate receptor is a mutant that is unable to bind compounds at a canonical binding site, e.g., an E3 ligase binding modulator described herein. In some embodiments, the E3 ligase substrate receptor is a mutant of CRBN (SEQ ID NO: 4), VHL (SEQ ID NO: 31), BIRC1 (SEQ ID NO: 32), BIRC2 (SEQ ID NO: 33), BIRC3 (SEQ ID NO: 34), BIRC4 (SEQ ID NO: 35), BIRC5 (SEQ ID NO: 36), BIRC6 (SEQ ID NO: 37), BIRC7 (SEQ ID NO: 38), BIRC8 (SEQ ID NO: 39), KEAP1 (SEQ ID NO: 40), DCAF15 (SEQ ID NO: 41), RNF4 (SEQ ID NO: 42) RNF4 isoform 2 (SEQ ID NO: 43), RNF114 (SEQ ID NO: 44), RNF114 isoform 2 (SEQ ID NO: 45), DCAF16 (SEQ ID NO: 46) AHR (SEQ ID NO: 47), MDM2 (SEQ ID NO: 48), UBR2 (SEQ ID NO: 49), SPOP (SEQ ID NO: 50), KLHL3 (SEQ ID NO: 51), KLHL12 (SEQ ID NO: 52), KLHL20 (SEQ ID NO: 53), KLHDC2 (SEQ ID NO: 54), SPSB1 (SEQ ID NO: 55), SPSB2 (SEQ ID NO: 56), SBSB4 (SEQ ID NO: 57), SOCS2 (SEQ ID NO: 58), SOCS6 (SEQ ID NO: 59), FBXO4 (SEQ ID NO: 60), FBX031 (SEQ ID NO: 61), BTRC (SEQ ID NO: 62), FBW7 (SEQ ID NO: 63), CDC20 (SEQ ID NO: 64), ITCH (SEQ ID NO: 65), PML (SEQ ID NO: 66), TRIM21 (SEQ ID NO: 67), TRIM24 (SEQ ID NO: 68), TRIM33 (SEQ ID NO: 69), GID4 (SEQ ID NO: 70), and DCAF11 (SEQ ID NO: 71) that is unable to bind compounds at a canonical binding site, e.g., an E3 ligase binding modulator described herein.

### Cereblon

The cereblon protein, encoded by the gene CRBN, is the substrate recognition component of a DCX (DDB1-CUL4-X-box) E3 protein ligase complex that mediates the ubiquitination and subsequent proteasomal degradation of target proteins.

The human cereblon protein (NCBI Gene ID 51185; UniProt ID Q96SW2) encodes the transcripts and isoforms shown in Table 1, of which NM_016302.4 (SEQ ID NO: 4, transcript 1) is the canonical transcript.

TABLE 1
Cereblon Transcripts and Isoforms

| | Length | | Length | SEQ | Iso- |
|---|---|---|---|---|---|
| Transcript | (nt) | Protein | (aa) | ID NO: | form |
| XR_940448.3 | 2667 | | | | |
| XM_011533791.3 | 3586 | XP_011532093.1 | 398 | SEQ ID | X1 |
| | | | | NO: 6 | |
| XM_011533793.2 | 2927 | XP_011532095.1 | 278 | SEQ ID | X4 |
| | | | | NO: 7 | |
| XM_011533794.2 | 2798 | XP_011532096.1 | 278 | SEQ ID | X4 |
| | | | | NO: 8 | |
| NM_001173482.1 | 2593 | NP_001166953.1 | 441 | SEQ ID | 2 |
| | | | | NO: 3 | |
| XM_005265202.4 | 2472 | XP_005265259.1 | 379 | SEQ ID | X2 |
| | | | | NO: 5 | |
| NM_016302.4 | 2187 | NP_057386.2 | 442 | SEQ ID | 1 |
| | | | | NO: 4 | |
| XM_024453551.1 | 1458 | XP_024309319.1 | 284 | SEQ ID | X3 |
| | | | | NO: 9 | |

Isoform 1 of human CRBN (SEQ ID NO: 4) has the features shown in Table 2.

TABLE 2
Isoform 1 of Human CRBN Features.

| Feature | Position(s) | Reference |
|---|---|---|
| Zinc binding | 323 | Chamberlain et al. Nat. Struct. Mol. |
| Zinc binding | 326 | Biol. 21:803-9 (2014) |
| Zinc binding | 391 | |
| Zinc binding | 394 | |

Known mutants of human CRBN isoform 1 (SEQ ID NO: 4) have the features shown in Table 3.

TABLE 3
Features of Known Mutants of Human CRBN Isoform 1.

| Feature key | Position(s) | Description | Reference(s) |
|---|---|---|---|
| Mutagenesis | 384 | Y → A: Abolishes | Ito et al., |
| | | thalidomide-binding | Science 327:1345- |
| | | without affecting | 50 (2010) |
| | | DCX protein ligase | |
| | | complex activity; when | |
| | | associated with A-386. | |
| Mutagenesis | 386 | W → A: Abolishes | Ito et al., |
| | | thalidomide-binding | Science 327:1345- |
| | | without affecting | 50 (2010); |
| | | DCX protein ligase | Chamberlain et |
| | | complex activity; when | al. Nat. Struct. |
| | | associated with A-384. | Mol. Biol. |
| | | Abolishes pomalidomide- | 21:803-9 (2014) |
| | | induced change in substrate | |
| | | specificity and abolishes | |
| | | pomalidomide-induced | |
| | | decrease in cell viability | |
| | | that is brought | |
| | | about by increased | |
| | | degradation of MYC, IRF4 | |
| | | and IKZF3. | |
| Mutagenesis | 419-442 | Missing: Fails to rescue | Choi et al., |
| | | increased BK channel | J. Neurosci. |
| | | activity and decreased | 38:3571-83 (2018) |
| | | probability of | |
| | | neurotransmission in | |
| | | a mouse hippocampal | |
| | | neuron model. | |

Isoform 1 of human CRBN (SEQ ID NO: 4) comprises a Lon N-terminal domain at positions 81-317, the canonical binding domain CULT (cereblon domain of unknown activity, binding cellular Ligands and; Thalomide) at positions 318-426, and canonical thalomide binding region at positions 378-386 (Chamberlain et al. Nat. Struct. Mol. Biol. 21:803-9 (2014)). The CULT domain binds thalidomide and related drugs, such as pomalidomide and lenalidomide. Drug binding leads to a change in substrate specificity of the human DCX (DDB1-CUL4-X-box) E3 protein ligase complex, while no such change is observed in rodents (Chamberlain et al. Nat. Struct. Mol. Biol. 21:803-9 (2014)).

In some embodiments, the cereblon protein is human cereblon protein. In some embodiments, the cereblon protein comprises or consists of SEQ ID NO: 3, SEQ ID NO: 4, SEQ ID NO: 5, SEQ ID NO: 6, SEQ ID NO: 7, SEQ ID NO: 8, or SEQ ID NO: 9. In some embodiments, the cerebelon protein is at least 80% identical to SEQ ID NO: 3, SEQ ID NO: 4, SEQ ID NO: 5, SEQ ID NO: 6, SEQ ID NO: 7, SEQ ID NO: 8, or SEQ ID NO: 9, e.g., at least 90%, at least 95% or at least 99% identical to SEQ ID NO: 3, SEQ ID NO: 4, SEQ ID NO: 5, SEQ ID NO: 6, SEQ ID NO: 7, SEQ ID NO: 8, or SEQ ID NO: 9.

In some embodiments, the cereblon protein is human cereblon protein without the leading methionine (M). In some embodiments, the cereblon protein comprises or consists of SEQ ID NO: 3, SEQ ID NO: 4, SEQ ID NO: 5, SEQ ID NO: 6, SEQ ID NO: 7, SEQ ID NO: 8, or SEQ ID NO: 9 without the leading methionine (M). In some embodiments, the cerebelon protein is at least 80% identical to SEQ ID NO: 3, SEQ ID NO: 4, SEQ ID NO: 5, SEQ ID NO: 6, SEQ ID NO: 7, SEQ ID NO: 8, or SEQ ID NO: 9 without the leading methionine (M), e.g., at least 90%, at least 95% or at least 99% identical to SEQ ID NO: 3, SEQ ID NO: 4, SEQ ID NO: 5, SEQ ID NO: 6, SEQ ID NO: 7, SEQ ID NO: 8, or SEQ ID NO: 9 without the leading methionine (M).

In some embodiments, the cereblon protein is a mutant that is unable to bind compounds, e.g., an E3 ligase binding modulator, e.g., a cereblon binding modulator described herein, at a canonical binding site.

In some embodiments, the cereblon protein, e.g., a cereblon protein described herein, comprises point mutations at the positions corresponding to Y384 and/or W386 of SEQ ID NO: 4. In some embodiments, the cereblon protein, e.g., a cereblon protein described herein, comprises point mutations at the positions corresponding to Y384 and W386 of SEQ ID NO: 4. In some embodiments, the mutations are Y384A and/or W386A.

In some embodiments, the cereblon protein comprises or consists of SEQ ID NO: 4 with point mutations at Y384 and/or W386. In some embodiments, the cereblon protein comprises or consists of SEQ ID NO: 4 with point mutations at both Y384 and W386. In some embodiments, the mutations are Y384A and/or W386A.

### Proximity Labeling Enzymes

In some cases, the systems and methods described herein utilize proximity labeling enzymes. Proximity Labeling Enzyme(s) (PLEs), upon addition of a small-molecule substrate, such as biotin, initiate covalent tagging of endogenous proteins within a few nanometers of the promiscuous enzyme. PLEs are described, e.g., in Branon et al., “Efficient Proximity Labeling in Living Cells and Organisms with TurboID,” Nature Biotechnology (2018) doi: 10.1038/nbt.4201.

### Promiscuous Biotinylation Enzymes

In some cases, the proximity labeling enzyme is a promiscuous biotinylation enzyme.

Bifunctional ligase/repressor BirA, e.g., E. coli BirA acts both as a biotin--[acetyl-CoA-carboxylase] ligase and a biotin-operon repressor. In the presence of ATP, BirA activates biotin to form the BirA-biotinyl-5′-adenylate (BirA-bio-5′-AMP or holoBirA) complex. HoloBirA can either transfer the biotinyl moiety to the biotin carboxyl carrier protein (BCCP) subunit of acetyl-CoA carboxylase, or bind to the biotin operator site and inhibit transcription of the operon. The wild type E. coli BirA biotinylates only a single cellular protein. See, e.g., Choi-Rhee et al., “Promiscuous Protein Biotinylation by Escherichia coli biotin protein ligase,” Protein Science 13(11):3043-50 (2004).

Wild-type E. coli BirA has the amino acid sequence of SEQ ID NO: 14.

In some embodiments, the proximity labeling enzyme is a promiscuous biotin ligase, e.g., a mutant of E. coli BirA that attaches biotin to more proteins than does the wild-type BirA, preferably a large number of cellular proteins, preferably in vivo, e.g., as described in Branon et al., “Efficient Proximity Labeling in Living Cells and Organisms with TurboID,” Nature Biotechnology (2018) doi: 10.1038/nbt.4201.

In some embodiments, the promiscuous biotin ligase is BioID (e.g., SEQ ID NO: 14 with mutation R118G). In some embodiments, the promiscuous biotin ligase comprises or consists of SEQ ID NO: 14 with mutation R118G. In some embodiments, the promiscuous biotin ligase comprises or consists of an amino acid sequence at least 80% identical, e.g., at least 90%, at least 95%, or at least 99% identical to SEQ ID NO: 14 and has the mutation corresponding to R118G of SEQ ID NO: 14.

In some embodiments, the promiscuous biotin ligase is BioID2 (SEQ ID NO: 15). See Kim et al., “an improved smaller biotin ligase for BioID proximity labeling,” Mol. Biol. Cell 27:1188-96 (2016). In some embodiments, the promiscuous biotin ligase comprises or consists of SEQ ID NO: 15. In some embodiments, the promiscuous biotin ligase comprises or consists of an amino acid sequence at least 80%, e.g., at least 90%, at least 95%, or at least 99% identical to SEQ ID NO: 15.

In some embodiments, the promiscuous biotin ligase is BASU (SEQ ID NO: 17). See Ramanathan et al., “RNA-protein interaction detection in living cells,” Nat. Methods 15:207-12 (2018). In some embodiments, the promiscuous biotin ligase comprises or consists of SEQ ID NO: 17. In some embodiments, the promiscuous biotin ligase comprises or consists of an amino acid sequence at least 80%, e.g., at least 90%, at least 95%, or at least 99% identical to SEQ ID NO: 17.

In some embodiments, the promiscuous biotin ligase is TurboID (SEQ ID NO: 20). See Branon et al., “Efficient Proximity Labeling in Living Cells and Organisms with TurboID,” Nature Biotechnology (2018) doi: 10.1038/nbt.4201. In some embodiments, the promiscuous biotin ligase comprises or consists of SEQ ID NO: 20. In some embodiments, the promiscuous biotin ligase comprises or consists of an amino acid sequence at least 80%, e.g., at least 90%, at least 95%, or at least 99% identical to SEQ ID NO: 20.

In some embodiments, the promiscuous biotin ligase is miniTurbo (SEQ ID NO: 18). See Branon et al., “Efficient Proximity Labeling in Living Cells and Organisms with TurboID,” Nature Biotechnology (2018) doi: 10.1038/nbt.4201. In some embodiments, the promiscuous biotin ligase comprises or consists of SEQ ID NO: 18. In some embodiments, the promiscuous biotin ligase comprises or consists of an amino acid sequence at least 80%, e.g., at least 90%, at least 95%, or at least 99% identical to SEQ ID NO: 18.

In some embodiments, the promiscuous biotin ligase is AirID (SEQ ID NO: 22). See Kido et al., “AirID, a novel proximity biotinylation enzyme, for analysis of protein-protein interactions,” Elife 9:e54983 (2020). In some embodiments, the promiscuous biotin ligase comprises or consists of SEQ ID NO: 22. In some embodiments, the promiscuous biotin ligase comprises or consists of an amino acid sequence at least 80%, e.g., at least 90%, at least 95%, or at least 99% identical to SEQ ID NO: 22.

In some embodiments, the promiscuous biotin ligase is AAVA (SEQ ID NO: 23). See Kido et al., “AirID, a novel proximity biotinylation enzyme, for analysis of protein-protein interactions,” Elife 9:e54983 (2020). In some embodiments, the promiscuous biotin ligase comprises or consists of SEQ ID NO: 23. In some embodiments, the promiscuous biotin ligase comprises or consists of an amino acid sequence at least 80%, e.g., at least 90%, at least 95%, or at least 99% identical to SEQ ID NO: 23. In some embodiments, the promiscuous biotin ligase comprises or consists of SEQ ID NO: 23 with the mutation corresponding to R118G of SEQ ID NO: 14. In some embodiments, the promiscuous biotin ligase comprises or consists of SEQ ID NO: 23 with the mutation corresponding to R118S of SEQ ID NO: 14. In some embodiments, the promiscuous biotin ligase comprises or consists of an amino acid sequence at least 80%, e.g., at least 90%, at least 95%, or at least 99% identical to SEQ ID NO: 23 with the mutation corresponding to R118G of SEQ ID NO: 14. In some embodiments, the promiscuous biotin ligase comprises or consists of an amino acid sequence at least 80%, e.g., at least 90%, at least 95%, or at least 99% identical to SEQ ID NO: 23 with the mutation corresponding to R118S of SEQ ID NO: 14.

In some embodiments, the promiscuous biotin ligase is AHLA (SEQ ID NO: 25). See Kido et al., “AirID, a novel proximity biotinylation enzyme, for analysis of protein-protein interactions,” Elife 9:e54983 (2020). In some embodiments, the promiscuous biotin ligase comprises or consists of SEQ ID NO: 25. In some embodiments, the promiscuous biotin ligase comprises or consists of an amino acid sequence at least 80%, e.g., at least 90%, at least 95%, or at least 99% identical to SEQ ID NO: 25. In some embodiments, the promiscuous biotin ligase comprises or consists of SEQ ID NO: 25 with the mutation corresponding to R118G of SEQ ID NO: 14. In some embodiments, the promiscuous biotin ligase comprises or consists of SEQ ID NO: 25 with the mutation corresponding to R118S of SEQ ID NO: 14. In some embodiments. the promiscuous biotin ligase comprises or consists of an amino acid sequence at least 80%, e.g., at least 90%, at least 95%, or at least 99% identical to SEQ ID NO: 25 with the mutation corresponding to R118G of SEQ ID NO: 14. In some embodiments, the promiscuous biotin ligase comprises or consists of an amino acid sequence at least 80%, e.g., at least 90%, at least 95%, or at least 99% identical to SEQ ID NO: 25 with the mutation corresponding to R118S of SEQ ID NO: 14.

In some embodiments, the promiscuous biotin ligase is GVFA (SEQ ID NO: 27). See Kido et al., “AirID, a novel proximity biotinylation enzyme, for analysis of protein-protein interactions,” Elife 9:e54983 (2020). In some embodiments, the promiscuous biotin ligase comprises or consists of SEQ ID NO: 27. In some embodiments, the promiscuous biotin ligase comprises or consists of an amino acid sequence at least 80%, e.g., at least 90%, at least 95%, or at least 99% identical to SEQ ID NO: 27. In some embodiments, the promiscuous biotin ligase comprises or consists of SEQ ID NO: 27 with the mutation corresponding to R118G of SEQ ID NO: 14. In some embodiments, the promiscuous biotin ligase comprises or consists of SEQ ID NO: 27 with the mutation corresponding to R118S of SEQ ID NO: 14. In some embodiments, the promiscuous biotin ligase comprises or consists of an amino acid sequence at least 80%, e.g., at least 90%, at least 95%, or at least 99% identical to SEQ ID NO: 27 with the mutation corresponding to R118G of SEQ ID NO: 14. In some embodiments, the promiscuous biotin ligase comprises or consists of an amino acid sequence at least 80%, e.g., at least 90%, at least 95%, or at least 99% identical to SEQ ID NO: 27 with the mutation corresponding to R118S of SEQ ID NO: 14.

In some embodiments, the promiscuous biotin ligase is All (SEQ ID NO: 29). See Kido et al., “AirID, a novel proximity biotinylation enzyme, for analysis of protein-protein interactions,” Elife 9:e54983 (2020). In some embodiments, the promiscuous biotin ligase comprises or consists of SEQ ID NO: 29. In some embodiments, the promiscuous biotin ligase comprises or consists of an amino acid sequence at least 80%, e.g., at least 90%, at least 95%, or at least 99% identical to SEQ ID NO: 29. In some embodiments, the promiscuous biotin ligase comprises or consists of SEQ ID NO: 29 with the mutation corresponding to R118G of SEQ ID NO: 14. In some embodiments, the promiscuous biotin ligase comprises or consists of SEQ ID NO: 29 with the mutation corresponding to R118S of SEQ ID NO: 14. In some embodiments, the promiscuous biotin ligase comprises or consists of an amino acid sequence at least 80%, e.g., at least 90%, at least 95%, or at least 99% identical to SEQ ID NO: 29 with the mutation corresponding to R118G of SEQ ID NO: 14. In some embodiments, the promiscuous biotin ligase comprises or consists of an amino acid sequence at least 80%, e.g., at least 90%, at least 95%, or at least 99% identical to SEQ ID NO: 29 with the mutation corresponding to R118S of SEQ ID NO: 14.

### Expression Systems

To use the fusion proteins described herein, it may be desirable to express them from a nucleic acid that encodes them. This can be performed in a variety of ways. For example, the nucleic acid encoding the fusion protein can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the fusion protein. The nucleic acid encoding the fusion protein can also be cloned into an expression vector, for administration to a plant cell, fungal cell, bacterial cell, protozoan cell, or animal cell, preferably a mammalian cell or a human cell.

Thus, described herein are nucleic acid(s) encoding the fusion protein(s) described herein, vectors comprising the nucleic acid(s), and cells comprising the vector(s).

In some embodiments, the vector is a lentivirus vector. See, e.g., Milone et al., “Clinical Use of Lentiviral Vectors,” Leukemia 32:1529-41 (2018). In some embodiments, the vector is a retrovirus vector. In some embodiments, the vector is a gamma retroviral vector. In some embodiments, the vector is a non-viral vector, e.g., a piggyback non-viral vector (PB transposon, see, e.g., Wu et al., “piggy back is a Flexible and Highly Active Transposon as Compared to Sleeping Beauty, Tol2, and Mos1 in Mammalian Cells,” PNAS 103(41): 15008-13 (2006)), a sleeping beauty non-viral vector (SB transposon, see, e.g., Hudecek et al., “Going Non-Viral: the Sleeping Beauty Transposon System Breaks on Through to the Clinical Side,” Critical Reviews in Biochemistry and Molecular Biology 52(4):355-380 (2017)), or an mRNA vector.

To obtain expression, a sequence encoding a fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

In some embodiments, the promoter is a constitutive promoter. In some embodiments. the constitutive promoter is selected from the group consisting of SV40, CMV, UBC, EFIA, PGK, and CAGG.

In some embodiments, the promoter is an inducible promoter. See, e.g., Kallunki et al., “How to Choose the Right Inducible Gene Expression System for Mammalian Studies?” Cells 8:796 (2019).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the fusion protein and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the fusion protein, e.g., expression in plants, animals, bacteria, fungus, protozoa, etc.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the split fusion protein.

In some embodiments, the cell(s) are stably transfected. In some embodiments, the cell(s) are transiently transfected.

In some embodiments, the cell(s) are selected from the group consisting of HEK293T cells, CAL51 cells, HCT116 cells, MCF7 cells, SKMEL28 cells, THPI cells, U937 cells, and combinations thereof.

In some cases, the cell(s) are adherent. In some cases, the cell(s) are non-adherent. In some embodiments, the cell(s) are cancer cells. In some embodiments, the cell(s) are selected from the group consisting of NIHOVCAR3, HL60, CACO2, HEL, HEL9217, MONOMAC6, LS513, A101D, C2BBE1, NCIH2077, 253J, HCC827, ONCODG1, HS294T, NCIH1581, SLR21, SKBR3, T24, MCF7, MHHCALL2, NCIH1693, PATU8988S, PATU8988T, OPM2, CH157MN, 253JBV, GOS3, KPL1, HCC827GR5, PC14, PANC0213, MHHCALL3, NCIH1819, PLB985, NCIH1650, U343, S117, EHEB, SKNMC, U118MG, RDES, PANC0203, HS895T, MDAMB134VI, MV411, ACHN, GCIY, TOV112D, HEKTE, NCIH929, TE617T, A673. KARPAS299, HT1080, D283MED, DOHH2, OPM1, ML1, SUPB15, PANC1005, HH, RERFLCMS, HS616T, SALE, OCIAML5, HCC4006, HS683, REC1, HS611T, 697, HS706T, MEG01, GRANTA519, KU812, U87MG, NCO2, MJ, MHHNB11, TE125T, BDCM, GDMI, G292CLONEA141B1, HS281T, MUTZ3, T3M4, ACCMESO1, SKES1, HS172T, NCIH684, PC3, OV56, NCIH2452, PANC0504, HPAFII, D341, G401, ZR751, GAMG, SIMA, RH41, KE37, GMS10, CAOV4, LOUCY, ALLSIL, JVM2, CAPAN2, KP3, NCIH3255, NCCSTCK140, HCC1187, SIGM5, OCIAML2, SU8686, VCAP, OAW28, EFM192A, HUPT3, HS863T, CHP212, NCIH2405, SUPT11, COV434, OCILY19, TO175T, KG1C, SLR20, LN319, NCIH1341, NALM19, HS229T, JHOS2, HS729, HS274T, HS940T, CHP126, 8MGBA, CFPAC1, PANC0327, PFEIFFER, SNU308, CAL29, HCC2429, RERFGC1B, SKLMS1, THP1, T47D, HS578T, SKNSH, HCC2935, JM1, M059K, NCIH2052, HS888T, SW1990, MHHCALL4, A4FUK, OCILY3, OSRC2, BT12, CORL105, GA10, SW579, PANC1, HS751T, KASUMI6, KE97, NOMO1, RD, PRECLH, VMRCRCZ, TM87, JHUEM3, CAL62, TE159T, LOUNH91, NCIH660, HS766T, NCIH1618, HS839T, SCC9, SNU869, L363, HS343T, HS737T, NCIH2444, CORL311, SCC25, RCC10RGB, HDMYZ, BHT101, MFE280, KARPAS620, HS934T, SET2, HCC1599, TALL1, EOL1, HS255T, NMCG1, A204, COLO320, NH6, LP1, PK59, C8166, DETROIT562, U178, SNU1079, CADOES1, DAOY, CAL120, HUPT4, HS675T, LN382, JHESOAD1, JHH6, PL21, A375, MINO, SNU398, ASPC1, HCC1937, HS819T, ECC12, SUPM2, KPNYN, BICR31, HS822T, HS742T, KALS1, U251MG, DEL, CAKI2, PANC0403, SW1417, JHOM1, SCC4, HUG1N, HS600T, JK1, RT4, DANG, DKMG, BL41, SLR23, OCUM1, AU565, CL11, KMRC20, NCIH2887, LS1034, COLO201, SCC15, LMSU, COV318, CORL279, DU4475, KELLY, SKNAS, RERFLCAI, UOK101, KASUMI1, CALU6, KP4, SNU213, HDLM2, SNU245, AM38, HPAC, SUDHL10, SLR24, SF539, HS852T, HS834T, HCC38, HCC1419, COV362, EWS502, SNU840, KP2, NCIH1755, A1207, HS840T, TOLEDO, SNU1033, NUDUL1, BT549, SNU466, NCIH209, OV90, NCIH841, KLE, NB4, EM2, OUMS23, NCIH889, NCIH2029, HNT34, SLR25, LAMA84, SNU1077, SNU5, WM115, ECGI10, HS688AT, PK1, EFO21, SKLU1, IMR32, NCIH2122, SKNBE2, KMRC3, HCC2108, KARPAS422, SNU886, TUHR14TKB, TE10, MPP89, PSN1, MOLM6, HT144, 42MGBA, JHOC5, SNU620, JURLMK1, NCIH1395, LN215, CCFSTTG1, EFM19, ISTMES2, YAPC, JHOM2B, DB, MSTO211H, OCIAML3, NCIH3122, SR786, HCC461, HS870T, SKNFI, CL14, NCIH522, SNU668, KPNRTBM1, JVM3, QGP1, RPMI7951, HCC1500, COLO678, MKN1, HCC1428, TE15, CAPAN1, NCIH82, MKN45, JEKO1, NCIH69, MG63, NCIH508, SKHEP1, MOLM13, SKMM2, U2OS, SUDHL4, SKNDZ, NCIH226, SNU1105, MOLM16, SNU626, RL, P12ICHIKAWA, SKM1, HCC1143, G402, SF295, SNU478, NCIH647, NCIH1781, KMS12BM, T84, CORL24, OE33, SW780, SKRC20, KG1, TF1, NUDHL1, H4, LUDLU1, MHHES1, CALU3, HLF, NCIH2081, NCIH520, J82, TEN, RI1, NCIH2196, SKCO1, COLO800, BL70, NCIH747, K029AX, MEC1, U937, SNU685, TE5, OVSAHO, SAOS2, 769P, SNU1197, HS739T, NCIH1944, BICR6, NCIH838, PANC0813, SW1353, KMS28BM, SNU449, SW837, SNU475, SKMEL3, TC71, UACC62, KMS20, NCIN87, UO31, A704, TYKNU, NCIH1694, BV173, CAKI1, NCIH1915, EFE184, OCIMY7, SW1088, LU65, ME1, CA46, SH4, RERFLCSQ1, OVKATE, LU99, KNS60, KPNSI9S, NCIH2228, NCIH1666, MESSA, MELHO, NCIH2085, TE8, MOLP2, HCC95, LN428, BCPAP, CAL54, CJM, TUHR10TKB, SNU8, SNU1196, NALM1, NCIH460, CAS1, SKMEL1, SNU216, HCC56, PK45H, YH13, SW1463, LI7, HSC2, RT112, HUH1, JHH4, MALME3M, SNU387, KNS81, HUH7, NCIH2170, RERFLCKJ, SNU182, VMRCRCW, GSU, KU1919, F36P, TE11, SW1116, SF767, NCIH716, MUTZ5, SNU423, OELE, TUHR4TKB, NCIH1792, KO52, EW8, SNU46, LS123, TCCPAN2, BICR16, SNB75, RKN, NCIH146, KE39, CORL88, HUT78, NCIH1299, CALU1, INA6, SNU1272, NCIH1092, HCC33, CAL78, SNU410, CAL33, PEER, 59M, NCIH2030, UMUC3, NCIH1184, KURAMOCHI, NCIH2171, HS821T, OVISE, ABC1, T173, DMS114, RS5,SNU61, NCIH2004RT, WSUDLCL2, BXPC3, BT20, SNU761, HUTU80, HS618T, HS606T, KMS34, HEYA8, SNU489, OE21, VMCUB1, HSC4, HT1197, BHY, SNU1076, IGR39, K562, HT29, SQ1, UACC893, A498, SIHA, AML193, A172, NCIH1836, ECC10, TDOTT, HCC78, EBC1, KHM1B, RCM1, SW1710, ST486, UACC812, ISTMES1, YKG1,T98G, G361, MDAMB436, FUOV1, HCC364, KMS27, JHH2, HCC1171, UACC257, C32, SNU16, COLO741, MC116, JHOS4, EPLC272H, NCIH1876, NCIH1975, KMS26, NCIH1437, NCIH2073, LN235, TM31, BC3C, DMS153, LN229, LCLC97TM1, TTC709, KMS21BM, PATU8902, SLR26, MIAPACA2, M07E, BEN, KYO1, TE6, PECAPJ34CLONEC12, KYM1, COV644, SF126, NCIH2227, SUDHL6, HUT102, HOS, RVH421, SKMEL28, HS746T, OVCAR4, SNU1041, PECAPJ15, JHH1, MDAMB157, KNS42, SNU201, HCC1806, HEP3B217, U266B1, LCLC103H, NCIH596, IOMMLEE, YD8, KS1, HS944T, FU97, LN340, SNU119, RPMI8402, KYSE520, NCIH441, NCIH211, SKMEL31, CMK, HMEL, HDQP1, COLO829, JL1, OVMANA, TE1, NCIH28, 786O, IGR37, SW620, SUIT2, JJN3, RAJI, SF268, SUDHL8, A2780, KMS18, SCLC21H, SUDHL5, WM1799, CORL23, OVTOKO, SUDHL1, SKMES1, NCIH1355, HCC44, HCC70, SW900, SBC5, HUH6, IALM, LN443, NUGC4, NCIH1734, LN464, SW1573, MKN7, OE19, SW948, A549, SNU1066, SNU503, KMRC1, L33, SNU878, CI1, OV7, RH18, HCC2814, HCC2157, SNU899, KYSE180, TE9, CORL47, OVCAR8, A3KAW, DMS53, HCC1395, NCIH2882, RMUGS, L1236, DMS79, OAW42, LC1F, EKVX, P3HR1, SNU283, KMRC2, NCIH854, JIMT1, HCC1833, CAOV3, KMS11, SNU1214, TT2609C02, COLO680N, NCIH2291, RMGI, TCCSUP, HMC18, SNUC1, YD10B, HT1376, HCC202, TE14, NCIH2066, KASUMI2, NCIH1963, SKMEL5, HCC2279, PECAPJ41CLONED2, NCIH1838, JHH5, PECAPJ49, SNU601, NCIH1385, GB1, HEPG2, A253, UBLC1, DM3, CORL95, NCIH1623, MOLP8, GSS, NCIH1703, SJSA1, DMS273, LOXIMVI, OCIM1, NCIH196, JMSU1, L428, HCC2218, GI1, A427, MKN74, MDAMB175VII, LNZ308, NUGC2, YD38, MM1S, SH10TC, WM983B, NCIH1648, NCIH526, MDAMB231, LK2, P31FUJ, BICR56, TE441T, KIJK, RERFLCAD2, NCIH727, ONS76, KYSE30, HSC3, PC9, NCIH1105, NCIH2023, SEM, CAMA1, KYSE70, NCIH2126, DAUDI, LXF289, A2058, NCIH810, SHP77, RERFLCAD1, BFTC909, KATOIII, BICR22, MOLT13, MCAS, HLFA, CL40, HS695T, NCIH446, HS936T, BFTC905, COLO668, NB1, COLO679, L540, SNU738, HUH28, KYSE410, SKMEL30, SKOV3, COLO783, T3M10, HS939T, KMH2, NCIH524, RPMI8226, BT483, LN18, SW403, EJM, SKMEL24, KYSE140, KYSE510, HOP92, CAL12T, WM793, ZR7530, HUNS1, NCIH1436, HEC50B, CAL27, RH30, UMUC1, GCT, YD15, NCIH322, AMO1, SCABER, HCC366, NCIH2087, SW480, HARA, DMS454, NCIH1373, FADU, HGC27, JHH7, MDAMB468, HS698T, MORCPR, NCIH1435, NCIH661, OCIMY5, KYSE150, CAL51, CAL851, KNS62, HCC1954, NCIH358, HOP62, KMBC2, DBTRG05MG, COLO684, KYSE450, NCIH1048, CHAGOK1, HCC1195, NCIH1568, NCIH1930, NCIH510, HCC515, KYSE270, RS411, NCIH2347, MDAMB415, EB1, HCC15, MFE296, AGS, MELJUSO, IGR1, SW1783, MDAMB435S, TOV21G, NCIH2009, SF172, NCIH1793, KMM1, SW1271, HCC1438, NCIH1563, NCIH1651, NCIH1869, CL34, 647V, FTC238, SNU719, WM88, NCIH23, HCC1359, CAL148, FTC133, NCIH2106, 5637, ES2, SNU349, SNU520, JHUEM2, MDAMB453, NUGC3, NCIH2286, ESS1, HT, IPC298, NCIH1573, TE4, MOLTI6, IM95, CMLT1, NCIH1339, RCHACV, BCP1, NCIH2172, DV90, HT55, BT474, JHUEM1, NCIH2110, HCC1569, HMCB, SNU1, SNU324, MDAMB361, MDST8, EFO27, PF382, NALM6, SKUT1, AN3CA, HEC1B, HPBALL, RKO, NAMALWA, NCIH650, HEC265, OVK18, 2313287, TGBC11TKB, LOVO, NCIH2342, MDAPCA2B, SUPT1, HEC1A, SNU407, 22RV1, LS180, SW48, SNUC4, REH, ISHIKAWAHERAKLIO02ER, OC314, CCK81, MOLT3, RL952, IGROV1, SNUC2A, COLO792, KM12, SNUC5, HCT116, HEC151, 639V, SNGM, HCC2450, HUCCT1, LNCAPCLONEFGC, EN, DU145, NCIH1155, DND41, GP2D, KCL22, HEC6, LS411N, HT115, MEWO, MFE319, SNU175, HEC108, SNU81, BICR18, JHUEM7, HEC59, JURKAT, HEC251, HCT15, CW2, SNU1040, 1321N1, 143B, 451LU, A673STAG2KO16, A673STAG2KO45, A673STAG2NT14, A673STAG2NT23, ACCS, AZ521, BECKER, BGC823, BJHTERT, BT16, C3A, CBAGPN, CGTHW1, CHL1, CHLA06ATRT, CHLA10, CHLA218, CHLA266, CHLA32, CHLA57, CHLA9, CHLA99, CMK115, CMK86, COGE352, COLO205, COLO699, COLO704, COLO775, COLO818, COLO849, CORL51, COV504, CPCN, CW9019, D384, D425, D458, D556, DERL2, DL, DL40, DLD1, DOV13, EB2, EVSAT, EWS834, F5, FEPD, GLC82, GRM, NCIH292, HCC1588, HCC1897, HCC2998, HCT8, HELA, HK2, HLC1, HLE, HN, HRT18, HS571T, HS604T, HTK, JR, KARPAS384, KCIMOH1, KD, KHYG, KLM1, KOPN8, KP1N, KP1NL, KPMRTRY, L82, LC1SQSF, M059J, MAC2A, MEC2, MKL1, MKL2, MOGGCCM, MOGGUVW, MOLT4, MON, MONOMAC1, MOTN1, MSDASH1, MTA, MYLA, NCIH187, NCIH1993, NCIH2141, NHAHTDD, NKL, OC315, OC316, OCILY10, OCILY 12, OCILY 132, OUMS27, OVCAR5, PCM6, CCLFPEDS0001T, CCLFPEDS0003T, PETA, PL45, R256, R262, RCC4, RPMI6666, RT11284, SCMCRM2, SF8657, SHSY5Y, SJRH30, SKMEL2, SKNEP1, SKPNDW, SKRC31, SMSCTR, SMZ1, SNB19, SNUC2B, STM9101, SUMB002, SUPHD1, TC32, TTC466, TIG3TD, TK10,TTC1240, TTC549, TTC642, U138MG, UMRC2, UMRC6NEO, UPCISCC090, UPCISCC152, UPCISCC154, UT7, UW228, VMRCLCD, VMRCLCP, WM2664, YMB1, 127399, FUJI, SW982, SYO1, YAMATO, BIN67, SCCOHT1, SCS214, CHLA258, TC106, COGAR359, Y79, CHLA15, COGN278, COGN305, NB1643, 8305C, 8505C, HA1E, PLCPRF5, TT, CME1, A431, ANGMCSS, BICR10, BICR78, C33A, C4I, C4II, CASKI, CHP134, COLO794, COV413A, DOTC24510, GIMEN, GP5D, H103, H157, HTCC3, JOPACA1, LAN2, LAN6, MB1, MCF10A, MDAMB330, ME180, MS751, NCIH1770, NCIH2135, NCIH345, NCIH847, NGP, NMB, OACM51, OCIC5X, OCIP5X, OV17R, PA1, PACADD119, PACADD135, PACADD137, PACADD159, PACADD161, PACADD165, PACADD188, PWR1E, RO82W1, RPMI2650, SCLC22H, SUM102PT, SUM1315MO2, SUM149PT, SUM159PT, SUM185PE, SUM190PT, SUM229PE, SUM44PE, SUM52PE, SUM225CWN, SW156, SW626, SW954, SW13, SW756, TO14, UMUC13, UMUC14, UMUC16, UMUC4, UMUC5, UMUC10, UMUC11, UMUC6, UMUC7, UMUC9, UMC11, UWB1289, VP229, WERIRB1, WPEINA22, TC138, TC205, CCLFPEDS0008T, 921, A388, ASH3, BLUE1, BOKU, BONNA12, BPH1, C10, C125PM, C75, C80, C84, C99, CI, CII, CORL32, CORL321, EGI1, EMTOKA, ESO26, ESO51, FARAGE, FLO1, H357, H376, H413, HCA1, HCC1008, HCS2, HCSC1, HEC1, HEC116, HEMCSS, HG3, HKA1, HMY1, HSC1, HSC5, HT3, HUO9, IHH4, JAR, JEG3, JMURTK2, KKU100, KKU213, KML1, KMLS1, KMS28PE, KON, KOSC2CL343, KYAE1, LS, LU135, MCC13, MCC142, MCC26, MEL202, MERO14, MERO25, MERO41, MERO48A, MERO82, MERO83, MERO84, MERO95, MM127, MM370, MM383, MM386, MM415, MM426, MOLM1, MUTZ8, NCCIT, NCIH1417, NCIH64, NH12, NO10, NO11, NOZ, NP2, NP3, NP5, NP8, OCIAML4, OCILY18, OCILY7, OCIM2, OCUG1, ONDA7, ONDA8, ONDA9, OSC19, OSC20, P4E6, PEA1, PEO1, PEO4, PGA1, RAMOS, RCK8, ROS50, SAT, SCC3, SEKI, SHI1, SHMAC4, SHMAC5, SISO, SKGI, SKGII, SKGT2, SKGT4, SKN, SKNO1, SNU638, SUSA, TASK1, TFK1, TGW, U2904, U698M, UHO1, UMRC3, UMRC7, UPCISCC026, UPCISCC040, UPCISCC074, UPCISCC116, UPCISCC131, VAESBJ, VAL, VMRCMELG, WSUNHL, PFSK1, HS860T, CAL72, GOTO, OCIC4P, SEMK2, HB1119, HSB2, CCRFCEM, RH28, RMS13, RC2, RHJT, TTC442, RH36, RH4, CCLFPEDS0018T, SNU1544, RH18DM, LPS6, LPS27, 93T449, 94T778, 95T1000, LPS141, LPS853, LPS510, LPS067, OS252, C396, MFM223, COLO824, SW527, 184B5, ICC10, ICC106, ICC108, ICC12, ICC137, ICC15, ICC2, ICC3, ICC4, ICC5, ICC6, ICC8, ICC9, G415, HKGZCC, KMCH1, RBE, SG231, SSP25, TGBC1TKB, TGBC52TKB, TKKK, YSCCC, JHC7, MUGCHOR1, UMCHOR1, MS1, CCLP1, CCSW1, GB2, NZOV9, NALM16, ECC2, 9505BIK, A375SKINCJ1, A375SKINCJ2, A375SKINCJ3, UACC62SKINCJ1, SKMEL19, MP46, MEL285, MEL290, OMM1, OMM25, HOKUG, SKGIIIA, PMFKO14, TGBC18TKB, ECC4, TT1TKB, HHUA, HOUA1, SAS, HSKTC, PK8, HMVII, HOTHC, T3M5, CA922, HSQ89, HO1U1, HTMMT, LU134A, LU139, ATN1, P30OHK, SLVL, HSSCH2, NOS1, HSOS1, LU165, NB69, HSSYII, 201T, BB65RCC, CAL39, CHSA0011, CHSA0108, CHSA8926, CORL303, CP50MELB, CP66MEL, CP67MEL, CS1, DJM1, EMCBAC1, EMCBAC2, ES1, ES3, ES4, ES5, ES6, ES7, ES8, EW1, EW11, EW12, EW13, EW16, EW18, EW22, EW24, EW3, EW7, GAK, GMEL, GT3TKB, H2369, H2373, H2461, H2591, H2595, H2722, H2731, H2795, H2803, H2804, H2810, H2818, H2869, H290, H513, HA7RCC, HEY, HSC39, HUO3N1, ISTMEL1, ISTSL1, ISTSL2, JHOS3, K2, K5, KGN, LB1047RCC, LB2241RCC, LB2518MEL, LB373MELD, LB647SCLC, LB996RCC, LC1SQ, LC2AD, LU99A, M14, MCIXC, MKN28, MMACSF, MRKNU1, MZ1PC, MZ2MEL, MZ7MEL, NCC010, NCC021, NCIH1304, NCIH1688, NCIH250, NCIH322M, NCIH378, NCIH720, NCIH740, NCIH748, NCIH835, NY, OCUBM, OMC1, OVCA420, OVCA433, OVMIU, PC3JPC3, PL18, PL4, RCCAB, RCCER, RCCFG2, RCCJF, RCCJW, RCCMF, RERFLCFM, RH1, RXF393, SBC1, SBC3, SCH, SN12C, SW962, TCYIK, TMK1, UCH2, WM1552C, WM278, WM35, YMB1E, ALLPO, ARH77, BALL1, BB30HNC, BB49HNC, BC1, BC3, BE13, BE2M17, CESS, COLO320HSR, CROAP2, CTB1, CTV1, D245MG, D247MG, D263MG, D336MG, D392MG, D423MG, D502MG, D542MG, D566MG, DG75, DIFI, DOK, DSH1, EB3, ETK1, GRST, H3118, H9, HAL01, HC1, HCE4, HO1N1, HS445, HS633T, IM9, IMR5, JHU011, JHU022, JHU029, JIYOYEP2003, JSC1, KARPAS1106P, KARPAS231, KARPAS45, KINGS1, KMOE2, KNS81FD, KOSC2, KPNYS, KY821, KYSE220, KYSE50, LB771HNC, LB831BLC, LC41, LN405, LNZTA3WT4, MCCAR, MFHINO, MHHPREB1, ML2, MLMA, MN60, MYM12, NBTU110, NB10, NB12, NB13, NB14, NB17, NB5, NB6, NB7, NCIH128, NCIH630, NEC8, NK92MI, NKM1, NTERA2CLD1, OACP4C, P32ISH, PCI15A, PCI30, PCI38, PCI4B, PCI6A, QIMRWIL, RAMOS2G64C10, RF48, RPMI8866, SKMG1, SKN3, STS0421, SUDHL16, SUPB8, SW684, SW872, TE12, TGBC24TKB, TK, TUR, WIL2NS, YT, 184A1, 600MPE, HBL100, HCC2185, HCC2688, HCC3153, LY2, MACLS2, MCF12A, MX1, SKBR5, SKBR7, ZR75B, GISTT1, HCC827GR, SS1A, UCH1, HCET, JHU028, M980513, MOT, NBSUSSR, BB30PBL, BB49EBV, BB65EBV, CAR1, CP50EBV, CP66EBV, DIPG007, GBM001, HA7EBV, L542, LB1047EBV, LB2241EBV, LB2518EBV, LB373EBV, LB647PBL, LB771PBL, LB831EBV, LB996EBV, MZ1B, MZ7B, NCIBL128, NCIBL1395, NCIBL1437, NCIBL1770, NCIBL2009, NCIBL2052, NCIBL2087, NCIBL209, NCIBL2122, NCIBL2126, NCIBL2171, HCC1187BL, HCC1599BL, HCC1937BL, LS1034PBL, HCC38BL, HCC1143BL, J82EBV, COLO829BL, HCC2157BL, HCC1395BL, HCC2218BL, HCC1954BL, M00921, M1203273, MET2B, ACN, MC1010, UDSCC2, SC1, CROAP3, GEO, HUH6CLONE5, SARC9371, KMHDASH2, CCLFUPGI0005T, HT144SKINFV1, HT144SKINFV3, HT144SKINFV2, RVH421SKINFV1, HAP1, WM3211, WM4235, M040416, and M140325.

In some embodiments, the fusion protein includes a nuclear localization domain which provides for the protein to be translocated to the nucleus. Several nuclear localization sequences (NLS) are known, and any suitable NLS can be used. For example, many NLSs have a plurality of basic amino acids, referred to as a bipartite basic repeats (reviewed in Garcia-Bustos et al, 1991, Biochim. Biophys. Acta. 1071:83-101). An NLS containing bipartite basic repeats can be placed in any portion of chimeric protein and results in the chimeric protein being localized inside the nucleus. In preferred embodiments a nuclear localization domain is incorporated into the final fusion protein, as the ultimate functions of the fusion proteins described herein will typically require the proteins to be localized in the nucleus. However, it may not be necessary to add a separate nuclear localization domain in cases where the DBD domain itself, or another functional domain within the final chimeric protein, has intrinsic nuclear translocation function.

### Variants

In some embodiments, the fusion protein(s) or components thereof described herein, or the polynucleotides encoding the fusion protein(s) or components thereof described herein, are at least 80%, e.g., at least 85%, 90%, 95%, 98%, or 100% identical to the amino acid sequence of an exemplary sequence (e.g., as described herein), e.g., have differences at up to 1%, 2%, 5%, 10%, 15%, or 20% of the residues of the exemplary sequence replaced, e.g., with conservative mutations, e.g., including or in addition to the mutations described herein. In preferred embodiments, the variant retains desired activity of the parent.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology.”). The percent identity between the two sequences is a function of the number of identical positions shared by the sequences, taking into account the number of gaps, and the length of each gap, which need to be introduced for optimal alignment of the two sequences.

Percent identity between a subject polypeptide or nucleic acid sequence (i.e. a query) and a second polypeptide or nucleic acid sequence (i.e. target) is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for target proteins or nucleic acids, the length of comparison can be any length, up to and including full length of the target (e.g., 5%, 10%, 20%, 30%, 40%, 50%, 60%, 70%, 80%, 90%, 95%, or 100%). For the purposes of the present disclosure, percent identity is relative to the full length of the query sequence.

For purposes of the present disclosure, the comparison of sequences and determination of percent identity between two sequences is accomplished using Smith Waterman Alignment with a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

### E3 Ligase Binding Modulators

The methods described herein are useful, for example, for identifying compound (e.g., drug)-dependent proximity interactions. In some embodiments, the methods are used to validate and/or identify targets that selectively interact with an E3 ligase, e.g., cereblon, in the presence of a compound, e.g., an E3 ligase binding modulator, e.g., a cereblon binding modulator.

E3 ligase binding modulators, e.g., E3 ligase substrate receptor binding modulators, e.g., cereblon binding modulators, are described, for example, in WO2021/069705 and WO2021/053555, which are hereby incorporated by reference in their entirety.

In some embodiments, the E3 ligase binding modulator, e.g., E3 ligase substrate receptor binding modulator, e.g., cereblon binding modulator, is a compound shown in Tables 4 and 5, below, or pharmaceutically acceptable salts thereof, or a stereoisomers thereof.

TABLE 4
Examples of E3 Ligase Binding Modulators

| Compound | No. |
|---|---|
| 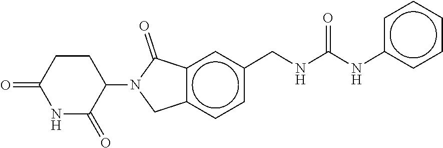 | 1 |
| 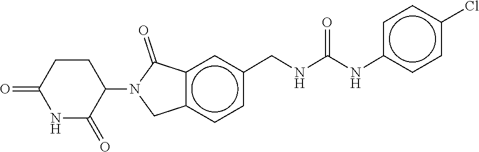 | 2 |
| 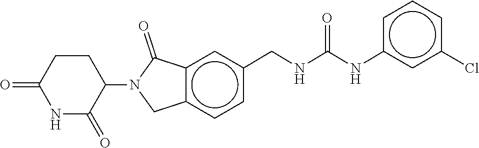 | 3 |
| 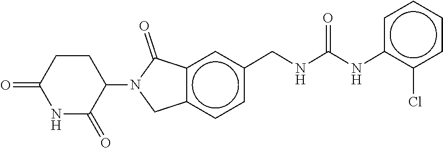 | 4 |
| 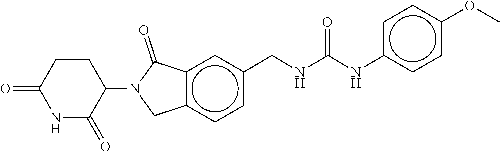 | 5 |
| 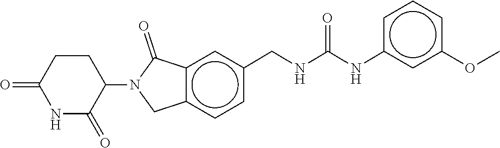 | 6 |
| 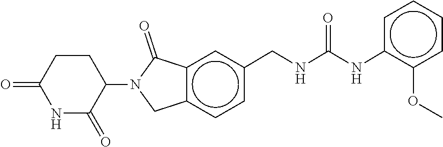 | 7 |
| 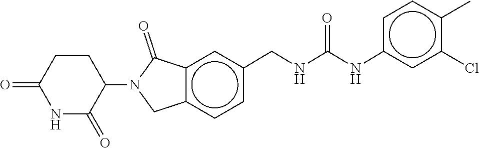 | 8 |
| 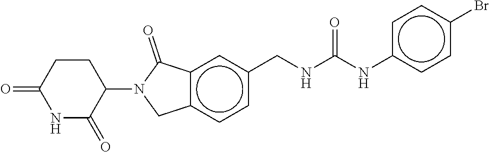 | 9 |
| 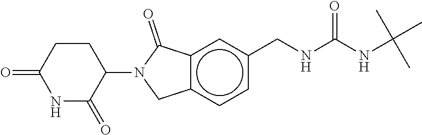 | 10 |
| 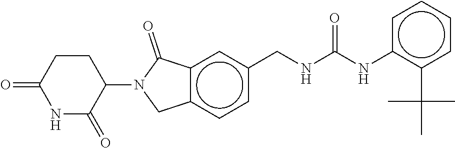 | 11 |
| 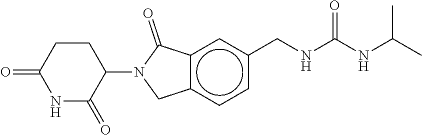 | 12 |
| 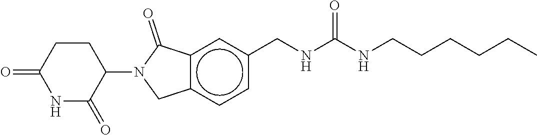 | 13 |
| 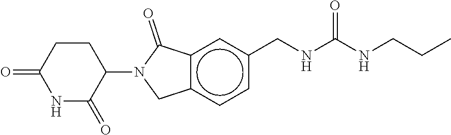 | 14 |
| 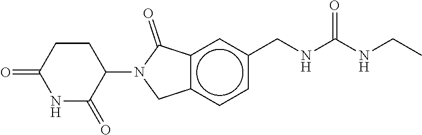 | 15 |
| 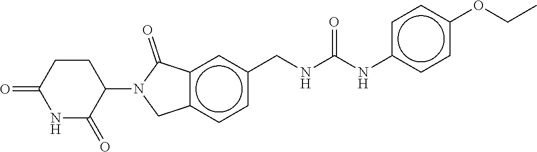 | 16 |
| 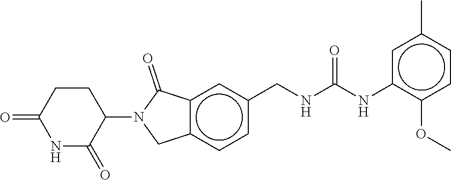 | 17 |
| 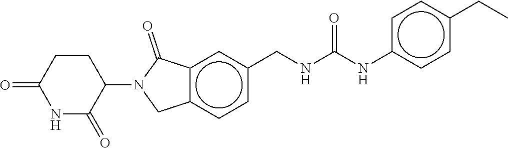 | 18 |
| 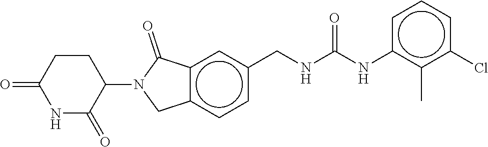 | 19 |
| 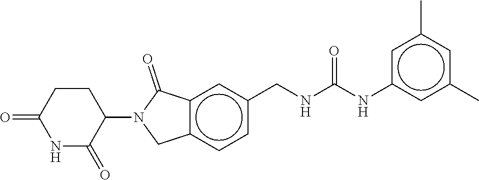 | 20 |
| 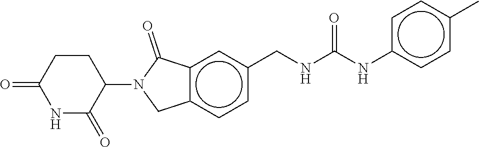 | 21 |
| 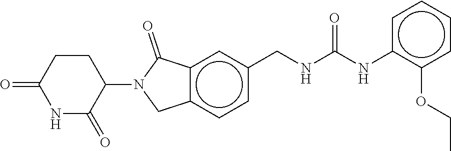 | 22 |
| 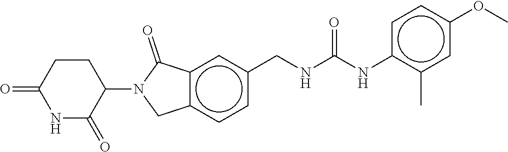 | 23 |
| 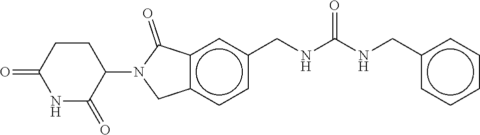 | 24 |
| 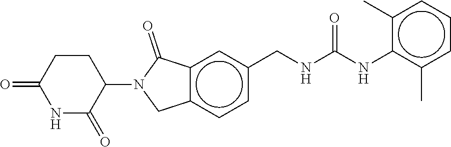 | 25 |
| 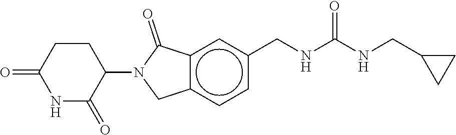 | 26 |
| 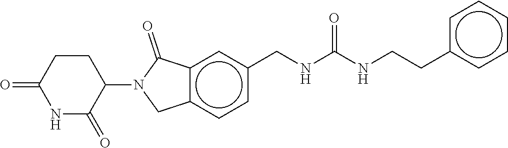 | 27 |
| 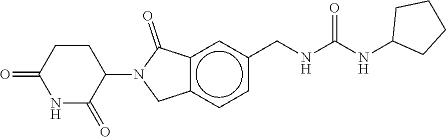 | 28 |
| 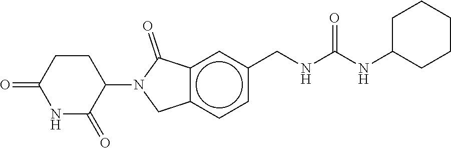 | 29 |
| 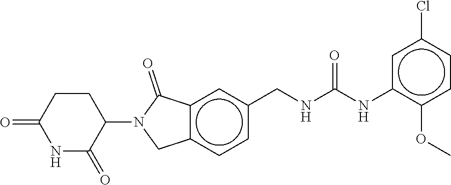 | 30 |
| 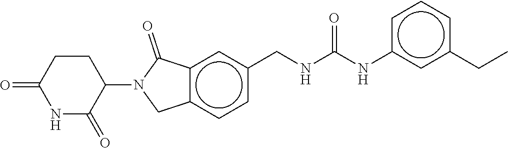 | 31 |
| 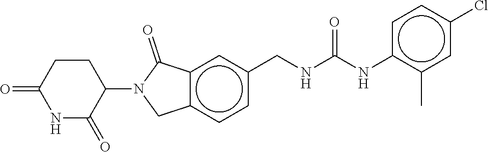 | 32 |
| 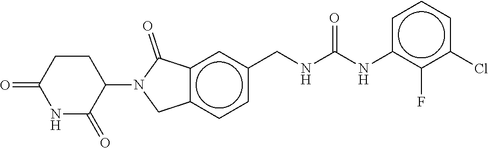 | 33 |
| 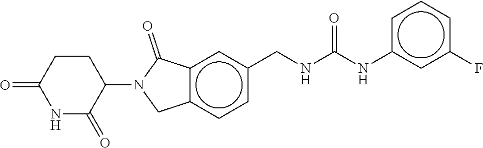 | 34 |
| 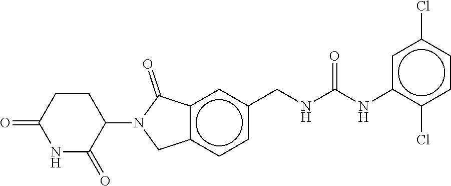 | 35 |
| 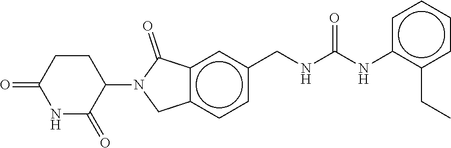 | 36 |
| 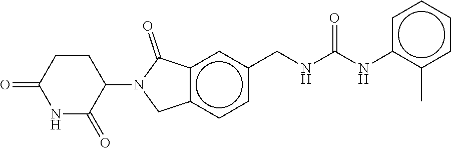 | 37 |
| 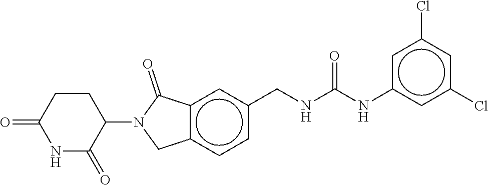 | 38 |
| 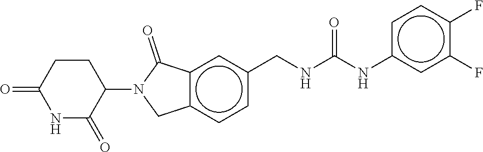 | 39 |
| 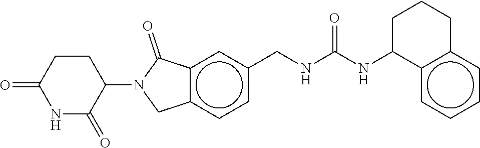 | 40 |
| 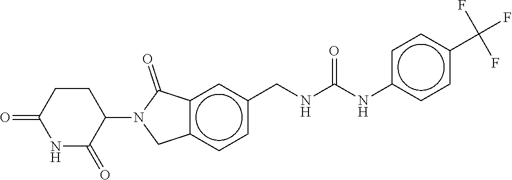 | 41 |
| 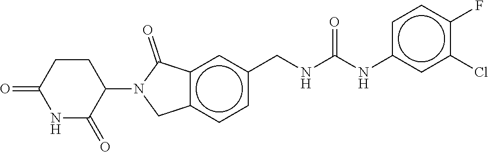 | 42 |
| 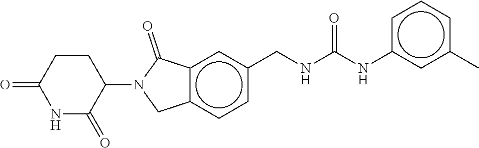 | 43 |
| 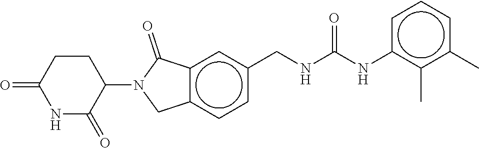 | 44 |
| 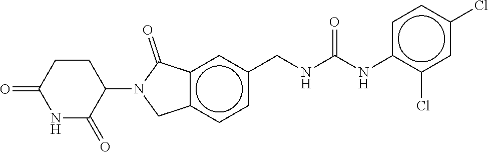 | 45 |
| 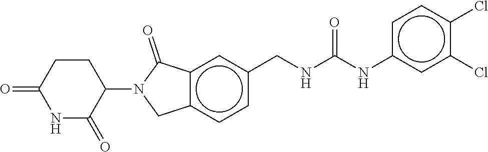 | 46 |
| 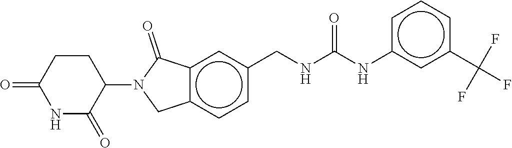 | 47 |
| 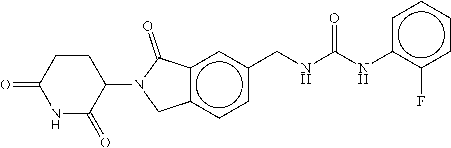 | 48 |
| 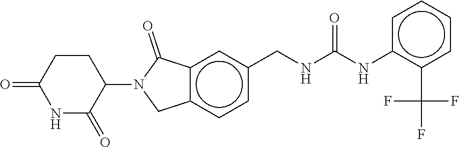 | 49 |
| 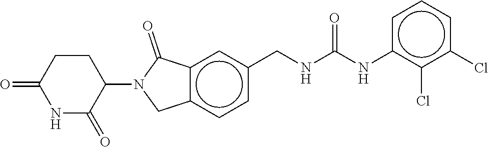 | 50 |
| 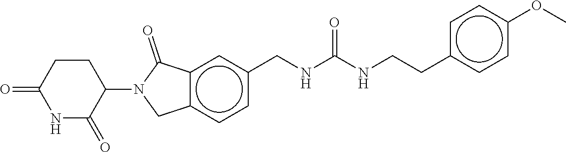 | 51 |
| 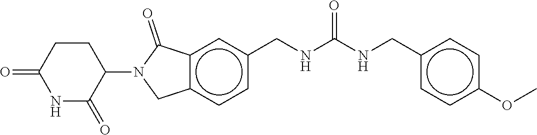 | 52 |
| 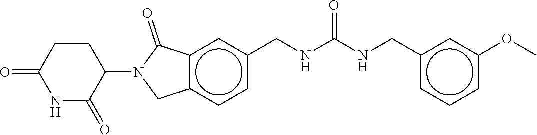 | 53 |
| 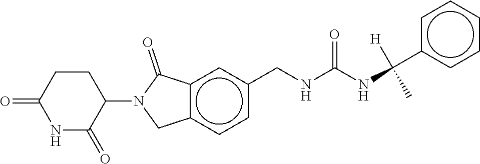 | 54 |
| 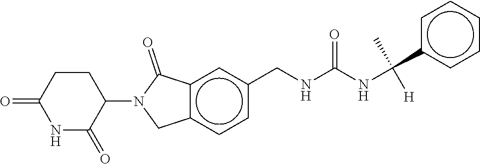 | 55 |
| 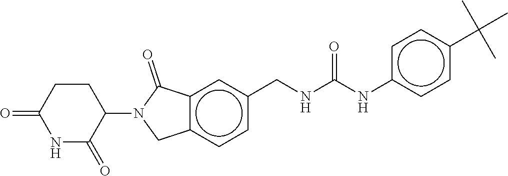 | 56 |
| 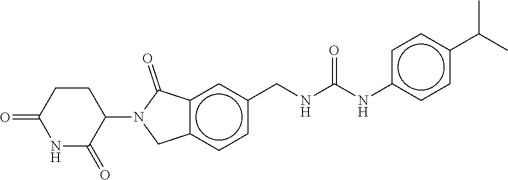 | 57 |
| 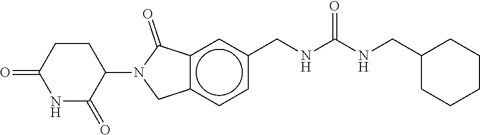 | 58 |
| 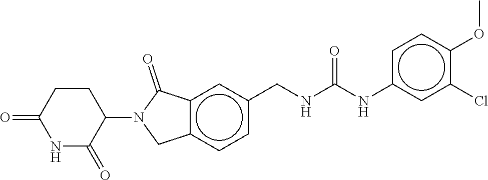 | 59 |
| 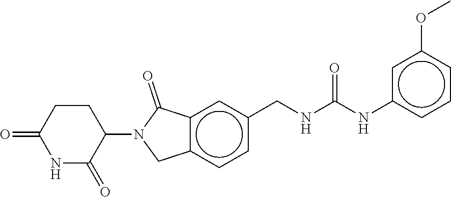 | 60 |
| 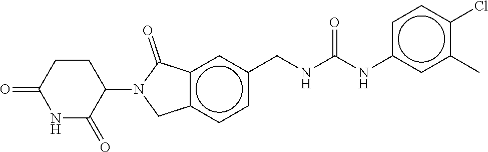 | 61 |
| 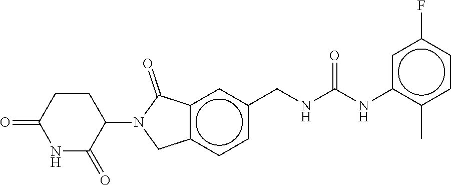 | 62 |
| 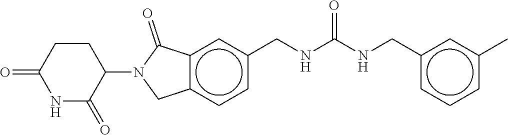 | 63 |
| 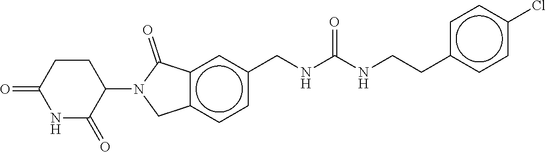 | 64 |
| 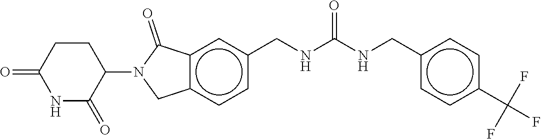 | 65 |
| 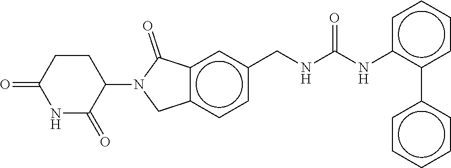 | 66 |
| 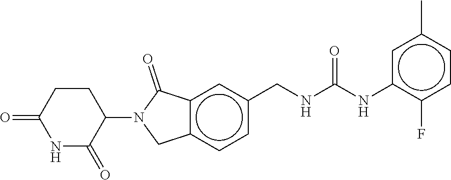 | 67 |
| 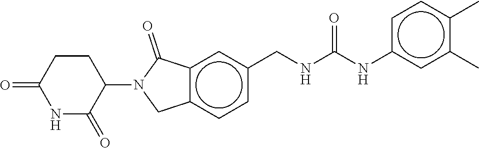 | 68 |
| 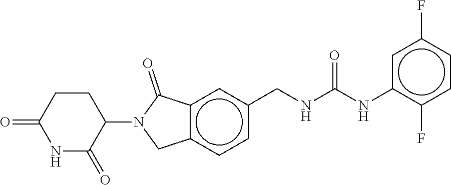 | 69 |
| 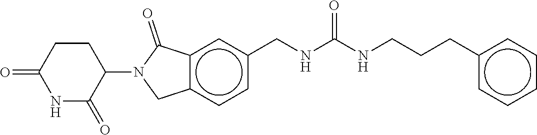 | 70 |
| 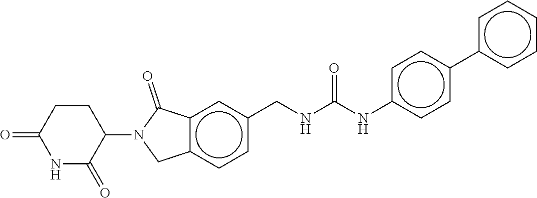 | 71 |
| 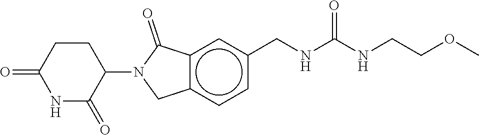 | 72 |
| 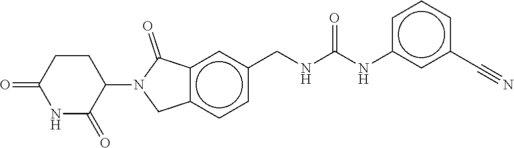 | 73 |
| 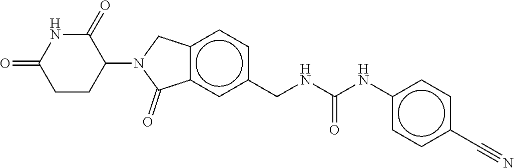 | 74 |
| 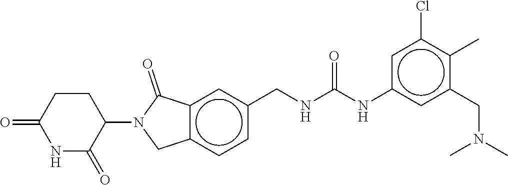 | 75 |
| 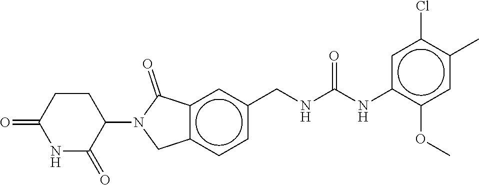 | 76 |
| 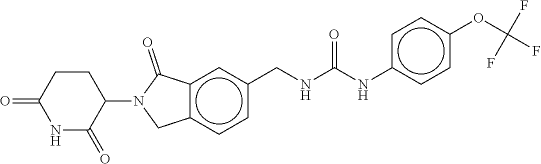 | 77 |
| 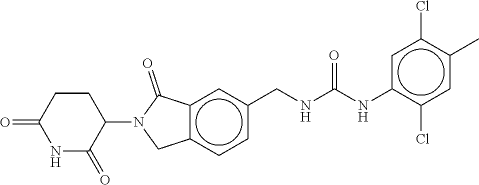 | 78 |
| 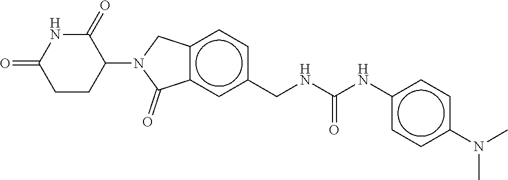 | 79 |
| 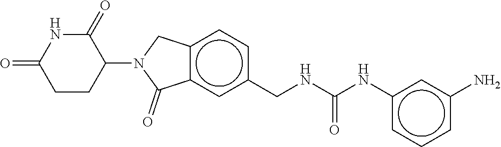 | 80 |
| 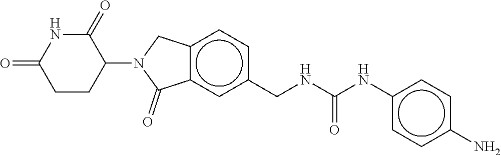 | 81 |
| 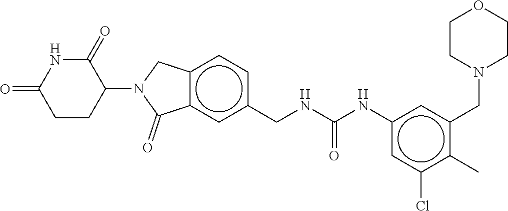 | 82 |
| 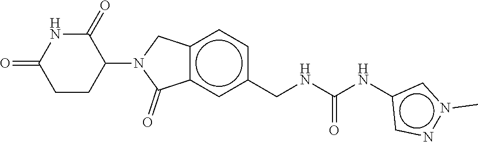 | 83 |
| 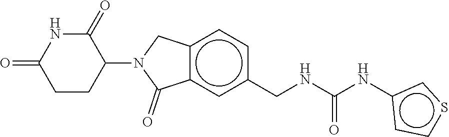 | 84 |
| 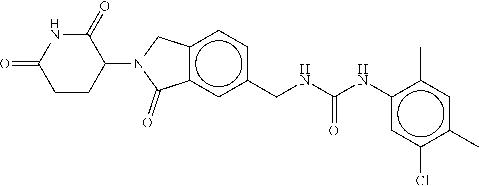 | 85 |
| 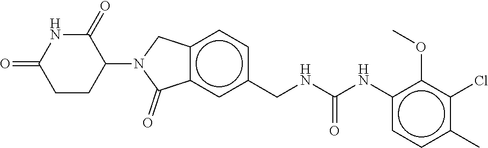 | 86 |
| 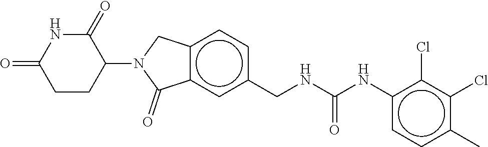 | 87 |
| 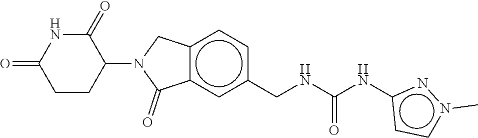 | 88 |
| 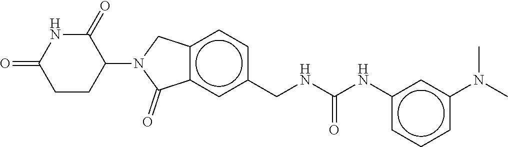 | 89 |
| 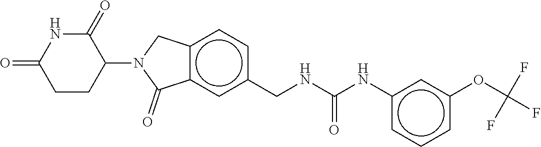 | 90 |
| 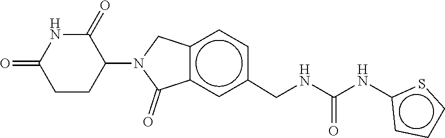 | 91 |
| 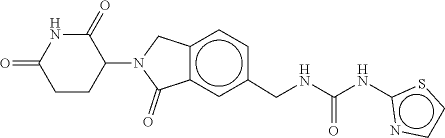 | 92 |
| 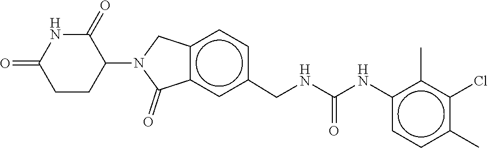 | 93 |
| 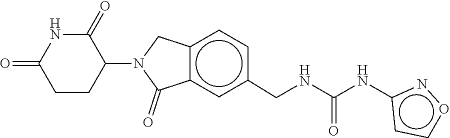 | 94 |
| 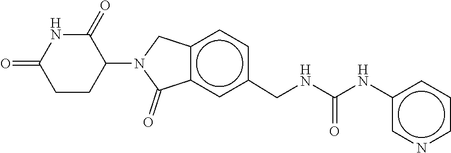 | 95 |
| 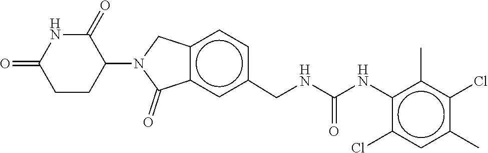 | 96 |
| 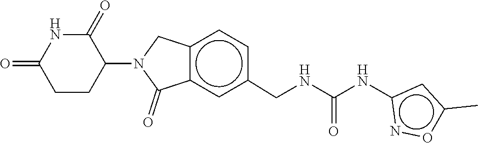 | 97 |
| 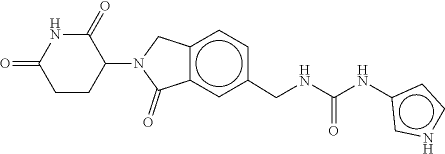 | 98 |
| 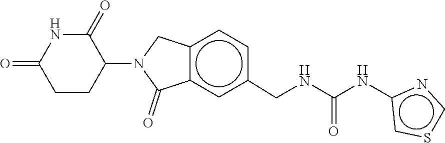 | 99 |
| 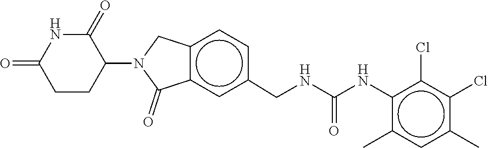 | 100 |
| 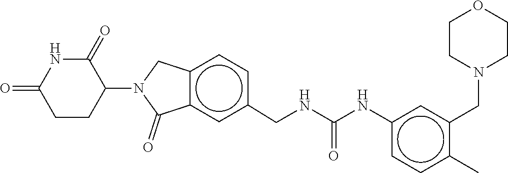 | 101 |
| 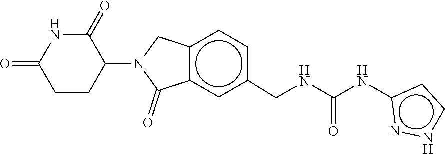 | 102 |
| 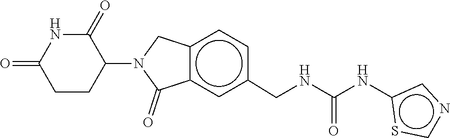 | 103 |
| 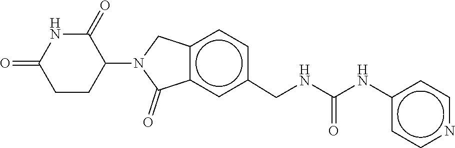 | 104 |
| 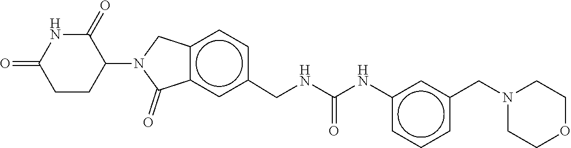 | 105 |
| 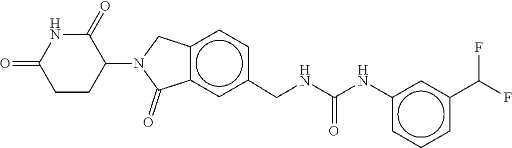 | 106 |
| 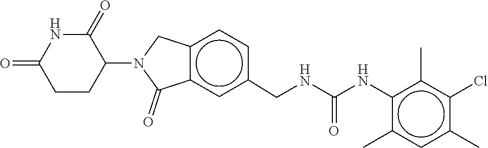 | 107 |
| 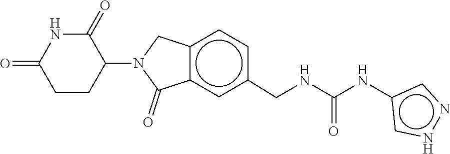 | 108 |
| 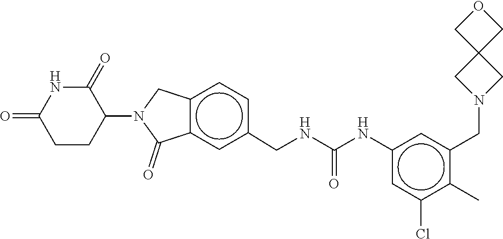 | 109 |
| 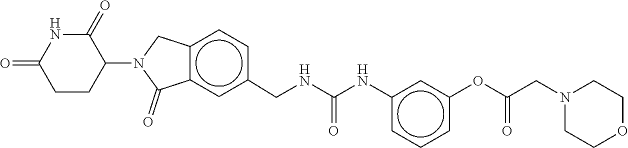 | 110 |
| 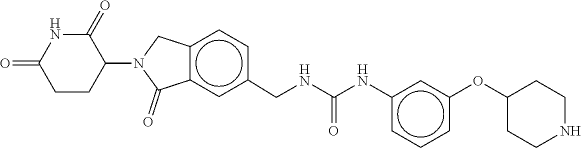 | 111 |
| 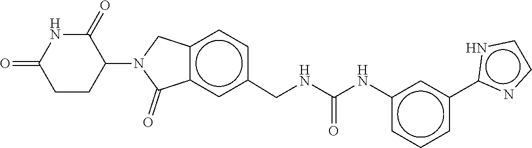 | 112 |
| 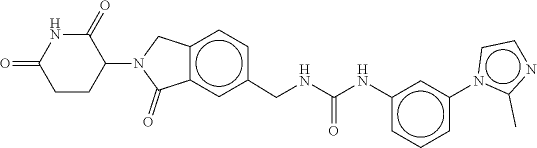 | 113 |
| 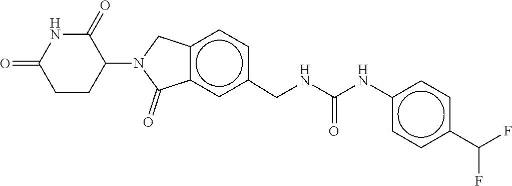 | 114 |
| 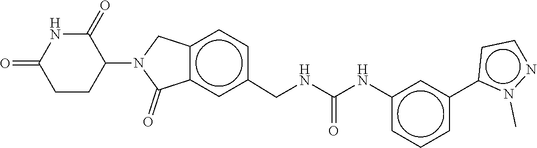 | 115 |
| 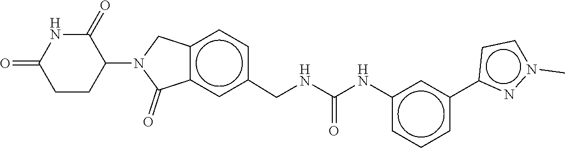 | 116 |
| 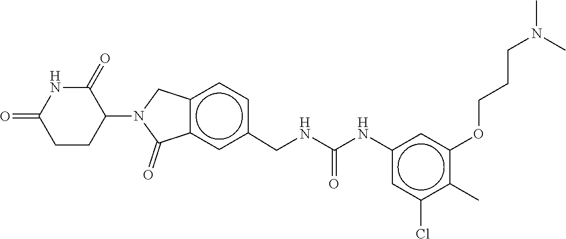 | 117 |
| 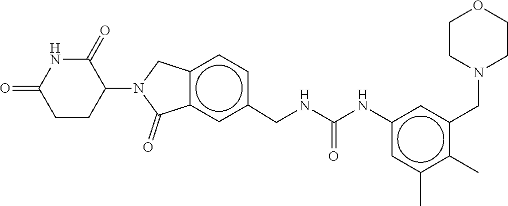 | 118 |
| 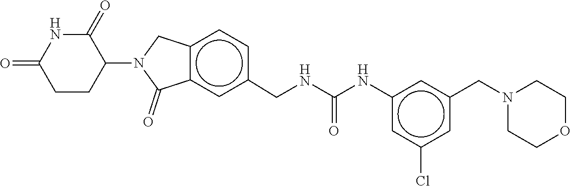 | 119 |
| 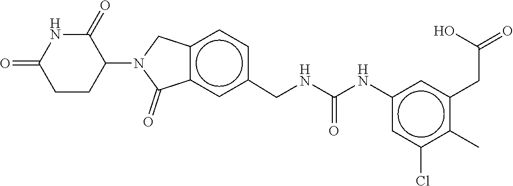 | 120 |
| 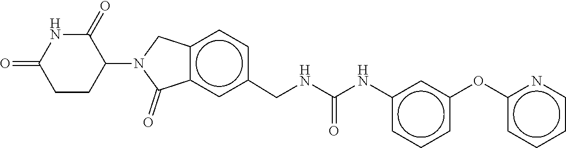 | 121 |
| 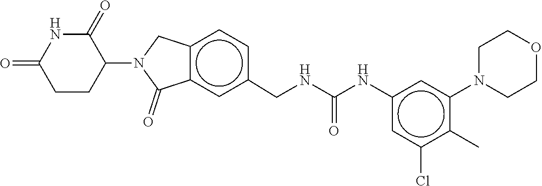 | 122 |
| 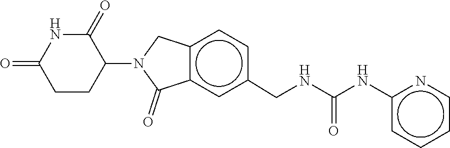 | 123 |
| 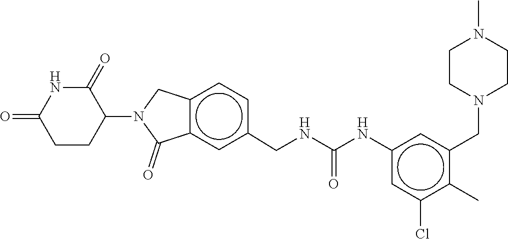 | 124 |
| 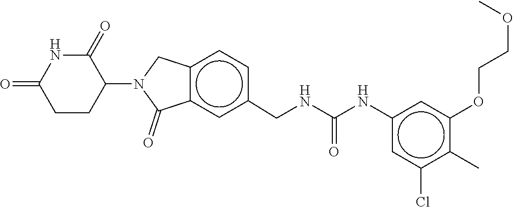 | 125 |
| 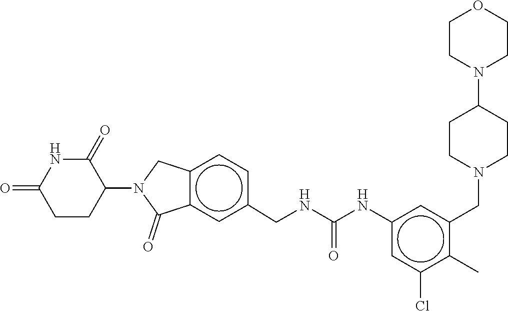 | 126 |
| 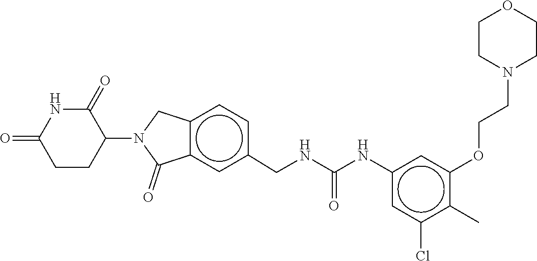 | 127 |
| 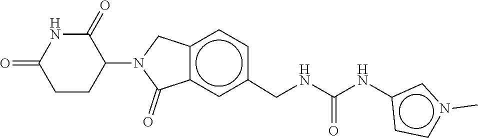 | 128 |
| 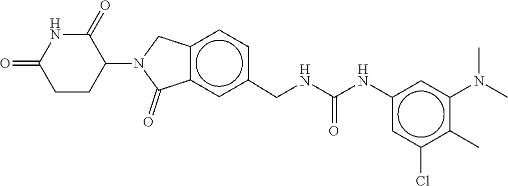 | 129 |
| 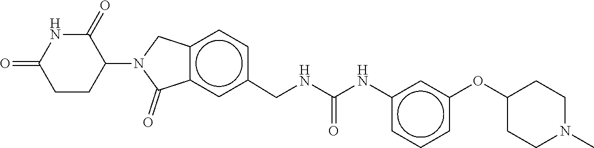 | 130 |
| 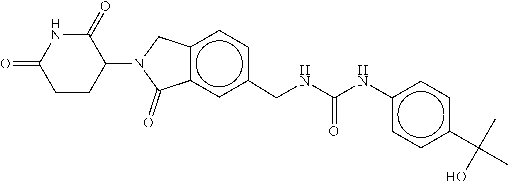 | 131 |
| 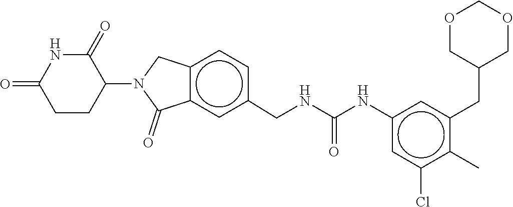 | 132 |
| 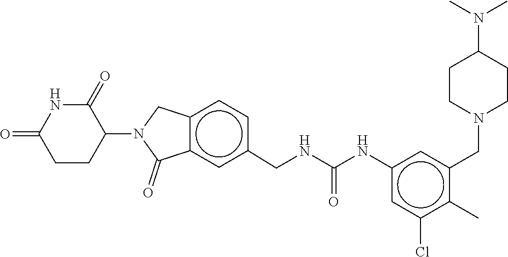 | 133 |
| 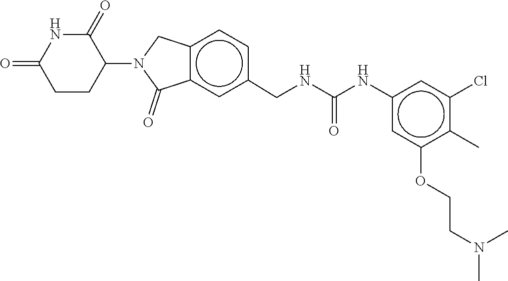 | 134 |
| 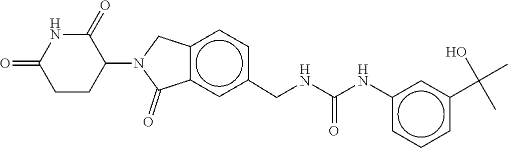 | 135 |
| 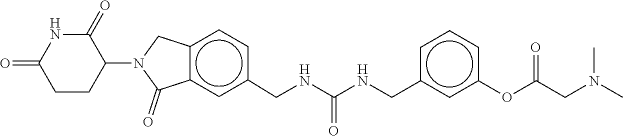 | 136 |
| 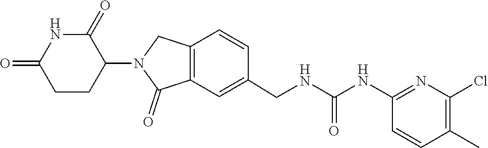 [SMILES: Cc1ccc(NC(=O)NCc2ccc3c(c2)C(=O)N(C2CCC(=O)NC2=O)C3)nc1Cl] | 137 |
| 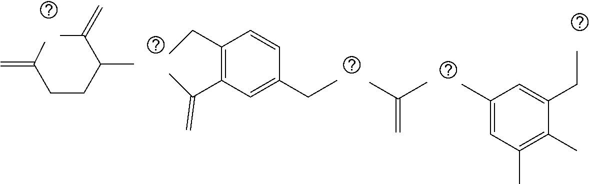 | 138 |
| 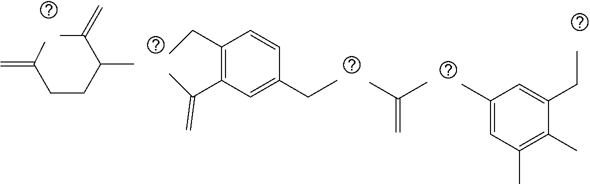 | 139 |
| 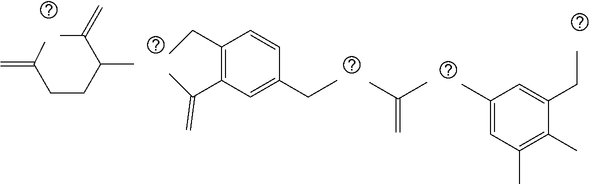 | 140 |
| 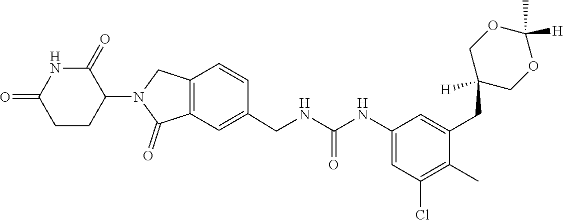 [SMILES: Cc1c(Cl)cc(NC(=O)NCc2ccc3c(c2)C(=O)N(C2CCC(=O)NC2=O)C3)cc1C[C@H]1CO[C@H](C)OC1] | 141 |
| 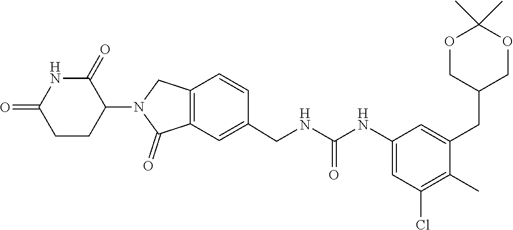 [SMILES: Cc1c(Cl)cc(NC(=O)NCc2ccc3c(c2)C(=O)N(C2CCC(=O)NC2=O)C3)cc1CC1COC(C)(C)OC1] | 142 |
| 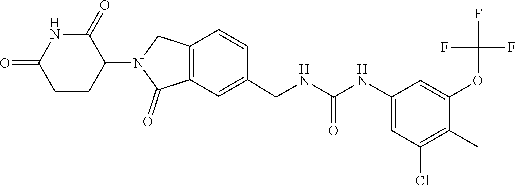 [SMILES: Cc1c(Cl)cc(NC(=O)NCc2ccc3c(c2)C(=O)N(C2CCC(=O)NC2=O)C3)cc1OC(F)(F)F] | 143 |
| 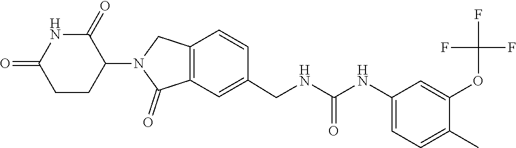 [SMILES: Cc1ccc(NC(=O)NCc2ccc3c(c2)C(=O)N(C2CCC(=O)NC2=O)C3)cc1OC(F)(F)F] | 144 |
| 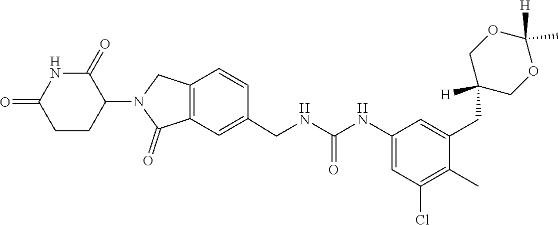 [SMILES: Cc1c(Cl)cc(NC(=O)NCc2ccc3c(c2)C(=O)N(C2CCC(=O)NC2=O)C3)cc1C[C@H]1CO[C@@H](C)OC1] | 145 |
| 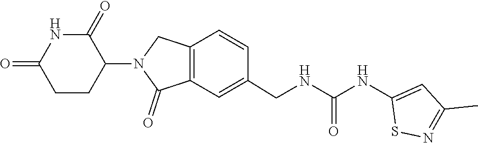 [SMILES: Cc1cc(NC(=O)NCc2ccc3c(c2)C(=O)N(C2CCC(=O)NC2=O)C3)sn1] | 146 |
| 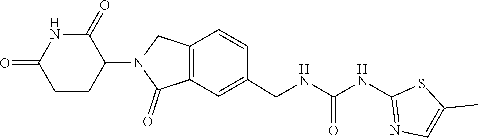 [SMILES: Cc1cnc(NC(=O)NCc2ccc3c(c2)C(=O)N(C2CCC(=O)NC2=O)C3)s1] | 147 |
| 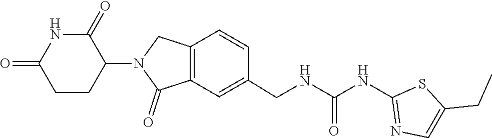 [SMILES: CCc1cnc(NC(=O)NCc2ccc3c(c2)C(=O)N(C2CCC(=O)NC2=O)C3)s1] | 148 |
| 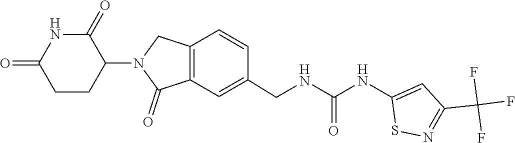 [SMILES: O=C1CCC(N2Cc3ccc(CNC(=O)Nc4cc(C(F)(F)F)ns4)cc3C2=O)C(=O)N1] | 149 |
| 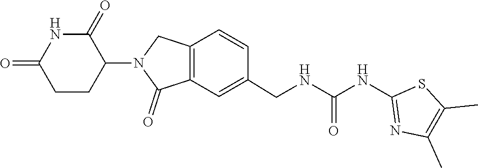 [SMILES: Cc1nc(NC(=O)NCc2ccc3c(c2)C(=O)N(C2CCC(=O)NC2=O)C3)sc1C] | 150 |
| 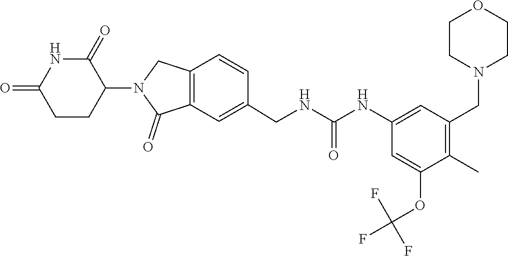 [SMILES: Cc1c(CN2CCOCC2)cc(NC(=O)NCc2ccc3c(c2)C(=O)N(C2CCC(=O)NC2=O)C3)cc1OC(F)(F)F] | 151 |
| 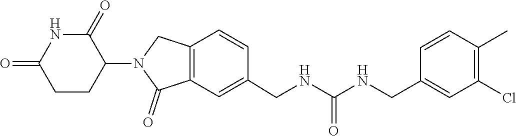 [SMILES: Cc1ccc(CNC(=O)NCc2ccc3c(c2)C(=O)N(C2CCC(=O)NC2=O)C3)cc1Cl] | 152 |
| 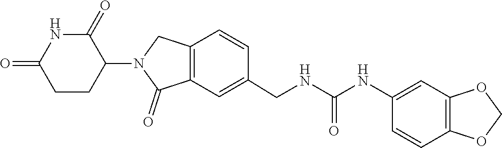 [SMILES: O=C1CCC(N2Cc3ccc(CNC(=O)Nc4ccc5c(c4)OCO5)cc3C2=O)C(=O)N1] | 153 |
| 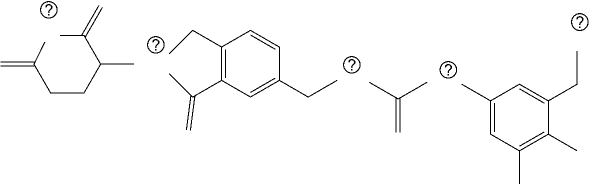 | 154 |
| 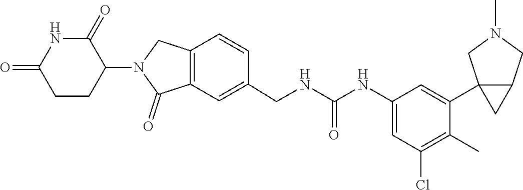 [SMILES: Cc1c(Cl)cc(NC(=O)NCc2ccc3c(c2)C(=O)N(C2CCC(=O)NC2=O)C3)cc1C12CC1CN(C)C2] | 155 |
| 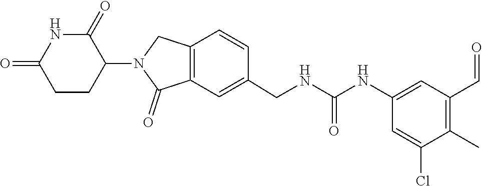 [SMILES: Cc1c(Cl)cc(NC(=O)NCc2ccc3c(c2)C(=O)N(C2CCC(=O)NC2=O)C3)cc1C=O] | 156 |
| 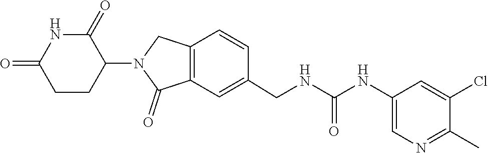 [SMILES: Cc1ncc(NC(=O)NCc2ccc3c(c2)C(=O)N(C2CCC(=O)NC2=O)C3)cc1Cl] | 157 |
| 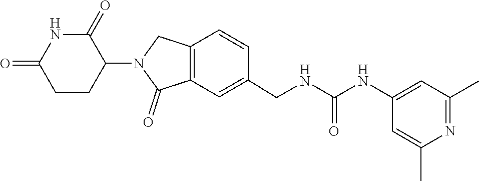 [SMILES: Cc1cc(NC(=O)NCc2ccc3c(c2)C(=O)N(C2CCC(=O)NC2=O)C3)cc(C)n1] | 158 |
| 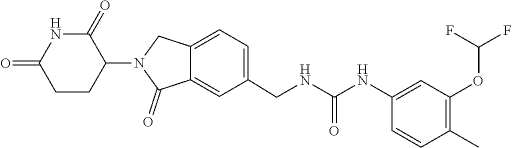 [SMILES: Cc1ccc(NC(=O)NCc2ccc3c(c2)C(=O)N(C2CCC(=O)NC2=O)C3)cc1OC(F)F] | 159 |
| 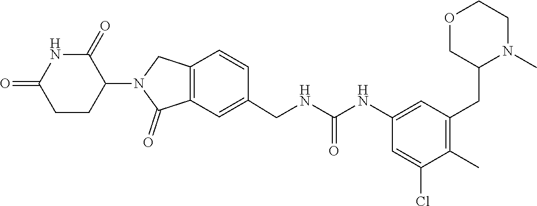 [SMILES: Cc1c(Cl)cc(NC(=O)NCc2ccc3c(c2)C(=O)N(C2CCC(=O)NC2=O)C3)cc1CC1COCCN1C] | 160 |
| 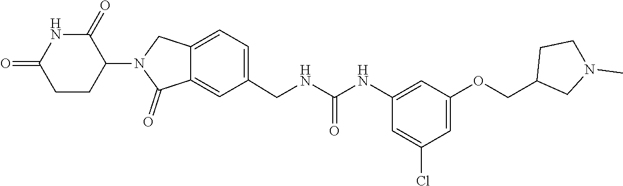 [SMILES: CN1CCC(COc2cc(Cl)cc(NC(=O)NCc3ccc4c(c3)C(=O)N(C3CCC(=O)NC3=O)C4)c2)C1] | 161 |
| 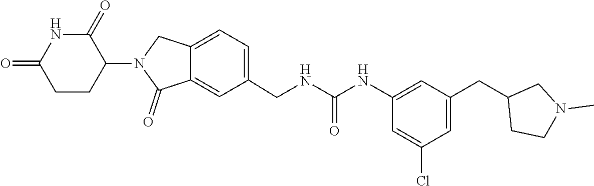 [SMILES: CN1CCC(Cc2cc(Cl)cc(NC(=O)NCc3ccc4c(c3)C(=O)N(C3CCC(=O)NC3=O)C4)c2)C1] | 162 |
| 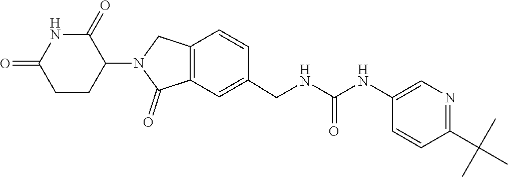 [SMILES: CC(C)(C)c1ccc(NC(=O)NCc2ccc3c(c2)C(=O)N(C2CCC(=O)NC2=O)C3)cn1] | 163 |
| 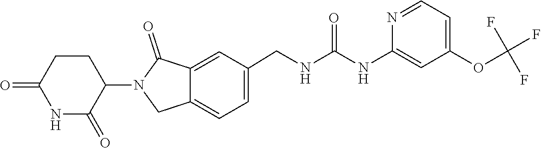 [SMILES: O=C1CCC(N2Cc3ccc(CNC(=O)Nc4cc(OC(F)(F)F)ccn4)cc3C2=O)C(=O)N1] | 164 |
| 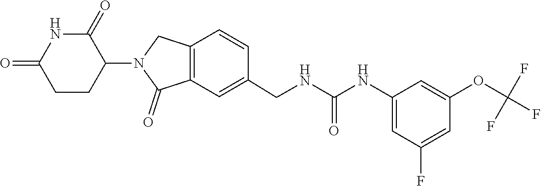 [SMILES: O=C1CCC(N2Cc3ccc(CNC(=O)Nc4cc(F)cc(OC(F)(F)F)c4)cc3C2=O)C(=O)N1] | 165 |

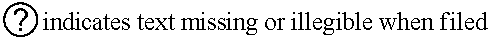

TABLE 5
Examples of E3 Ligase Binding Modulators

| Cmpd | | |
|---|---|---|
| No. | Structure | Compound Name |
| I-1 | 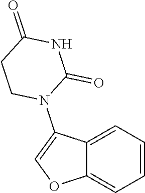 [SMILES: O=C1CCN(c2coc3ccccc23)C(=O)N1] | 1-(benzofuran-3-yl)dihydropyrimidine- 2,4(1H,3H)-dione; |
| I-2 | 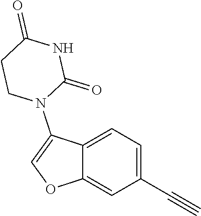 [SMILES: C#Cc1ccc2c(N3CCC(=O)NC3=O)coc2c1] | 1-(6-ethynylbenzofuran-3-yl)dihydro- pyrimidine-2,4(1H,3H)-dione; |
| I-3 | 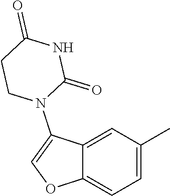 [SMILES: Cc1ccc2occ(N3CCC(=O)NC3=O)c2c1] | 1-(5-methylbenzofuran-3-yl)dihydro- pyrimidine-2,4(1H,3H)-dione; |
| I-4 | 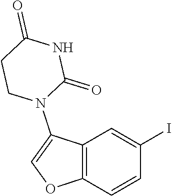 [SMILES: O=C1CCN(c2coc3ccc(I)cc23)C(=O)N1] | 1-(5-iodobenzofuran-3-yl)dihydro- pyrimidine-2,4(1H,3H)-dione; |
| I-5 | 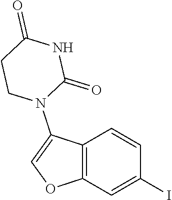 [SMILES: O=C1CCN(c2coc3cc(I)ccc23)C(=O)N1] | 1-(6-iodobenzofuran-3-yl)dihydro- pyrimidine-2,4(1H,3H)-dione; |
| I-6 | 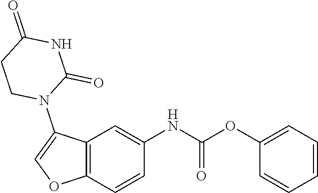 [SMILES: O=C1CCN(c2coc3ccc(NC(=O)Oc4ccccc4)cc23)C(=O)N1] | phenyl(3-(2,4-diosotetrahydropyrim- idin-1(2H)-yl)benzofuran-5-yl)- carbamate; |
| I-7 | 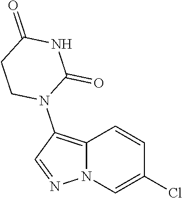 [SMILES: O=C1CCN(c2cnn3cc(Cl)ccc23)C(=O)N1] | 1-(6-chloropyrazolo[1,5-a]pyridin-3- yl)dihydropyrimidine-2,4(1H,3H)- dione |
| I-8 | 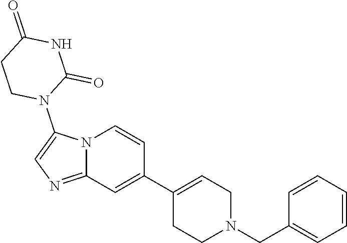 [SMILES: O=C1CCN(c2cnc3cc(C4=CCN(Cc5ccccc5)CC4)ccn23)C(=O)N1] | 1-(7-(1-benzyl-1,2,3,6-tetrahydropyri- din-4-yl)imidazo[1,2-a]pyridin-3-yl)- dihydropyrimidine-2,4(1H,3H)-dione; |
| I-9 | 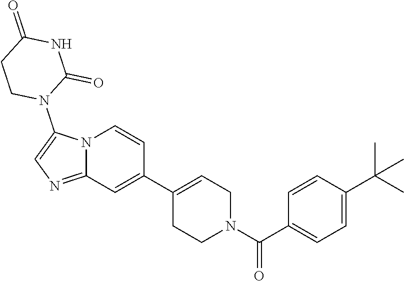 [SMILES: CC(C)(C)c1ccc(C(=O)N2CC=C(c3ccn4c(N5CCC(=O)NC5=O)cnc4c3)CC2)cc1] | 1-(7-(1-(4-tert-butyl)benzoyl)-1,2,3,6- tetrahydropyridin-4-yl)imidazo[1,2-a]- pyridin-3-yl)dihydropyrimidine- 2,4(1H,3H)-dione; and |
| I-10 | 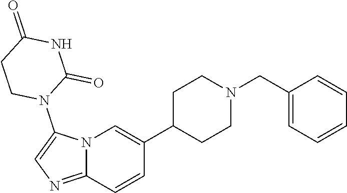 [SMILES: O=C1CCN(c2cnc3ccc(C4CCN(Cc5ccccc5)CC4)cn23)C(=O)N1] | 1-(6-(1-benzylpiperidin-4-yl)imidazol- [1,2-a]pyridin-3-yl)dihydropyrimidine- 2,4(1H,3H)-dione. |
| I-11 | 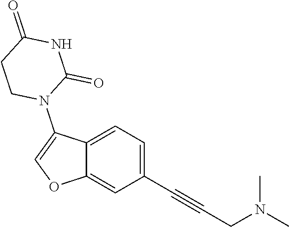 [SMILES: CN(C)CC#Cc1ccc2c(N3CCC(=O)NC3=O)coc2c1] | 1-(6-(3-(dimethylamino)prop-1-yn-1- yl)benzofuran-3-yl)dihydropyrimidine- 2,4(1H,3H)-dione; |
| I-12 | 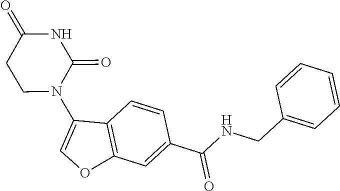 [SMILES: O=C1CCN(c2coc3cc(C(=O)NCc4ccccc4)ccc23)C(=O)N1] | N-benzyl-3-(2,4-dioxotetrahydropyrim- idin-1(2H)-yl)benzofuran-6-carbox- amide; |
| I-13 | 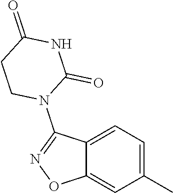 [SMILES: Cc1ccc2c(N3CCC(=O)NC3=O)noc2c1] | 1-(6-methylbenzo[d]isoxazol-3-yl)- dihydropyrimidine-2,4(1H,3H)-dione; |
| I-14 | 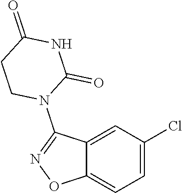 [SMILES: O=C1CCN(c2noc3ccc(Cl)cc23)C(=O)N1] | 1-(5-chlorobenzo[d]isoxazol-3-yl)- dihydropyrimidine-2,4(1H,3H)-dione; |
| I-15 | 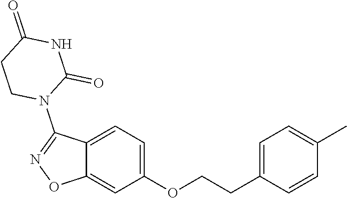 [SMILES: Cc1ccc(CCOc2ccc3c(N4CCC(=O)NC4=O)noc3c2)cc1] | 1-(6-(4-methylphenethoxy)benzo[d]- isoxazol-3-yl)dihydropyrimidine- 2,4(1H,3H)-dione; |
| I-16 | 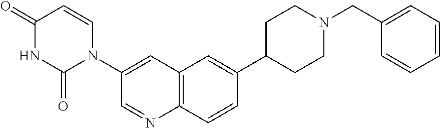 [SMILES: O=c1ccn(-c2cnc3ccc(C4CCN(Cc5ccccc5)CC4)cc3c2)c(=O)[nH]1] | 1-(6-(1-benzylpiperidin-3-yl)quinolin- 3-yl)pyrimidine-2,4(1H,3H)-dione; |
| I-17 | 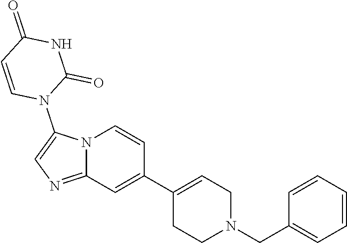 [SMILES: O=c1ccn(-c2cnc3cc(C4=CCN(Cc5ccccc5)CC4)ccn23)c(=O)[nH]1] | 1-(7-(1-benzyl-1,2,3,6-tetrahydropyri- din-4-yl)imidazo[1,2-a]pyridin-3-yl)- pyrimidine-2,4(1H,3H)-dione; and |
| I-18 | 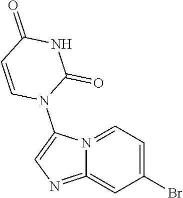 | 1-(7-bromoimidazol[1,2-a]pyridin-3- yl)pyrimidine-2,4(1H,3H)-dione. |

### E3 Ligase Binding Targets

The methods described herein are useful, for example, for identifying compound (e.g., drug)-dependent proximity interactions. In some embodiments, the methods are used to validate targets that selectively interact with an E3 ligase, e.g., cereblon, in the presence of a compound, e.g., an E3 ligase binding modulator, e.g., a cereblon binding modulator. The methods described herein are also useful, for example, for identifying E3 ligases that selectively interact with an E3 ligase binding target.

In some embodiments, the E3 ligase binding target is a protein comprising a structural feature on its surface that mediates its recruitment and degradation by an E3 ligase complex (i.e., a degron).

In some embodiments, the E3 ligase binding target is a protein comprising an E3 ligase-accessible loop, e.g., a cereblon-accessible loop, e.g., a G-loop.

In some embodiments, the E3 ligase binding target is a protein listed in Table 6 or a variant, derivative, ortholog, or homolog thereof.

TABLE 6
Examples of E3 Ligase Binding Targets

| Target | | |
|---|---|---|
| Protein | | |
| Symbol | Uniprot Name | Target Protein Name |
| A2M | A2MG_HUMAN | Alpha-2-macroglobulin |
| AADAT | AADAT_HUMAN | Kynurenine/alpha-aminoadipate aminotransferase, mitochondrial |
| AAKI | AAKI_HUMAN | AP2-associated protein kinase I |
| AAMDC | AAMDC_HUMAN | Mth938 domain-containing protein |
| AARS | SYAC_HUMAN | Alanine--tRNA ligase, cytoplasmic |
| AASDHPPT | ADPPT_HUMAN | L-aminoadipate-semialdehyde dehydrogenase-phosphopantetheiny |
| | | I transferase |
| AASS | AASS_HUMAN | Saccharopine dehydrogenase |
| ABLI | ABLI_HUMAN | Tyrosine-protein kinase ABL I |
| ABL2 | ABL2_HUMAN | Tyrosine-protein kinase ABL2 |
| ABLIM2 | ABLM2_HUMAN | Actin-binding LIM protein 2 |
| ACAAI | THIK_HUMAN | 3-ketoacyl-CoA thiolase, peroxisomal |
| ACAA2 | THIM_HUMAN | 3-ketoacyl-CoA thiolase, mitochondrial |
| ACACA | ACACA_HUMAN | Biotin carboxylase |
| ACACB | ACACB_HUMAN | Biotin carboxylase |
| ACADVL | ACADV_HUMAN | Very long-chain specific acyl-CoA dehydrogenase, mitochondrial |
| ACAPI | ACAPI_HUMAN | Arf-GAP with coiled-coil, ANK repeat and PH domain-containing protein |
| | | I |
| ACAP2 | ACAP2_HUMAN | Arf-GAP with coiled-coil, ANK repeat and PH domain-containing protein |
| | | 2 |
| ACAP3 | ACAP3_HUMAN | Arf-GAP with coiled-coil, ANK repeat and PH domain-containing protein |
| | | 3 |
| ACAT2 | THIC_HUMAN | Acety 1-CoA acety Itransferase, cytosolic |
| ACE | ACE_HUMAN | Angiotensin-converting enzyme, soluble form |
| ACHE | ACES_HUMAN | Acetylcholinesterase |
| ACLY | ACLY_HUMAN | ATP-citrate synthase |
| ACOI | ACOC_HUMAN | Cytoplasmic aconitate hydratase |
| ACOT12 | ACO12_HUMAN | Acetyl-coenzyme A thioesterase |
| ACOT13 | ACO13_HUMAN | Acyl-coenzyme A thioesterase 13, N-terminally processed |
| ACOT2 | ACOT2_HUMAN | Acyl-coenzyme A thioesterase 2, mitochondrial |
| ACOT4 | ACOT4_HUMAN | Peroxisomal succinyl-coenzyme A thioesterase |
| ACP5 | PPA5_HUMAN | Tartrate-resistant acid phosphatase type 5 |
| ACP6 | PPA6_HUMAN | Lysophosphatidic acid phosphatase type 6 |
| ACSM2A | ACS2A_HUMAN | Acyl-coenzyme A synthetase ACSM2A, mitochondrial |
| ACTB | ACTB_HUMAN | Actin, cytoplasmic 1, N-terminally processed |
| ACTGl | ACTG_HUMAN | Actin, cytoplasmic 2, N-terminally processed |
| ACVRl | ACVR1_HUMAN | Activin receptor type-1 |
| ACVRlB | ACV1B_HUMAN | Activin receptor type-1B |
| ACVR2A | AVR2A_HUMAN | Activin receptor type-2A |
| ACVR2B | AVR2B_HUMAN | Activin receptor type-2B |
| ACY1 | ACY1_HUMAN | Aminoacylase-1 |
| ADA2 | ADA2_HUMAN | Adenosine deaminase 2 |
| ADAM10 | ADA10_HUMAN | Disintegrin and metalloproteinase domain-containing protein 10 |
| ADAM17 | ADA17_HUMAN | Disintegrin and metalloproteinase domain-containing protein 17 |
| ADAP1 | ADAP1_HUMAN | Arf-GAP with dual PH domain-containing protein 1 |
| ADAP2 | ADAP2_HUMAN | Arf-GAP with dual PH domain-containing protein 2 |
| ADAR | DSRAD_HUMAN | Double-stranded RNA-specific adenosine deaminase |
| ADARB1 | RED1_HUMAN | Double-stranded RNA-specific editase 1 |
| ADCY10 | ADCYA_HUMAN | Adenylate cyclase type 10 |
| ADCYAP1R1 | PACR_HUMAN | Pituitary adenylate cyclase-activating polypeptide type I receptor |
| ADGRB3 | AGRB3_HUMAN | Adhesion G protein-coupled receptor B3 |
| ADGRL3 | AGRL3_HUMAN | Adhesion G protein-coupled receptor L3 |
| AD1POQ | AD1PO_HUMAN | Adiponectin |
| ADORA2A | AA2AR_HUMAN | Adenosine receptor A2a |
| ADRB2 | ADRB2_HUMAN | Beta-2 adrenergic receptor |
| ADRM1 | ADRM1_HUMAN | Proteasomal ubiquitin receptor ADRM1 |
| ADSS | PURA2_HUMAN | Adenylosuccinate synthetase isozyme 2 |
| AEBP2 | AEBP2_HUMAN | Zinc finger protein AEBP2 |
| AGA | ASPG_HUMAN | Glycosylasparaginase beta chain |
| AGAP2 | AGAP2_HUMAN | Arf-GAP with GTPase, ANK repeat and PH domain-containing protein 2 |
| AGER | RAGE_HUMAN | Advanced glycosylation end product-specific receptor |
| AGFG1 | AGFG1_HUMAN | Arf-GAP domain and FG repeat-containing protein 1 |
| AGO1 | AGO1_HUMAN | Protein argonaute-1 |
| AGO2 | AGO2_HUMAN | Protein argonaute-2 |
| AGO3 | AGO3_HUMAN | Protein argonaute-3 |
| AGRP | AGRP_HUMAN | Agouti-related protein |
| AGTR2 | AGTR2_HUMAN | Type-2 angiotensin II receptor |
| AGXT | SPYA_HUMAN | Serine--pyruvate aminotransferase |
| AHCY | SAHH_HUMAN | Adenosylhomocysteinase |
| AHCYL1 | SAHH2_HUMAN | S-adenosylhomocysteine hydrolase-like protein 1 |
| AHCYL2 | SAHH3_HUMAN | Adenosylhomocysteinase 3 |
| A1FM1 | A1FM1_HUMAN | Apoptosis-inducing factor 1, mitochondrial |
| A1M2 | AIM2_HUMAN | Interferon-inducible protein A1M2 |
| A1MP1 | A1MP1_HUMAN | Endothelial monocyte-activating polypeptide 2 |
| A1P | A1P_HUMAN | AH receptor-interacting protein |
| A1RE | A1RE_HUMAN | Autoimmune regulator |
| AK2 | KAD2_HUMAN | Adenylate kinase 2, mitochondrial, N-terminally processed |
| AK3 | KAD3_HUMAN | GTP:AMP phosphotransferase AK3, mitochondrial |
| AK4 | KAD4_HUMAN | Adenylate kinase 4, mitochondrial |
| AKAP13 | AKP13_HUMAN | A-kinase anchor protein 13 |
| AKR1A1 | AK1A1_HUMAN | Aldo-keto reductase family 1 member A1 |
| AKR1B1 | ALDR_HUMAN | Aldo-keto reductase family 1 member B1 |
| AKR1C1 | AK1C1_HUMAN | Aldo-keto reductase family 1 member C1 |
| AKR1C2 | AK1C2_HUMAN | Aldo-keto reductase family 1 member C2 |
| AKR1C3 | AK1C3_HUMAN | Aldo-keto reductase family 1 member C3 |
| AKT1 | AKT1_HUMAN | RAC-alpha serine/threonine-protein kinase |
| AKT2 | AKT2_HUMAN | RAC-beta serine/threonine-protein kinase |
| AKT3 | AKT3_HUMAN | RAC-gamma serine/threonine-protein kinase |
| ALAS2 | HEM0_HUMAN | 5-aminolevulinate synthase, erythroid-specific, mitochondrial |
| ALCAM | CD166_HUMAN | CD 166 antigen |
| ALDH1A2 | AL1A2_HUMAN | Retinal dehydrogenase 2 |
| ALDH1L1 | AL1L1_HUMAN | Cytosolic 10-formyltetrahydrofolate dehydrogenase |
| ALDH2 | ALDH2_HUMAN | Aldehyde dehydrogenase, mitochondrial |
| ALDH5A1 | SSDH_HUMAN | Succinate-semialdehyde dehydrogenase, mitochondrial |
| ALDH7A1 | AL7A1_HUMAN | Alpha-aminoadipic semialdehyde dehydrogenase |
| ALDOB | ALDOB_HUMAN | Fructose-bisphosphate aldolase B |
| ALK | ALK_HUMAN | ALK tyrosine kinase receptor |
| ALKBH8 | ALKB8_HUMAN | Alkylated DNA repair protein alkB homolog 8 |
| ALOX12 | LOX12_HUMAN | Arachidonate 12-lipoxygenase, 12S-type |
| ALOX15B | LX15B_HUMAN | Arachidonate 15-lipoxygenase B |
| ALOX5 | LOX5_HUMAN | Arachidonate 5-lipoxygenase |
| AMBP | AMBP_HUMAN | Trypstatin |
| AMD1 | DCAM_HUMAN | S-adenosylmethionine decarboxylase beta chain |
| AMFR | AMFR_HUMAN | E3 ubiquitin-protein ligase AMFR |
| AMT | GCST_HUMAN | Aminomethyltransferase, mitochondrial |
| AMY1A \| | AMY1_HUMAN | Alpha-amylase 1 |
| AMY1B \| | | |
| AMY1C | | |
| AMY2A | AMYP_HUMAN | Pancreatic alpha-amylase |
| ANAPC1 | APC1_HUMAN | Anaphase-promoting complex subunit 1 |
| ANAPC4 | APC4_HUMAN | Anaphase-promoting complex subunit 4 |
| ANGPT1 | ANGP1_HUMAN | Angiopoietin-1 |
| ANGPT2 | ANGP2_HUMAN | Angiopoietin-2 |
| ANGPTL3 | ANGL3_HUMAN | ANGPTL3(17-224) |
| ANGPTL4 | ANGL4_HUMAN | ANGPTL4 C-terminal chain |
| ANK1 | ANK1_HUMAN | Ankyrin-1 |
| ANK2 | ANK2_HUMAN | Ankyrin-2 |
| ANKFY1 | ANFY1_HUMAN | Rabankyrin-5 |
| ANKMY1 | ANKY1_HUMAN | Ankyrin repeat and MYND domain-containing protein 1 |
| ANKMY2 | ANKY2_HUMAN | Ankyrin repeat and MYND domain-containing protein 2 |
| ANKRA2 | ANRA2_HUMAN | Ankyrin repeat family A protein 2 |
| ANKRD27 | ANR27_HUMAN | Ankyrin repeat domain-containing protein 27 |
| ANLN | ANLN_HUMAN | Anillin |
| ANO10 | ANOl0_HUMAN | Anoctamin-10 |
| ANOS1 | KALM_HUMAN | Anosmin-1 |
| ANPEP | AMPN_HUMAN | Aminopeptidase N |
| ANTXR1 | ANTR1_HUMAN | Anthrax toxin receptor 1 |
| AOAH | AOAH_HUMAN | Acyloxyacyl hydrolase large subunit |
| AOC1 | AOC1_HUMAN | Amiloride-sensitive amine oxidase [copper containing] |
| AOC3 | AOC3_HUMAN | Membrane primary amine oxidase |
| AOX1 | AOXA_HUMAN | Aldehyde oxidase |
| AP1S3 | AP1S3_HUMAN | AP-1 complex subunit sigma-3 |
| AP2B1 | AP2B1_HUMAN | AP-2 complex subunit beta |
| AP4B1 | AP4B1_HUMAN | AP-4 complex subunit beta-1 |
| AP4M1 | AP4M1_HUMAN | AP-4 complex subunit mu-1 |
| APAF1 | APAF_HUMAN | Apoptotic protease-activating factor 1 |
| APBB1 | APBB1_HUMAN | Amyloid-beta A4 precursor protein-binding family B member 1 |
| APBB3 | APBB3_HUMAN | Amyloid-beta A4 precursor protein-binding family B member 3 |
| APCS | SAMP_HUMAN | Serum amyloid P-component(1-203) |
| APEX1 | APEX1_HUMAN | DNA-(apurinic or apyrimidinic site) lyase, mitochondrial |
| AP1P | MTNB_HUMAN | Methylthioribulose-1-phosphate dehydratase |
| APLF | APLF_HUMAN | Aprataxin and PNK-like factor |
| APLNR | APJ_HUMAN | Apelin receptor |
| APLP2 | APLP2_HUMAN | Amyloid-like protein 2 |
| APOBEC3A | ABC3A_HUMAN | DNA dC->dU-editing enzyme APOBEC-3A |
| APOD | APOD_HUMAN | Apolipoprotein D |
| APOH | APOH_HUMAN | Beta-2-glycoprotein 1 |
| APOM | APOM_HUMAN | Apolipoprotein M |
| APP | A4_HUMAN | C31 |
| APPL1 | DP13A_HUMAN | DCC-interacting protein 13-alpha |
| APRT | APT_HUMAN | Adenine phosphoribosyltransferase |
| APTX | APTX_HUMAN | Aprataxin |
| AQR | AQR_HUMAN | RNA helicase aquarius |
| AR | ANDR_HUMAN | Androgen receptor |
| ARAF | ARAF_HUMAN | Serine/threonine-protein kinase A-Raf |
| ARAP1 | ARAP1_HUMAN | Arf-GAP with Rho-GAP domain, ANK repeat and PH domain-containing |
| | | protein 1 |
| ARAP3 | ARAP3_HUMAN | Arf-GAP with Rho-GAP domain, ANK repeat and PH domain-containing |
| | | protein 3 |
| ARF1 | ARF1_HUMAN | ADP-ribosylation factor 1 |
| ARF6 | ARF6_HUMAN | ADP-ribosylation factor 6 |
| ARFGAP1 | ARFG1_HUMAN | ADP-ribosylation factor GTPase-activating protein 1 |
| ARFGAP2 | ARFG2_HUMAN | ADP-ribosylation factor GTPase-activating protein 2 |
| ARFGAP3 | ARFG3_HUMAN | ADP-ribosylation factor GTPase-activating protein 3 |
| ARHGAP10 | RHG10_HUMAN | Rho GTPase-activating protein 10 |
| ARHGAP11A | RHGBA_HUMAN | Rho GTPase-activating protein 11A |
| ARHGAP26 | RHG26_HUMAN | Rho GTPase-activating protein 26 |
| ARHGAP27 | RHG27_HUMAN | Rho GTPase-activating protein 27 |
| ARHGAP9 | RHG09_HUMAN | Rho GTPase-activating protein 9 |
| ARHGEF12 | ARHGC_HUMAN | Rho guanine nucleotide exchange factor 12 |
| ARHGEF16 | ARHGG_HUMAN | Rho guanine nucleotide exchange factor 16 |
| ARHGEF18 | ARHG1_HUMAN | Rho guanine nucleotide exchange factor 18 |
| ARHGEF2 | ARHG2_HUMAN | Rho guanine nucleotide exchange factor 2 |
| ARHGEF28 | ARG28_HUMAN | Rho guanine nucleotide exchange factor 28 |
| ARHGEF4 | ARHG4_HUMAN | Rho guanine nucleotide exchange factor 4 |
| AR1D4A | AR14A_HUMAN | AT-rich interactive domain-containing protein 4A |
| ARlH1 | ARl1_HUMAN | E3 ubiquitin-protein ligase ARlH1 |
| ARNT | ARNT_HUMAN | Aryl hydrocarbon receptor nuclear translocator |
| ARNTL2 | BMAL2_HUMAN | Ary I hydrocarbon receptor nuclear translocator like protein 2 |
| ARSB | ARSB_HUMAN | Arylsulfatase B |
| ASAH1 | ASAH1_HUMAN | Acid ceramidase subunit beta |
| ASAH2 | ASAH2_HUMAN | Neutral ceramidase soluble form |
| ASAP1 | ASAP1_HUMAN | Arf-GAP with SH3 domain, ANK repeat and PH domain-containing |
| | | protein 1 |
| ASAP3 | ASAP3_HUMAN | Arf-GAP with SH3 domain, ANK repeat and PH domain-containing |
| | | protein 3 |
| ASB11 | ASB11_HUMAN | Ankyrin repeat and SOCS box protein 11 |
| ASB9 | ASB9_HUMAN | Ankyrin repeat and SOCS box protein 9 |
| ASH1L | ASH1L_HUMAN | Histone-lysine N-methyltransferase ASH1L |
| ASH2L | ASH2L_HUMAN | Setl/Ash2 histone methyltransferase complex subunit ASH2 |
| ASPA | ACY2_HUMAN | Aspartoacylase |
| ASRGL1 | ASGL1_HUMAN | Isoaspartyl peptidase/L-asparaginase beta chain |
| ASS1 | ASSY_HUMAN | Argininosuccinate synthase |
| ASTN2 | ASTN2_HUMAN | Astrotactin-2 |
| ASXL1 | ASXL1_HUMAN | Putative Polycomb group protein ASXL1 |
| ASXL2 | ASXL2_HUMAN | Putative Polycomb group protein ASXL2 |
| ASXL3 | ASXL3_HUMAN | Putative Polycomb group protein ASXL3 |
| ATG101 | ATGA1_HUMAN | Autophagy-related protein 101 |
| ATG13 | ATG13_HUMAN | Autophagy-related protein 13 |
| ATG16L1 | Al6L1_HUMAN | Autophagy-related protein 16-1 |
| ATG5 | ATG5_HUMAN | Autophagy protein 5 |
| ATL1 | ATLA1_HUMAN | Atlastin-1 |
| ATL3 | ATLA3_HUMAN | Atlastin-3 |
| ATM | ATM_HUMAN | Serine-protein kinase ATM |
| ATP7A | ATP7A_HUMAN | Copper-transporting ATPase 1 |
| ATP7B | ATP7B_HUMAN | WND/140 kDa |
| ATR | ATR_HUMAN | Serine/threonine-protein kinase ATR |
| ATRX | ATRX_HUMAN | Transcriptional regulator ATRX |
| ATXN1 | ATX1_HUMAN | Ataxin-1 |
| AURKA | AURKA_HUMAN | Aurora kinase A |
| AXL | UFO_HUMAN | Tyrosine-protein kinase receptor UFO |
| AZGP1 | ZA2G_HUMAN | Zinc-alpha-2-glycoprotein |
| AZU1 | CAP7_HUMAN | Azurocidin |
| B2M | B2MG_HUMAN | Beta-2-microglobulin form pl 5.3 |
| B4GALT1 | B4GT1_HUMAN | Processed beta-1,4-galactosyltransferase 1 |
| BACE1 | BACE1_HUMAN | Beta-secretase 1 |
| BACE2 | BACE2_HUMAN | Beta-secretase 2 |
| BAK1 | BAK_HUMAN | Bcl-2 homologous antagonist/killer |
| BARD1 | BARD1_HUMAN | BRCA1-associated RING domain protein 1 |
| BAX | BAX_HUMAN | Apoptosis regulator BAX |
| BAZ2A | BAZ2A_HUMAN | Bromodomain adjacent to zinc finger domain protein 2A |
| BBS9 | PTHB1_HUMAN | Protein PTHB1 |
| BCAM | BCAM_HUMAN | Basal cell adhesion molecule |
| BCAT1 | BCAT1_HUMAN | Branched-chain-amino-acid aminotransferase, cytosolic |
| BCAT2 | BCAT2_HUMAN | Branched-chain-amino-acid aminotransferase, mitochondrial |
| BCHE | CHLE_HUMAN | Cholinesterase |
| BCL11A | BC11A_HUMAN | B-cell lymphoma/leukemia 11A |
| BCL11B | BC11B_HUMAN | B-cell lymphoma/leukemia 11B |
| BCL3 | BCL3_HUMAN | B-cell lymphoma 3 protein |
| BCL6 | BCL6_HUMAN | B-cell lymphoma 6 protein |
| BCL6B | BCL6B_HUMAN | B-cell CLL/lymphoma 6 member B protein |
| BCR | BCR_HUMAN | Breakpoint cluster region protein |
| BDNF | BDNF_HUMAN | Brain-derived neurotrophic factor |
| BECN1 | BECN1_HUMAN | Beclin-1-C 37 kDa |
| BHMT | BHMT1_HUMAN | Betaine--homocysteine S-methyltransferase 1 |
| BIRC2 | BIRC2_HUMAN | Baculoviral 1AP repeat-containing protein 2 |
| BIRC3 | BIRC3_HUMAN | Baculoviral 1AP repeat-containing protein 3 |
| BIRC6 | BIRC6_HUMAN | Baculoviral 1AP repeat-containing protein 6 |
| BIRC7 | BIRC7_HUMAN | Baculoviral 1AP repeat-containing protein 7 30 kDa subunit |
| BIRC8 | BIRC8_HUMAN | Baculoviral 1AP repeat-containing protein 8 |
| BLMH | BLMH_HUMAN | Bleomycin hydrolase |
| BM11 | BM11_HUMAN | Polycomb complex protein BMl-1 |
| BMP2K | BMP2K_HUMAN | BMP-2-inducible protein kinase |
| BMPR1A | BMR1A_HUMAN | Bone morphogenetic protein receptor type-1A |
| BMPR1B | BMR1B_HUMAN | Bone morphogenetic protein receptor type-1B |
| BMPR2 | BMPR2_HUMAN | Bone morphogenetic protein receptor type-2 |
| BMX | BMX_HUMAN | Cytoplasmic tyrosine-protein kinase BMX |
| BNC2 | BNC2_HUMAN | Zinc finger protein basonuclin-2 |
| BOC | BOC_HUMAN | Brother of CDO |
| BOLA3 | BOLA3_HUMAN | BolA-like protein 3 |
| BP1 | BP1_HUMAN | Bactericidal permeability-increasing protein |
| BPIFA1 | BP1A1_HUMAN | BPI fold-containing family A member 1 |
| BRAF | BRAF_HUMAN | Serine/threonine-protein kinase B-raf |
| BRAP | BRAP_HUMAN | BRCA1-associated protein |
| BRD1 | BRD1_HUMAN | Bromodomain-containing protein 1 |
| BRF1 | TF3B_HUMAN | Transcription factor IIIB 90 kDa subunit |
| BRF2 | BRF2_HUMAN | Transcription factor IIIB 50 kDa subunit |
| BROX | BROX_HUMAN | BRO 1 domain-containing protein BROX |
| BSG | BAS1_HUMAN | Basigin |
| BSN | BSN_HUMAN | Protein bassoon |
| BSPRY | BSPRY_HUMAN | B box and SPRY domain-containing protein |
| BTBD2 | BTBD2_HUMAN | BTB/POZ domain-containing protein 2 |
| BTG2 | BTG2_HUMAN | Protein BTG2 |
| BTK | BTK_HUMAN | Tyrosine-protein kinase BTK |
| BTN3A1 | BT3A1_HUMAN | Butyrophilin subfamily 3 member A1 |
| BTN3A2 | BT3A2_HUMAN | Butyrophilin subfamily 3 member A2 |
| BTN3A3 | BT3A3_HUMAN | Butyrophilin subfamily 3 member A3 |
| BTRC | FBW1A_HUMAN | F-box/WD repeat-containing protein IA |
| BUD31 | BUD31_HUMAN | Protein BUD31 homolog |
| C11orf54 | CK054_HUMAN | Ester hydrolase C11orf54 |
| C11orf68 | CK068_HUMAN | UPF0696 protein C11orf68 |
| C1QA | C1QA_HUMAN | Complement C1q subcomponent subunit A |
| C1QB | C1QB_HUMAN | Complement C1q subcomponent subunit B |
| C1QBP | C1QBP_HUMAN | Complement component 1 Q subcomponent binding protein, |
| | | mitochondrial |
| C1QC | C1QC_HUMAN | Complement C1q subcomponent subunit C |
| C1QTNF5 | C1QT5_HUMAN | Complement C1q tumor necrosis factor-related protein 5 |
| C1R | C1R_HUMAN | Complement C1r subcomponent light chain |
| C1S | C1S_HUMAN | Complement C1s subcomponent light chain |
| C2 | CO2_HUMAN | Complement C2a fragment |
| C2CD2L | C2C2L_HUMAN | Phospholipid transfer protein C2CD2L |
| C3 | CO3_HUMAN | Complement C3c alpha′ chain fragment 2 |
| C4A | CO4A_HUMAN | Complement C4 gamma chain |
| C4B | CO4B_HUMAN | Complement C4 gamma chain |
| C4B_2 | | |
| C4BPA | C4BPA_HUMAN | C4b-binding protein alpha chain |
| C5 | CO5_HUMAN | Complement C5 alpha′ chain |
| C6 | CO6_HUMAN | Complement component C6 |
| C7 | CO7_HUMAN | Complement component C7 |
| CSA | CO8A_HUMAN | Complement component C8 alpha chain |
| C8B | CO8B_HUMAN | Complement component C8 beta chain |
| C8G | CO8G_HUMAN | Complement component C8 gamma chain |
| C9 | CO9_HUMAN | Complement component C9b |
| CA2 | CAH2_HUMAN | Carbonic anhydrase 2 |
| CA6 | CAH6_HUMAN | Carbonic anhydrase 6 |
| CABP1 | CABP1_HUMAN | Calcium-binding protein 1 |
| CACNG2 | CCG2_HUMAN | Voltage-dependent calcium channel gamma-2 subunit |
| CALCOCO2 | CACO2_HUMAN | Calcium-binding and coiled-coil domain containing protein 2 |
| CALM1 | CALM1_HUMAN | Calmodulin-1 |
| CALM2 | CALM2_HUMAN | Calmodulin-2 |
| CAMK1D | KCC1D_HUMAN | Calcium/calmodulin-dependent protein kinase type 1D |
| CAMK1G | KCC1G_HUMAN | Calcium/calmodulin-dependent protein kinase type 1G |
| CAMK2A | KCC2A_HUMAN | Calcium/calmodulin-dependent protein kinase type II subunit alpha |
| CAMK2B | KCC2B_HUMAN | Calcium/calmodulin-dependent protein kinase type II subunit beta |
| CAMK2D | KCC2D_HUMAN | Calcium/calmodulin-dependent protein kinase type II subunit delta |
| CAMKK1 | KKCC1_HUMAN | Calcium/calmodulin-dependent protein kinase kinase 1 |
| CAMKK2 | KKCC2_HUMAN | Calcium/calmodulin-dependent protein kinase kinase 2 |
| CANT1 | CANT1_HUMAN | Soluble calcium-activated nucleotidase 1 |
| CAPN15 | CAN15_HUMAN | Calpain-15 |
| CAPN2 | CAN2_HUMAN | Calpain-2 catalytic subunit |
| CAPN9 | CAN9_HUMAN | Calpain-9 |
| CAPNS1 | CPNS1_HUMAN | Calpain small subunit 1 |
| CAPR1N2 | CAPR2_HUMAN | Caprin-2 |
| CARHSP1 | CHSP1_HUMAN | Calcium-regulated heat-stable protein 1 |
| CARM1 | CARM1_HUMAN | Histone-arginine methyltransferase CARM1 |
| CASK | CSKP_HUMAN | Peripheral plasma membrane protein CASK |
| CASP1 | CASP1_HUMAN | Caspase-1 subunit p10 |
| CASP2 | CASP2_HUMAN | Caspase-2 subunit p12 |
| CASP3 | CASP3_HUMAN | Caspase-3 subunit p12 |
| CASP6 | CASP6_HUMAN | Caspase-6 subunit p11 |
| CASP7 | CASP7_HUMAN | Caspase-7 subunit p11 |
| CASP8 | CASP8_HUMAN | Caspase-8 subunit p10 |
| CASP9 | CASP9_HUMAN | Caspase-9 subunit p10 |
| CASR | CASR_HUMAN | Extracellular calcium-sensing receptor |
| CAT | CATA_HUMAN | Catalase |
| CBFA2T2 | MTG8R_HUMAN | Protein CBF A2T2 |
| CBFA2T3 | MTG16_HUMAN | Protein CBF A2T3 |
| CBFB | PEBB_HUMAN | Core-binding factor subunit beta |
| CBL | CBL_HUMAN | E3 ubiquitin-protein ligase CBL |
| CBLB | CBLB_HUMAN | E3 ubiquitin-protein ligase CBL-B |
| CBLC | CBLC_HUMAN | E3 ubiquitin-protein ligase CBL-C |
| CBLL1 | HAKA1_HUMAN | E3 ubiquitin-protein ligase Hakai |
| CBS | CBS_HUMAN | Cystathionine beta-synthase |
| CCL13 | CCL13_HUMAN | C-C motif chemokine 13, short chain |
| CCL14 | CCL14_HUMAN | HCC-1(9-74) |
| CCL17 | CCL17_HUMAN | C-C motif chemokine 17 |
| CCL18 | CCL18_HUMAN | CCL18(4-69) |
| CCL19 | CCL19_HUMAN | C-C motif chemokine 19 |
| CCL23 | CCL23_HUMAN | CCL23(30-99) |
| CCL24 | CCL24_HUMAN | C-C motif chemokine 24 |
| CCL26 | CCL26_HUMAN | C-C motif chemokine 26 |
| CCL8 | CCL8_HUMAN | MCP-2(6-76) |
| CCNB11P1 | C1P1_HUMAN | E3 ubiquitin-protein ligase CCNB11P1 |
| CCNT2 | CCNT2_HUMAN | Cyclin-T2 |
| CCR2 | CCR2_HUMAN | C-C chemokine receptor type 2 |
| CCR5 | CCR5_HUMAN | C-C chemokine receptor type 5 |
| CCS | CCS_HUMAN | Copper chaperone for superoxide dismutase |
| CCT5 | TCPE_HUMAN | T-complex protein 1 subunit epsilon |
| CD19 | CD19_HUMAN | B-lymphocyte antigen CD19 |
| CD1A | CD1A_HUMAN | T-cell surface glycoprotein CD1a |
| CD1B | CD1B_HUMAN | T-cell surface glycoprotein CD1b |
| CD1C | CD1C_HUMAN | T-cell surface glycoprotein CD1c |
| CD1D | CD1D_HUMAN | Antigen-presenting glycoprotein CD1d |
| CD1E | CD1E_HUMAN | T-cell surface glycoprotein CD1e, soluble |
| CD2 | CD2_HUMAN | T-cell surface antigen CD2 |
| CD207 | CLC4K_HUMAN | C-type lectin domain family 4 member K |
| CD22 | CD22_HUMAN | B-cell receptor CD22 |
| CD226 | CD226_HUMAN | CD226 antigen |
| CD2AP | CD2AP_HUMAN | CD2-associated protein |
| CD302 | CD302_HUMAN | CD302 antigen |
| CD320 | CD320_HUMAN | CD320 antigen |
| CD33 | CD33_HUMAN | Myeloid cell surface antigen CD33 |
| CD36 | CD36_HUMAN | Platelet glycoprotein 4 |
| CD4 | CD4_HUMAN | T-cell surface glycoprotein CD4 |
| CD44 | CD44_HUMAN | CD44 antigen |
| CD48 | CD48_HUMAN | CD48 antigen |
| CD5 | CD5_HUMAN | T-cell surface glycoprotein CD5 |
| CD55 | DAF_HUMAN | Complement decay-accelerating factor |
| CD58 | LFA3_HUMAN | Lymphocyte function-associated antigen 3 |
| CD74 | HG2A_HUMAN | HLA class II histocompatibility antigen gamma chain |
| CD86 | CD86_HUMAN | T-lymphocyte activation antigen CD86 |
| CD96 | TACT_HUMAN | T-cell surface protein tactile |
| CDA | CDD_HUMAN | Cytidine deaminase |
| CDC20 | CDC20_HUMAN | Cell division cycle protein 20 homolog |
| CDC40 | PRP17_HUMAN | Pre-mRNA-processing factor 17 |
| CDC42BPA | MRCKA_HUMAN | Serine/threonine-protein kinase MRCK alpha |
| CDC42BPB | MRCKB_HUMAN | Serine/threonine-protein kinase MRCK beta |
| CDC42BPG | MRCKG_HUMAN | Serine/threonine-protein kinase MRCK gamma |
| CDC45 | CDC45_HUMAN | Cell division control protein 45 homolog |
| CDH1 | CADH1_HUMAN | E-Cad/CTF3 |
| CDH13 | CAD13_HUMAN | Cadherin-13 |
| CDH23 | CAD23_HUMAN | Cadherin-23 |
| CDH3 | CADH3_HUMAN | Cadherin-3 |
| CDHR2 | CDHR2_HUMAN | Cadherin-related family member 2 |
| CDK1 | CDK1_HUMAN | Cyclin-dependent kinase 1 |
| CDK12 | CDK12_HUMAN | Cyclin-dependent kinase 12 |
| CDK13 | CDK13_HUMAN | Cyclin-dependent kinase 13 |
| CDK16 | CDK16_HUMAN | Cyclin-dependent kinase 16 |
| CDK2 | CDK2_HUMAN | Cyclin-dependent kinase 2 |
| CDK4 | CDK4_HUMAN | Cyclin-dependent kinase 4 |
| CDK5 | CDK5_HUMAN | Cyclin-dependent-like kinase 5 |
| CDK6 | CDK6_HUMAN | Cyclin-dependent kinase 6 |
| CDK7 | CDK7_HUMAN | Cyclin-dependent kinase 7 |
| CDK9 | CDK9_HUMAN | Cyclin-dependent kinase 9 |
| CDKL1 | CDKL1_HUMAN | Cyclin-dependent kinase-like 1 |
| CDKL2 | CDKL2_HUMAN | Cyclin-dependent kinase-like 2 |
| CDKL3 | CDKL3_HUMAN | Cyclin-dependent kinase-like 3 |
| CDKN2A | CDN2A_HUMAN | Cyclin-dependent kinase inhibitor 2A |
| CDKN2C | CDN2C_HUMAN | Cyclin-dependent kinase 4 inhibitor C |
| CDKN2D | CDN2D_HUMAN | Cyclin-dependent kinase 4 inhibitor D |
| CDO1 | CDO1_HUMAN | Cysteine dioxygenase type 1 |
| CDYL | CDYL_HUMAN | Chromodomain Y-like protein |
| CDYL2 | CDYL2_HUMAN | Chromodomain Y-like protein 2 |
| CEACAM5 | CEAM5_HUMAN | Carcinoembryonic antigen-related cell adhesion molecule 5 |
| CEACAM7 | CEAM7_HUMAN | Carcinoembryonic antigen-related cell adhesion molecule 7 |
| CEBPA | CEBPA_HUMAN | CCAAT/enhancer-binding protein alpha |
| CEL | CEL_HUMAN | Bile salt-activated lipase |
| CELF6 | CELF6_HUMAN | CUGBP Elav-like family member 6 |
| CEP104 | CE104_HUMAN | Centrosomal protein of 104 kDa |
| CEP170 | CE170_HUMAN | Centrosomal protein of 170 kDa |
| CES1 | ESTl_HUMAN | Liver carboxy lesterase 1 |
| CETP | CETP_HUMAN | Cholesteryl ester transfer protein |
| CFB | CFAB_HUMAN | Complement factor B Bb fragment |
| CFD | CFAD_HUMAN | Complement factor D |
| CFH | CFAH_HUMAN | Complement factor H |
| CFl | CFA1_HUMAN | Complement factor l light chain |
| CFP | PROP_HUMAN | Properdin |
| CFTR | CFTR_HUMAN | Cystic fibrosis transmembrane conductance regulator |
| CGA | GLHA_HUMAN | Glycoprotein hormones alpha chain |
| CHAMP1 | CHAP1_HUMAN | Chromosome alignment-maintaining phosphoprotein 1 |
| CHD1 | CHD1_HUMAN | Chromodomain-helicase-DNA-binding protein 1 |
| CHD4 | CHD4_HUMAN | Chromodomain-helicase-DNA-binding protein 4 |
| CHD6 | CHD6_HUMAN | Chromodomain-helicase-DNA-binding protein 6 |
| CHD7 | CHD7_HUMAN | Chromodomain-helicase-DNA-binding protein 7 |
| CHD8 | CHD8_HUMAN | Chromodomain-helicase-DNA-binding protein 8 |
| CHEK1 | CHK1_HUMAN | Serine/threonine-protein kinase Chk1 |
| CHFR | CHFR_HUMAN | E3 ubiquitin-protein ligase CHFR |
| CH1D1 | CH1D1_HUMAN | Chitinase domain-containing protein 1 |
| CHN1 | CH1N_HUMAN | N-chimaerin |
| CHN2 | CH1O_HUMAN | Beta-chimaerin |
| CHRM1 | ACM1_HUMAN | Muscarinic acetylcholine receptor M1 |
| CHRNA1 | ACHA_HUMAN | Acetylcholine receptor subunit alpha |
| CHRNA2 | ACHA2_HUMAN | Neuronal acetylcholine receptor subunit alpha-2 |
| CHRNA3 | ACHA3_HUMAN | Neuronal acetylcholine receptor subunit alpha-3 |
| CHRNA4 | ACHA4_HUMAN | Neuronal acetylcholine receptor subunit alpha-4 |
| CHRNA7 | ACHA7_HUMAN | Neuronal acetylcholine receptor subunit alpha-7 |
| CHRNA9 | ACHA9_HUMAN | Neuronal acetylcholine receptor subunit alpha-9 |
| CHRNB2 | ACHB2_HUMAN | Neuronal acetylcholine receptor subunit beta-2 |
| CHUK | IKKA_HUMAN | Inhibitor of nuclear factor kappa-B kinase subunit alpha |
| C1AO1 | C1AO1_HUMAN | Probable cytosolic iron-sulfur protein assembly protein C1AO1 |
| C1DEA | C1DEA_HUMAN | Cell death activator C1DE-A |
| C1DEB | C1DEB_HUMAN | Cell death activator C1DE-B |
| CKB | KCRB_HUMAN | Creatine kinase B-type |
| CKM | KCRM_HUMAN | Creatine kinase M-type |
| CKMTlA | KCRU_HUMAN | Creatine kinase U-type, mitochondrial |
| CKMTlB | | |
| CKMT2 | KCRS_HUMAN | Creatine kinase S-type, mitochondrial |
| CLDN2 | CLD2_HUMAN | Claudin-2 |
| CLDN4 | CLD4_HUMAN | Claudin-4 |
| CLEC2A | CLC2A_HUMAN | C-type lectin domain family 2 member A |
| CLEC2D | CLC2D_HUMAN | C-type lectin domain family 2 member D |
| CLEC4D | CLC4D_HUMAN | C-type lectin domain family 4 member D |
| CLEC4E | CLC4E_HUMAN | C-type lectin domain family 4 member E |
| CLEC4M | CLC4M_HUMAN | C-type lectin domain family 4 member M |
| CLEC6A | CLC6A_HUMAN | C-type lectin domain family 6 member A |
| CLEC9A | CLC9A_HUMAN | C-type lectin domain family 9 member A |
| CLK1 | CLK1_HUMAN | Dual specificity protein kinase CLKl |
| CLK2 | CLK2_HUMAN | Dual specificity protein kinase CLK2 |
| CLK3 | CLK3_HUMAN | Dual specificity protein kinase CLK3 |
| CLPP | CLPP_HUMAN | ATP-dependent Clp protease proteolytic subunit, mitochondrial |
| CLPX | CLPX_HUMAN | ATP-dependent Clp protease ATP-binding subunit clpX-like, |
| | | mitochondrial |
| CLTC | CLH1_HUMAN | Clathrin heavy chain 1 |
| CMA1 | CMA1_HUMAN | Chymase |
| CNBP | CNBP_HUMAN | Cellular nucleic acid-binding protein |
| CNDP2 | CNDP2_HUMAN | Cytosolic non-specific dipeptidase |
| CNNM2 | CNNM2_HUMAN | Metal transporter CNNM2 |
| CNNM3 | CNNM3_HUMAN | Metal transporter CNNM3 |
| CNOT4 | CNOT4_HUMAN | CCR4-NOT transcription complex subunit 4 |
| CNOT7 | CNOT7_HUMAN | CCR4-NOT transcription complex subunit 7 |
| CNP | CN37_HUMAN | 2′,3′-cyclic-nucleotide 3′-phosphodiesterase |
| CNR2 | CNR2_HUMAN | Cannabinoid receptor 2 |
| CNTFR | CNTFR_HUMAN | Ciliary neurotrophic factor receptor subunit alpha |
| CNTN1 | CNTN1_HUMAN | Contactin-1 |
| CNTN2 | CNTN2_HUMAN | Contactin-2 |
| CNTN3 | CNTN3_HUMAN | Contactin-3 |
| CNTN5 | CNTN5_HUMAN | Contactin-5 |
| COL10A1 | COAA1_HUMAN | Collagen alpha- I(X) chain |
| COL1A1 | CO1A1_HUMAN | Collagen alpha-1(1) chain |
| COL20A1 | COKA1_HUMAN | Collagen alpha-1(XX) chain |
| COL3A1 | CO3A1_HUMAN | Collagen alpha-1(III) chain |
| COL4A1 | CO4A1_HUMAN | Arresten |
| COL4A2 | CO4A2_HUMAN | Canstatin |
| COL4A3 | CO4A3_HUMAN | Tnmstatin |
| COL4A4 | CO4A4_HUMAN | Collagen alpha-4(1V) chain |
| COL4A5 | CO4A5_HUMAN | Collagen alpha-5(1V) chain |
| COLEC11 | COL11_HUMAN | Collectin-11 |
| COLEC12 | COL12_HUMAN | Collectin-12 |
| COMP | COMP_HUMAN | Cartilage oligomeric matrix protein |
| COP1 | COP1_HUMAN | E3 ubiquitin-protein ligase COP1 |
| COPG1 | COPG1_HUMAN | Coatomer subunit gamma-1 |
| COPS3 | CSN3_HUMAN | COP9 signalosome complex subunit 3 |
| COPS4 | CSN4_HUMAN | COP9 signalosome complex subunit 4 |
| COQ8A | COQ8A_HUMAN | Atypical kinase COQ8A, mitochondrial |
| COX5B | COX5B_HUMAN | Cytochrome c oxidase subunit 5B, mitochondrial |
| CPA1 | CBPA1_HUMAN | Carboxypeptidase A1 |
| CPB1 | CBPB1_HUMAN | Carboxypeptidase B |
| CPD | CBPD_HUMAN | Carboxypeptidase D |
| CPM | CBPM_HUMAN | Carboxypeptidase M |
| CPN1 | CBPN_HUMAN | Carboxypeptidase N catalytic chain |
| CPOX | HEM6_HUMAN | Oxygen-dependent coproporphyrinogen-111 oxidase, mitochondrial |
| CPS1 | CPSM_HUMAN | Carbamoyl-phosphate synthase [ammonia], mitochondrial |
| CPSF1 | CPSF1_HUMAN | Cleavage and polyadenylation specificity factor subunit 1 |
| CPSF3 | CPSF3_HUMAN | Cleavage and polyadenylation specificity factor subunit 3 |
| CPSF4 | CPSF4_HUMAN | Cleavage and polyadenylation specificity factor subunit 4 |
| CPSF6 | CPSF6_HUMAN | Cleavage and polyadenylation specificity factor subunit 6 |
| CPSF7 | CPSF7_HUMAN | Cleavage and polyadenylation specificity factor subunit 7 |
| CR1 | CR1_HUMAN | Complement receptor type 1 |
| CR2 | CR2_HUMAN | Complement receptor type 2 |
| CRABP2 | RABP2_HUMAN | Cellular retinoic acid-binding protein 2 |
| CRBN | CRBN_HUMAN | Protein cereblon |
| CREBBP | CBP_HUMAN | CREB-binding protein |
| CRHR1 | CRFR1_HUMAN | Corticotropin-releasing factor receptor 1 |
| CRK | CRK_HUMAN | Adapter molecule erk |
| CRKL | CRKL_HUMAN | Crk-like protein |
| CRP | CRP_HUMAN | C-reactive protein(I-205) |
| CRTAM | CRTAM_HUMAN | Cytotoxic and regulatory T-cell molecule |
| CRYAB | CRYAB_HUMAN | Alpha-crystallin B chain |
| CRYM | CRYM_HUMAN | Ketimine reductase mu-crystallin |
| CS | C1SY_HUMAN | Citrate synthase, mitochondrial |
| CSAD | CSAD_HUMAN | Cysteine sulfinic acid decarboxylase |
| CSDE1 | CSDE1_HUMAN | Cold shock domain-containing protein E1 |
| CSF1R | CSF1R_HUMAN | Macrophage colony-stimulating factor 1 receptor |
| CSF3R | CSF3R_HUMAN | Granulocyte colony-stimulating factor receptor |
| CSK | CSK_HUMAN | Tyrosine-protein kinase CSK |
| CSNK1A1 | KC1A_HUMAN | Casein kinase 1 isoform alpha |
| CSNK1D | KC1D_HUMAN | Casein kinase 1 isoform delta |
| CSNK1E | KC1E_HUMAN | Casein kinase 1 isoform epsilon |
| CSNK1G3 | KC1G3_HUMAN | Casein kinase 1 isoform gamma-3 |
| CSRP3 | CSRP3_HUMAN | Cysteine and glycine-rich protein 3 |
| CST3 | CYTC_HUMAN | Cystatin-C |
| CSTF1 | CSTF1_HUMAN | Cleavage stimulation factor subunit 1 |
| CSTF2 | CSTF2_HUMAN | Cleavage stimulation factor subunit 2 |
| CTCF | CTCF_HUMAN | Transcriptional repressor CTCF |
| CTCFL | CTCFL_HUMAN | Transcriptional repressor CTCFL |
| CTLA4 | CTLA4_HUMAN | Cytotoxic T-lymphocyte protein 4 |
| CTPS1 | PYRG1_HUMAN | CTP synthase 1 |
| CTPS2 | PYRG2_HUMAN | CTP synthase 2 |
| CTRC | CTRC_HUMAN | Chymotrypsin-C |
| CTSA | PPGB_HUMAN | Lysosomal protective protein 20 kDa chain |
| CTSC | CATC_HUMAN | Dipeptidyl peptidase 1 light chain |
| CTSD | CATD_HUMAN | Cathepsin D heavy chain |
| CTSE | CATE_HUMAN | Cathepsin E form 11 |
| CUL4B | CUL4B_HUMAN | Cullin-4B |
| CUL5 | CUL5_HUMAN | Cullin-5 |
| CUL7 | CUL7_HUMAN | Cullin-7 |
| CUL9 | CUL9_HUMAN | Cullin-9 |
| CUTC | CUTC_HUMAN | Copper homeostasis protein cutC homolog |
| CWC27 | CWC27_HUMAN | Spliceosome-associated protein CWC27 homolog |
| CWF19L2 | C19L2_HUMAN | CWF19-like protein 2 |
| CXADR | CXAR_HUMAN | Coxsackievirus and adenovirus receptor |
| CXCL10 | CXL10_HUMAN | CXCL 10(I-73) |
| CXCL2 | CXCL2_HUMAN | GRO-beta(5-73) |
| CXCL5 | CXCL5_HUMAN | EN A-78(9-78) |
| CXCL8 | 1L8_HUMAN | lL-8(9-77) |
| CXCR4 | CXCR4_HUMAN | C-X-C chemokine receptor type 4 |
| CYC1 | CY1_HUMAN | Cytochrome cl, heme protein, mitochondrial |
| CYHR1 | CYHR1_HUMAN | Cysteine and histidine-rich protein 1 |
| CYLD | CYLD_HUMAN | Ubiquitin carboxyl-terminal hydrolase CYLD |
| CYP51A1 | CP51A_HUMAN | Lanosterol 14-alpha demethylase |
| CYP7A1 | CP7A1_HUMAN | Cholesterol 7-alpha-monooxygenase |
| CYTH3 | CYH3_HUMAN | Cytohesin-3 |
| CZ1B | CZ1B_HUMAN | CXXC motif containing zinc binding protein |
| DAG1 | DAG1_HUMAN | Beta-dystroglycan |
| DAPK1 | DAPK1_HUMAN | Death-associated protein kinase 1 |
| DAPK2 | DAPK2_HUMAN | Death-associated protein kinase 2 |
| DAPK3 | DAPK3_HUMAN | Death-associated protein kinase 3 |
| DARS2 | SYDM_HUMAN | Aspartate--tRNA ligase, mitochondrial |
| DAW1 | DAW1_HUMAN | Dynein assembly factor with WDR repeat domains 1 |
| DBH | DOPO_HUMAN | Soluble dopamine beta-hydroxylase |
| DBNL | DBNL_HUMAN | Drebrin-like protein |
| DCAF1 | DCAF1_HUMAN | DDB1- and CUL4-associated factor 1 |
| DCC | DCC_HUMAN | Netrin receptor DCC |
| DCDC2 | DCDC2_HUMAN | Doublecortin domain-containing protein 2 |
| DCLK1 | DCLK1_HUMAN | Serine/threonine-protein kinase DCLK1 |
| DCLRE1A | DCR1A_HUMAN | DNA cross-link repair 1A protein |
| DCLRE1B | DCR1B_HUMAN | 5′ exonuclease Apollo |
| DCTN1 | DCTN1_HUMAN | Dynactin subunit 1 |
| DCTN5 | DCTN5_HUMAN | Dynactin subunit 5 |
| DCUN1D1 | DCNL1_HUMAN | DCN1-like protein 1 |
| DCX | DCX_HUMAN | Neuronal migration protein doublecortin |
| DDAH1 | DDAH1_HUMAN | N(G),N(G)-dimethylarginine dimethylaminohydrolase 1 |
| DDB1 | DDB1_HUMAN | DNA damage-binding protein 1 |
| DDB2 | DDB2_HUMAN | DNA damage-binding protein 2 |
| DD11 | DD11_HUMAN | Protein DD11 homolog 1 |
| DD12 | DDl2_HUMAN | Protein DD11 homolog 2 |
| DDR1 | DDR1_HUMAN | Epithelial discoidin domain-containing receptor 1 |
| DDX1 | DDX1_HUMAN | ATP-dependent RNA helicase DDX1 |
| DDX39B | DX39B_HUMAN | Spliceosome RNA helicase DDX39B |
| DDX41 | DDX41_HUMAN | Probable ATP-dependent RNA helicase DDX41 |
| DDX58 | DDX58_HUMAN | Probable ATP-dependent RNA helicase DDX58 |
| DDX59 | DDX59_HUMAN | Probable ATP-dependent RNA helicase DDX59 |
| DEAF1 | DEAF1_HUMAN | Deformed epidermal autoregulatory factor 1 homolog |
| DEFA1 \| | DEF1_HUMAN | Neutrophil defensin 2 |
| DEFA1 B | | |
| DEFB4A \| | DFB4A_HUMAN | Beta-defensin 4A |
| DEFB4B | | |
| DES11 | DES11_HUMAN | Desumoylating isopeptidase 1 |
| DFFA | DFFA_HUMAN | DNA fragmentation factor subunit alpha |
| DFFB | DFFB_HUMAN | DNA fragmentation factor subunit beta |
| DGKE | DGKE_HUMAN | Diacylglycerol kinase epsilon |
| DGK1 | DGK1_HUMAN | Diacylglycerol kinase iota |
| DGKK | DGKK_HUMAN | Diacylglycerol kinase kappa |
| DGKQ | DGKQ_HUMAN | Diacylglycerol kinase theta |
| DGKZ | DGKZ_HUMAN | Diacylglycerol kinase zeta |
| DHFR | DYR_HUMAN | Dihydrofolate reductase |
| DHX16 | DHX16_HUMAN | Pre-mRNA-splicing factor ATP-dependent RNA helicase DHX16 |
| DHX58 | DHX58_HUMAN | Probable ATP-dependent RNA helicase DHX58 |
| DHX8 | DHX8_HUMAN | ATP-dependent RNA helicase DHX8 |
| DHX9 | DHX9_HUMAN | ATP-dependent RNA helicase A |
| DICER1 | DICER_HUMAN | Endoribonuclease Dicer |
| D1S3 | RRP44_HUMAN | Exosome complex exonuclease RRP44 |
| D1XDC1 | D1XC1_HUMAN | Dixin |
| DLAT | ODP2_HUMAN | Dihydrolipoyllysine-residue acetyltransferase component of pyruvate |
| | | dehydrogenase complex, mitochondrial |
| DLD | DLDH_HUMAN | Dihydrolipoyl dehydrogenase, mitochondrial |
| DLG5 | DLG5_HUMAN | Disks large homolog 5 |
| DLL1 | DLL1_HUMAN | Delta-like protein 1 |
| DLL4 | DLL4_HUMAN | Delta-like protein 4 |
| DMC1 | DMC1_HUMAN | Meiotic recombination protein DMC1/LIM15 homolog |
| DMGDH | M2GD_HUMAN | Dimethylglycine dehydrogenase, mitochondrial |
| DMPK | DMPK_HUMAN | Myotonin-protein kinase |
| DNAJA1 | DNJA1_HUMAN | DnaJ homolog subfamily A member 1 |
| DNAJA3 | DNJA3_HUMANV | DnaJ homolog subfamily A member 3, mitochondrial |
| DNAJB1 | DNJB1_HUMAN | DnaJ homolog subfamily B member 1 |
| DNAJC24 | DJC24_HUMAN | DnaJ homolog subfamily C member 24 |
| DNLZ | DNLZ_HUMAN | DNL-type zinc finger protein |
| DNMT1 | DNMT1_HUMAN | DNA (cytosine-5)-methyltransferase 1 |
| DNMT3A | DNM3A_HUMAN | DNA (cytosine-5)-methyltransferase 3A |
| DNMT3B | DNM3B_HUMAN | DNA (cytosine-5)-methyltransferase 3B |
| DNMT3L | DNM3L_HUMAN | DNA (cytosine-5)-methyltransferase 3-like |
| DNPEP | DNPEP_HUMAN | Aspartyl aminopeptidase |
| DOK2 | DOK2_HUMAN | Docking protein 2 |
| DPAGT1 | GPT_HUMAN | UDP-N-acetylglucosamine--dolichyl-phosphate N- |
| | | acetylglucosaminephosphotransferase |
| DPF1 | DPF1_HUMAN | Zinc finger protein neuro-d4 |
| DPF2 | REQU_HUMAN | Zinc finger protein ubi-d4 |
| DPF3 | DPF3_HUMAN | Zinc finger protein DPF3 |
| DPP10 | DPP10_HUMAN | Inactive dipeptidyl peptidase 10 |
| DPP3 | DPP3_HUMAN | Dipeptidyl peptidase 3 |
| DPP4 | DPP4_HUMAN | Dipeptidyl peptidase 4 soluble form |
| DPP6 | DPP6_HUMAN | Dipeptidyl aminopeptidase-like protein 6 |
| DPP8 | DPP8_HUMAN | Dipeptidyl peptidase 8 |
| DPP9 | DPP9_HUMAN | Dipeptidyl peptidase 9 |
| DRD2 | DRD2_HUMAN | D(2) dopamine receptor |
| DRD3 | DRD3_HUMAN | D(3) dopamine receptor |
| DROSHA | RNC_HUMAN | Ribonuclease 3 |
| DSC1 | DSC1_HUMAN | Desmocollin-1 |
| DSC2 | DSC2_HUMAN | Desmocollin-2 |
| DSG2 | DSG2_HUMAN | Desmoglein-2 |
| DSG3 | DSG3_HUMAN | Desmoglein-3 |
| DSP | DESP_HUMAN | Desmoplakin |
| DTD1 | DTD1_HUMAN | D-aminoacyl-tRNA deacylase 1 |
| DTX3 | DTX3_HUMAN | Probable E3 ubiquitin-protein ligase DTX3 |
| DTX3L | DTX3L_HUMAN | E3 ubiquitin-protein ligase DTX3L |
| DUSP14 | DUS14_HUMAN | Dual specificity protein phosphatase 14 |
| DVL2 | DVL2_HUMAN | Segment polarity protein dishevelled homolog DVL-2 |
| DYNC1H1 | DYHC1_HUMAN | Cytoplasmic dynein 1 heavy chain 1 |
| DYNC112 | DC112_HUMAN | Cytoplasmic dynein 1 intermediate chain 2 |
| DYNC2H1 | DYHC2_HUMAN | Cytoplasmic dynein 2 heavy chain 1 |
| DYNLRB1 | DLRB1_HUMAN | Dynein light chain roadblock-type 1 |
| DYRK1A | DYR1A_HUMAN | Dual specificity tyrosine-phosphorylation regulated-kinase 1A |
| DYRK2 | DYRK2_HUMAN | Dual specificity tyrosine-phosphorylation-regulated kinase 2 |
| DYRK3 | DYRK3_HUMAN | Dual specificity tyrosine-phosphorylation-regulated kinase 3 |
| DYSF | DYSF_HUMAN | Dysferlin |
| DZANK1 | DZAN1_HUMAN | Double zinc ribbon and ankyrin repeat-containing protein 1 |
| E4F1 | E4F1_HUMAN | Transcription factor E4F1 |
| EBF1 | COE1_HUMAN | Transcription factor COE1 |
| ECE1 | ECE1_HUMAN | Endothelin-converting enzyme 1 |
| EC11 | EC11_HUMAN | Enoyl-CoA delta isomerase 1, mitochondrial |
| EDA | EDA_HUMAN | Ectodysplasin-A, secreted form |
| EDC3 | EDC3_HUMAN | Enhancer of mRNA-decapping protein 3 |
| EDNRB | EDNRB_HUMAN | Endothelin receptor type B |
| EEA1 | EEA1_HUMAN | Early endosome antigen 1 |
| EED | EED_HUMAN | Polycomb protein EED |
| EEF1G | EF1G_HUMAN | Elongation factor 1-gamma |
| EEFSEC | SELB_HUMAN | Selenocysteine-specific elongation factor |
| EFEMP2 | FBLN4_HUMAN | EGF-containing fibulin-like extracellular matrix protein 2 |
| EFL1 | EFL1_HUMAN | Elongation factor-like GTPase 1 |
| EFTUD2 | U5S1_HUMAN | 116 kDa U5 small nuclear ribonucleoprotein component |
| EGFR | EGFR_HUMAN | Epidermal growth factor receptor |
| EGLN1 | EGLN1_HUMAN | Egl nine homolog 1 |
| EGR1 | EGR1_HUMAN | Early growth response protein 1 |
| EGR2 | EGR2_HUMAN | E3 SUMO-protein ligase EGR2 |
| EGR3 | EGR3_HUMAN | Early growth response protein 3 |
| EGR4 | EGR4_HUMAN | Early growth response protein 4 |
| EHMT1 | EHMT1_HUMAN | Histone-lysine N-methyltransferase EHMT1 |
| EHMT2 | EHMT2_HUMAN | Histone-lysine N-methyltransferase EHMT2 |
| E1F1 | E1F1_HUMAN | Eukaryotic translation initiation factor 1 |
| E1F1AD | E1F1A_HUMAN | Probable RNA-binding protein E1F1AD |
| E1F2AK2 | E2AK2_HUMAN | Interferon-induced, double-stranded RNA-activated protein kinase |
| E1F2AK3 | E2AK3_HUMAN | Eukaryotic translation initiation factor 2-alpha kinase 3 |
| E1F2B1 | E12BA_HUMAN | Translation initiation factor e1F-2B subunit alpha |
| E1F2B2 | E12BB_HUMAN | Translation initiation factor e1F-2B subunit beta |
| E1F2B4 | E12BD_HUMAN | Translation initiation factor e1F-2B subunit delta |
| E1F2D | E1F2D_HUMAN | Eukaryotic translation initiation factor 2D |
| E1F2S1 | 1F2A_HUMAN | Eukaryotic translation initiation factor 2 subunit 1 |
| E1F3B | E1F3B_HUMAN | Eukaryotic translation initiation factor 3 subunit B |
| E1F3E | E1F3E_HUMAN | Eukaryotic translation initiation factor 3 subunit E |
| E1F3G | E1F3G_HUMAN | Eukaryotic translation initiation factor 3 subunit G |
| E1F4EBP2 | 4EBP2_HUMAN | Eukaryotic translation initiation factor 4E-binding protein 2 |
| E1F4G1 | IF4G1_HUMAN | Eukaryotic translation initiation factor 4 gamma 1 |
| E1F5 | 1FS_HUMAN | Eukaryotic translation initiation factor 5 |
| E1F5A | 1F5A1_HUMAN | Eukaryotic translation initiation factor 5A-1 |
| ELAC1 | RNZ1_HUMAN | Zinc phosphodiesterase ELAC protein 1 |
| ELAVL1 | ELAV1_HUMAN | ELA V-like protein 1 |
| ELAVL4 | ELAV4_HUMAN | ELA V-like protein 4 |
| ELF5 | ELF5_HUMAN | ETS-related transcription factor Elf-5 |
| ELK1 | ELK1_HUMAN | ETS domain-containing protein Elk-1 |
| ELK4 | ELK4_HUMAN | ETS domain-containing protein Elk-4 |
| ELL | ELL_HUMAN | RNA polymerase II elongation factor ELL |
| ELOC | ELOC_HUMAN | Elongin-C |
| EM1L1N1 | EM1L1_HUMAN | EMILIN-1 |
| EML1 | EMAL1_HUMAN | Echinoderm rnicrotubule-associated protein-like 1 |
| ENO1 | ENOA_HUMAN | Alpha-enolase |
| ENO2 | ENOG_HUMAN | Gamma-enolase |
| ENO3 | ENOB_HUMAN | Beta-enolase |
| ENPEP | AMPE_HUMAN | Glutamyl arninopeptidase |
| EP300 | EP300_HUMAN | Histone acetyltransferase p300 |
| EPAS1 | EPAS1_HUMAN | Endothelial PAS domain-containing protein 1 |
| EPB41 | 41_HUMAN | Protein 4.1 |
| EPB41L3 | E41L3_HUMAN | Band 4.1-like protein 3, N-terminally processed |
| EPCAM | EPCAM_HUMAN | Epithelial cell adhesion molecule |
| EPDR1 | EPDR1_HUMAN | Mammalian ependymin-related protein 1 |
| EPHA2 | EPHA2_HUMAN | Ephrin type-A receptor 2 |
| EPHA3 | EPHA3_HUMAN | Ephrin type-A receptor 3 |
| EPHA4 | EPHA4_HUMAN | Ephrin type-A receptor 4 |
| EPHA5 | EPHA5_HUMAN | Ephrin type-A receptor 5 |
| EPHB4 | EPHB4_HUMAN | Ephrin type-B receptor 4 |
| EPM2A | EPM2A_HUMAN | Laforin |
| EPOR | EPOR_HUMAN | Erythropoietin receptor |
| EPRS | SYEP_HUMAN | Proline--tRNA ligase |
| EPS8L1 | ES8L1_HUMAN | Epidermal growth factor receptor kinase substrate 8-like protein 1 |
| EPS8L2 | ES8L2_HUMAN | Epidermal growth factor receptor kinase substrate 8-like protein 2 |
| EPS8L3 | ES8L3_HUMAN | Epidermal growth factor receptor kinase substrate 8-like protein 3 |
| ERAP1 | ERAP1_HUMAN | Endoplasmic reticulum aminopeptidase 1 |
| ERAP2 | ERAP2_HUMAN | Endoplasmic reticulum aminopeptidase 2 |
| ERBB2 | ERBB2_HUMAN | Receptor tyrosine-protein kinase erbB-2 |
| ERBB3 | ERBB3_HUMAN | Receptor tyrosine-protein kinase erbB-3 |
| ERCC6L2 | ER6L2_HUMAN | DNA excision repair protein ERCC-6-like 2 |
| ERCC8 | ERCC8_HUMAN | DNA excision repair protein ERCC-8 |
| ERG | ERG_HUMAN | Transcriptional regulator ERG |
| ERN1 | ERN1_HUMAN | Endoribonuclease |
| ERVK-10 | GAK10_HUMAN | Endogenous retrovirus group K member 10 Gag polyprotein |
| ERVK-19 | GAK19_HUMAN | Endogenous retrovirus group K member 19 Gag polyprotein |
| ERVK-21 | GAK21_HUMAN | Endogenous retrovirus group K member 21 Gag polyprotein |
| ERVK-24 | GAK24_HUMAN | Endogenous retrovirus group K member 24 Gag polyprotein |
| ERVK-5 | GAK5_HUMAN | Endogenous retrovirus group K member 5 Gag polyprotein |
| ERVK-6 | GAK5_HUMAN | Endogenous retrovirus group K member 6 Gag polyprotein |
| ERVK-7 | GAK7_HUMAN | Endogenous retrovirus group K member 7 Gag polyprotein |
| ERVK-8 | GAK8_HUMAN | Endogenous retrovirus group K member 8 Gag polyprotein |
| ERVK-9 | POK9_HUMAN | Reverse transcriptase/ribonuclease H |
| ERVK-9 | GAK9_HUMAN | Endogenous retrovirus group K member 9 Gag polyprotein |
| ESCO1 | ESCO1_HUMAN | N-acetyltransferase ESCO1 |
| ESCO2 | ESCO2_HUMAN | N-acetyltransferase ESCO2 |
| ESRRA | ERR1_HUMAN | Steroid hormone receptor ERR1 |
| ESRRB | ERR2_HUMAN | Steroid hormone receptor ERR2 |
| ESRRG | ERR3_HUMAN | Estrogen-related receptor gamma |
| ETF1 | ERF1_HUMAN | Eukaryotic peptide chain release factor subunit 1 |
| ETFB | ETFB_HUMAN | Electron transfer flavoprotein subunit beta |
| EVPL | EVPL_HUMAN | Envoplakin |
| EWSR1 | EWS_HUMAN | RNA-binding protein EWS |
| EXO1 | EXO1_HUMAN | Exonuclease 1 |
| EXOG | EXOG_HUMAN | Nuclease EXOG, mitochondrial |
| EXOSC2 | EXOS2_HUMAN | Exosome complex component RRP4 |
| EXOSC4 | EXOS4_HUMAN | Exosome complex component RRP41 |
| EXOSC5 | EXOS5_HUMAN | Exosome complex component RRP46 |
| EXOSC7 | EXOS7_HUMAN | Exosome complex component RRP42 |
| EXOSC9 | EXOS9_HUMAN | Exosome complex component RRP45 |
| EZH2 | EZH2_HUMAN | Histone-lysine N-methyltransferase EZH2 |
| EZR | EZR1_HUMAN | Ezrin |
| F10 | FA10_HUMAN | Activated factor Xa heavy chain |
| F11 | FA11_HUMAN | Coagulation factor X1a light chain |
| F11R | JAM1_HUMAN | Junctional adhesion molecule A |
| F12 | FA12_HUMAN | Coagulation factor XIIa light chain |
| F13A1 | Fl3A_HUMAN | Coagulation factor XIII A chain |
| F2 | THRB_HUMAN | Thrombin heavy chain |
| F2R | PAR1_HUMAN | Proteinase-activated receptor 1 |
| F2RL1 | PAR2_HUMAN | Proteinase-activated receptor 2, alternate cleaved 2 |
| F3 | TF_HUMAN | Tissue factor |
| F5 | FA5_HUMAN | Coagulation factor V light chain |
| F7 | FA7_HUMAN | Factor VII heavy chain |
| F8 | FA8_HUMAN | Factor VIIa light chain |
| F9 | FA9_HUMAN | Coagulation factor IXa heavy chain |
| FABP1 | FABPL_HUMAN | Fatty acid-binding protein, liver |
| FABP2 | FABPI_HUMAN | Fatty acid-binding protein, intestinal |
| FABP5 | FABP5_HUMAN | Fatty acid-binding protein 5 |
| FABP6 | FABP6_HUMAN | Gastrotropin |
| FAF1 | FAF1_HUMAN | FAS-associated factor 1 |
| FAIM | FAIM1_HUMAN | Fas apoptotic inhibitory molecule 1 |
| FAM3C | FAM3C_HUMAN | Protein FAM3C |
| FAM83A | FA83A_HUMAN | Protein FAM83A |
| FAM83B | FA83B_HUMAN | Protein FAM83B |
| FAN1 | FAN1_HUMAN | Fanconi-associated nuclease 1 |
| FANCF | FANCF_HUMAN | Fanconi anemia group F protein |
| FANCL | FANCL_HUMAN | E3 ubiquitin-protein ligase FANCL |
| FAP | SEPR_HUMAN | Antiplasmin-cleaving enzyme F AP, soluble form |
| FARSB | SYFB_HUMAN | Phenylalanine--tRNA ligase beta subunit |
| FASN | FAS_HUMAN | Oleoyl-[acyl-carrier-protein] hydrolase |
| FBL | FBRL_HUMAN | rRNA 2′-0-methyltransferase fibrillarin |
| FBN1 | FBN1_HUMAN | Asprosin |
| FBP1 | F16P1_HUMAN | Fmctose-1,6-bisphosphatase 1 |
| FBP2 | F16P2_HUMAN | Fmctose-1,6-bisphosphatase isozyme 2 |
| FBXL19 | FXL19_HUMAN | F-box/LRR-repeat protein 19 |
| FBX03 | FBX3_HUMAN | F-box only protein 3 |
| FBX031 | FBX31_HUMAN | F-box only protein 31 |
| FBX043 | FBX43_HUMAN | F-box only protein 43 |
| FBXW7 | FBXW7_HUMAN | F-box/WD repeat-containing protein 7 |
| FCER2 | FCER2_HUMAN | Low affinity immunoglobulin epsilon Fe receptor soluble form |
| FCGRT | FCGRN_HUMAN | IgG receptor FcRn large subunit p51 |
| FCHSD2 | FCSD2_HUMAN | F-BAR and double SH3 domains protein 2 |
| FCN1 | FCN1_HUMAN | Ficolin-1 |
| FCN3 | FCN3_HUMAN | Ficolin-3 |
| FDX1 | ADX_HUMAN | Adrenodoxin, mitochondrial |
| FDX2 | FDX2_HUMAN | Ferredoxin-2, mitochondrial |
| FEN1 | FEN1_HUMAN | Flap endonuclease 1 |
| FER | FER_HUMAN | Tyrosine-protein kinase Fer |
| FES | FES_HUMAN | Tyrosine-protein kinase Fes/Fps |
| FEV | FEV_HUMAN | Protein FEV |
| FEZF1 | FEZF1_HUMAN | Fez family zinc finger protein 1 |
| FEZF2 | FEZF2_HUMAN | Fez family zinc finger protein 2 |
| FFAR1 | FFAR1_HUMAN | Free fatty acid receptor 1 |
| FGA | FIBA_HUMAN | Fibrinogen alpha chain |
| FGB | FIBB_HUMAN | Fibrinogen beta chain |
| FGD1 | FGD1_HUMAN | FYVE, RhoGEF and PH domain-containing protein 1 |
| FGD2 | FGD2_HUMAN | FYVE, RhoGEF and PH domain-containing protein 2 |
| FGD3 | FGD3_HUMAN | FYVE, RhoGEF and PH domain-containing protein 3 |
| FGD4 | FGD4_HUMAN | FYVE, RhoGEF and PH domain-containing protein 4 |
| FGD5 | FGD5_HUMAN | FYVE, RhoGEF and PH domain-containing protein 5 |
| FGD6 | FGD6_HUMAN | FYVE, RhoGEF and PH domain-containing protein 6 |
| FGF1 | FGF1_HUMAN | Fibroblast growth factor 1 |
| FGF10 | FGF10_HUMAN | Fibroblast growth factor 10 |
| FGF12 | FGF12_HUMAN | Fibroblast growth factor 12 |
| FGF13 | FGF13_HUMAN | Fibroblast growth factor 13 |
| FGF18 | FGF18_HUMAN | Fibroblast growth factor 18 |
| FGF19 | FGF19_HUMAN | Fibroblast growth factor 19 |
| FGF2 | FGF2_HUMAN | Fibroblast growth factor 2 |
| FGF20 | FGF20_HUMAN | Fibroblast growth factor 20 |
| FGF23 | FGF23_HUMAN | Fibroblast growth factor 23 C-terminal peptide |
| FGF4 | FGF4_HUMAN | Fibroblast growth factor 4 |
| FGF8 | FGF8_HUMAN | Fibroblast growth factor 8 |
| FGF9 | FGF9_HUMAN | Fibroblast growth factor 9 |
| FGFR1 | FGFR1_HUMAN | Fibroblast growth factor receptor 1 |
| FGFR2 | FGFR2_HUMAN | Fibroblast growth factor receptor 2 |
| FGFR3 | FGFR3_HUMAN | Fibroblast growth factor receptor 3 |
| FGFR4 | FGFR4_HUMAN | Fibroblast growth factor receptor 4 |
| FGG | FIBG_HUMAN | Fibrinogen gamma chain |
| FH | FUMH_HUMAN | Fumarate hydratase, mitochondrial |
| FHL2 | FHL2_HUMAN | Four and a half LIM domains protein 2 |
| FHL3 | FHL3_HUMAN | Four and a half LIM domains protein 3 |
| FHOD1 | FHOD1_HUMAN | FH1/FH2 domain-containing protein 1 |
| FIBCD1 | FBCD1_HUMAN | Fibrinogen C domain-containing protein 1 |
| FIZ1 | FIZ1_HUMAN | Flt3-interacting zinc finger protein 1 |
| FKBP14 | FKB14_HUMAN | Peptidyl-prolyl cis-trans isomerase FKBP14 |
| FKBP1A | FKB1A_HUMAN | Peptidyl-prolyl cis-trans isomerase FKBP1A |
| FKBP3 | FKBP3_HUMAN | Peptidyl-prolyl cis-trans isomerase FKBP3 |
| FKBP4 | FKBP4_HUMAN | Peptidyl-prolyl cis-trans isomerase FKBP4, N-terminally processed |
| FKBP5 | FKBP5_HUMAN | Peptidyl-prolyl cis-trans isomerase FKBP5 |
| FKBP8 | FKBP8_HUMAN | Peptidyl-prolyl cis-trans isomerase FKBP8 |
| FLI1 | FLI1_HUMAN | Friend leukemia integration 1 transcription factor |
| FLNA | FLNA_HUMAN | Filamin-A |
| FLNB | FLNB_HUMAN | Filamin-B |
| FLNC | FLNC_HUMAN | Filamin-C |
| FLT1 | VGFR1_HUMAN | Vascular endothelial growth factor receptor 1 |
| FLT3 | FLT3_HUMAN | Receptor-type tyrosine-protein kinase FLT3 |
| FLT4 | VGFR3_HUMAN | Vascular endothelial growth factor receptor 3 |
| FLYWCH1 | FWCH1_HUMAN | FLYWCH-type zinc finger-containing protein 1 |
| FMR1 | FMR1_HUMAN | Synaptic functional regulator FMRI |
| FN1 | FINC_HUMAN | Ugl-Y3 |
| FNDC3A | FND3A_HUMAN | Fibronectin type-III domain-containing protein 3A |
| FNTB | FNTB_HUMAN | Protein famesyltransferase subunit beta |
| FOLH1 | FOLH1_HUMAN | Glutamate carboxypeptidase 2 |
| FOXO3 | FOXO3_HUMAN | Forkhead box protein 03 |
| FOXP2 | FOXP2_HUMAN | Forkhead box protein P2 |
| FOXP3 | FOXP3_HUMAN | Forkhead box protein P3 41 kDa form |
| FRS2 | FRS2_HUMAN | Fibroblast growth factor receptor substrate 2 |
| FRS3 | FRS3_HUMAN | Fibroblast growth factor receptor substrate 3 |
| FSCN1 | FSCN1_HUMAN | Fascin |
| FST | FST_HUMAN | Follistatin |
| FSTL3 | FSTL3_HUMAN | Follistatin-related protein 3 |
| FTO | FTO_HUMAN | Alpha-ketoglutarate-dependent dioxygenase FTO |
| FURIN | FURIN_HUMAN | Furin |
| FUS | FUS_HUMAN | RNA-binding protein FUS |
| FUT8 | FUT8_HUMAN | Alpha-(1,6)-fucosy Itransferase |
| FXN | FRDA_HUMAN | Frataxin mature form |
| FXR1 | FXR1_HUMAN | Fragile X mental retardation syndrome-related protein 1 |
| FXR2 | FXR2_HUMAN | Fragile X mental retardation syndrome-related protein 2 |
| FYB1 | FYB1_HUMAN | FYN-binding protein 1 |
| FYCO1 | FYCO1_HUMAN | FYVE and coiled-coil domain-containing protein 1 |
| FYN | FYN_HUMAN | Tyrosine-protein kinase Fyn |
| FZD4 | FZD4_HUMAN | Frizzled-4 |
| FZR1 | FZR1_HUMAN | Fizzy-related protein homolog |
| G2E3 | G2E3_HUMAN | G2/M phase-specific E3 ubiquitin-protein ligase |
| G3BP1 | G3BP1_HUMAN | Ras GTPase-activating protein-binding protein 1 |
| GAA | LYAG_HUMAN | 70 kDa lysosomal alpha-glucosidase |
| GABBR1 | GABR1_HUMAN | Gamma-aminobutyric acid type B receptor subunit 1 |
| GABRA1 | GBRA1_HUMAN | Gamma-aminobutyric acid receptor subunit alpha-1 |
| GABRA5 | GBRA5_HUMAN | Gamma-aminobutyric acid receptor subunit alpha-5 |
| GABRB2 | GBRB2_HUMAN | Gamma-aminobutyric acid receptor subunit beta-2 |
| GABRB3 | GBRB3_HUMAN | Gamma-aminobutyric acid receptor subunit beta-3 |
| GABRG2 | GBRG2_HUMAN | Gamma-aminobutyric acid receptor subunit gamma-2 |
| GAD1 | DCE1_HUMAN | Glutamate decarboxylase 1 |
| GAD2 | DCE2_HUMAN | Glutamate decarboxylase 2 |
| GAK | GAK_HUMAN | Cyclin-G-associated kinase |
| GALM | GALM_HUMAN | Aldose 1-epimerase |
| GALNS | GALNS_HUMAN | N-acetylgalactosamine-6-sulfatase |
| GALNT10 | GLT10_HUMAN | Polypeptide N-acetylgalactosaminyltransferase 10 |
| GALNT4 | GALT4_HUMAN | Polypeptide N-acetylgalactosaminyltransferase 4 |
| GALNT7 | GALT7_HUMAN | N-acetylgalactosaminyltransferase 7 |
| GALT | GALT_HUMAN | Galactose-1-phosphate uridylyltransferase |
| GARS | GARS_HUMAN | Glycine--tRNA ligase |
| GART | PUR2_HUMAN | Phosphoribosylglycinamide formyltransferase |
| GAS7 | GAS7_HUMAN | Growth arrest-specific protein 7 |
| GATA1 | GATA1_HUMAN | Erythroid transcription factor |
| GATA2 | GATA2_HUMAN | Endothelial transcription factor GATA-2 |
| GATA3 | GATA3_HUMAN | Trans-acting T-cell-specific transcription factor GATA-3 |
| GATA4 | GATA4_HUMAN | Transcription factor GATA-4 |
| GATA5 | GATA5_HUMAN | Transcription factor GATA-5 |
| GATA6 | GATA6_HUMAN | Transcription factor GATA-6 |
| GBA | GLCM_HUMAN | Lysosomal acid glucosylceramidase |
| GBA3 | GBA3_HUMAN | Cytosolic beta-glucosidase |
| GBE1 | GLGB_HUMAN | 1,4-alpha-glucan-branching enzyme |
| GCA | GRAN_HUMAN | Grancalcin |
| GCGR | GLR_HUMAN | Glucagon receptor |
| GCK | HXK4_HUMAN | Glucokinase |
| GDF15 | GDF15_HUMAN | Growth/differentiation factor 15 |
| GDF2 | GDF2_HUMAN | Growth/differentiation factor 2 |
| GEMIN5 | GEM15_HUMAN | Gem-associated protein 5 |
| GEMIN7 | GEM17_HUMAN | Gem-associated protein 7 |
| GFI1 | GFI1_HUMAN | Zinc finger protein Gfi-1 |
| GFI1B | GFI1B_HUMAN | Zinc finger protein Gfi-Ib |
| GFM1 | EFGM_HUMAN | Elongation factor G, mitochondrial |
| GFRA3 | GFRA3_HUMAN | GDNF family receptor alpha-3 |
| GGCT | GGCT_HUMAN | Gamma-glutamylcyclotransferase |
| GGT1 | GGT1_HUMAN | Glutathione hydrolase 1 light chain |
| GHR | GHR_HUMAN | Growth hormone-binding protein |
| GINS2 | PSF2_HUMAN | DNA replication complex GINS protein PSF2 |
| GIPC2 | GIPC2_HUMAN | PDZ domain-containing protein GIPC2 |
| GLDN | GLDN_HUMAN | Gliomedin shedded ectodomain |
| GLI4 | GLI4_HUMAN | Zinc finger protein GLI4 |
| GLIPR2 | GAPR1_HUMAN | Golgi-associated plant pathogenesis-related protein 1 |
| GLIS2 | GLIS2_HUMAN | Zinc finger protein GLIS2 |
| GLO1 | LGUL_HUMAN | Lactoylglutathione lyase |
| GLOD4 | GLOD4_HUMAN | Glyoxalase domain-containing protein 4 |
| GLP1R | GLP1R_HUMAN | Glucagon-like peptide 1 receptor |
| GLRA1 | GLRA1_HUMAN | Glycine receptor subunit alpha-I |
| GLRA3 | GLRA3_HUMAN | Glycine receptor subunit alpha-3 |
| GLS | GLSK_HUMAN | Glutaminase kidney isoform, mitochondrial |
| GLS2 | GLSL_HUMAN | Glutaminase liver isoform, mitochondrial |
| GLUD1 | DHE3_HUMAN | Glutamate dehydrogenase 1, mitochondrial |
| GMDS | GMDS_HUMAN | GDP-mannose 4,6 dehydratase |
| GMFG | GMFG_HUMAN | Glia maturation factor gamma |
| GNB1 | GBB1_HUMAN | Guanine nucleotide-binding protein G(I)/G(S)/G(T) subunit beta-l |
| GNE | GLCNE_HUMAN | N-acetylmannosamine kinase |
| GNPDA1 | GNPI1_HUMAN | Glucosamine-6-phosphate isomerase 1 |
| GNPNAT1 | GNA1_HUMAN | Glucosamine 6-phosphate N-acetyltransferase |
| GOT1 | AATC_HUMAN | Aspartate aminotransferase, cytoplasmic |
| GOT2 | AATM_HUMAN | Aspartate aminotransferase, mitochondrial |
| GPD1 | GPDA_HUMAN | Glycerol-3-phosphate dehydrogenase [NAD(+)], cytoplasmic |
| GPD1L | GPD1L_HUMAN | Glycerol-3-phosphate dehydrogenase I-like protein |
| GPI | G6PI_HUMAN | Glucose-6-phosphate isomerase |
| GPIHBP1 | HDBP1_HUMAN | Glycosylphosphatidy !inositol-anchored high density lipoprotein-binding |
| | | protein 1 |
| GPT2 | ALAT2_HUMAN | Alanine aminotransferase 2 |
| GPX1 | GPX1_HUMAN | Glutathione peroxidase 1 |
| GPX2 | GPX2_HUMAN | Glutathione peroxidase 2 |
| GPX4 | GPX4_HUMAN | Phospholipid hydroperoxide glutathione peroxidase |
| GPX7 | GPX7_HUMAN | Glutathione peroxidase 7 |
| GPX8 | GPX8_HUMAN | Probable glutathione peroxidase 8 |
| GRAP2 | GRAP2_HUMAN | GRB2-related adapter protein 2 |
| GRB10 | GRB10_HUMAN | Growth factor receptor-bound protein 10 |
| GRB14 | GRB14_HUMAN | Growth factor receptor-bound protein 14 |
| GRB2 | GRB2_HUMAN | Growth factor receptor-bound protein 2 |
| GRB7 | GRB7_HUMAN | Growth factor receptor-bound protein 7 |
| GRIA2 | GRIA2_HUMAN | Glutamate receptor 2 |
| GRIK1 | GRIK1_HUMAN | Glutamate receptor ionotropic, kainate 1 |
| GRIK2 | GRIK2_HUMAN | Glutamate receptor ionotropic, kainate 2 |
| GRIN2A | NMDE1_HUMAN | Glutamate receptor ionotropic, NMDA 2A |
| GRK2 | ARBK1_HUMAN | Beta-adrenergic receptor kinase 1 |
| GRK4 | GRK4_HUMAN | G protein-coupled receptor kinase 4 |
| GRK5 | GRK5_HUMAN | G protein-coupled receptor kinase 5 |
| GRK6 | GRK6_HUMAN | G protein-coupled receptor kinase 6 |
| GRM1 | GRM1_HUMAN | Metabotropic glutamate receptor 1 |
| GRM2 | GRM2_HUMAN | Metabotropic glutamate receptor 2 |
| GRM3 | GRM3_HUMAN | Metabotropic glutamate receptor 3 |
| GRM5 | GRM5_HUMAN | Metabotropic glutamate receptor 5 |
| GRM7 | GRM7_HUMAN | Metabotropic glutamate receptor 7 |
| GRM8 | GRM8_HUMAN | Metabotropic glutamate receptor 8 |
| GRN | GRN_HUMAN | Granulin-7 |
| GSK3B | GSK3B_HUMAN | Glycogen synthase kinase-3 beta |
| GSN | GELS_HUMAN | Gelsolin |
| GSPT1 | ERF3A_HUMAN | Eukaryotic peptide chain release factor GTP-binding subunit ERF3A |
| GSR | GSHR_HUMAN | Glutathione reductase, mitochondrial |
| GSTOl | GSTO1_HUMAN | Glutathione S-transferase omega-1 |
| GTF2B | TF2B_HUMAN | Transcription initiation factor IIB |
| GTF2E1 | T2EA_HUMAN | General transcription factor IIE subunit 1 |
| GTF2F1 | T2FA_HUMAN | General transcription factor IIF subunit 1 |
| GTF2H1 | TF2H1_HUMAN | General transcription factor IIH subunit 1 |
| GTF3A | TF3A_HUMAN | Transcription factor IIIA |
| GUSB | BGLR_HUMAN | Beta-glucuronidase |
| GZF1 | GZF1_HUMAN | GDNF-inducible zinc finger protein 1 |
| GZMB | GRAB_HUMAN | Granzyme B |
| GZMM | GRAM_HUMAN | Granzyme M |
| H2AFY | H2AY_HUMAN | Core histone macro-H2A.I |
| H2AFY2 | H2AW_HUMAN | Core histone macro-H2A.2 |
| HADHA | ECHA_HUMAN | Long chain 3-hydroxyacyl-CoA dehydrogenase |
| HASPIN | HASP_HUMAN | Serine/threonine-protein kinase haspin |
| HAT1 | HAT1_HUMAN | Histone acetyltransferase type B catalytic subunit |
| HBP1 | HBP1_HUMAN | HMG box-containing protein 1 |
| HCFC1 | HCFC1_HUMAN | HCF C-terminal chain 6 |
| HCK | HCK_HUMAN | Tyrosine-protein kinase HCK |
| HDAC4 | HDAC4_HUMAN | Histone deacetylase 4 |
| HDAC6 | HDAC6_HUMAN | Histone deacetylase 6 |
| HDAC7 | HDAC7_HUMAN | Histone deacetylase 7 |
| HDHD2 | HDHD2_HUMAN | Haloacid dehalogenase-like hydrolase domain containing protein 2 |
| HECTD1 | HECD1_HUMAN | E3 ubiquitin-protein ligase HECTD1 |
| HECWI | HECW1_HUMAN | E3 ubiquitin-protein ligase HECW1 |
| HECW2 | HECW2_HUMAN | E3 ubiquitin-protein ligase HECW2 |
| HERC1 | HERC1_HUMAN | Probable E3 ubiquitin-protein ligase HERC1 |
| HERC2 | HERC2_HUMAN | E3 ubiquitin-protein ligase HERC2 |
| HERVK 113 | GA113_HUMAN | Endogenous retrovirus group K member 113 Gag polyprotein |
| HEXA | HEXA_HUMAN | Beta-hexosaminidase subunit alpha |
| HEXB | HEXB_HUMAN | Beta-hexosaminidase subunit beta chain A |
| HFE | HFE_HUMAN | Hereditary hemochromatosis protein |
| HGD | HGD_HUMAN | Homogentisate 1,2-dioxygenase |
| HGS | HGS_HUMAN | Hepatocyte growth factor-regulated tyrosine kinase substrate |
| HHIP | HHIP_HUMAN | Hedgehog-interacting protein |
| HIC1 | HIC1_HUMAN | Hypermethylated in cancer 1 protein |
| HIC2 | HIC2_HUMAN | Hypermethylated in cancer 2 protein |
| HIF1A | HIF1A_HUMAN | Hypoxia-inducible factor 1-alpha |
| HIF3A | HIF3A_HUMAN | Hypoxia-inducible factor 3-alpha |
| HINFP | HINFP_HUMAN | Histone H4 transcription factor |
| HIRA | HIRA_HUMAN | Protein HIRA |
| HIVEPl | ZEP1_HUMAN | Zinc finger protein 40 |
| HIVEP2 | ZEP2_HUMAN | Transcription factor HIVEP2 |
| HIVEP3 | ZEP3_HUMAN | Transcription factor HIVEP3 |
| HMCES | HMCES_HUMAN | Abasic site processing protein HMCES |
| HMGCL | HMGCL_HUMAN | Hydroxymethylglutary 1-CoA lyase, mitochondrial |
| HNF4A | HNF4A_HUMAN | Hepatocyte nuclear factor 4-alpha |
| HNF4G | HNF4G_HUMAN | Hepatocyte nuclear factor 4-gamma |
| HNRNPA1 | ROA1_HUMAN | Heterogeneous nuclear ribonucleoprotein Al, N-terminally processed |
| HNRNPA2B1 | ROA2_HUMAN | Heterogeneous nuclear ribonucleoproteins A2/B1 |
| HNRNPAB | ROAA_HUMAN | Heterogeneous nuclear ribonucleoprotein A/B |
| HNRNPD | HNRPD_HUMAN | Heterogeneous nuclear ribonucleoprotein D0 |
| HNRNPH2 | HNRH2_HUMAN | Heterogeneous nuclear ribonucleoprotein H2, N-terminally processed |
| HPD | HPPD_HUMAN | 4-hydroxyphenylpymvate dioxygenase |
| HPN | HEPS_HUMAN | Serine protease hepsin catalytic chain |
| HRH1 | HRH1_HUMAN | Histamine H1 receptor |
| HS3ST1 | HS3S1_HUMAN | Heparan sulfate glucosamine 3-O-sulfotransferase 1 |
| HS3ST3Al | HS3SA_HUMAN | Heparan sulfate glucosamine 3-O-sulfotransferase 3A1 |
| HS3ST5 | HS3S5_HUMAN | Heparan sulfate glucosamine 3-O-sulfotransferase 5 |
| HSCB | HSC20_HUMAN | Iron-sulfur cluster co-chaperone protein HscB, mitochondrial |
| HSD17B10 | HCD2_HUMAN | 3-hydroxyacyl-CoA dehydrogenase type-2 |
| HSD17B4 | DHB4_HUMAN | Enoyl-CoA hydratase 2 |
| HSPA1A | HS71A_HUMAN | Heat shock 70 kDa protein 1A |
| HSPA5 | BIP_HUMAN | Endoplasmic reticulum chaperone BiP |
| HSPA8 | HSP7C_HUMAN | Heat shock cognate 71 kDa protein |
| HSPA9 | GRP75_HUMAN | Stress-70 protein, mitochondrial |
| HSPBI | HSPB1_HUMAN | Heat shock protein beta-1 |
| HSPB2 | HSPB2_HUMAN | Heat shock protein beta-2 |
| HSPB6 | HSPB6_HUMAN | Heat shock protein beta-6 |
| HSPDI | CH60_HUMAN | 60 kDa heat shock protein, mitochondrial |
| HSPG2 | PGBM_HUMAN | LG3 peptide |
| HTRA1 | HTRA1_HUMAN | Serine protease HTRAl |
| HTRA2 | HTRA2_HUMAN | Serine protease HTRA2, mitochondrial |
| HTRA3 | HTRA3_HUMAN | Serine protease HTRA3 |
| HTT | HD_HUMAN | Huntingtin |
| HUS1 | HUS1_HUMAN | Checkpoint protein HUS1 |
| HUWE1 | HUWE1_HUMAN | E3 ubiquitin-protein ligase HUWE1 |
| HYAL1 | HYAL1_HUMAN | Hyaluronidase-1 |
| HYDIN | HYDIN_HUMAN | Hydrocephalus-inducing protein homolog |
| ICAM1 | ICAM1_HUMAN | Intercellular adhesion molecule 1 |
| IDE | IDE_HUMAN | Insulin-degrading enzyme |
| IDH3G | IDH3G_HUMAN | Isocitrate dehydrogenase [NAD] subunit gamma, mitochondrial |
| IDO1 | 123O1_HUMAN | Indoleamine 2,3-dioxygenase 1 |
| IDS | IDS_HUMAN | Iduronate 2-sulfatase 14 kDa chain |
| IDUA | IDUA_HUMAN | Alpha-L-iduronidase |
| IFI16 | IF16_HUMAN | Gamma-interferon-inducible protein 16 |
| IFNAR1 | INAR1_HUMAN | Interferon alpha/beta receptor 1 |
| IFNGR1 | INGR1_HUMAN | Interferon gamma receptor 1 |
| IFNGR2 | INGR2_HUMAN | Interferon gamma receptor 2 |
| IFNLR1 | INLR1_HUMAN | Interferon lambda receptor 1 |
| IGF1R | IGF1R_HUMAN | Insulin-like growth factor 1 receptor beta chain |
| IGF2R | MPRI_HUMAN | Cation-independent mannose-6-phosphate receptor |
| IGFBP1 | IBP1_HUMAN | Insulin-like growth factor-binding protein 1 |
| IGFBP4 | IBP4_HUMAN | Insulin-like growth factor-binding protein 4 |
| IGFBP6 | IBP6_HUMAN | Insulin-like growth factor-binding protein 6 |
| IGHA1 | IGHA1_HUMAN | Immunoglobulin heavy constant alpha 1 |
| IGHE | IGHE_HUMAN | Immunoglobulin heavy constant epsilon |
| IGHG1 | IGHG1_HUMAN | Immunoglobulin heavy constant gamma 1 |
| IGHG4 | IGHG4_HUMAN | Immunoglobulin heavy constant gamma 4 |
| IGHM | IGHM_HUMAN | Immunoglobulin heavy constant mu |
| IGHV3-23 | HV323_HUMAN | Immunoglobulin heavy variable 3-23 |
| IGHV3-33 | HV333_HUMAN | Immunoglobulin heavy variable 3-33 |
| IGHV4-59 | HV459_HUMAN | Immunoglobulin heavy variable 4-59 |
| IGKC | IGKC_HUMAN | Immunoglobulin kappa constant |
| IGKV1-33 | KV133_HUMAN | Immunoglobulin kappa variable 1-33 |
| IKBKB | IKKB_HUMAN | Inhibitor of nuclear factor kappa-B kinase subunit beta |
| IKZF1 | IKZF1_HUMAN | DNA-binding protein Ikaros |
| IKZF2 | IKZF2_HUMAN | Zinc finger protein Helios |
| IKZF3 | IKZF3_HUMAN | Zinc finger protein Aiolos |
| IKZF4 | IKZF4_HUMAN | Zinc finger protein Eos |
| IKZF5 | IKZF5_HUMAN | Zinc finger protein Pegasus |
| IL12B | IL12B_HUMAN | Interleukin-12 subunit beta |
| IL13RA2 | l13R2_HUMAN | Interleukin-13 receptor subunit alpha-2 |
| IL17A | IL17_HUMAN | Interleukin-17A |
| IL17F | IL17F_HUMAN | Interleukin-17F |
| IL17RA | IL7RA_HUMAN | Interleukin-17 receptor A |
| IL18Rl | IL8R_HUMAN | Interleukin-18 receptor 1 |
| IL18RAP | IL8RA_HUMAN | Interleukin-18 receptor accessory protein |
| IL1F10 | IL1FA_HUMAN | Interleukin-I family member 10 |
| IL1RAP | IL1AP_HUMAN | Interleukin-I receptor accessory protein |
| IL20RB | I20RB_HUMAN | Interleukin-20 receptor subunit beta |
| IL22RA1 | I22R1_HUMAN | Interleukin-22 receptor subunit alpha-1 |
| IL23R | IL23R_HUMAN | Interleukin-23 receptor |
| IL4R | IL4RA_HUMAN | Soluble interleukin-4 receptor subunit alpha |
| IL5RA | IL5RA_HUMAN | Interleukin-5 receptor subunit alpha |
| IL6R | IL6RA_HUMAN | Interleukin-6 receptor subunit alpha |
| IL6ST | IL6RB_HUMAN | Interleukin-6 receptor subunit beta |
| ILK | ILK_HUMAN | Integrin-linked protein kinase |
| IMPAI | IMPA1_HUMAN | Inositol monophosphatase 1 |
| INHBA | INHBA_HUMAN | Inhibin beta A chain |
| INKAl | INKA1_HUMAN | P AK4-inhibitor INKAI |
| INO80B | IN80B_HUMAN | INO80 complex subunit B |
| INPPL1 | SHIP2_HUMAN | Phosphatidylinositol 3,4,5-trisphosphate 5-phosphatase 2 |
| INSM1 | INSM1_HUMAN | Insulinoma-associated protein 1 |
| INSM2 | INSM2_HUMAN | Insulinoma-associated protein 2 |
| INSR | INSR_HUMAN | Insulin receptor subunit beta |
| INTS11 | INT11_HUMAN | Integrator complex subunit 11 |
| IPMK | IPMK_HUMAN | Inositol polyphosphate multikinase |
| IQGAP1 | IQGA1_HUMAN | Ras GTPase-activating-like protein IQGAP1 |
| IQGAP2 | IQGA2_HUMAN | Ras GTPase-activating-like protein IQGAP2 |
| IQGAP3 | IQGA3_HUMAN | Ras GTPase-activating-like protein IQGAP3 |
| IQUB | IQUB_HUMAN | IQ and ubiquitin-like domain-containing protein |
| IRAKI | IRAKI_HUMAN | Interleukin-1 receptor-associated kinase 1 |
| IRAK4 | IRAK4_HUMAN | Interleukin-1 receptor-associated kinase 4 |
| ISCU | ISCU_HUMAN | Iron-sulfur cluster assembly enzyme ISCU, mitochondrial |
| ISG15 | ISG15_HUMAN | Ubiquitin-like protein ISG15 |
| ISG20 | ISG20_HUMAN | Interferon-stimulated gene 20 kDa protein |
| ITCH | ITCH_HUMAN | E3 ubiquitin-protein ligase Itchy homolog |
| ITGA2B | ITA2B_HUMAN | Integrin alpha-IIb light chain, form 2 |
| ITGA4 | ITA4_HUMAN | Integrin alpha-4 |
| ITGA5 | ITA5_HUMAN | Integrin alpha-5 light chain |
| ITGAL | ITAL_HUMAN | Integrin alpha-L |
| ITGAV | ITAV_HUMAN | Integrin alpha-V light chain |
| ITGAX | ITAX_HUMAN | Integrin alpha-X |
| ITGB1 | ITB1_HUMAN | Integrin beta-1 |
| ITGBIBPI | ITBP1_HUMAN | Integrin beta-1-binding protein 1 |
| ITGB2 | ITB2_HUMAN | Integrin beta-2 |
| ITGB3 | ITB3_HUMAN | Integrin beta-3 |
| ITGB4 | ITB4_HUMAN | Integrin beta-4 |
| ITGB6 | ITB6_HUMAN | Integrin beta-6 |
| ITIHl | ITIH1_HUMAN | Inter-alpha-trypsin inhibitor heavy chain Hl |
| ITK | ITK_HUMAN | Tyrosine-protein kinase ITK/TSK |
| ITLNl | ITLNl_HUMAN | Intelectin-1 |
| ITPA | ITPA_HUMAN | Inosine triphosphate pyrophosphatase |
| ITPKl | ITPKl_HUMAN | Inositol-tetrakisphosphate 1-kinase |
| ITPKA | IP3KA_HUMAN | Inositol-trisphosphate 3-kinase A |
| ITPKC | IP3KC_HUMAN | Inositol-trisphosphate 3-kinase C |
| ITSNl | ITSNl_HUMAN | Intersectin-1 |
| ITSN2 | ITSN2_HUMAN | Intersectin-2 |
| IYD | IYD1_HUMAN | lodotyrosine deiodinase 1 |
| JAG1 | JAGI_HUMAN | Protein jagged-1 |
| JAG2 | JAG2_HUMAN | Protein jagged-2 |
| JAKl | JAKl_HUMAN | Tyrosine-protein kinase JAKl |
| JAK2 | JAK2_HUMAN | Tyrosine-protein kinase JAK2 |
| JAK3 | JAK3_HUMAN | Tyrosine-protein kinase JAK3 |
| JMJDlC | JHD2C_HUMAN | Probable JmjC domain-containing histone demethylation protein 2C |
| JMJD6 | JMJD6_HUMAN | Bifunctional arginine demethylase and lysyl-hydroxylase JMJD6 |
| JMJD7 | JMJD7_HUMAN | Bifunctional peptidase and (3S)-lysyl hydroxylase JMJD7 |
| KANKl | KANKl_HUMAN | KN motif and ankyrin repeat domain-containing protein 1 |
| KANK2 | KANK2_HUMAN | KN motif and ankyrin repeat domain-containing protein 2 |
| KARS | SYK_HUMAN | Lysine--tRNA ligase |
| KAT2A | KAT2A_HUMAN | Histone acetyltransferase KAT2A |
| KAT2B | KAT2B_HUMAN | Histone acetyltransferase KAT2B |
| KAT6A | KAT6A_HUMAN | Histone acetyltransferase KAT6A |
| KAT6B | KAT6B_HUMAN | Histone acetyltransferase KAT6B |
| KCMFl | KCMFl_HUMAN | E3 ubiquitin-protein ligase KCMFl |
| KCNAB2 | KCAB2_HUMAN | Voltage-gated potassium channel subunit beta-2 |
| KCNH2 | KCNH2_HUMAN | Potassium voltage-gated channel subfamily H member 2 |
| KCNJ11 | KCJ11_HUMAN | ATP-sensitive inward rectifier potassium channel 11 |
| KCTD10 | BACD3_HUMAN | BTB/POZ domain-containing adapter for CUL3-mediated RhoA |
| | | degradation protein 3 |
| KCTD13 | BACDl_HUMAN | BTB/POZ domain-containing adapter for CUL3-mediated RhoA |
| | | degradation protein 1 |
| KCTD16 | KCD16_HUMAN | BTB/POZ domain-containing protein KCTD 16 |
| KCTD17 | KCD17_HUMAN | BTB/POZ domain-containing protein KCTD 17 |
| KCTD5 | KCTD5_HUMAN | BTB/POZ domain-containing protein KCTD5 |
| KCTD9 | KCTD9_HUMAN | BTB/POZ domain-containing protein KCTD9 |
| KDMIA | KDMIA_HUMAN | Lysine-specific histone demethylase 1A |
| KDMIB | KDMIB_HUMAN | Lysine-specific histone demethylase 1B |
| KDM2A | KDM2A_HUMAN | Lysine-specific demethylase 2A |
| KDM2B | KDM2B_HUMAN | Lysine-specific demethylase 2B |
| KDM3A | KDM3A_HUMAN | Lysine-specific demethylase 3A |
| KDM3B | KDM3B_HUMAN | Lysine-specific demethylase 3B |
| KDM4A | KDM4A_HUMAN | Lysine-specific demethylase 4A |
| KDM4B | KDM4B_HUMAN | Lysine-specific demethylase 4B |
| KDM4C | KDM4C_HUMAN | Lysine-specific demethylase 4C |
| KDM5A | KDM5A_HUMAN | Lysine-specific demethylase 5A |
| KDM5B | KDM5B_HUMAN | Lysine-specific demethylase 5B |
| KDR | VGFR2_HUMAN | Vascular endothelial growth factor receptor 2 |
| KEAP1 | KEAP1_HUMAN | Kelch-like ECH-associated protein 1 |
| KHDC4 | KHDC4_HUMAN | KH homology domain-containing protein 4 |
| KHK | KHK_HUMAN | Ketohexokinase |
| KIAA0391 | MRPP3_HUMAN | Mitochondrial ribonuclease P catalytic subunit |
| KIF11 | KIF11_HUMAN | Kinesin-like protein KIF11 |
| K1Fl3B | K113B_HUMAN | Kinesin-like protein KIF13B |
| KIFI5 | KIFI5_HUMAN | Kinesin-like protein KIFI5 |
| KIFI8A | Kll8A_HUMAN | Kinesin-like protein KIFI8A |
| KIFIA | KIFIA_HUMAN | Kinesin-like protein KIF IA |
| KIFlB | KIFIB_HUMAN | Kinesin-like protein KIF1B |
| KIFIC | KIFIC_HUMAN | Kinesin-like protein KIF1C |
| KIF22 | KIF22_HUMAN | Kinesin-like protein KIF22 |
| KIF23 | KIF23_HUMAN | Kinesin-like protein KIF23 |
| KIF2C | KIF2C_HUMAN | Kinesin-like protein KIF2C |
| KIF3B | KIF3B_HUMAN | Kinesin-like protein KIF3B, N-terminally processed |
| KIF3C | KIF3C_HUMAN | Kinesin-like protein KIF3C |
| KIF7 | KIF7_HUMAN | Kinesin-like protein KIF7 |
| KIF9 | KIF9_HUMAN | Kinesin-like protein KIF9 |
| KIFC1 | KIFC1_HUMAN | Kinesin-like protein KIFC1 |
| KIFC3 | KIFC3_HUMAN | Kinesin-like protein KIFC3 |
| KIN | KINI7_HUMAN | DNA/RNA-binding protein KINI7 |
| KIR2DS4 | Kl2S4_HUMAN | Killer cell immunoglobulin-like receptor 2DS4 |
| KIRREL3 | KIRR3_HUMAN | Processed kin of IRRE-like protein 3 |
| KIT | KIT_HUMAN | Mast/stem cell growth factor receptor Kit |
| KLB | KLOTB_HUMAN | Beta-klotho |
| KLFl | KLFl_HUMAN | Krueppel-like factor 1 |
| KLF10 | KLF10_HUMAN | Krueppel-like factor 10 |
| KLHDC2 | KLDC2_HUMAN | Kelch domain-containing protein 2 |
| KLHLll | KLH11_HUMAN | Kelch-like protein 11 |
| KLHL12 | KLH12_HUMAN | Kelch-like protein 12 |
| KLHL17 | KLH17_HUMAN | Kelch-like protein 17 |
| KLHL40 | KLH40_HUMAN | Kelch-like protein 40 |
| KLHL7 | KLHL7_HUMAN | Kelch-like protein 7 |
| KLK4 | KLK4_HUMAN | Kallikrein-4 |
| KLK6 | KLK6_HUMAN | Kallikrein-6 |
| KLKBl | KLKB1_HUMAN | Plasma kallikrein light chain |
| KLRDl | KLRD1_HUMAN | Natural killer cells antigen CD94 |
| KLRGl | KLRG1_HUMAN | Killer cell lectin-like receptor subfamily G member 1 |
| KLRG2 | KLRG2_HUMAN | Killer cell lectin-like receptor subfamily G member 2 |
| KLRKl | NKG2D_HUMAN | NKG2-D type II integral membrane protein |
| KMO | KMO_HUMAN | Kynurenine 3-monooxygenase |
| KMT2A | KMT2A_HUMAN | MLL cleavage product C 180 |
| KMT2B | KMT2B_HUMAN | Histone-lysine N-methyltransferase 2B |
| KMT2C | KMT2C_HUMAN | Histone-lysine N-methyltransferase 2C |
| KMT2D | KMT2D_HUMAN | Histone-lysine N-methyltransferase 2D |
| KMT2E | KMT2E_HUMAN | Inactive histone-lysine N-methyltransferase 2E |
| KMT5A | KMT5A_HUMAN | N-lysine methyltransferase KMT5A |
| KREMEN1 | KREMl_HUMAN | Kremen protein 1 |
| KRlTl | KRlTl_HUMAN | Krev interaction trapped protein 1 |
| KSR2 | KSR2_HUMAN | Kinase suppressor of Ras 2 |
| KYAT1 | KAT1_HUMAN | Kynurenine--oxoglutarate transaminase 1 |
| KYNU | KYNU_HUMAN | Kynureninase |
| L3MBTL2 | LMBL2_HUMAN | Lethal(3)malignant brain tumor-like protein 2 |
| LAMA5 | LAMA5_HUMAN | Laminin subunit alpha-5 |
| LAMP3 | LAMP3_HUMAN | Lysosome-associated membrane glycoprotein 3 |
| LAMTOR2 | LTOR2_HUMAN | Ragulator complex protein LAMTOR2 |
| LAMTOR3 | LTOR3_HUMAN | Ragulator complex protein LAMTOR3 |
| LAMTOR5 | LTOR5_HUMAN | Ragulator complex protein LAMTOR5 |
| LANCLl | LANC1_HUMAN | Glutathione S-transferase LANCLl |
| LARP7 | LARP7_HUMAN | La-related protein 7 |
| LARS | SYLC_HUMAN | Leucine--tRNA ligase, cytoplasmic |
| LASPl | LASP1_HUMAN | LIM and SH3 domain protein 1 |
| LBR | LBR_HUMAN | Delta(14)-sterol reductase |
| LCAT | LCAT_HUMAN | Phosphatidylcholine-sterol acyltransferase |
| LCK | LCK_HUMAN | Tyrosine-protein kinase Lek |
| LCNl | LCNl_HUMAN | Lipocalin-1 |
| LCNl5 | LCN15_HUMAN | Lipocalin-15 |
| LCN2 | NGAL_HUMAN | Neutrophil gelatinase-associated lipocalin |
| LDLR | LDLR_HUMAN | Low-density lipoprotein receptor |
| LEOl | LEO1_HUMAN | RNA polymerase-associated protein LEOl |
| LEPR | LEPR_HUMAN | Leptin receptor |
| LGALSl | LEGl_HUMAN | Galectin-1 |
| LGALS2 | LEG2_HUMAN | Galectin-2 |
| LGALS3 | LEG3_HUMAN | Galectin-3 |
| LGALS4 | LEG4_HUMAN | Galectin-4 |
| LGALS7 \| | LEG7_HUMAN | Galectin-7 |
| LGALS7B | | |
| LGALS8 | LEG8_HUMAN | Galectin-8 |
| LGALS9 | LEG9_HUMAN | Galectin-9 |
| LG11 | LG11_HUMAN | Leucine-rich glioma-inactivated protein 1 |
| LGMN | LGMN_HUMAN | Legumain |
| LGR4 | LGR4_HUMAN | Leucine-rich repeat-containing G-protein coupled receptor 4 |
| LIFR | LIFR_HUMAN | Leukemia inhibitory factor receptor |
| LIGl | DNL11_HUMAN | DNA ligase 1 |
| LIG3 | DNLl3_HUMAN | DNA ligase 3 |
| LIG4 | DNL14_HUMAN | DNA ligase 4 |
| LILRA5 | LIRA5_HUMAN | Leukocyte immunoglobulin-like receptor subfamily A member 5 |
| LILRB4 | LIRB4_HUMAN | Leukocyte immunoglobulin-like receptor subfamily B member 4 |
| LIMKl | LlMKl_HUMAN | LIM domain kinase 1 |
| LIMK2 | LlMK2_HUMAN | LIM domain kinase 2 |
| LIMSl | LlMSl_HUMAN | LIM and senescent cell antigen-like-containing domain protein 1 |
| LIN28A | LN28A_HUMAN | Protein lin-28 homolog A |
| LIN28B | LN28B_HUMAN | Protein lin-28 homolog B |
| LINGOl | L1GOl_HUMAN | Leucine-rich repeat and immunoglobulin-like domain-containing nogo |
| | | receptor-interacting protein 1 |
| LIPF | LIPG_HUMAN | Gastric triacylglycerol lipase |
| LMNBl | LMNBl_HUMAN | Lamin-Bl |
| LMO2 | RBTN2_HUMAN | Rhombotin-2 |
| LMO4 | LMO4_HUMAN | LIM domain transcription factor LM04 |
| LNPEP | LCAP_HUMAN | Leucyl-cystinyl aminopeptidase, pregnancy serum form |
| LNXl | LNXl_HUMAN | E3 ubiquitin-protein ligase LNX |
| LNX2 | LNX2_HUMAN | Ligand of Numb protein X 2 |
| LONPl | LONM_HUMAN | Lon protease homolog, mitochondrial |
| LONRF3 | LONF3_HUMAN | LON peptidase N-terminal domain and RING finger protein 3 |
| LRBA | LRBA_HUMAN | Lipopolysaccharide-responsive and beige-like anchor protein |
| LRFN5 | LRFN5_HUMAN | Leucine-rich repeat and fibronectin type-III domain-containing protein 5 |
| LR1Gl | LR1Gl_HUMAN | Leucine-rich repeats and immunoglobulin-like domains protein 1 |
| LRPl | LRPl_HUMAN | Low-density lipoprotein receptor-related protein 1 intracellular domain |
| LRP6 | LRP6_HUMAN | Low-density lipoprotein receptor-related protein 6 |
| LRP8 | LRP8_HUMAN | Low-density lipoprotein receptor-related protein 8 |
| LRRC32 | LRC32_HUMAN | Transforming growth factor beta activator LRRC32 |
| LRRC4 | LRRC4_HUMAN | Leucine-rich repeat-containing protein 4 |
| LRRC4C | LRC4C_HUMAN | Leucine-rich repeat-containing protein 4C |
| LRRK2 | LRRK2_HUMAN | Leucine-rich repeat serine/threonine-protein kinase 2 |
| LSM4 | LSM4_HUMAN | U6 snRNA-associated Sm-like protein LSm4 |
| LSM6 | LSM6_HUMAN | U6 snRNA-associated Sm-like protein LSm6 |
| LSM7 | LSM7_HUMAN | U6 snRNA-associated Sm-like protein LSm7 |
| LSM8 | LSM8_HUMAN | U6 snRNA-associated Sm-like protein LSm8 |
| LSS | ERG7_HUMAN | Lanosterol synthase |
| LTF | TRFL_HUMAN | Lactoferroxin-C |
| LXN | LXN_HUMAN | Latexin |
| LY86 | LY86_HUMAN | Lymphocyte antigen 86 |
| LYAR | LYAR_HUMAN | Cell growth-regulating nucleolar protein |
| LYPD6 | LYPD6_HUMAN | Ly6/PLAUR domain-containing protein 6 |
| LYZ | LYSC_HUMAN | Lysozyme C |
| MAD2Ll | MD2L1_HUMAN | Mitotic spindle assembly checkpoint protein MAD2A |
| MAGll | MAG11_HUMAN | Membrane-associated guanylate kinase, WW and PDZ domain- |
| | | containing protein 1 |
| MAGOH | MGN_HUMAN | Protein mago nashi homolog |
| MAGOHB | MGN2_HUMAN | Protein mago nashi homolog 2 |
| MALTl | MALTl_HUMAN | Mucosa-associated lymphoid tissue lymphoma |
| | | translocation protein 1 |
| MANlBl | MAlBl_HUMAN | Endoplasmic reticulum mannosy 1-oligosaccharide 1,2-alpha- |
| | | mannosidase |
| MAP2Kl | MP2Kl_HUMAN | Dual specificity mitogen-activated protein kinase kinase 1 |
| MAP2K2 | MP2K2_HUMAN | Dual specificity mitogen-activated protein kinase kinase 2 |
| MAP2K4 | MP2K4_HUMAN | Dual specificity mitogen-activated protein kinase kinase 4 |
| MAP2K5 | MP2K5_HUMAN | Dual specificity mitogen-activated protein kinase kinase 5 |
| MAP2K6 | MP2K6_HUMAN | Dual specificity mitogen-activated protein kinase kinase 6 |
| MAP2K7 | MP2K7_HUMAN | Dual specificity mitogen-activated protein kinase kinase 7 |
| MAP3K10 | M3K10_HUMAN | Mitogen-activated protein kinase kinase kinase 10 |
| MAP3K11 | M3K11_HUMAN | Mitogen-activated protein kinase kinase kinase 11 |
| MAP3K12 | M3K12_HUMAN | Mitogen-activated protein kinase kinase kinase 12 |
| MAP3K14 | M3K14_HUMAN | Mitogen-activated protein kinase kinase kinase 14 |
| MAP3K20 | M3K20_HUMAN | Mitogen-activated protein kinase kinase kinase 20 |
| MAP3K5 | M3K5_HUMAN | Mitogen-activated protein kinase kinase kinase 5 |
| MAP3K7 | M3K7_HUMAN | Mitogen-activated protein kinase kinase kinase 7 |
| MAP3K9 | M3K9_HUMAN | Mitogen-activated protein kinase kinase kinase 9 |
| MAP4K1 | M4K1_HUMAN | Mitogen-activated protein kinase kinase kinase kinase 1 |
| MAP4K3 | M4K3_HUMAN | Mitogen-activated protein kinase kinase kinase kinase 3 |
| MAP4K4 | M4K4_HUMAN | Mitogen-activated protein kinase kinase kinase kinase 4 |
| MAPK1 | MK0l_HUMAN | Mitogen-activated protein kinase 1 |
| MAPK10 | MKl0_HUMAN | Mitogen-activated protein kinase 10 |
| MAPK12 | MK12_HUMAN | Mitogen-activated protein kinase 12 |
| MAPK13 | MK13_HUMAN | Mitogen-activated protein kinase 13 |
| MAPK14 | MK14_HUMAN | Mitogen-activated protein kinase 14 |
| MAPK3 | MK03_HUMAN | Mitogen-activated protein kinase 3 |
| MAPK7 | MK07_HUMAN | Mitogen-activated protein kinase 7 |
| MAPK8 | MK08_HUMAN | Mitogen-activated protein kinase 8 |
| MAPK9 | MK09_HUMAN | Mitogen-activated protein kinase 9 |
| MAPKAPK2 | MAPK2_HUMAN | MAP kinase-activated protein kinase 2 |
| MAPKAPK3 | MAPK3_HUMAN | MAP kinase-activated protein kinase 3 |
| MARC1 | MARC1_HUMAN | Mitochondrial amidoxime-reducing component 1 |
| MARK1 | MARK1_HUMAN | Serine/threonine-protein kinase MARK1 |
| MARK2 | MARK2_HUMAN | Serine/threonine-protein kinase MARK2 |
| MARK3 | MARK3_HUMAN | MAP/microtubule affinity-regulating kinase 3 |
| MARK4 | MARK4_HUMAN | MAP/microtubule affinity-regulating kinase 4 |
| MARS | SYMC_HUMAN | Methionine--tRNA ligase, cytoplasmic |
| MASP1 | MASP1_HUMAN | Mannan-binding lectin serine protease 1 light chain |
| MASP2 | MASP2_HUMAN | Mannan-binding lectin serine protease 2 B chain |
| MASTL | GWL_HUMAN | Serine/threonine-protein kinase greatwall |
| MATK | MATK_HUMAN | Megakaryocyte-associated tyrosine-protein kinase |
| MAZ | MAZ_HUMAN | Myc-associated zinc finger protein |
| MBD1 | MBD1_HUMAN | Methyl-CpG-binding domain protein 1 |
| MBD2 | MBD2_HUMAN | Methyl-CpG-binding domain protein 2 |
| MBD3 | MBD3_HUMAN | Methyl-CpG-binding domain protein 3 |
| MBD4 | MBD4_HUMAN | Methyl-CpG-binding domain protein 4 |
| MBL2 | MBL2_HUMAN | Mannose-binding protein C |
| MBLAC1 | MBLC1_HUMAN | Metallo-beta-lactamase domain-containing protein 1 |
| MBTD1 | MBTD1_HUMAN | MBT domain-containing protein 1 |
| MCAT | FABD_HUMAN | Malonyl-CoA-acyl carrier protein transacylase, mitochondrial |
| MCEE | MCEE_HUMAN | Methylmalony 1-CoA epimerase, mitochondrial |
| MCOLN1 | MCLN1_HUMAN | Mucolipin-1 |
| MCTS1 | MCTS1_HUMAN | Malignant T-cell-amplified sequence 1 |
| MCU | MCU_HUMAN | Calcium uniporter protein, mitochondrial |
| MDM2 | MDM2_HUMAN | E3 ubiquitin-protein ligase Mdm2 |
| MDP1 | MGDP1_HUMAN | Magnesium-dependent phosphatase 1 |
| ME1 | MAOX_HUMAN | NADP-dependent malic enzyme |
| ME2 | MAOM_HUMAN | NAD-dependent malic enzyme, mitochondrial |
| MECOM | MECOM_HUMAN | Histone-lysine N-methyltransferase MECOM |
| MECP2 | MECP2_HUMAN | Methyl-CpG-binding protein 2 |
| MEFV | MEFV_HUMAN | Pyrin |
| MELK | MELK_HUMAN | Maternal embryonic leucine zipper kinase |
| MEN1 | MEN1_HUMAN | Menin |
| MEPIB | MEP1B_HUMAN | Meprin A subunit beta |
| MERTK | MERTK_HUMAN | Tyrosine-protein kinase Mer |
| MET | MET_HUMAN | Hepatocyte growth factor receptor |
| METAP2 | MAP2_HUMAN | Methionine aminopeptidase 2 |
| METTL16 | MET16_HUMAN | RNA N6-adenosine-methyltransferase METTL16 |
| METTL18 | MET18_HUMAN | Histidine protein methyltransferase 1 homolog |
| MEX3C | MEX3C_HUMAN | RNA-binding E3 ubiquitin-protein ligase MEX3C |
| MGAM | MGA_HUMAN | Glucoamylase |
| MGLL | MGLL_HUMAN | Monoglyceride lipase |
| MGMT | MGMT_HUMAN | Methylated-DNA--protein-cysteine methyltransferase |
| M1A | M1A_HUMAN | Melanoma-derived growth regulatory protein |
| M1Bl | M1Bl_HUMAN | E3 ubiquitin-protein ligase M1B1 |
| M1B2 | M1B2_HUMAN | E3 ubiquitin-protein ligase M1B2 |
| M1CAL1 | M1CA1_HUMAN | [F-actin]-monooxygenase M1CAL1 |
| M1CU1 | M1CU1_HUMAN | Calcium uptake protein 1, mitochondrial |
| MINDY1 | M1NY1_HUMAN | Ubiquitin carboxyl-terminal hydro lase MINDY-1 |
| MKNK1 | MKNK1_HUMAN | MAP kinase-interacting serine/threonine-protein kinase 1 |
| MLH1 | MLH1_HUMAN | DNA mismatch repair protein MIhl |
| MLLT1 | ENL_HUMAN | Protein ENL |
| MLLT10 | AF10_HUMAN | Protein AF-10 |
| MLLT3 | AF9_HUMAN | Protein AF -9 |
| MLLT6 | AF17_HUMAN | Protein AF -17 |
| MLPH | MELPH_HUMAN | Melanophilin |
| MLST8 | LST8_HUMAN | Target of rapamycin complex subunit LST8 |
| MMAB | MMAB_HUMAN | Corrinoid adenosyltransferase |
| MMADHC | MMAD_HUMAN | Methylmalonic aciduria and homocystinuria type D protein, |
| | | mitochondrial |
| MME | NEP_HUMAN | Neprilysin |
| MMP1 | MMP1_HUMAN | 27 kDa interstitial collagenase |
| MMP13 | MMP13_HUMAN | Collagenase 3 |
| MMP14 | MMP14_HUMAN | Matrix metalloproteinase-14 |
| MMP2 | MMP2_HUMAN | PEX |
| MMUT | MUTA_HUMAN | Methylmalonyl-CoA mutase, mitochondrial |
| MNAT1 | MAT1_HUMAN | CDK-activating kinase assembly factor MATI |
| MPG | 3MG_HUMAN | DNA-3-methyladenine glycosylase |
| MPP7 | MPP7_HUMAN | MAGUK p55 subfamily member 7 |
| MPST | THTM_HUMAN | 3-mercaptopyruvate sulfurtransferase |
| MR1 | HMR1_HUMAN | Major histocompatibility complex class I-related gene protein |
| MRC1 | MRC1_HUMAN | Macrophage mannose receptor 1 |
| MRC2 | MRC2_HUMAN | C-type mannose receptor 2 |
| MR11 | MTNA_HUMAN | Methylthioribose-1-phosphate isomerase |
| MRPL13 | RM13_HUMAN | 39S ribosomal protein Ll3, mitochondrial |
| MRPL18 | RM18_HUMAN | 39S ribosomal protein Ll8, mitochondrial |
| MRPL24 | RM24_HUMAN | 39S ribosomal protein L24, mitochondrial |
| MRPL28 | RM28_HUMAN | 39S ribosomal protein L28, mitochondrial |
| MRPL3 | RM03_HUMAN | 39S ribosomal protein L3, mitochondrial |
| MRPL30 | RM30_HUMAN | 39S ribosomal protein L30, mitochondrial |
| MRPL32 | RM32_HUMAN | 39S ribosomal protein L32, mitochondrial |
| MRPL35 | RM35_HUMAN | 39S ribosomal protein L35, mitochondrial |
| MRPL43 | RM43_HUMAN | 39S ribosomal protein L43, mitochondrial |
| MRPL45 | RM45_HUMAN | 39S ribosomal protein L45, mitochondrial |
| MRPL46 | RM46_HUMAN | 39S ribosomal protein L46, mitochondrial |
| MRPL47 | RM47_HUMAN | 39S ribosomal protein L47, mitochondrial |
| MRPL49 | RM49_HUMAN | 39S ribosomal protein L49, mitochondrial |
| MRPL53 | RM53_HUMAN | 39S ribosomal protein L53, mitochondrial |
| MRPL55 | RM55_HUMAN | 39S ribosomal protein L55, mitochondrial |
| MRPS18A | RT18A_HUMAN | 39S ribosomal protein Sl8a, mitochondrial |
| MSH2 | MSH2_HUMAN | DNA mismatch repair protein Msh2 |
| MSH3 | MSH3_HUMAN | DNA mismatch repair protein Msh3 |
| MSH6 | MSH6_HUMAN | DNA mismatch repair protein Msh6 |
| MSL2 | MSL2_HUMAN | E3 ubiquitin-protein ligase MSL2 |
| MSL3 | MS3LI_HUMAN | Male-specific lethal 3 homolog |
| MSMB | MSMB_HUMAN | Beta-microseminoprotein |
| MSN | MOES_HUMAN | Moesin |
| MSRB1 | MSRB1_HUMAN | Methionine-R-sulfoxide reductase Bl |
| MST1R | RON_HUMAN | Macrophage-stimulating protein receptor beta chain |
| MSTN | GDF8_HUMAN | Growth/differentiation factor 8 |
| MT-CO2 | COX2_HUMAN | Cytochrome c oxidase subunit 2 |
| MTERF4 | MTEF4_HUMAN | mTERF domain-containing protein 2 processed |
| MTF1 | MTF1_HUMAN | Metal regulatory transcription factor 1 |
| MTF2 | MTF2_HUMAN | Metal-response element-binding transcription factor 2 |
| MTHFR | MTHR_HUMAN | Methylenetetrahydrofolate reductase |
| MTHFS | MTHFS_HUMAN | 5-formyltetrahydrofolate cyclo-ligase |
| MT1F3 | IF3M_HUMAN | Translation initiation factor lF-3, mitochondrial |
| MTMR1 | MTMR1_HUMAN | Myotubularin-related protein 1 |
| MTMR2 | MTMR2_HUMAN | Myotubularin-related protein 2 |
| MTMR3 | MTMR3_HUMAN | Myotubularin-related protein 3 |
| MTMR4 | MTMR4_HUMAN | Myotubularin-related protein 4 |
| MTOR | MTOR_HUMAN | Serine/threonine-protein kinase mTOR |
| MTPAP | PAPD1_HUMAN | Poly(A) RNA polymerase, mitochondrial |
| MTR | METH_HUMAN | Methionine synthase |
| MVK | K1ME_HUMAN | Mevalonate kinase |
| MYBPC3 | MYPC3_HUMAN | Myosin-binding protein C, cardiac-type |
| MYCBP2 | MYCB2_HUMAN | E3 ubiquitin-protein ligase MYCBP2 |
| MYH10 | MYH10_HUMAN | Myosin-10 |
| MYH14 | MYH14_HUMAN | Myosin-14 |
| MYH7 | MYH7_HUMAN | Myosin-7 |
| MYL3 | MYL3_HUMAN | Myosin light chain 3 |
| MYL6B | MYL6B_HUMAN | Myosin light chain 6B |
| MYL1P | MYLIP_HUMAN | E3 ubiquitin-protein ligase MYL1P |
| MYLK4 | MYLK4_HUMAN | Myosin light chain kinase family member 4 |
| MYNN | MYNN_HUMAN | Myoneurin |
| MYOl0 | MYOl0_HUMAN | Unconventional myosin-X |
| MYO1C | MYOlC_HUMAN | Unconventional myosin-Ic |
| MYO5C | MYO5C_HUMAN | Unconventional myosin-Vc |
| MYO7A | MYO7A_HUMAN | Unconventional myosin-VIIa |
| MYO7B | MYO7B_HUMAN | Unconventional myosin-VIIb |
| MYOC | MYOC_HUMAN | Myocilin, C-terminal fragment |
| MYOF | MYOF_HUMAN | Myoferlin |
| MYOM1 | MYOM1_HUMAN | Myomesin-1 |
| MYOT | MYOT1_HUMAN | Myotilin |
| MYRF | MYRF_HUMAN | Myelin regulatory factor, C-terminal |
| MYZAP | MYZAP_HUMAN | Myocardial zonula adherens protein |
| MZF1 | MZF1_HUMAN | Myeloid zinc finger 1 |
| NAA10 | NAA10_HUMAN | N-alpha-acetyltransferase 10 |
| NAAA | NAAA_HUMAN | N-acylethanolamine-hydrolyzing acid amidase subunit beta |
| NAALADLl | NALDL_HUMAN | Aminopeptidase NAALADL1 |
| NABP2 | SOSBl_HUMAN | SOSS complex subunit B1 |
| NAE1 | ULAl_HUMAN | NEDD8-activating enzyme El regulatory subunit |
| NAGA | NAGAB_HUMAN | Alpha-N-acety Igalactosaminidase |
| NAGK | NAGK_HUMAN | N-acetyl-D-glucosamine kinase |
| NA1P | B1RC1_HUMAN | Baculoviral IAP repeat-containing protein 1 |
| NAMPT | NAMPT_HUMAN | Nicotinamide phosphoribosyltransferase |
| NANOS1 | NANO1_HUMAN | Nanos homolog 1 |
| NANOS2 | NANO2_HUMAN | Nanos homolog 2 |
| NANOS3 | NANO3_HUMAN | Nanos homolog 3 |
| NARS | SYNC_HUMAN | Asparagine--tRNA ligase, cytoplasmic |
| NCAM1 | NCAM1_HUMAN | Neural cell adhesion molecule 1 |
| NCAM2 | NCAM2_HUMAN | Neural cell adhesion molecule 2 |
| NCF4 | NCF4_HUMAN | Neutrophil cytosol factor 4 |
| NCK1 | NCK1_HUMAN | Cytoplasmic protein NCK1 |
| NCK2 | NCK2_HUMAN | Cytoplasmic protein NCK2 |
| NCL | NUCL_HUMAN | Nucleolin |
| NCOA1 | NCOA1_HUMAN | Nuclear receptor coactivator 1 |
| NCR2 | NCTR2_HUMAN | Natural cytotoxicity triggering receptor 2 |
| NCR3 | NCTR3_HUMAN | Natural cytotoxicity triggering receptor 3 |
| NCR3LG1 | NR3Ll_HUMAN | Natural cytotoxicity triggering receptor 3 ligand 1 |
| NDP | NDP_HUMAN | Norrin |
| NDRG2 | NDRG2_HUMAN | Protein NDRG2 |
| NDSTl | NDSTl_HUMAN | Heparan sulfate N-sulfotransferase 1 |
| NDUFA2 | NDUA2_HUMAN | NADH dehydrogenase [ubiquinone] 1 alpha subcomplex subunit 2 |
| NDUFS1 | NDUSl_HUMAN | NADH-ubiquinone oxidoreductase 75 kDa subunit, mitochondrial |
| NDUFS4 | NDUS4_HUMAN | NADH dehydrogenase [ubiquinone] iron-sulfur protein 4, mitochondrial |
| NDUFS6 | NDUS6_HUMAN | NADH dehydrogenase [ubiquinone] iron-sulfur protein 6, mitochondrial |
| NDUFVl | NDUVl_HUMAN | NADH dehydrogenase [ubiquinone] flavoprotein 1, mitochondrial |
| NEB | NEBU_HUMAN | Nebulin |
| NEBL | NEBL_HUMAN | Nebulette |
| NECTIN1 | NECT1_HUMAN | Nectin-1 |
| NECTIN2 | NECT2_HUMAN | Nectin-2 |
| NECTIN3 | NECT3_HUMAN | Nectin-3 |
| NECTIN4 | NECT4_HUMAN | Processed poliovirus receptor-related protein 4 |
| NEDD4 | NEDD4_HUMAN | E3 ubiquitin-protein ligase NEDD4 |
| NEDD4L | NED4L_HUMAN | E3 ubiquitin-protein ligase NEDD4-like |
| NEDD8 | NEDD8_HUMAN | NEDD8 |
| NEIL1 | NEIL1_HUMAN | Endonuclease 8-like 1 |
| NEK1 | NEK1_HUMAN | Serine/threonine-protein kinase Nekl |
| NEK2 | NEK2_HUMAN | Serine/threonine-protein kinase Nek2 |
| NEK7 | NEK7_HUMAN | Serine/threonine-protein kinase Nek7 |
| NEO1 | NEO1_HUMAN | Neogenin |
| NET1 | ARHG8_HUMAN | Neuroepithelial cell-transforming gene 1 protein |
| NEU2 | NEUR2_HUMAN | Sialidase-2 |
| NEURL1 | NEULl_HUMAN | E3 ubiquitin-protein ligase NEURL1 |
| NEURL1B | NEU1B_HUMAN | E3 ubiquitin-protein ligase NEURL1B |
| NEURL4 | NEUL4_HUMAN | Neuralized-like protein 4 |
| NF1 | NF1_HUMAN | Neurofibromin truncated |
| NF2 | MERL_HUMAN | Merlin |
| NFASC | NFASC_HUMAN | Neurofascin |
| NFATC1 | NFAC1_HUMAN | Nuclear factor of activated T-cells, cytoplasmic 1 |
| NFATC2 | NFAC2_HUMAN | Nuclear factor of activated T-cells, cytoplasmic 2 |
| NFE2L2 | NF2L2_HUMAN | Nuclear factor erythroid 2-related factor 2 |
| NFKB1 | NFKB1_HUMAN | Nuclear factor NF-kappa-B p50 subunit |
| NFKB2 | NFKB2_HUMAN | Nuclear factor NF-kappa-B p52 subunit |
| NFKBlA | lKBA_HUMAN | NF-kappa-B inhibitor alpha |
| NFS1 | NFS1_HUMAN | Cysteine desulfurase, mitochondrial |
| NGF | NGF_HUMAN | Beta-nerve growth factor |
| NHLRC2 | NHLC2_HUMAN | NHL repeat-containing protein 2 |
| NKTR | NKTR_HUMAN | NK-tumor recognition protein |
| NLGN1 | NLGN1_HUMAN | Neuroligin-1 |
| NLGN2 | NLGN2_HUMAN | Neuroligin-2 |
| NLGN4X | NLGNX_HUMAN | Neuroligin-4, X-linked |
| NLN | NEUL_HUMAN | Neurolysin, mitochondrial |
| NMRKl | NRK1_HUMAN | Nicotinamide riboside kinase 1 |
| NMTl | NMT1_HUMAN | Glycylpeptide N-tetradecanoyltransferase 1 |
| NNMT | NNMT_HUMAN | Nicotinamide N-methyltransferase |
| NOBl | NOBI_HUMAN | RNA-binding protein NOB1 |
| NOCT | NOCT_HUMAN | Nocturnin |
| NONO | NONO_HUMAN | Non-POU domain-containing octamer-binding protein |
| NOSl | NOSI_HUMAN | Nitric oxide synthase, brain |
| NOS2 | NOS2_HUMAN | Nitric oxide synthase, inducible |
| NOS3 | NOS3_HUMAN | Nitric oxide synthase, endothelial |
| NOTCH1 | NOTCl_HUMAN | Notch 1 intracellular domain |
| NOTUM | NOTUM_HUMAN | Palmitoleoyl-protein carboxylesterase NOTUM |
| NPC1 | NPCl_HUMAN | NPC intracellular cholesterol transporter 1 |
| NPHP1 | NPHPl_HUMAN | Nephrocystin-1 |
| NPM1 | NPM_HUMAN | Nucleophosmin |
| NPR1 | ANPRA_HUMAN | Atrial natriuretic peptide receptor 1 |
| NPR2 | ANPRB_HUMAN | Atrial natriuretic peptide receptor 2 |
| NPR3 | ANPRC_HUMAN | Atrial natriuretic peptide receptor 3 |
| NPRL2 | NPRL2_HUMAN | GATOR complex protein NPRL2 |
| NPTN | NPTN_HUMAN | Neuroplastin |
| NPY1R | NPY1R_HUMAN | Neuropeptide Y receptor type 1 |
| NR1DI | NR1D1_HUMAN | Nuclear receptor subfamily 1 group D member 1 |
| NR1D2 | NR1D2_HUMAN | Nuclear receptor subfamily 1 group D member 2 |
| NR1H2 | NR1H2_HUMAN | Oxysterols receptor LXR-beta |
| NR1H3 | NR1H3_HUMAN | Oxysterols receptor LXR-alpha |
| NR1H4 | NR1H4_HUMAN | Bile acid receptor |
| NR112 | NR112_HUMAN | Nuclear receptor subfamily 1 group 1 member 2 |
| NR113 | NR113_HUMAN | Nuclear receptor subfamily 1 group 1 member 3 |
| NR2CI | NR2CI_HUMAN | Nuclear receptor subfamily 2 group C member 1 |
| NR2C2 | NR2C2_HUMAN | Nuclear receptor subfamily 2 group C member 2 |
| NR2El | NR2El_HUMAN | Nuclear receptor subfamily 2 group E member 1 |
| NR2E3 | NR2E3_HUMAN | Photoreceptor-specific nuclear receptor |
| NR2Fl | COT1_HUMAN | COUP transcription factor 1 |
| NR2F2 | COT2_HUMAN | COUP transcription factor 2 |
| NR2F6 | NR2F6_HUMAN | Nuclear receptor subfamily 2 group F member 6 |
| NR3Cl | GCR_HUMAN | Glucocorticoid receptor |
| NR3C2 | MCR_HUMAN | Mineralocorticoid receptor |
| NR4AI | NR4Al_HUMAN | Nuclear receptor subfamily 4 group A member 1 |
| NR4A2 | NR4A2_HUMAN | Nuclear receptor subfamily 4 group A member 2 |
| NR4A3 | NR4A3_HUMAN | Nuclear receptor subfamily 4 group A member 3 |
| NR5Al | STFI_HUMAN | Steroidogenic factor 1 |
| NR5A2 | NR5A2_HUMAN | Nuclear receptor subfamily 5 group A member 2 |
| NR6Al | NR6Al_HUMAN | Nuclear receptor subfamily 6 group A member 1 |
| NRCAM | NRCAM_HUMAN | Neuronal cell adhesion molecule |
| NSDI | NSDl_HUMAN | Histone-lysine N-methyltransferase, H3 lysine-36 and H4 lysine-20 |
| | | specific |
| NSD2 | NSD2_HUMAN | Histone-lysine N-methyltransferase NSD2 |
| NSD3 | NSD3_HUMAN | Histone-lysine N-methyltransferase NSD3 |
| NSFL1C | NSF1C_HUMAN | NSFLI cofactor p47 |
| NSMCE1 | NSEl_HUMAN | Non-structural maintenance of chromosomes element 1 homolog |
| NSMCE2 | NSE2_HUMAN | E3 SUMO-protein ligase NSE2 |
| NT5C2 | 5NTC_HUMAN | Cytosolic purine 5′-nucleotidase |
| NT5E | 5NTD_HUMAN | 5′-nucleotidase |
| NTF3 | NTF3_HUMAN | Neurotrophin-3 |
| NTF4 | NTF4_HUMAN | Neurotrophin-4 |
| NTN1 | NET1_HUMAN | Netrin-1 |
| NTNG1 | NTNG1_HUMAN | Netrin-Gl |
| NTNG2 | NTNG2_HUMAN | Netrin-G2 |
| NTPCR | NTPCR_HUMAN | Cancer-related nucleoside-triphosphatase |
| NTRK1 | NTRKIlHUMAN | High affinity nerve growth factor receptor |
| NTRK2 | NTRK2_HUMAN | BDNF/NT-3 growth factors receptor |
| NTRK3 | NTRK3_HUMAN | NT-3 growth factor receptor |
| NUDT1 | 8ODP_HUMAN | 7,8-dihydro-8-oxoguanine triphosphatase |
| NUDT14 | NUD14_HUMAN | Uridine diphosphate glucose pyrophosphatase |
| NUDT16 | NUD16_HUMAN | U8 snoRNA-decapping enzyme |
| NUDT4 | NUDT4_HUMAN | Diphosphoinositol polyphosphate phosphohydrolase 2 |
| NUDT5 | NUDT5_HUMAN | ADP-sugar pyrophosphatase |
| NUDT6 | NUDT6_HUMAN | Nucleoside diphosphate-linked moiety X motif 6 |
| NUDT7 | NUDT7_HUMAN | Peroxisomal coenzyme A diphosphatase NUDT7 |
| NUDT9 | NUDT9_HUMAN | ADP-ribose pyrophosphatase, mitochondrial |
| NUMB | NUMB_HUMAN | Protein numb homolog |
| NUP133 | NU133_HUMAN | Nuclear pore complex protein Nupl33 |
| NUP155 | NU155_HUMAN | Nuclear pore complex protein Nupl55 |
| NUP160 | NU160_HUMAN | Nuclear pore complex protein Nupl60 |
| NUP214 | NU214_HUMAN | Nuclear pore complex protein Nup2 \| 4 |
| NUP37 | NUP37_HUMAN | Nucleoporin Nup37 |
| NUP43 | NUP43_HUMAN | Nucleoporin Nup43 |
| NUP50 | NUP50_HUMAN | Nuclear pore complex protein Nup50 |
| NUP54 | NUP54_HUMAN | Nucleoporin p54 |
| NUP98 | NUP98_HUMAN | Nuclear pore complex protein Nup96 |
| NXF1 | NXF1_HUMAN | Nuclear RNA export factor 1 |
| OAS1 | OAS1_HUMAN | 2′-5′-oligoadenylate synthase 1 |
| OASL | OASL_HUMAN | 2′-5′-oligoadenylate synthase-like protein |
| OAT | OAT_HUMAN | Ornithine aminotransferase, renal form |
| OBP2A | OBP2A_HUMAN | Odorant-binding protein 2a |
| OBSCN | OBSCN_HUMAN | Obscurin |
| OBSL1 | OBSL1_HUMAN | Obscurin-like protein 1 |
| OLFM1 | NOE1_HUMAN | Noelin |
| OPCML | OPCM_HUMAN | Opioid-binding protein/cell adhesion molecule |
| OPRK1 | OPRK_HUMAN | Kappa-type opioid receptor |
| OPTN | OPTN_HUMAN | Optineurin |
| ORC2 | ORC2_HUMAN | Origin recognition complex subunit 2 |
| ORM1 | A1AG1_HUMAN | Alpha- I-acid glycoprotein 1 |
| ORM2 | AlAG2_HUMAN | Alpha- I-acid glycoprotein 2 |
| OS9 | OS9_HUMAN | Protein OS-9 |
| OSBPL11 | OSB11_HUMAN | Oxysterol-binding protein-related protein 11 |
| OSBPL1A | OSBL1_HUMAN | Oxysterol-binding protein-related protein 1 |
| OSBPL2 | OSBL2_HUMAN | Oxysterol-binding protein-related protein 2 |
| OSBPL8 | OSBL8_HUMAN | Oxysterol-binding protein-related protein 8 |
| OSR1 | OSRI_HUMAN | Protein odd-skipped-related 1 |
| OSR2 | OSR2_HUMAN | Protein odd-skipped-related 2 |
| OSTF1 | OSTFl_HUMAN | Osteoclast-stimulating factor 1 |
| OTUD1 | OTUDl_HUMAN | OTU domain-containing protein 1 |
| OVOL1 | OVOLl_HUMAN | Putative transcription factor Ovo-like 1 |
| OVOL2 | OVOL2_HUMAN | Transcription factor Ovo-like 2 |
| OVOL3 | OVOL3_HUMAN | Putative transcription factor ovo-like protein 3 |
| OXCT1 | SCOTl_HUMAN | Succinyl-CoA:3-ketoacid coenzyme A transferase 1, mitochondrial |
| OXSM | OXSM_HUMAN | 3-oxoacy 1-[acyl-carrier-protein] synthase, mitochondrial |
| OXSR1 | OXSR1_HUMAN | Serine/threonine-protein kinase OSRI |
| P2RX3 | P2RX3_HUMAN | P2X purinoceptor 3 |
| P2RY1 | P2RY1_HUMAN | P2Y purinoceptor 1 |
| PABPCl | PABP1_HUMAN | Polyadeny late-binding protein 1 |
| PACSlN1 | PACN1_HUMAN | Protein kinase C and casein kinase substrate in neurons protein 1 |
| PACS1N2 | PACN2_HUMAN | Protein kinase C and casein kinase substrate in neurons protein 2 |
| PAD12 | PAD12_HUMAN | Protein-arginine deiminase type-2 |
| PAD14 | PAD14_HUMAN | Protein-arginine deiminase type-4 |
| PAFl | PAF1_HUMAN | RNA polymerase II-associated factor 1 homolog |
| PAlP1 | PAlPl_HUMAN | Polyadenylate-binding protein-interacting protein 1 |
| PAKl | PAK1_HUMAN | Serine/threonine-protein kinase PAK 1 |
| PAK2 | PAK2_HUMAN | PAK-2p34 |
| PAK3 | PAK3_HUMAN | Serine/threonine-protein kinase PAK 3 |
| PAK4 | PAK4_HUMAN | Serine/threonine-protein kinase PAK 4 |
| PAK5 | PAK5_HUMAN | Serine/threonine-protein kinase PAK 5 |
| PAK6 | PAK6_HUMAN | Serine/threonine-protein kinase PAK 6 |
| PALB2 | PALB2_HUMAN | Partner and localizer of BRCA2 |
| PALLD | PALLD_HUMAN | Palladin |
| PANK1 | PANK1_HUMAN | Pantothenate kinase 1 |
| PANK2 | PANK2_HUMAN | Pantothenate kinase 2, mitochondrial |
| PANK3 | PANK3_HUMAN | Pantothenate kinase 3 |
| PAPSS1 | PAPS1_HUMAN | Adenyly-sulfate kinase |
| PARD3 | PARD3_HUMAN | Partitioning defective 3 homolog |
| PARD6A | PAR6A_HUMAN | Partitioning defective 6 homolog alpha |
| PARP1 | PARP1_HUMAN | Poly [ADP-ribose] polymerase 1 |
| PARP10 | PAR10_HUMAN | Protein mono-ADP-ribosyltransferase PARP10 |
| PARP11 | PAR11_HUMAN | Protein mono-ADP-ribosyltransferase PARP11 |
| PARP14 | PAR14_HUMAN | Protein mono-ADP-ribosyltransferase PARP14 |
| PARP15 | PAR15_HUMAN | Protein mono-ADP-ribosyltransferase PARP15 |
| PASK | PASK_HUMAN | PAS domain-containing serine/threonine-protein ckinase |
| PATJ | INADL_HUMAN | InaD-like protein |
| PATZ1 | PATZ1_HUMAN | POZ-, AT hook-, and zinc finger-containing protein 1 |
| PAX5 | PAX5_HUMAN | Paired box protein Pax-5 |
| PAX6 | PAX6_HUMAN | Paired box protein Pax-6 |
| PBRM1 | PB1_HUMAN | Protein polybromo-1 |
| PC | PYC_HUMAN | Pyruvate carboxylase, mitochondrial |
| PCBD2 | PHS2_HUMAN | Pterin-4-alpha-carbinolamine dehydratase 2 |
| PCDH1 | PCDH1_HUMAN | Protocadherin-1 |
| PCDH15 | PCD15_HUMAN | Protocadherin-15 |
| PCDH7 | PCDH7_HUMAN | Protocadherin-7 |
| PCDH9 | PCDH9_HUMAN | Protocadherin-9 |
| PCDHGB3 | PCDGF_HUMAN | Protocadherin gamma-B3 |
| PCGF2 | PCGF2_HUMAN | Polycomb group RING finger protein 2 |
| PCGF5 | PCGF5_HUMAN | Polycomb group RING finger protein 5 |
| PCK1 | PCKGC_HUMAN | Phosphoenolpymvate carboxykinase, cytosolic [GTP] |
| PCMT1 | PIMT_HUMAN | Protein-L-isoaspartate(D-aspartate) 0-methy Itransferase |
| PCNA | PCNA_HUMAN | Proliferating cell nuclear antigen |
| PCOLCE | PCOC1_HUMAN | Procollagen C-endopeptidase enhancer 1 |
| PCSK9 | PCSK9_HUMAN | Proprotein convertase subtilisin/kexin type 9 |
| PCTP | PPCT_HUMAN | Phosphatidylcholine transfer protein |
| PDCD1 | PDCD1_HUMAN | Programmed cell death protein 1 |
| PDCD11 | RRP5_HUMAN | Protein RRP5 homolog |
| PDCD2 | PDCD2_HUMAN | Programmed cell death protein 2 |
| PDCD6 | PDCD6_HUMAN | Programmed cell death protein 6 |
| PDE4B | PDE4B_HUMAN | CAMP-specific 3′,5′-cyclic phosphodiesterase 4B |
| PDE4D | PDE4D_HUMAN | CAMP-specific 3′,5′-cyclic phosphodiesterase 4D |
| PDE5A | PDE5A_HUMAN | CGMP-specific 3′,5′-cyclic phosphodiesterase |
| PDE6D | PDE6D_HUMAN | Retinal rod rhodopsin-sensitive cGMP 3′,5′-cyclic phosphodiesterase |
| | | subunit delta |
| PDF | DEFM_HUMAN | Peptide deformylase, mitochondrial |
| PDGFRB | PGFRB_HUMAN | Platelet-derived growth factor receptor beta |
| PD1A3 | PD1A3_HUMAN | Protein disulfide-isomerase A3 |
| PDK2 | PDK2_HUMAN | [Pymvate dehydrogenase (acetyl-transferring)] kinase isozyme 2, |
| | | mitochondrial |
| PDK4 | PDK4_HUMAN | [Pymvate dehydrogenase (acetyl-transferring)] kinase isozyme 4, |
| | | mitochondrial |
| PDL1Ml | PDLI1_HUMAN | PDZ and LIM domain protein 1 |
| PDXK | PDXK_HUMAN | Pyridoxal kinase |
| PDZD3 | NHRF4_HUMAN | Na(+)/H(+) exchange regulatory cofactor NHERF4 |
| PDZRN3 | PZRN3_HUMAN | E3 ubiquitin-protein ligase PDZRN3 |
| PDZRN4 | PZRN4_HUMAN | PDZ domain-containing RING finger protein 4 |
| PEG10 | PEG10_HUMAN | Retrotransposon-derived protein PEG 10 |
| PEG3 | PEG3_HUMAN | Paternally-expressed gene 3 protein |
| PEL12 | PELl2_HUMAN | E3 ubiquitin-protein ligase pellino homolog 2 |
| PEPD | PEPD_HUMAN | Xaa-Pro dipeptidase |
| PEX2 | PEX2_HUMAN | Peroxisome biogenesis factor 2 |
| PEX5 | PEX5_HUMAN | Peroxisomal targeting signal 1 receptor |
| PF4 | PLF4_HUMAN | Platelet factor 4, short form |
| PF4Vl | PF4V_HUMAN | Platelet factor 4 variant(6-7 4) |
| PFKFBl | F261_HUMAN | Fmctose-2,6-bisphosphatase |
| PGA4 | PEPA4_HUMAN | PepsinA-4 |
| PGAM5 | PGAM5_HUMAN | Serine/threonine-protein phosphatase PGAM5, mitochondrial |
| PGC | PEPC_HUMAN | Gastricsin |
| PGD | 6PGD_HUMAN | 6-phosphogluconate dehydrogenase, decarboxylating |
| PGK1 | PGK1_HUMAN | Phosphoglycerate kinase 1 |
| PGLYRP3 | PGRP3_HUMAN | Peptidoglycan recognition protein 3 |
| PGLYRP4 | PGRP4_HUMAN | Peptidoglycan recognition protein 4 |
| PGM1 | PGM1_HUMAN | Phosphoglucomutase-1 |
| PGR | PRGR_HUMAN | Progesterone receptor |
| PHC1 | PHC1_HUMAN | Polyhomeotic-like protein 1 |
| PHC2 | PHC2_HUMAN | Polyhomeotic-like protein 2 |
| PHC3 | PHC3_HUMAN | Polyhomeotic-like protein 3 |
| PHF1 | PHF1_HUMAN | PHD finger protein 1 |
| PHF14 | PHF14_HUMAN | PHD finger protein 14 |
| PHF19 | PHF19_HUMAN | PHD finger protein 19 |
| PHF20 | PHF20_HUMAN | PHD finger protein 20 |
| PHF20L1 | P20Ll_HUMAN | PHD finger protein 20-like protein 1 |
| PHF23 | PHF23_HUMAN | PHD finger protein 23 |
| PHF5A | PHF5A_HUMAN | PHD finger-like domain-containing protein 5A |
| PHF6 | PHF6_HUMAN | PHD finger protein 6 |
| PHF7 | PHF7_HUMAN | PHD finger protein 7 |
| PHKG2 | PHKG2_HUMAN | Phosphorylase b kinase gamma catalytic chain, liver/testis isoform |
| PHRF1 | PHRF1_HUMAN | PHD and RING finger domain-containing protein 1 |
| Pl4K2A | P4K2A_HUMAN | Phosphatidylinositol 4-kinase type 2-alpha |
| Pl4K2B | P4K2B_HUMAN | Phosphatidylinositol 4-kinase type 2-beta |
| Pl4KA | Pl4KA_HUMAN | Phosphatidylinositol 4-kinase alpha |
| Pl4KB | Pl4KB_HUMAN | Phosphatidylinositol 4-kinase beta |
| PlAS3 | PIAS3_HUMAN | E3 SUMO-protein ligase PIAS3 |
| PIFl | PIFl_HUMAN | ATP-dependent DNA helicase PIFl |
| PIGR | PIGR_HUMAN | Secretory component |
| PIHlDl | PIHDl_HUMAN | PIH1 domain-containing protein 1 |
| PIK3C3 | PK3C3_HUMAN | Phosphatidylinositol 3-kinase catalytic subunit type 3 |
| PIK3CA | PK3CA_HUMAN | Phosphatidylinositol 4,5-bisphosphate 3-kinase catalytic subunit alpha |
| | | isoform |
| PIK3CD | PK3CD_HUMAN | Phosphatidylinositol 4,5-bisphosphate 3-kinase catalytic subunit delta |
| | | isoform |
| PIK3CG | PK3CG_HUMAN | Phosphatidylinositol 4,5-bisphosphate 3-kinase catalytic subunit gamma |
| | | isoform |
| PIK3Rl | P85A_HUMAN | Phosphatidylinositol 3-kinase regulatory subunit alpha |
| PIKFYVE | FYV1_HUMAN | 1-phosphatidylinositol 3-phosphate 5-kinase |
| PILRA | PILRA_HUMAN | Paired immunoglobulin-like type 2 receptor alpha |
| PILRB | PILRB_HUMAN | Paired immunoglobulin-like type 2 receptor beta |
| PIM1 | PIM1_HUMAN | Serine/threonine-protein kinase pim-1 |
| PIM2 | PIM2_HUMAN | Serine/threonine-protein kinase pim-2 |
| PIN1 | PIN1_HUMAN | Peptidyl-prolyl cis-trans isomerase NIMA-interacting 1 |
| PIN4 | PIN4_HUMAN | Peptidyl-prolyl cis-trans isomerase NIMA-interacting 4 |
| PIP4K2B | Pl42B_HUMAN | Phosphatidylinositol 5-phosphate 4-kinase type-2 beta |
| PIR | PIR_HUMAN | Pirin |
| PITPNA | PIPNA_HUMAN | Phosphatidylinositol transfer protein alpha isoform |
| PlTRM1 | PREP_HUMAN | Presequence protease, mitochondrial |
| PlWlL1 | PlWL1_HUMAN | Piwi-like protein 1 |
| PlWlL2 | PlWL2_HUMAN | Piwi-like protein 2 |
| PKD1 | PKD1_HUMAN | Polycystin-1 |
| PKD2 | PKD2_HUMAN | Polycystin-2 |
| PKD2Ll | PK2Ll_HUMAN | Polycystic kidney disease 2-like 1 protein |
| PKLR | KPYR_HUMAN | Pymvate kinase PKLR |
| PKM | KPYM_HUMAN | Pymvate kinase PKM |
| PKMYT1 | PMYT1_HUMAN | Membrane-associated tyrosine- and threonine-specific cdc2-inhibitory |
| | | kinase |
| PKN1 | PKN1_HUMAN | Serine/threonine-protein kinase Nl |
| PKNZ | PKN2_HUMAN | Serine/threonine-protein kinase N2 |
| PLA2G2E | PA2GE_HUMAN | Group IIE secretory phospholipase A2 |
| PLA2G4A | PA24A_HUMAN | Lysophospholipase |
| PLA2G4D | PA24D_HUMAN | Cytosolic phospholipase A2 delta |
| PLAA | PLAP_HUMAN | Phospholipase A-2-activating protein |
| PLAG1 | PLAG1_HUMAN | Zinc finger protein PLAG1 |
| PLAGL1 | PLAL1_HUMAN | Zinc finger protein PLAGLl |
| PLAGL2 | PLAL2_HUMAN | Zinc finger protein PLAGL2 |
| PLAU | UROK_HUMAN | Urokinase-type plasminogen activator chain B |
| PLAUR | UPAR_HUMAN | Urokinase plasminogen activator surface receptor |
| PLCG1 | PLCG1_HUMAN | 1-phosphatidy linositol 4,5-bisphosphate phosphodiesterase gamma-l |
| PLCG2 | PLCG2_HUMAN | 1-phosphatidy linositol 4,5-bisphosphate phosphodiesterase gamma-2 |
| PLEC | PLEC_HUMAN | Plectin |
| PLEKHB2 | PKHB2_HUMAN | Pleckstrin homology domain-containing family B member 2 |
| PLEKHF1 | PKHF1_HUMAN | Pleckstrin homology domain-containing family F member 1 |
| PLEKHF2 | PKHF2_HUMAN | Pleckstrin homology domain-containing family F member 2 |
| PLEKHM3 | PKHM3_HUMAN | Pleckstrin homology domain-containing family M member 3 |
| PLG | PLMN_HUMAN | Plasmin light chain B |
| PLK1 | PLK1_HUMAN | Serine/threonine-protein kinase PLK1 |
| PLK2 | PLK2_HUMAN | Serine/threonine-protein kinase PLK2 |
| PLK3 | PLK3_HUMAN | Serine/threonine-protein kinase PLK3 |
| PLK4 | PLK4_HUMAN | Serine/threonine-protein kinase PLK4 |
| PLRG1 | PLRG1_HUMAN | Pleiotropic regulator 1 |
| PLXNA4 | PLXA4_HUMAN | Plexin-A4 |
| PLXNB1 | PLXB1_HUMAN | Plexin-B1 |
| PLXNB2 | PLXB2_HUMAN | Plexin-B2 |
| PLXNC1 | PLXC1_HUMAN | Plexin-Cl |
| PLXND1 | PLXD1_HUMAN | Plexin-Dl |
| PMS2 | PMS2_HUMAN | Mismatch repair endonuclease PMS2 |
| PNLIP | LIPP_HUMAN | Pancreatic triacylglycerol lipase |
| PNLIPRP1 | LIPR1_HUMAN | Inactive pancreatic lipase-related protein 1 |
| PNLIPRP2 | LIPR2_HUMAN | Pancreatic lipase-related protein 2 |
| PNMA3 | PNMA3_HUMAN | Paraneoplastic antigen Ma3 |
| PNPO | PNPO_HUMAN | Pyridoxine-5′-phosphate oxidase |
| PNPT1 | PNPT1_HUMAN | Polyribonucleotide nucleotidy Itransferase 1, mitochondrial |
| POGLUT2 | PLGT2_HUMAN | Protein O-glucosy Itransferase 2 |
| POLA1 | DPOLA_HUMAN | DNA polymerase alpha catalytic subunit |
| POLB | DPOLB_HUMAN | DNA polymerase beta |
| POLE2 | DPOE2_HUMAN | DNA polymerase epsilon subunit 2 |
| POLG | DPOG1_HUMAN | DNA polymerase subunit gamma-1 |
| POLG2 | DPOG2_HUMAN | DNA polymerase subunit gamma-2, mitochondrial |
| POLH | POLH_HUMAN | DNA polymerase eta |
| POLL | DPOLL_HUMAN | DNA polymerase lambda |
| POLM | DPOLM_HUMAN | DNA-directed DNA/RNA polymerase mu |
| POLN | DPOLN_HUMAN | DNA polymerase nu |
| POLQ | DPOLQ_HUMAN | DNA polymerase theta |
| POLR1B | RPA2_HUMAN | DNA-directed RNA polymerase I subunit RPA2 |
| POLR2A | RPB1_HUMAN | DNA-directed RNA polymerase II subunit RPB1 |
| POLR2B | RPB2_HUMAN | DNA-directed RNA polymerase II subunit RPB2 |
| POLR2E | RPAB1_HUMAN | DNA-directed RNA polymerases 1, II, and Ill subunit RPABC1 |
| POLR2G | RPB7_HUMAN | DNA-directed RNA polymerase II subunit RPB7 |
| POLR21 | RPB9_HUMAN | DNA-directed RNA polymerase II subunit RPB9 |
| POLR2K | RPAB4_HUMAN | DNA-directed RNA polymerases 1, II, and Ill subunit RPABC4 |
| POLR2L | RPAB5_HUMAN | DNA-directed RNA polymerases 1, II, and Ill subunit RPABC5 |
| POLR3B | RPC2_HUMAN | DNA-directed RNA polymerase Ill subunit RPC2 |
| POLR3C | RPC3_HUMAN | DNA-directed RNA polymerase Ill subunit RPC3 |
| POLR3K | RPC10_HUMAN | DNA-directed RNA polymerase Ill subunit RPC10 |
| POLRMT | RPOM_HUMAN | DNA-directed RNA polymerase, mitochondrial |
| POMGNT1 | PMGT1_HUMAN | Protein O-linked-mannose beta-1,2-Nacetylglucosaminyltransferase 1 |
| POP1 | POPI_HUMAN | Ribonucleases P/MRP protein subunit POP1 |
| POP5 | POP5_HUMAN | Ribonuclease P/MRP protein subunit POP5 |
| POR | NCPR_HUMAN | NADPH--cytochrome P450 reductase |
| POSTN | POSTN_HUMAN | Periostin |
| POT1 | POTE1_HUMAN | Protection of telomeres protein 1 |
| PPA1 | IPYR_HUMAN | Inorganic pyrophosphatase |
| PPARA | PPARA_HUMAN | Peroxisome proliferator-activated receptor alpha |
| PPARD | PPARD_HUMAN | Peroxisome proliferator-activated receptor delta |
| PPARG | PPARG_HUMAN | Peroxisome proliferator-activated receptor gamma |
| PPBP | CXCL7_HUMAN | Neutrophil-activating peptide 2(1-63) |
| PPIA | PP1A_HUMAN | Peptidyl-prolyl cis-trans isomerase A, N-terminally processed |
| PPIE | PPIE_HUMAN | Peptidyl-prolyl cis-trans isomerase E |
| PPIL1 | PPILI_HUMAN | Peptidyl-prolyl cis-trans isomerase-like 1 |
| PPIL3 | PPIL3_HUMAN | Peptidyl-prolyl cis-trans isomerase-like 3 |
| PPL | PEPL_HUMAN | Periplakin |
| PPM1K | PPM1K_HUMAN | Protein phosphatase lK, mitochondrial |
| PPME1 | PPME1_HUMAN | Protein phosphatase methylesterase 1 |
| PPOX | PPOX_HUMAN | Protoporphyrinogen oxidase |
| PPP1Rl3L | IASPP_HUMAN | RelA-associated inhibitor |
| PPP2R2A | 2ABA_HUMAN | Serine/threonine-protein phosphatase 2A 55 kDa regulatory subunit B |
| | | alpha isoform |
| PPP3CA | PP2BA_HUMAN | Serine/threonine-protein phosphatase 2B catalytic subunit alpha |
| | | isoform |
| PPP3CB | PP2BB_HUMAN | Serine/threonine-protein phosphatase 2B catalytic subunit beta isoform |
| PRDM1 | PRDM1_HUMAN | PR domain zinc finger protein 1 |
| PRDM10 | PRD10_HUMAN | PR domain zinc finger protein 10 |
| PRDM11 | PRD11_HUMAN | PR domain-containing protein 11 |
| PRDM12 | PRD12_HUMAN | PR domain zinc finger protein 12 |
| PRDM13 | PRD13_HUMAN | PR domain zinc finger protein 13 |
| PRDM14 | PRD14_HUMAN | PR domain zinc finger protein 14 |
| PRDM15 | PRD15_HUMAN | PR domain zinc finger protein 15 |
| PRDM16 | PRD16_HUMAN | Histone-lysine N-methyltransferase PRDM16 |
| PRDM2 | PRDM2_HUMAN | PR domain zinc finger protein 2 |
| PRDM5 | PRDM5_HUMAN | PR domain zinc finger protein 5 |
| PRDM6 | PRDM6_HUMAN | Putative histone-lysine N-methyltransferase PRDM6 |
| PRDM9 | PRDM9_HUMAN | Histone-lysine N-methyltransferase PRDM9 |
| PRDX1 | PRDX1_HUMAN | Peroxiredoxin-1 |
| PRDX2 | PRDX2_HUMAN | Peroxiredoxin-2 |
| PRDX3 | PRDX3_HUMAN | Thioredoxin-dependent peroxide reductase, mitochondrial |
| PRDX4 | PRDX4_HUMAN | Peroxiredoxin-4 |
| PRDX5 | PRDX5_HUMAN | Peroxiredoxin-5, mitochondrial |
| PRDX6 | PRDX6_HUMAN | Peroxiredoxin-6 |
| PREB | PREB_HUMAN | Prolactin regulatory element-binding protein |
| PREP | PPCE_HUMAN | Prolyl endopeptidase |
| PREX2 | PREX2_HUMAN | Phosphatidylinositol 3,4,5-trisphosphate-dependent Rae exchanger 2 |
| | | protein |
| PRG2 | PRG2_HUMAN | Eosinophil granule major basic protein |
| PRIM1 | PRI1_HUMAN | DNA primase small subunit |
| PR1MPOL | PR1PO_HUMAN | DNA-directed primase/polymerase protein |
| PRKAA1 | AAPK1_HUMAN | 5′-AMP-activated protein kinase catalytic subunit alpha-1 |
| PRKAA2 | AAPK2_HUMAN | 5′-AMP-activated protein kinase catalytic subunit alpha-2 |
| PRKAB1 | AAKB1_HUMAN | 5′-AMP-activated protein kinase subunit beta-1 |
| PRKAB2 | AAKB2_HUMAN | 5′-AMP-activated protein kinase subunit beta-2 |
| PRKACA | KAPCA_HUMAN | CAMP-dependent protein kinase catalytic subunit alpha |
| PRKAG1 | AAKG1_HUMAN | 5′-AMP-activated protein kinase subunit gamma-1 |
| PRKCA | KPCA_HUMAN | Protein kinase C alpha type |
| PRKCB | KPCB_HUMAN | Protein kinase C beta type |
| PRKCD | KPCD_HUMAN | Protein kinase C delta type catalytic subunit |
| PRKCE | KPCE_HUMAN | Protein kinase C epsilon type |
| PRKCG | KPCG_HUMAN | Protein kinase C gamma type |
| PRKCH | KPCL_HUMAN | Protein kinase C eta type |
| PRKC1 | KPC1_HUMAN | Protein kinase C iota type |
| PRKCQ | KPCT_HUMAN | Protein kinase C iota type |
| PRKD1 | KPCD1_HUMAN | Serine/threonine-protein kinase DI |
| PRKD2 | KPCD2_HUMAN | Serine/threonine-protein kinase D2 |
| PRKD3 | KPCD3_HUMAN | Serine/threonine-protein kinase D3 |
| PRKDC | PRKDC_HUMAN | DNA-dependent protein kinase catalytic subunit |
| PRKG1 | KGP1_HUMAN | cGMP-dependent protein kinase 1 |
| PRKN | PRKN_HUMAN | E3 ubiquitin-protein ligase parkin |
| PRLR | PRLR_HUMAN | Prolactin receptor |
| PRMT5 | ANM5_HUMAN | Protein arginine N-methyltransferase 5, N-terminally processed |
| PRNP | PR10_HUMAN | Major prion protein |
| PROS1 | PROS_HUMAN | Vitamin K-dependent protein S |
| PROZ | PROZ_HUMAN | Vitamin K-dependent protein Z |
| PRPF19 | PRP19_HUMAN | Pre-mRNA-processing factor 19 |
| PRPF38A | PR38A_HUMAN | Pre-mRNA-splicing factor 38A |
| PRPF4 | PRP4_HUMAN | U4/U6 small nuclear ribonucleoprotein Prp4 |
| PRPF40A | PR40A_HUMAN | Pre-mRNA-processing factor 40 homolog A |
| PRPF8 | PRP8_HUMAN | Pre-mRNA-processing-splicing factor 8 |
| PRPSAP1 | KPRA_HUMAN | Phosphoribosyl pyrophosphate synthase-associated protein 1 |
| PSAT1 | SERC_HUMAN | Phosphoserine aminotransferase |
| PSMA1 | PSA1_HUMAN | Proteasome subunit alpha type-1 |
| PSMA2 | PSA2_HUMAN | Proteasome subunit alpha type-2 |
| PSMA3 | PSA3_HUMAN | Proteasome subunit alpha type-3 |
| PSMA4 | PSA4_HUMAN | Proteasome subunit alpha type-4 |
| PSMA5 | PSA5_HUMAN | Proteasome subunit alpha type-5 |
| PSMA6 | PSA6_HUMAN | Proteasome subunit alpha type-6 |
| PSMA7 | PSA7_HUMAN | Proteasome subunit alpha type-7 |
| PSMB1 | PSB1_HUMAN | Proteasome subunit beta type-1 |
| PSMB10 | PSB10_HUMAN | Proteasome subunit beta type-10 |
| PSMB2 | PSB2_HUMAN | Proteasome subunit beta type-2 |
| PSMB3 | PSB3_HUMAN | Proteasome subunit beta type-3 |
| PSMB4 | PSB4_HUMAN | Proteasome subunit beta type-4 |
| PSMB5 | PSB5_HUMAN | Proteasome subunit beta type-5 |
| PSMB6 | PSB6_HUMAN | Proteasome subunit beta type-6 |
| PSMB7 | PSB7_HUMAN | Proteasome subunit beta type-7 |
| PSMB8 | PSB8_HUMAN | Proteasome subunit beta type-8 |
| PSMB9 | PSB9_HUMAN | Proteasome subunit beta type-9 |
| PSMC1 | PRS4_HUMAN | 26S proteasome regulatory subunit 4 |
| PSMC4 | PRS6B_HUMAN | 26S proteasome regulatory subunit 6B |
| PSMC5 | PRS8_HUMAN | 26S proteasome regulatory subunit 8 |
| PSMC6 | PRS10_HUMAN | 26S proteasome regulatory subunit 10B |
| PSMD1 | PSMD1_HUMAN | 26S proteasome non-ATPase regulatory subunit 1 |
| PSMD10 | PSD10_HUMAN | 26S proteasome non-ATPase regulatory subunit 10 |
| PSMD11 | PSD11_HUMAN | 26S proteasome non-ATPase regulatory subunit 11 |
| PSMD12 | PSD12_HUMAN | 26S proteasome non-ATPase regulatory subunit 12 |
| PSMD14 | PSDE_HUMAN | 26S proteasome non-ATPase regulatory subunit 14 |
| PSMD3 | PSMD3_HUMAN | 26S proteasome non-ATPase regulatory subunit 3 |
| PSPC1 | PSPC1_HUMAN | Paraspeckle component 1 |
| PTCRA | PTCRA_HUMAN | Pre T-cell antigen receptor alpha |
| PTGDS | PTGDS_HUMAN | Prostaglandin-H2 D-isomerase |
| PTGER3 | PE2R3_HUMAN | Prostaglandin E2 receptor EP3 subtype |
| PTGS2 | PGH2_HUMAN | Prostaglandin G/H synthase 2 |
| PTK2 | FAK1_HUMAN | Focal adhesion kinase 1 |
| PTK2B | FAK2_HUMAN | Protein-tyrosine kinase 2-beta |
| PTK6 | PTK6_HUMAN | Protein-tyrosine kinase 6 |
| PTPN11 | PTN11_HUMAN | Tyrosine-protein phosphatase non-receptor type 11 |
| PTPN12 | PTN12_HUMAN | Tyrosine-protein phosphatase non-receptor type 12 |
| PTPN13 | PTN13_HUMAN | Tyrosine-protein phosphatase non-receptor type 13 |
| PTPN14 | PTN14_HUMAN | Tyrosine-protein phosphatase non-receptor type 14 |
| PTPN2 | PTN2_HUMAN | Tyrosine-protein phosphatase non-receptor type 2 |
| PTPN23 | PTN23_HUMAN | Tyrosine-protein phosphatase non-receptor type 23 |
| PTPN3 | PTN3_HUMAN | Tyrosine-protein phosphatase non-receptor type 3 |
| PTPN5 | PTN5_HUMAN | Tyrosine-protein phosphatase non-receptor type 5 |
| PTPN6 | PTN6_HUMAN | Tyrosine-protein phosphatase non-receptor type 6 |
| PTPN7 | PTN7_HUMAN | Tyrosine-protein phosphatase non-receptor type 7 |
| PTPRD | PTPRD_HUMAN | Receptor-type tyrosine-protein phosphatase delta |
| PTPRF | PTPRF_HUMAN | Receptor-type tyrosine-protein phosphatase F |
| PTPRM | PTPRM_HUMAN | Receptor-type tyrosine-protein phosphatase mu |
| PTPRR | PTPRR_HUMAN | Receptor-type tyrosine-protein phosphatase R |
| PTPRS | PTPRS_HUMAN | Receptor-type tyrosine-protein phosphatase S |
| PTPRZ1 | PTPRZ_HUMAN | Receptor-type tyrosine-protein phosphatase zeta |
| PTS | PTPS_HUMAN | 6-pymvoyl tetrahydrobiopterin synthase |
| PUF60 | PUF60_HUMAN | Poly(U)-binding-splicing factor PUF60 |
| PUS7 | PUS7_HUMAN | Pseudouridylate synthase 7 homolog |
| PVR | PVR_HUMAN | Poliovirus receptor |
| PWWP2B | PWP2B_HUMAN | PWWP domain-containing protein 2B |
| PYGL | PYGL_HUMAN | Glycogen phosphorylase, liver form |
| QARS | SYQ_HUMAN | Glutamine--tRNA ligase |
| QPCT | QPCT_HUMAN | Glutaminyl-peptide cyclotransferase |
| QSOX1 | QSOX1_HUMAN | Sulfhydryl oxidase 1 |
| QTRT1 | TGT_HUMAN | Queuine tRNA-ribosyltransferase catalytic subunit |
| RAB3IP | RAB31_HUMAN | Rab-3A-interacting protein |
| RABIF | MSS4_HUMAN | Guanine nucleotide exchange factor MSS4 |
| RAC1 | RAC1_HUMAN | Ras-related C3 botulinum toxin substrate 1 |
| RACGAP1 | RGAP1_HUMAN | Rae GTPase-activating protein 1 |
| RACKI | RACK1_HUMAN | Receptor of activated protein C kinase 1, N-terminally processed |
| RAD1 | RAD1_HUMAN | Cell cycle checkpoint protein RAD1 |
| RAD18 | RAD18_HUMAN | E3 ubiquitin-protein ligase RAD18 |
| RAD51 | RAD51_HUMAN | DNA repair protein RAD51 homolog 1 |
| RAD52 | RAD52_HUMAN | DNA repair protein RAD52 homolog |
| RAE1 | RAE1L_HUMAN | mRNA export factor |
| RAET1L | ULBP6_HUMAN | UL16-binding protein 6 |
| RAF1 | RAF1_HUMAN | RAF proto-oncogene serine/threonine-protein kinase |
| RALGDS | GNDS_HUMAN | Ral guanine nucleotide dissociation stimulator |
| RAN | RAN_HUMAN | GTP-binding nuclear protein Ran |
| RANBP1 | RANG_HUMAN | Ran-specific GTPase-activating protein |
| RANBP2 | RBP2_HUMAN | E3 SUMO-protein ligase RanBP2 |
| RANBP3 | RANB3_HUMAN | Ran-binding protein 3 |
| RANBP9 | RANB9_HUMAN | Ran-binding protein 9 |
| RAP1GAP | RPGP1_HUMAN | Rap1 GTPase-activating protein 1 |
| RAPGEF5 | RPGF5_HUMAN | Rap guanine nucleotide exchange factor 5 |
| RAPGEFL1 | RPGFL_HUMAN | Rap guanine nucleotide exchange factor-like 1 |
| RAPH1 | RAPH1_HUMAN | Ras-associated and pleckstrin homology domains-containing protein 1 |
| RAPSN | RAPSN_HUMAN | 43 kDa receptor-associated protein of the synapse |
| RARA | RARA_HUMAN | Retinoic acid receptor alpha |
| RARB | RARB_HUMAN | Retinoic acid receptor beta |
| RARG | RARG_HUMAN | Retinoic acid receptor gamma |
| RARS | SYRC_HUMAN | Arginine--tRNA ligase, cytoplasmic |
| RASA1 | RASA1_HUMAN | Ras GTPase-activating protein 1 |
| RASGRP1 | GRP1_HUMAN | RAS guanyl-releasing protein 1 |
| RASGRP2 | GRP2_HUMAN | RAS guanyl-releasing protein 2 |
| RASGRP3 | GRP3_HUMAN | Ras guanyl-releasing protein 3 |
| RASGRP4 | GRP4_HUMAN | RAS guanyl-releasing protein 4 |
| RASSF1 | RASF1_HUMAN | Ras association domain-containing protein 1 |
| RASSF5 | RASF5_HUMAN | Ras association domain-containing protein 5 |
| RAVER1 | RAVR1_HUMAN | Ribonucleoprotein PTB-binding 1 |
| RBAK | RBAK_HUMAN | RB-associated KRAB zinc finger protein |
| RBBP4 | RBBP4_HUMAN | Histone-binding protein RBBP4 |
| RBBP6 | RBBP6_HUMAN | E3 ubiquitin-protein ligase RBBP6 |
| RBBP8 | CT1P_HUMAN | DNA endonuclease RBBP8 |
| RBKS | RBSK_HUMAN | Ribokinase |
| RBM10 | RBMl10_HUMAN | RNA-binding protein 10 |
| RBM11 | RBM11_HUMAN | Splicing regulator RBM11 |
| RBM22 | RBM22_HUMAN | Pre-mRNA-splicing factor RBM22 |
| RBM23 | RBM23_HUMAN | Probable RNA-binding protein 23 |
| RBM38 | RBM38_HUMAN | RNA-binding protein 38 |
| RBM39 | RBM39_HUMAN | RNA-binding protein 39 |
| RBM4 | RBM4_HUMAN | RNA-binding protein 4 |
| RBM4B | RBM4B_HUMAN | RNA-binding protein 4B |
| RBM5 | RBM5_HUMAN | RNA-binding protein 5 |
| RBM7 | RBM7_HUMAN | RNA-binding protein 7 |
| RBM8A | RBM8A_HUMAN | RNA-binding protein 8A |
| RBMX2 | RBMX2_HUMAN | RNA-binding motif protein, X-linked 2 |
| RBP4 | RET4_HUMAN | Plasma retinol-binding protein(1-176) |
| RBP5 | RET5_HUMAN | Retinol-binding protein 5 |
| RBPJ | SUH_HUMAN | Recombining binding protein suppressor of hairless |
| RBSN | RBNS5_HUMAN | Rabenosyn-5 |
| RCC1 | RCC1_HUMAN | Regulator of chromosome condensation |
| RCC1L | RCC1L_HUMAN | RCC1-like G exchanging factor-like protein |
| RCC2 | RCC2_HUMAN | Protein RCC2 |
| RCHY1 | ZN363_HUMAN | RING finger and CHY zinc finger domain-containing protein 1 |
| RECQL4 | RECQ4_HUMAN | ATP-dependent DNA helicase Q4 |
| REN | REN1_HUMAN | Renin |
| REP1N1 | REP11_HUMAN | Replication initiator 1 |
| REST | REST_HUMAN | RE1-silencing transcription factor |
| RET | RET_HUMAN | Extracellular cell-membrane anchored RET cadherin 120 kDa fragment |
| RFFL | RFFL_HUMAN | E3 ubiquitin-protein ligase rififylin |
| RFK | RIFK_HUMAN | Riboflavin kinase |
| RFPL4A | RFPLA_HUMAN | Ret finger protein-like 4A |
| RFWD3 | RFWD3_HUMAN | E3 ubiquitin-protein ligase RFWD3 |
| RFXANK | RFXK_HUMAN | DNA-binding protein RFXANK |
| RGCC | RFXK_HUMAN | Regulator of cell cycle RGCC |
| RGMB | RGMB_HUMAN | RGM domain family member B |
| RGN | RGN_HUMAN | Regucalcin |
| RHEB | RHEB_HUMAN | GTP-binding protein Rheb |
| RHO | OPSD_HUMAN | Rhodopsin |
| R1DA | RIDA_HUMAN | 2-iminobutanoate/2-iminopropanoate deaminase |
| RIMBP2 | RIMB2_HUMAN | RIMS-binding protein 2 |
| RIMBP3 | RIM3A_HUMAN | RIMS-binding protein 3A |
| RIMS1 | RIMS1_HUMAN | Regulating synaptic membrane exocytosis protein 1 |
| RIMS2 | RIMS2_HUMAN | Regulating synaptic membrane exocytosis protein 2 |
| RIOK1 | RIOK1_HUMAN | Serine/threonine-protein kinase RIO1 |
| RIOK2 | RIOK2_HUMAN | Serine/threonine-protein kinase RIO2 |
| RIPK1 | RIPK1_HUMAN | Receptor-interacting serine/threonine-protein kinase 1 |
| RIPK2 | RIPK2_HUMAN | Receptor-interacting serine/threonine-protein kinase 2 |
| RLBP1 | RLBP1_HUMAN | Retinaldehyde-binding protein 1 |
| RM12 | RM12_HUMAN | RecQ-mediated genome instability protein 2 |
| RNASE4 | RNAS4_HUMAN | Ribonuclease 4 |
| RNASEH2B | RNH2B_HUMAN | Ribonuclease H2 subunit B |
| RNASEH2C | RNH2C_HUMAN | Ribonuclease H2 subunit C |
| RNASEL | RN5A_HUMAN | 2-5A-dependent ribonuclease |
| RNF121 | RN121_HUMAN | RING finger protein 121 |
| RNF123 | RN123_HUMAN | E3 ubiquitin-protein ligase RNF123 |
| RNF125 | RN125_HUMAN | E3 ubiquitin-protein ligase RNF125 |
| RNF14 | RNF14_HUMAN | E3 ubiquitin-protein ligase RNF14 |
| RNF166 | RN166_HUMAN | RING finger protein 166 |
| RNF17 | RNF17_HUMAN | RING finger protein 17 |
| RNF170 | RN170_HUMAN | E3 ubiquitin-protein ligase RNFl 70 |
| RNF175 | RN175_HUMAN | RING finger protein 175 |
| RNF19A | RN19A_HUMAN | E3 ubiquitin-protein ligase RNF19A |
| RNF19B | RN19B_HUMAN | E3 ubiquitin-protein ligase RNF19B |
| RNF2 | RING2_HUMAN | E3 ubiquitin-protein ligase RING2 |
| RNF207 | RN207_HUMAN | RING finger protein 207 |
| RNF208 | RN208_HUMAN | RING finger protein 208 |
| RNF212B | R212B_HUMAN | RING finger protein 212B |
| RNF216 | RN216_HUMAN | E3 ubiquitin-protein ligase RNF216 |
| RNF31 | RNF31_HUMAN | E3 ubiquitin-protein ligase RNF3 l |
| RNF34 | RNF34_HUMAN | E3 ubiquitin-protein ligase RNF34 |
| RNF39 | RNF39_HUMAN | RING finger protein 39 |
| RNF4 | RNF4_HUMAN | E3 ubiquitin-protein ligase RNF4 |
| RNF8 | RNF8_HUMAN | E3 ubiquitin-protein ligase RNF8 |
| RNGTT | MCEI_HUMAN | mRN A guany ly ltransferase |
| ROBOl | ROBOl_HUMAN | Roundabout homolog 1 |
| ROBO2 | ROBO2_HUMAN | Roundabout homolog 2 |
| ROCKl | ROCKl_HUMAN | Rho-associated protein kinase 1 |
| ROCK2 | ROCK2_HUMAN | Rho-associated protein kinase 2 |
| ROR2 | ROR2_HUMAN | Tyrosine-protein kinase transmembrane receptor |
| | | ROR2 |
| RORA | RORA_HUMAN | Nuclear receptor ROR-alpha |
| RORB | RORB_HUMAN | Nuclear receptor ROR-beta |
| RORC | RORG_HUMAN | Nuclear receptor ROR-gamma |
| RPAl | RFAl_HUMAN | Replication protein A 70 kDa DNA-binding |
| | | subunit, N-terminally processed |
| RPA3 | RFA3_HUMAN | Replication protein A 14 kDa subunit |
| RPGR | RPGR_HUMAN | X-linked retinitis pigmentosa GTPase regulator |
| RPH3A | RP3A_HUMAN | Rabphilin-3A |
| RPH3AL | RPH3L_HUMAN | Rab effector Noc2 |
| RPLll | RLll_HUMAN | 60S ribosomal protein Ll 1 |
| RPL37 | RL37_HUMAN | 60S ribosomal protein L37 |
| RPL37A | RL37A_HUMAN | 60S ribosomal protein L37a |
| RPL37AP8 | RL37L_HUMAN | Putative 60S ribosomal protein L37a-like protein |
| RPS12 | RS12_HUMAN | 40S ribosomal protein S 12 |
| RPS15A | RS15A_HUMAN | 40S ribosomal protein Sl5a |
| RPS18 | RS18_HUMAN | 40S ribosomal protein Sl8 |
| RPS19 | RS19_HUMAN | 40S ribosomal protein Sl9 |
| RPS21 | RS21_HUMAN | 40S ribosomal protein S21 |
| RPS23 | RS23_HUMAN | 40S ribosomal protein S23 |
| RPS24 | RS24_HUMAN | 40S ribosomal protein S24 |
| RPS27A | RS27A_HUMAN | 40S ribosomal protein S27a |
| RPS3A | RS3A_HUMAN | 40S ribosomal protein S3a |
| RPS4X | RS4X_HUMAN | 40S ribosomal protein S4, X isoform |
| RPS4YI | RS4YI_HUMAN | 40S ribosomal protein S4, Y isoform I |
| RPS6 | RS6_HUMAN | 40S ribosomal protein S6 |
| RPS6KAI | KS6AI_HUMAN | Ribosomal protein S6 kinase alpha-I |
| RPS6KA3 | KS6A3_HUMAN | Ribosomal protein S6 kinase alpha-3 |
| RPS6KA5 | KS6A5_HUMAN | Ribosomal protein S6 kinase alpha-5 |
| RPS6KBI | KS6BI_HUMAN | Ribosomal protein S6 kinase beta-I |
| RPS7 | RS7_HUMAN | 40S ribosomal protein S7 |
| RPS8 | RS8_HUMAN | 40S ribosomal protein S8 |
| RPSA | RSSA_HUMAN | 40S ribosomal protein SA |
| RPTOR | RPTOR_HUMAN | Regulatory-associated protein ofmTOR |
| RREBI | RREBI_HUMAN | Ras-responsive element-binding protein I |
| RRMI | RlRI_HUMAN | Ribonucleoside-diphosphate reductase large |
| | | subunit |

### Methods of Using the Fusion Proteins, Vectors, and Expression Systems

The fusion proteins, vectors, and expression systems described herein, in various combinations, for example, with E3 ligase binding modulators, e.g., the E3 ligase binding modulators described herein and/or E3 ligase binding targets, e.g., the E3 ligase binding targets described herein are useful in a variety of methods, e.g., as described herein.

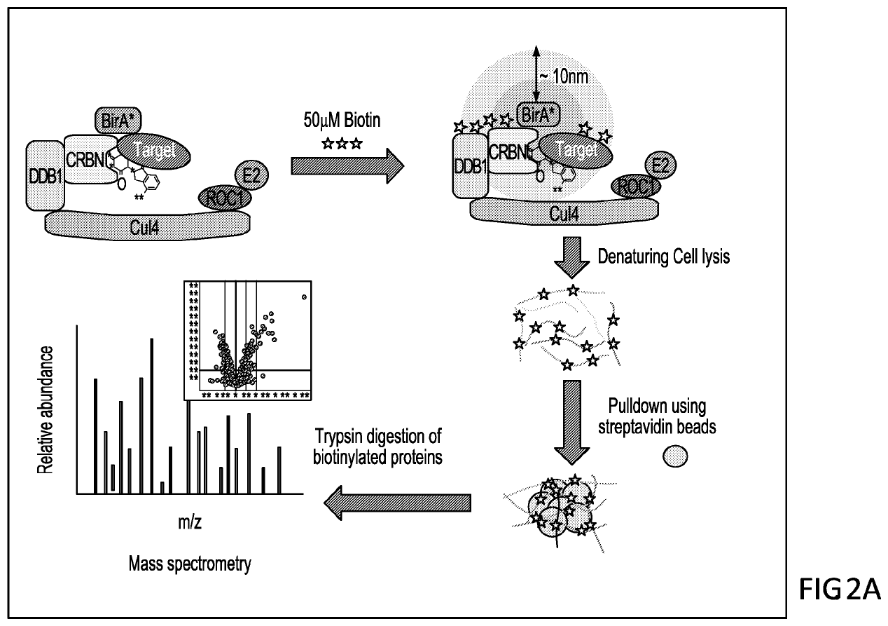

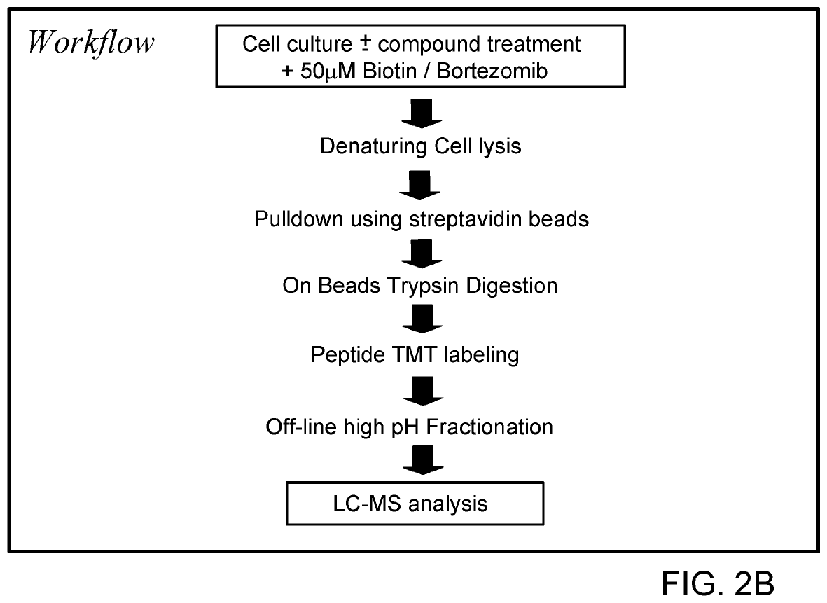

FIGS. 2A and 2B show an overview of an exemplary method for target detection and/or identification with an E3 ligase/Proximity Labeling Enzyme fusion protein in the presence of an E3 ligase binding modulator.

Described herein are methods for identifying interaction between an E3 ligase and an E3 ligase binding target (hereafter also referred to as “target”), e.g., for identifying targets that interact with an E3 ligase, e.g., in the presence of an E3 ligase binding modulator (hereafter also referred to as “modulator”) or not.

The methods described herein are useful, for example, for identifying previously unknown targets, e.g., targets that interact with the E3 ligase in a modulator-dependent manner. They are also useful, for example, for validating predicted target(s) that interact with an E3 ligase, e.g., in a modulator-dependent manner. They are also useful, for example, in identifying previously unknown E3 ligases and/or modulators that interact with known targets. They are also useful, for example, when using an E3 ligase that is unable to bind compounds at a canonical binding site, e.g., cereblon mutant Y384A/W386A, for identifying non-canonical E3 binding sites that interact with a modulator and/or target.

Thus, provided herein is a method for detecting the interaction of an E3 ligase and a target, the method comprises: a) providing cell(s) expressing a fusion protein described herein, e.g., a fusion protein comprising an E3 ligase, e.g., cereblon, a proximity labeling enzyme, e.g., a promiscuous biotinylation enzyme; and, optionally, a modulator; b) incubating the cell(s), under conditions effective for the proximity labeling enzyme to label protein(s) in the proximity of the fusion protein, e.g., in an incubation composition with a substrate for the proximity labeling enzyme; c) detecting the presence and/or amount of labeled protein(s), thereby detecting the interaction of an E3 ligase and a target.

Suitable fusion proteins and components thereof, targets, and cells/expression systems are described herein.

In some embodiments, the incubation composition comprises a cell culture medium. Suitable cell culture media are known and described in the art. See, e.g., Yang et al., “Culture Conditions and Types of Growth Media for Mammalian cells,” Intech dx.doi.org/10.5772/52301.

In some embodiments, e.g., when the proximity labeling enzyme is a promiscuous biotinylation enzyme, biotin (5-[(3aS,4S,6aR)-2-oxo-1,3,3a,4,6,6a-hexahydrothieno[3,4-d]imidazol-4-yl]pentanoic acid) is present in or added to the incubation composition. In some embodiments, the amount of biotin in the composition, e.g., at the beginning of the incubation or at the time the biotin is added to the composition, is from or from about 0.01 to or to about 0.10 mM. In some embodiments, the amount of biotin in the composition, e.g., at the beginning of the incubation, is or is about 0.05 mM.

In some embodiments, the cell(s) are incubated in the incubation medium without the substrate for the proximity labeling enzyme before adding the substrate for the proximity labeling enzyme to the composition. In some embodiments, the cell(s) are incubated for from or from about 5 minutes to or to about 10 hours before adding the substrate for the proximity labeling enzyme to the composition.

In some embodiments, a 26S proteasome inhibitor is present in or added to the incubation composition. In some embodiments, the 26S proteasome inhibitor is selected from the group consisting of bortezomib (([(1R)-3-methyl-1-[[(2S)-3-phenyl-2-(pyrazine-2-carbonylamino)propanoyl]amino]butyl]boronic acid)), ixazomib ([(IR)-1-[[2-[(2,5-dichlorobenzoyl)amino]acetyl]amino]-3-methylbutyl]boronic acid), carfilzomib ((2S)-4-methyl-N-[(2S)-1-[[(2S)-4-methyl-1-[(2R)-2-methyloxiran-2-yl]-1-oxopentan-2-yl]amino]-1-oxo-3-phenylpropan-2-yl]-2-[[(2S)-2-[(2-morpholin-4-ylacetyl)amino]-4-phenylbutanoyl]amino]pentanamide), MG-132 (benzyl N-[(2S)-4-methyl-1-[[(2S)-4-methyl-1-[[(2S)-4-methyl-1-oxopentan-2-yl]amino]-1-oxopentan-2-yl]amino]-1-oxopentan-2-yl]carbamate), MG-115 (benzyl N-[(2S)-4-methyl-1-[[(2S)-4-methyl-1-oxo-1-[[(2S)-1-oxopentan-2-yl]amino]pentan-2-yl]amino]-1-oxopentan-2-yl]carbamate), Proteasome Inhibitor I (tert-butyl (4S)-5-[[(2S)-1-[[(2S)-4-methyl-1-oxopentan-2-yl]amino]-1-oxopropan-2-yl]amino]-4-[[(2S)-3-methyl-2-(phenylmethoxycarbonylamino)pentanoyl]amino]-5-oxopentanoate), oprozomib (N-[(2S)-3-methoxy-1-[[(2S)-3-methoxy-1-[[(2S)-1-[(2R)-2-methyloxiran-2-yl]-1-oxo-3-phenylpropan-2-yl]amino]-1-oxopropan-2-yl]amino]-1-oxopropan-2-yl]-2-methyl-1,3-thiazole-5-carboxamide), marizomib ((1R,4R,5S)-4-(2-chloroethyl)-1-[(S)-[(1S)-cyclohex-2-en-1-yl]-hydroxymethyl]-5-methyl-6-oxa-2-azabicyclo[3.2.0]heptane-3,7-dione), MLN9708 (4-(carboxymethyl)-2-[(1R)-1-[[2-[(2,5-dichlorobenzoyl)amino]acetyl]amino]-3-methylbutyl]-6-oxo-1,3,2-dioxaborinane-4-carboxylic acid), and combinations thereof. In some embodiments, the amount of proteasome inhibitor in the incubation composition, e.g., at the beginning of the incubation or at the time it is added to the composition, is from or from about 0.02 μM to or to about 2.0 μM. In some embodiments, the amount of proteasome inhibitor in the incubation composition, e.g., at the beginning of the incubation or at the time it is added to the composition is or is about 0.2 μM.

In some embodiments, a modulator, e.g., as described herein, is present in or added to the incubation composition. In some embodiments, the modulator is provided as part of a composition comprising DMSO. In some embodiments, the amount of modulator in the composition, e.g., at the beginning of the incubation or at the time it is added to the composition, is from or from about 1 to or to about 50 mM. In some embodiments, the amount of modulator in the composition, e.g., at the beginning of the incubation or at the time it is added to the composition, is or is about 10 mM.

In some embodiments, the cell(s) are incubated in the incubation medium without the substrate for the proximity labeling enzyme before adding the modulator to the composition. In some embodiments, the cell(s) are incubated for from or from about 5 minutes to or to about 10 hours before adding the modulator to the composition.

In some embodiments, the proximity labeling enzyme substrate and the modulator are added to the composition at the same time or about the same time.

Detecting the presence and/or amount of labeled protein(s), e.g., labeled target(s), can be carried out by any suitable means, which are known in the art.

In some embodiments of any of the methods described herein, e.g., in particular for target validation and/or identification of E3 ligases and/or non-canonical E3 ligase binding sites, the target(s) and/or fusion protein(s) may be tagged, e.g., in a manner that is not dependent on the proximity labeling enzyme. In some embodiments, the affinity tag is selected from the group consisting of polyhistidine, glutathione S-transferase (GST), maltose-binding protein (MBP), chitin binding protein, a streptavidin tag (e.g., Trp-Ser-His-Pro-Gln-Phe-Glu-Lys, FLAG-tag (e.g., DYKDDDDK (SEQ ID NO: 72)), a biotin tag, and combinations thereof.

In some embodiments, detecting the presence and/or amount of protein(s), e.g., target(s) comprises a step of selectively isolating the target(s), e.g., by affinity chromatography.

In some embodiments of any of the methods described herein, detecting the presence and/or amount of protein(s), e.g., target(s), comprises an immunoprecipitation step. In some embodiments, e.g., when the proximity labeling enzyme is a promiscuous biotinylation enzyme, immunoprecipitation comprises streptavidin based immunoprecipitation, e.g., streptavidin bead based immunoprecipitation.

In some embodiments of any of the methods described herein, the cells are harvested and pelleted, e.g., prior to detecting the presence and/or amount of protein(s), e.g., target(s), e.g., prior to immunoprecipitation.

In some embodiments of any of the methods described herein, immunoprecipitation comprises incubating the cell(s), e.g., the harvested cell pellet, in a lysis buffer. In some embodiments, the lysis buffer is a urea buffer. In some embodiments, the lysis buffer comprises a protease inhibitor. In some embodiments, the protease inhibitor is selected from the group consisting of AEBSF, Bestatin, E-64, Pepstatin A, Phosphoramidon, Leupeptin, Aprotinin, 1,10-Phenanthroline, and combinations thereof. Following lysis, the labeled protein(s) can be harvested, e.g., with streptavidin beads, and analyzed, for example, but Western Blot and/or Mass spectrometry, e.g., quantitative mass spectrometry.

In some embodiments of any of the methods described herein, a target is identified as having a modulator-dependent interaction with an E3 ligase, or vice-versa, when the amount of the target protein that is labeled after incubation with the modulator, e.g., as described herein, is greater than the amount of the target protein that is labeled after incubation under the same conditions except without a modulator (e.g., with DMSO as a negative control). In some embodiments, the log2 fold change of the target protein when incubated with the modulator versus the control (e.g., DMSO) is at least 0.5, at least 1, at least 1.5, at least 2, or at least 3. In some embodiments, the p-value of detecting a given log2 fold change across sample conditions is 0.1 or less, e.g., 0.05 or less, e.g., 0.001 or less, e.g., 0.0001, 0.00001, 0.000001, 0.0000001, 0.00000001, 0.000000001 or less.

In some embodiments of any of the methods described herein, a target is identified as having a modulator-dependent interaction with an E3 ligase when the amount of the target protein that is labeled after incubation with a modulator, e.g., as described herein, is greater than the amount of the target protein that is labeled after incubation under the same conditions except where the E3 ligase is a mutant that is unable to bind the modulator at a canonical binding site. In some embodiments, the log2 fold change of the target protein when incubated with the modulator versus the control (e.g., E3 ligase mutant unable to bind the modulator at a canonical binding site) is at least 0.5, at least 1, at least 1.5, at least 2, or at least 3. In some embodiments, the p-value of detecting a given log2 fold change across sample conditions is 0.1 or less, e.g., 0.05 or less, e.g., 0.001 or less, e.g., 0.0001, 0.00001, 0.000001, 0.0000001, 0.00000001, 0.000000001 or less.

### EXAMPLES

The following examples are included for illustrative purposes only and are not intended to limit the scope of the invention.

### Example 1: TurboID Immunoprecipitation

TurboID is a mutant form of the E. coli biotin ligase enzyme BirA made by phage display. It is the key component of a proximity-dependent biotin identification method which is able in living cells to identify the proteins that are in close proximity (around 10 nm) of the protein of interest. In fact, the protein of interest is fused with the TurboID and, in presence of biotin, this enzyme attaches a biotin tag on proximal and potentially interacting proteins. This labelling system is efficient when only treating the samples with biotin for, e.g., 1 to 6 h, instead of 18 to 24 h with the previously developed BioID mutant version of BirA.

These biotinylated proteins can then be extracted and purified by immunoprecipitation, e.g., using streptavidin beads, and identified by western-blot or mass spectrometry.

Immunoprecipitation is an assay which aims at extracting and purifying proteins, for example from cell lysate, by using specific antibodies immobilized to a solid support such as magnetic beads.

In one example, streptavidin magnetic beads are used. Streptavidin beads are made of a recombinant form of streptavidin which is covalently coupled to the magnetic beads surface. Streptavidin and biotin have a high affinity and thus biotinylated proteins are able to bind to these streptavidin beads. These beads are first equilibrated with the lysis buffer and samples with biotinylated proteins are added to the beads. During this time, the binding between biotinylated proteins and the beads can occur. Several washes with different type of buffers are then performed to eliminate all proteins that did not bind to the beads and ensure the purity of the final protein sample. Finally, as the binding between streptavidin and biotin is very strong, the target biotinylated proteins are eluted within harsh conditions. In this example, immunoprecipitation using magnetic beads instead of agarose beads makes the aspiration of the cell lysate easier thanks to the use of a magnet which enable to separate the beads from the rest of the solution within the tube. Consequently, it avoids the centrifugation steps which can disrupt weak interactions between the proteins and the beads and lead to the loss of some target proteins.

### Example 2: TurboID E3 Ligase Fusion Proteins

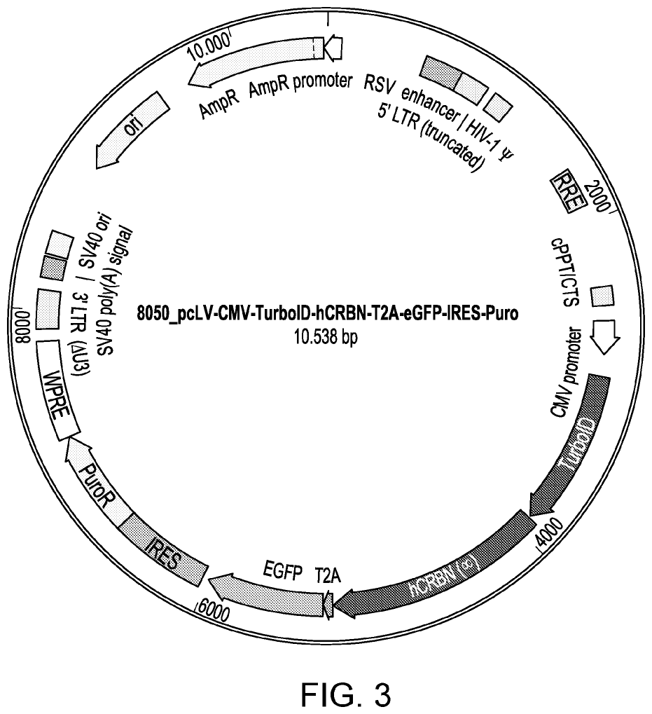

The polynucleotide encoding the TurboID hCRBN fusion protein along with a T2A element and eGFP coding region (SEQ ID NO: 12) was cloned into the expression plasmid (pcLV-CMV-MCS-T2A-eGFP-IRES-Puro). The plasmid construct is shown in FIG. 3. The vector includes a CMB promoter for fusion protein expression (constitutive), a puromycin resistance gene, and an eGFP.

### Example 3: TurboID CRBN Coupled Cell Lines

### Overview

### TurboID CRBN Cell Lines With Respective Growth Medium.

| Cell line | Medium |
|---|---|
| HEK293T TurboID | DMEM (Cat# 31966-021, |
| CRBN | Gibco) + 10% FBS |
| CAL51 TurboID CRBN | DMEM (Cat. No. 11965-092, |
| | Gibco) + 10% FBS |
| HCT116 TurboID CRBN | McCoy′s 5A (26600-023, |
| | Gibco) + 10% FBS |
| MCF7 TurboID CRBN | MEM (Sigma,Cat # |
| | M8042-500 ml) + 10% FBS + |
| | 0.01 mg/ml insuline (Gibco, |
| | Cat. # 11508856) |
| SKMEL28 TurboID | EMEM (ATCC, Cat # |
| CRBN | 30-2003) + 10% FBS |
| THP1 TurboID CRBN | RPMI (Cat. No. 61870-010, |
| | Gibco) + 10% FBS |
| U937 TurboID CRBN | RPMI (Cat. No. 21875-034, |
| | Gibco) + 10% FBS |

### HEK293T TurboID CRBN

HEK293T cells were genetically modified by transduction with the following lentiviral particle: cLV-CMV-TurboID-hCRBN-IRES-PuroR with a puromycin resistant gene to select the mutant cells expressing the TurboID CRBN construct.

HEK293T TurboID CRBN cells were cultured in DMEM (Cat #31966-021, Gibco) supplemented with 10% FBS (Cat #P30-1909, PAN Biotech) and 0.5 ug/mL puromycin (gibco, #A11138-03) for the two first passages. Then they are cultured without puromycin.

Subculture them by washing once with DPBS-/-, trypsinizing with 2 ml TrypLE Express (Cat #12604013, Gibco) until they detach followed by neutralizing with 8 ml culture medium. Count cells and reseed 3×10⁶ cells in a T150 flask. For freezing add 10% DMSO (Sigma, #41639-100 mL) to the culture medium and freeze 1×10⁶ up to 1×10⁷ cells per vial.

### CAL51 TurboID CRBN

CAL51 cells were genetically modified by transduction with the following lentiviral particle: cLV-CMV-TurboID-hCRBN-IRES-PuroR with a puromycin resistant gene to select the mutant cells expressing the TurboID CRBN construct. The MOI obtained was 0.5.

CAL51 TurboID CRBN cells were cultured in DMEM (Cat. No. 11965-092, Gibco) supplemented with 10% FBS (Cat #P30-1909, PAN Biotech) and 0.5 ug/mL puromycin (gibco, #A11138-03) for the two first passages. Then they are cultured without puromycin.

Subculture them by washing once with DPBS-/-, trypsinizing with 2 ml TrypLE Express (Cat #12604013, Gibco) until they detach followed by neutralizing with 8 ml culture medium. Count cells and reseed 5×10⁶ cells in a T150 flask. For freezing add 10% DMSO (Sigma, #41639-100 mL) to the culture medium and freeze 1×10⁶ up to 1×10⁷ cells per vial.

### HCT116 TurboID CRBN

HCT116 cells were genetically modified by transduction with the following lentiviral particle: cLV-CMV-TurboID-hCRBN-IRES-PuroR with a puromycin resistant gene to select the mutant cells expressing the TurboID CRBN construct. The MOI obtained was 0.3.

HCT116 TurboID CRBN cells were cultured in McCoy's 5A (26600-023, Gibco) supplemented with 10% FBS (Cat #P30-1909, PAN Biotech) and 0.5 ug/mL puromycin (gibco, #A11138-03) for the two first passages. Then they are cultured without puromycin.

Subculture them by washing once with DPBS-/-, trypsinizing with 2 ml TrypLE Express (Cat #12604013, Gibco) until they detach followed by neutralizing with 8 ml culture medium. Count cells and reseed 4×10⁶ cells in a T150 flask. For freezing add 10% DMSO (Sigma, #41639-100 mL) to the culture medium and freeze 1×10⁶ up to 1×10⁷ cells per vial.

### MCF7 TurboID CRBN

MCF7 cells were genetically modified by transduction with the following lentiviral particle: cLV-CMV-TurboID-hCRBN-IRES-PuroR with a puromycin resistant gene to select the mutant cells expressing the TurboID CRBN construct. The MOI obtained was 0.4.

MCF7 TurboID CRBN cells were cultured in MEM (Sigma, Cat #M8042-500 ml) supplemented with 10% FBS (Cat #P30-1909, PAN Biotech), 0.01 mg/ml insuline (Gibco, Cat. #11508856) and 0.5 ug/mL puromycin (gibco, #A11138-03) for the two first passages. Then they are cultured without puromycin.

Subculture them by washing once with DPBS^{-/-}, trypsinizing with 2 ml TrypLE Express (Cat #12604013, Gibco) until they detach followed by neutralizing with 8 ml culture medium. Count cells and reseed 6×10⁶ cells in a T150 flask. For freezing add 10% DMSO (Sigma, #41639-100 mL) to the culture medium and freeze 1×10⁶ up to 1×10⁷ cells per vial.

### SKMEL28 TurboID CRBN

SKMEL28 cells were genetically modified by transduction with the following lentiviral particle: cLV-CMV-TurboID-hCRBN-IRES-PuroR with a puromycin resistant gene to select the mutant cells expressing the TurboID CRBN construct. The MOI obtained was 0.8.

SKMEL28 TurboID CRBN cells were cultured in EMEM (ATCC, Cat #30-2003) supplemented with 10% FBS (Cat #P30-1909, PAN Biotech) and 0.5 ug/mL puromycin (gibco, #A11138-03) for the two first passages. Then they are cultured without puromycin.

Subculture them by washing once with DPBS^{-/-}, trypsinizing with 2 ml TrypLE Express (Cat #12604013, Gibco) until they detach followed by neutralizing with 8 ml culture medium. Count cells and reseed 5×10⁶ cells in a T150 flask. For freezing add 10% DMSO (Sigma, #41639-100 mL) to the culture medium and freeze 1×10⁶ up to 1×10⁷ cells per vial.

### THP1 TurboID CRBN

THP 1 cells were genetically modified by transduction with the following lentiviral particle: cLV-CMV-TurboID-hCRBN-T2A-eGFP-IRES-Puro with a puromycin resistant gene and an eGFP marker to select the mutant cells expressing the TurboID CRBN construct. One week after transduction, all GFP+ cells were sorted with a cell sorter.

THP1 TurboID CRBN cells were cultured in RPMI (Cat. No. 61870-010, Gibco) supplemented with 10% FBS (Cat #P30-1909, PAN Biotech) before cell sorting and also with 0.5 ug/mL puromycin (gibco, #A11138-03) three passages after cell sorting.

Subculture the cells by keeping them at a density of 0.5 million cells/mL. For freezing add 10% DMSO (Sigma, #41639-100 mL) to the culture medium and freeze 1×10⁶ up to 1×10⁷ cells per vial.

### U937 TurboID CRBN

U937 cells were genetically modified by transduction with the following lentiviral particle: cLV-CMV-TurboID-hCRBN-T2A-eGFP-IRES-Puro with a puromycin resistant gene and an eGFP marker to select the mutant cells expressing the TurboID CRBN construct. One week after transduction, all GFP+ cells were sorted with a cell sorter.

U937 TurboID CRBN cells were cultured in RPMI (Cat. No. 21875-034, Gibco) supplemented with 10% FBS (Cat #P30-1909, PAN Biotech) before cell sorting and also with 0.5 ug/mL puromycin (gibco, #A11138-03) three passages after cell sorting.

Subculture the cells by keeping them at a density of 0.5 million cells/mL. For freezing add 10% DMSO (Sigma, #41639-100 mL) to the culture medium and freeze 1×10⁶ up to 1×10⁷ cells per vial.

### Example 4: CRBN Proximity Assay

A CRBN proximity assay was carried out with the cell lines described above, as follows.

Cells were seeded in culture vessels (e.g., dishes or flasks) and, in some cases (e.g., for adherent cells), incubated overnight. Bortezomib was added either directly to the cells (e.g., for cell suspensions) or by medium exchange (e.g., for adherent cells).

Compounds (CP) were dissolved in DMSO to a concentration of 10 mM. Both 10 uM compound or DMSO (as a control) and 50 uM Biotin solution (stock solution at 50 mM) were directly added to the cultures at defined time points (e.g., 15 min, 1 h or 6 h).

Cells were harvested on ice by washing with PBS, centrifuging, discarding the supernatant, re-suspending the pellet in 1 mL cold PBS 1×, and centrifuging again. The pellet were stored at −80° C.

Urea buffer (2M urea +10 mM Tris HCl pH8) was prepared, filtered, and stored at 4° C. A solution of 10 mL lysis buffer (e.g., NP-40 or RIPA) with plus 100 μL each of Protease inhibitor cocktail 1 (Sigma, #P8340), Phosphatase inhibitor cocktail 2 (Sigma, #P5726), and Phosphatase inhibitor cocktail 3 (Sigma, #P0044), and 1 μL 0.2 μM Bortezomib was prepared fresh and kept on ice.

The harvested cell pellet was resuspended in 1 mL lysis buffer, vortexed, sonicated, and kept on ice for 20 min. The lysate was centrifuged at max speed for 10 min at 4° C. and the supernatant was transferred in a new 1.5 mL Eppendorf tube and put on ice.

Immunoprecipitation was carried out using magnetic beads in a 1.5 mL Eppendorf tube. To prepare the magnetic beads, lysis buffer was added to the beads and mixed before applying magnetic force to separate the beads from the supernatant. The supernatant was removed and discarded. This process was repeated.

Then, the cells were prepared for either Western Blot or Mass Spectrometry analysis, as follows.

For Western Blot, 900 μL of the cell lysate (protein concentration ˜0.5 mg/mL) was added to 40 μL of beads and incubated for 4 h at 4° C. on a rotating device. 50 μL of the cell lysate was put back into the tube with 15 μL 4× Laemmli Sample Buffer and 1.4 μL dithiothreitol (DTT) (stock solution at 1M to give a final concentration of 20 mM) and then heated 10 min at 70° C. The beads were collected with the magnetic stand, and the unbound sample was removed and saved for analysis. 50 uL of the unbound sample was put in an Eppendorf tube with 15 uL 4× Laemmli Sample Buffer and 1.4 uL DTT (stock solution at IM to have a final concentration of 20 mM) and then heated 10 min at 70° C. The beads were first washed twice with 1 mL RIPA buffer, then with 1 mL urea buffer, and finally once with 1 mL lysis buffer. 50 uL of lysis buffer solution (with protease inhibitors and bortezomib) was added to the beads to elute the samples, with 20 uL 4× Laemmli Sample Buffer, 1.6 uL DTT (20 mM) and 16 uL biotin (10 mM). The sample was vortexed to mix and heated at 95° C. for 15 min, and then stored at −80° C. ready to use for Western Blot analysis.

For Mass Spectrometry, 900 uL of the cell lysate (protein concentration around 1 mg/mL) was added to 50 uL of beads and incubated for 4 h at 4° C. on a rotating device. The beads were collected with the magnetic stand, and the unbound sample was removed and saved for analysis. The beads were washed twice with 1 mL RIPA buffer, then three times with 1 mL urea buffer, and finally with 1 mL 1×PBS. The supernatant was removed and the sample with the beads was frozen at −80° C.

### Example 5: Exemplary Protocol in 6-Well Plates Using HEK293 TurboID

| Material | Vendor | Catalog number |
|---|---|---|
| DMEM, high glucose | Gibco | 11965 |
| FBS | PAN Biotech | P30-1909 |
| TrypLE | Gibco | A12177 |
| DMSO | Fisher Bioreagents | BP231-100 |
| D-biotin 50 mM | Thermo Scientific | B20656 |
| RIPA buffer | Thermo Scientific | 89901 |
| NP-40, 70% in H₂O | Sigma | NP40S |
| Protease/phosphatase | Sigma | P8340, P5726, |
| inhibitors | | P0044 |
| Bortezomib 10 mM | Selleckchem | S1013 |
| Streptavidin beads | Thermo | 88817 |
| PBS-/- (DPBS, w/o: | PAN Biotech | P04-36500 |
| Ca and Mg) | | |
| Urea | Sigma | U1250 |
| 1M Tris-HCl | Alfa Aesar | J22638 |
| LDS Buffer | Thermo | NP0007 |
| Reducing Agent | Thermo | NP0009 |

### Cell Culture

Cells were seeded in 6-well plates to become 70-90% confluent the next day (For HEK293 TurboID CRBN cell line, seed ˜1.2-1.4×10\hat{ }6 cells per well in DMEM, high glucose+10% FBS; 2 mL/well) and incubated at 37° C., 5% CO2 for ca. 24 h.

A working dilution of Bortezomib was prepared by diluting the 10 mM stock 50× fold in DMSO to 200 μM.

The cells were treated according to the table below by adding the corresponding volumes of Biotin, Bortezomib and compounds (or DMSO respectively) into the cell medium and then incubated at 37° C., 5% CO₂ for 6 h. The cell medium was removed and cells were detached by adding 0.5 mL TrypLE 1×, and then transferred into 1.5 mL tubes, rinsed with PBS, collected by centrifugation, and frozen at −80° C.

### Cell Treatment:

| Substance | Volume per well of a 6-well plate |
|---|---|
| Cell medium | 2 mL |
| Compound in DMSO (10 mM) OR | 2 μL (final concentration |
| DMSO without compound (as control) | of compound 10 μM) |
| Biotin (50 mM) | 2 μL |
| Bortezomib pre-dilution (200 μM) | 2 μL (final concentration |
| | 200 nM) |

### Immunoprecipitation

Immunoprecipitation was carried out as described in Example 4. Briefly, the cell pellet was resuspended in 900 μL lysis buffer solution (as shown in the table below) and sonicated.

| Component | Volume |
|---|---|
| RIPA buffer (Thermo Scientific #89901) | 17 mL |
| Protease inhibitor (Sigma #P8340) | 170 μL |
| Phosphatase inhibitor (Sigma #P5726) | 170 μL |
| Protease inhibitor (Sigma #P0044) | 170 μL |
| 10 mM Bortezomib (Selleckchem #S1013) | 1.7 μL |

680 μL of streptavidin beads (Thermo, Cat. 88817) were prepared by washing twice with equal volumes of RIPA buffer.

Each sample was separately incubated with 40 μL of prepared beads and incubated at 4° C. for 4 h in a tube rotator before removing the supernatant and washing the beads twice with RIPA buffer, and then three times with urea buffer (as shown in the table below), and then two times with PBS before freezing at −80° C.

| Component | Volume/amount |
|---|---|
| MilliQ water | 297 mL |
| 1M Tris HCl pH 8.0 | 3 mL |
| Urea (MW: 60.06 g/mol) (Sigma #U1250) | 36 g |

Protease inhibitor cat. P8340 (Sigma-Aldrich): this mixture contains individual components, including AEBSF at 104 mM, Aprotinin at 80 μM, Bestatin at 4 mM, E-64 at 1.4 mM, Leupeptin at 2 mM and Pepstatin A at 1.5 mM. Each component has specific inhibitory properties. AEBSF and Aprotinin act to inhibit serine proteases, including trypsin, chymotrypsin, and plasmin amongst others. Bestatin inhibits aminpeptidases. E-64 acts against cystein proteases. Leupeptin acts against both serine and cystein proteases. Pepstatin A inhibits acid proteases.

Protease inhibitor cat. P5726 (Sigma-Aldrich): this mixture contains individual components with specific inhibitory properties. Sodium orthovanadate inhibits a number of ATPases, protein tyrosine phosphatases, and other phosphate-transferring enzymes. Sodium molybdate inhibits acid and phosphoprotein phosphatases. Sodium tartrate inhibits acid phosphatases. Imidazole inhibits alkaline phosphatases.

Protease inhibitor cat. P0044(Sigma-Aldrich): this mixture contains individual components with specific inhibitory properties. Cantharidin inhibits protein phosphatase 2A. (−)-p-Bromolevamisole oxalate inhibits L-isoforms of alkaline phosphatases. Calyculin A inhibits protein phosphatases 1 and 2A.

For Western blot, the proteins were eluted from the beads using LDS buffer (10 min, 70° C.). Then, the eluate was transferred into a new tube and 1× Reducing Agent was added (10 min, 70° C.)

### Example 6: Mass Spectrometry Analysis

### Cell Treatment

Turbo-ID cells were incubated for 6 h at 37° C. in fresh medium DMEM high Glucose (Gibco, 11965) +10% FBS (PAN Biotech, P30-109) with 50 μM D-biotin (Thermo Scientific, B20656), 0.2 μM of Bortezomib (Selleckchem, S1013) and with 10 μM COMPOUND (E3 ligase binding modulator) or DMSO (Fisher Bioreagents, BP231-100). Cells were washed once with PBS and collected with 1 mL of TrypLE (Gibco, #A12177) in 1.5 mL Eppendorf tube lobind. Cells were pelleted by centrifugation at 500×g for 5 min at 4° C., supernatants were removed, and cells were washed with 1 mL of PBS. After centrifugation, dried pellets were frozen at −80° C.

### Immunoprecipitation

Cell pellets were lysed in 900 μL of cold RIPA lysis buffer (25 mM Tris-HCl pH 7.6, 150 mM NaCl, 1% NP-40, 1% sodium deoxycholate, 0.1% SDS; Thermo Scientific, 89901), protease inhibitors (Sigma, P8340, P5726, P0044), 0.2 μM Bortezomib, then sonicated on ice (30 s at 50% power, UP200St ultrasonic, Huber lab) to disrupt visible aggregates. The lysate was centrifuged at max speed for 10 min. Supernatants were incubated with 40 μL of pre-washed streptavidin beads with cold RIPA buffer for 4 h in rotator at 4° C. Beads were collected by magnetic rack, washed with 1 mL RIPA buffer 2 times, 2M urea (Sigma, U1250) 10 mM Tris-HCl pH 8.0 buffer 3 times (Alfa Aesar, J22638) and cold PBS 3 times.

### Digestion

The streptavidin beads were incubated with 25 uL PreOmics iST-NHS lysis buffer (#P.O.00030) then processed using the PreOmics kit following their recommended protocol with minor modifications. In brief, the proteins were reduced, alkylated and digested for 3 h at 37° C. The peptides were then labelled with TMT reagent (1:4; peptide:TMT label) (Thermo Fisher Scientific #A4520). After quenching, the peptides from the 16 conditions were combined to a 1:1 ratio and purified.

### Fractionation

Mixed and labeled peptides were subjected to high-pH reversed-phase fractionation with Pierce™ High pH Reversed-Phase Peptide Fractionation Kit (Thermo Fischer, #84868), according to their recommended protocol. The dried 8 fractions were reconstituted in 0.1% formic acid for LC-SPS-MS3 analysis.

### Mass Spectrometry Analysis

Labelled peptides were loaded onto an Aurora column from Ionopticks (75 μm ID, 1.6 μm particles, 25 cm in length) in an EASY-nLC 1200 system. The peptides were separated using a 168 min gradient from 4% to 30% buffer B (80% acetonitrile in 0.1% formic acid) equilibrated with buffer A (0.1% formic acid) at a flow rate of 400 nl/min. Eluted TMT peptides were analyzed on an Orbitrap Eclipse mass spectrometer (Thermo Fisher Scientific).

MSI scans were acquired at resolution 120,000 with 400-1400 m/z scan range, AGC target 4×10⁵, maximum injection time 50 ms. Then, MS2 precursors were isolated using the quadrupole (0.7 m/z window) with AGC 1×10⁴ and maximum injection time 50 ms. Precursors were fragmented by CID at a normalized collision energy (NCE) of 35% and analyzed in the ion trap. Following MS2, synchronous precursor selection (SPS) MS3 scans were collected by using high energy collision-induced dissociation (HCD) and fragments were analyzed using the Orbitrap (NCE 55%, AGC target 1×10⁵, maximum injection time 120 ms, resolution 60,000).

Protein identification and quantification were performed using Proteome Discoverer 2.4.0.305 with the SEQUEST algorithm and Uniprot human database (2021 Jan. 29, 20614 protein sequences). Mass tolerance was set at 10 ppm for precursors and at 0.6 Da for fragment. Maximum of 3 missed cleavages were allowed. Methionine oxidation was set as dynamic modification, while TMT tags on peptide N termini/lysine residues and cysteine alkylation (+113.084) were set as static modifications.

The list of identified peptide spectrum matches (PSMs) was filtered to respect a 1% False Discovery Rate (FDR) after excluding PSMs with TMT reporter ion signal-to-noise value lower than 10 and a precursor interference level value higher than 50%. Subsequently, protein identifications were inferred from protein specific peptides, i.e. peptides matching multiple protein entries were excluded. Protein relative quantification was performed using an analysis including multiple steps; adjustment of reporter ion intensities for isotopic impurities according to the manufacturer's instructions, global data normalization by equalizing the total reporter ion intensity across all channels, summation of reporter ion intensities per protein and channel, calculation of protein abundance log2 fold changes (L2FC) and testing for differential abundance using moderated t-statistics where the resulting p-values reflect the probability of detecting a given L2FC across sample conditions by chance alone.

### Example 7: Identification of E3 Ligase Modulator Dependent Cereblon-Target Interactions

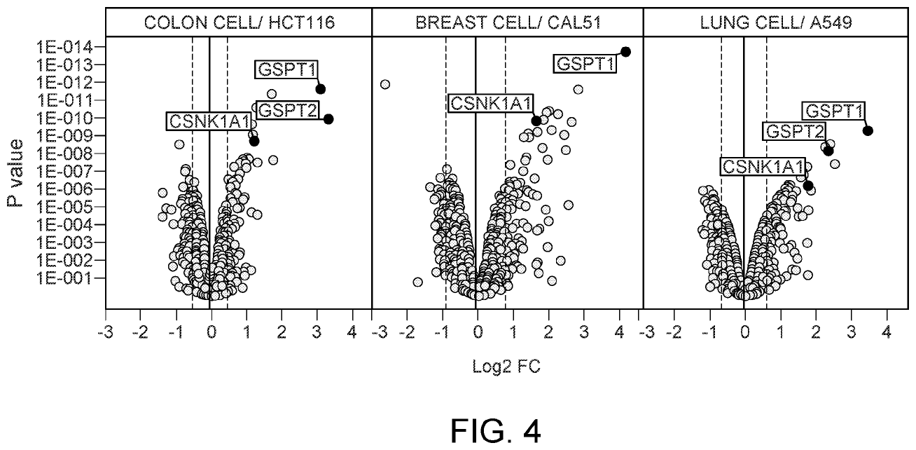

The method as described in Example 6 was carried out with Mass Spectrometry in three different cell lines (colon cell line HCT116, Breast cell line CAL51, and Lung cell line A549) each expressing pcLV-CMV-MCS-T2A-eGFP-IRES-Puro. As shown in FIG. 4, known protein targets well-known neo-substrates like GSPT1,2 and CK1a were identified interacting with cereblon in an E3 ligase binding modulator-dependent manner. The other proteins identified by mass spectrometry were interactors of the E3 ligase complex and potentially novel neo-substrates of cereblon. Most of these proteins showed a common profile across the three cell lines, though some were unique to a particular cancer cell line lineage. This method is an unbiased and powerful approach to identify drug-induced neo-substrates of the E3 ligase cereblon in cells.

### Example 8: Exemplary High Throughput Protocol

In this example, the CRBN proximity assay described in Example 4 is carried out in a high throughput fashion, e.g., in 96-well plates. Pull down of biotinylated proteins using streptavidin magnetic beads is carried out in a 96-well plate format and washing is automated on a liquid handling robot.

SEQUENCES

SEQ ID NO: 1 TurboID CRBN fusion protein
MKDNTVPLKLIALLANGEFHSGEQLGETLGMSRAAINKHIQTLRDWGVDVFTVPGKGYSLPEPI
PLLNAKQILGQLDGGSVAVLPVVDSTNQYLLDRIGELKSGDACIAEYQQAGRGSRGRKWFSPFG
ANLYLSMFWRLKRGPAAIGLGPVIGIVMAEALRKLGADKVRVKWPNDLYLQDRKLAGILVELAG
ITGDAAQIVIGAGINVAMRRVEESVVNQGWITLQEAGINLDRNTLAATLIRELRAALELFEQEG
LAPYLPRWEKLDNFINRPVKLIIGDKEIFGISRGIDKQGALLLEQDGVIKPWMGGEISLRSAEK
GSGAGEGDQQDAAHNMGNHLPLLPAESEEEDEMEVEDQDSKEAKKPNIINFDTSLPTSHTYLGA
DMEEFHGRTLHDDDSCQVIPVLPQVMMILIPGQTLPLQLFHPQEVSMVRNLIQKDRTFAVLAYS
NVQEREAQFGTTAEIYAYREEQDFGIEIVKVKAIGRQRFKVLELRTQSDGIQQAKVQILPECVL
PSTMSAVQLESLNKCQIFPSKPVSREDQCSYKWWQKYQKRKFHCANLTSWPRWLYSLYDAETLM
DRIKKQLREWDENLKDDSLPSNPIDFSYRVAACLPIDDVLRIQLLKIGSAIQRLRCELDIMNKC
TSLCCKQCQETEITTKNEIFSLSLCGPMAAYVNPHGYVHETLTVYKACNLNLIGRPSTEHSWEP
GYAWTVAQCKICASHIGWKFTATKKDMSPQKFWGLTRSALLPTIPDTEDEISPDKVILCL
SEQ ID NO: 2 TurboID CRBN fusion protein with T2A and GFP
MKDNTVPLKLIALLANGEFHSGEQLGETLGMSRAAINKHIQTLRDWGVDVFTVPGKGYSLPEPI
PLLNAKQILGQLDGGSVAVLPVVDSTNQYLLDRIGELKSGDACIAEYQQAGRGSRGRKWFSPFG
ANLYLSMFWRLKRGPAAIGLGPVIGIVMAEALRKLGADKVRVKWPNDLYLQDRKLAGILVELAG
ITGDAAQIVIGAGINVAMRRVEESVVNQGWITLQEAGINLDRNTLAATLIRELRAALELFEQEG
LAPYLPRWEKLDNFINRPVKLIIGDKEIFGISRGIDKQGALLLEQDGVIKPWMGGEISLRSAEK
GSGAGEGDQQDAAHNMGNHLPLLPAESEEEDEMEVEDQDSKEAKKPNIINFDTSLPTSHTYLGA
DMEEFHGRTLHDDDSCQVIPVLPQVMMILIPGQTLPLQLFHPQEVSMVRNLIQKDRTFAVLAYS
NVQEREAQFGTTAEIYAYREEQDFGIEIVKVKAIGRQRFKVLELRTQSDGIQQAKVQILPECVL
PSTMSAVQLESLNKCQIFPSKPVSREDQCSYKWWQKYQKRKFHCANLTSWPRWLYSLYDAETLM
DRIKKQLREWDENLKDDSLPSNPIDESYRVAACLPIDDVLRIQLLKIGSAIQRLRCELDIMNKC
TSLCCKQCQETEITTKNEIFSLSLCGPMAAYVNPHGYVHETLTVYKACNLNLIGRPSTEHSWFP
GYAWTVAQCKICASHIGWKFTATKKDMSPQKFWGLTRSALLPTIPDTEDEISPDKVILCLEGRG
SLLTCGDVEENPGPMVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIFFKDDGNYKTRAEVK
FEGDTLVNRIELKGIDFKEDGNILGHKLEYNYNSHNVYIMADKQKNGIKVNFKIRHNIEDGSVQ
LADHYQQNTPIGDGPVLLPDNHYLSTQSALSKDPNEKRDHMVLLEFVTAAGITLGMDELYK-
SEQ ID NO: 3 NP_001166953.1
>NP_001166953.1 CRBN [organism = Homo sapiens] [GeneID = 51185]
[isoform = 2]
MAGEGDQQDAAHNMGNHLPLLPESEEEDEMEVEDQDSKEAKKPNIINFDTSLPTSHTYLGADME
EFHGRTLHDDDSCQVIPVLPQVMMILIPGQTLPLQLFHPQEVSMVRNLIQKDRTFAVLAYSNVQ
EREAQFGTTAEIYAYREEQDFGIEIVKVKAIGRQRFKVLELRTQSDGIQQAKVQILPECVLPST
MSAVQLESLNKCQIFPSKPVSREDQCSYKWWQKYQKRKFHCANLTSWPRWLYSLYDAETLMDRI
KKQLREWDENLKDDSLPSNPIDESYRVAACLPIDDVLRIQLLKIGSAIQRLRCELDIMNKCTSL
CCKQCQETEITTKNEIFSLSLCGPMAAYVNPHGYVHETLTVYKACNLNLIGRPSTEHSWFPGYA
WTVAQCKICASHIGWKFTATKKDMSPQKFWGLTRSALLPTIPDTEDEISPDKVILCL
SEQ ID NO: 4 NP_057386.2
>NP_057386.2 CRBN [organism = Homo sapiens] [GeneID = 51185]
[isoform = 1]
MAGEGDQQDAAHNMGNHLPLLPAESEEEDEMEVEDQDSKEAKKPNIINFDTSLPTSHTYLGADM
EEFHGRTLHDDDSCQVIPVLPQVMMILIPGQTLPLQLFHPQEVSMVRNLIQKDRTFAVLAYSNV
QEREAQFGTTAEIYAYREEQDFGIEIVKVKAIGRQRFKVLELRTQSDGIQQAKVQILPECVLPS
TMSAVQLESLNKCQIFPSKPVSREDQCSYKWWQKYQKRKFHCANLTSWPRWLYSLYDAETLMDR
IKKQLREWDENLKDDSLPSNPIDESYRVAACLPIDDVLRIQLLKIGSAIQRLRCELDIMNKCTS
LCCKQCQETEITTKNEIFSLSLCGPMAAYVNPHGYVHETLTVYKACNLNLIGRPSTEHSWFPGY
AWTVAQCKICASHIGWKFTATKKDMSPQKFWGLTRSALLPTIPDTEDEISPDKVILCL
SEQ ID NO: 5 XP_005265259.1
>XP_005265259.1 CRBN [organism = Homo sapiens] [GeneID = 51185]
[isoform = X2]
MEEFHGRTLHDDDSCQVIPVLPQVMMILIPGQTLPLQLFHPQEVSMVRNLIQKDRTFAVLAYSN
VQEREAQFGTTAEIYAYREEQDFGIEIVKVKAIGRQRFKVLELRTQSDGIQQAKVQILPECVLP
STMSAVQLESLNKCQIFPSKPVSREDQCSYKWWQKYQKRKFHCANLTSWPRWLYSLYDAETLMD
RIKKQLREWDENLKDDSLPSNPIDFSYRVAACLPIDDVLRIQLLKIGSAIQRLRCELDIMNKCT
SLCCKQCQETEITTKNEIFSLSLCGPMAAYVNPHGYVHETLTVYKACNLNLIGRPSTEHSWFPG
YAWTVAQCKICASHIGWKFTATKKDMSPQKFWGLTRSALLPTIPDTEDEISPDKVILCL
SEQ ID NO: 6 XP_011532093.1
>XP_011532093.1 CRBN [organism = Homo sapiens] [GeneID = 51185]
[isoform = X1]
MAGEGDQQDAAHNMGNHLPLLPAESEEEDEMEVEDQDSKEAKKPNIINFDTSLPTSHTYLGADM
EEFHGRTLHDDDSCQVIPVLPQVMMILIPGQTLPLQLFHPQEVSMVRNLIQKDRTFAVLAYSNV
QEREAQFGTTAEIYAYREEQDFGIEIVKVKAIGRQRFKVLELRTQSDGIQQAKVQILPECVLPS
TMSAVQLESLNKCQIFPSKPVSREDQCSYKWWQKYQKRKFHCANLTSWPRWLYSLYDAETLMDR
IKKQLREWDENLKDDSLPSNPIDFSYRVAACLPIDDVLRIQLLKIGSAIQRLRCELDIMNKCTS
LCCKQCQETEITTKNEIFRYAWTVAQCKICASHIGWKFTATKKDMSPQKFWGLTRSALLPTIPD
TEDEISPDKVILCL
SEQ ID NO: 7 XP_011532095.1
>XP_011532095.1 CRBN [organism = Homo sapiens] [GeneID = 51185]
[isoform = X4]
MRLQHLLKMIFRIQQAKVQILPECVLPSTMSAVQLESLNKCQIFPSKPVSREDQCSYKWWQKYQ
KRKFHCANLTSWPRWLYSLYDAETLMDRIKKQLREWDENLKDDSLPSNPIDESYRVAACLPIDD
VLRIQLLKIGSAIQRLRCELDIMNKCTSLCCKQCQETEITTKNEIFSLSLCGPMAAYVNPHGYV
HETLTVYKACNLNLIGRPSTEHSWFPGYAWTVAQCKICASHIGWKFTATKKDMSPQKFWGLTRS
ALLPTIPDTEDEISPDKVILCL
SEQ ID NO: 8 XP_011532096.1
>XP_011532096.1 CRBN [organism = Homo sapiens] [GeneID = 51185]
[isoform = X4]
MRLQHLLKMIFRIQQAKVQILPECVLPSTMSAVQLESLNKCQIFPSKPVSREDQCSYKWWQKYQ
KRKFHCANLTSWPRWLYSLYDAETLMDRIKKQLREWDENLKDDSLPSNPIDESYRVAACLPIDD
VLRIQLLKIGSAIQRLRCELDIMNKCTSLCCKQCQETEITTKNEIFSLSLCGPMAAYVNPHGYV
HETLTVYKACNLNLIGRPSTEHSWFPGYAWTVAQCKICASHIGWKFTATKKDMSPQKFWGLTRS
ALLPTIPDTEDEISPDKVILCL
SEQ ID NO: 9 XP_024309319.1
>XP_024309319.1 CRBN [organism = Homo sapiens] [GeneID = 51185]
[isoform = X3]
MAGEGDQQDAAHNMGNHLPLLPAESEEEDEMEVEDQDSKEAKKPNIINFDTSLPTSHTYLGADM
EEFHGRTLHDDDSCQVIPVLPQVMMILIPGQTLPLQLFHPQEVSMVRNLIQKDRTFAVLAYSNV
QEREAQFGTTAEIYAYREEQDFGIEIVKVKAIGRQRFKVLELRTQSDGIQQAKVQILPECVLPS
TMSAVQLESLNKCQIFPSKPVSREDQCSYKWWQKYQKRKFHCANLTSWPRWLYSLYDAETLMDR
IKKQLREWDENLKDDSLPSNPIVYFPLL
SEQ ID NO: 10 TurboID CRBN vector GOI region
TCGCTAGCGCCGCCACCATGAAAGACAATACTGTGCCTCTGAAGCTGATCGCTCTCCTGGCTAA
TGGCGAGTTCCATAGTGGCGAACAGCTGGGAGAAACCCTGGGCATGTCCAGGGCCGCTATCAAC
AAGCACATTCAGACTCTGCGCGACTGGGGCGTGGACGTGTTCACCGTGCCCGGAAAGGGCTACT
CTCTGCCCGAGCCTATCCCGCTGCTGAACGCTAAACAGATTCTGGGACAGCTGGACGGCGGGAG
CGTGGCAGTCCTGCCTGTGGTCGACTCCACCAATCAGTACCTGCTGGATCGAATCGGCGAGCTG
AAGAGTGGGGATGCTTGCATTGCAGAATATCAGCAGGCAGGGAGAGGAAGCAGAGGGAGGAAAT
GGTTCTCTCCTTTTGGAGCTAACCTGTACCTGAGTATGTTTTGGCGCCTGAAGCGGGGACCAGC
AGCAATCGGCCTGGGCCCGGTCATCGGAATTGTCATGGCAGAAGCGCTGCGAAAGCTGGGAGCA
GACAAGGTGCGAGTCAAATGGCCCAATGACCTGTATCTGCAGGATAGAAAGCTGGCAGGCATCC
TGGTGGAGCTGGCCGGAATAACAGGCGATGCTGCACAGATCGTCATTGGCGCCGGGATTAACGT
GGCTATGAGGCGCGTGGAGGAAAGCGTGGTCAATCAGGGCTGGATCACACTGCAGGAAGCAGGG
ATTAACCTGGACAGGAATACTCTGGCCGCTACGCTGATCCGAGAGCTGCGGGCAGCCCTGGAAC
TGTTCGAGCAGGAAGGCCTGGCTCCATATCTGCCACGGTGGGAGAAGCTGGATAACTTCATCAA
TAGACCCGTGAAGCTGATCATTGGGGACAAAGAGATTTTCGGGATTAGCCGGGGGATTGATAAA
CAGGGAGCCCTGCTGCTGGAACAGGACGGAGTTATCAAACCCTGGATGGGCGGAGAAATCAGTC
TGCGGTCTGCCGAAAAGGGTTCTGGAGCCGGCGAGGGCGACCAGCAGGATGCCGCTCATAACAT
GGGCAACCATCTGCCTCTGCTGCCCGCAGAGAGCGAAGAAGAGGACGAGATGGAAGTGGAAGAT
CAGGACAGCAAAGAGGCCAAGAAGCCCAACATCATCAACTTCGACACCAGCCTGCCTACCAGCC
ACACATATCTGGGCGCCGACATGGAAGAGTTCCACGGCAGAACCCTGCACGACGATGACAGCTG
CCAAGTGATCCCCGTGCTGCCCCAAGTCATGATGATTCTGATCCCCGGCCAGACACTGCCCCTG
CAGCTGTTTCATCCTCAAGAGGTGTCCATGGTCCGAAACCTGATCCAGAAGGACCGGACCTTTG
CCGTGCTGGCCTACAGCAACGTGCAAGAGAGAGAGGCCCAGTTTGGCACCACCGCCGAGATCTA
CGCCTACAGAGAGGAACAGGACTTCGGCATCGAGATCGTGAAAGTGAAGGCCATCGGCCGGCAG
CGGTTCAAGGTTCTGGAACTGAGAACCCAGAGCGACGGCATCCAGCAGGCCAAGGTTCAGATCC
TGCCTGAGTGTGTGCTGCCCAGCACAATGTCTGCCGTGCAGCTGGAAAGCCTGAACAAGTGCCA
GATCTTCCCCAGCAAGCCCGTGTCCAGAGAAGATCAGTGCAGCTACAAGTGGTGGCAGAAGTAC
CAGAAGCGGAAGTTCCACTGCGCCAACCTGACCAGCTGGCCCAGATGGCTGTACTCCCTGTACG
ATGCCGAGACACTGATGGACCGGATCAAAAAGCAGCTGAGAGAGTGGGACGAGAACCTGAAGGA
CGACTCCCTGCCTAGCAACCCCATCGACTTCAGCTATAGAGTGGCCGCCTGCCTGCCTATCGAC
GACGTGCTGAGAATCCAGCTGCTGAAGATCGGCAGCGCCATCCAGAGACTGAGATGCGAGCTGG
ACATCATGAACAAATGTACCAGCCTGTGCTGCAAGCAGTGCCAAGAGACAGAGATCACCACCAA
GAACGAGATCTTTAGCCTGAGCCTGTGCGGCCCTATGGCCGCCTATGTGAATCCTCACGGCTAC
GTGCACGAAACCCTGACCGTGTACAAGGCCTGCAACCTGAACCTGATCGGCAGACCTAGCACCG
AGCACAGCTGGTTTCCAGGATACGCCTGGACAGTGGCCCAGTGCAAGATCTGTGCCTCTCACAT
CGGCTGGAAGTTCACCGCCACCAAAAAGGACATGAGCCCTCAGAAGTTCTGGGGCCTGACCAGA
AGTGCCCTGCTGCCTACAATCCCCGACACCGAGGATGAGATCAGCCCCGACAAAGTGATCCTGT
GCCTGGAAGGCAGGGGTTCTCTGCTGACCTGCGGAGATGTCGAAGAAAATCCCGGACCAATGGT
GTCCAAGGGCGAAGAACTGTTTACCGGCGTGGTGCCCATCCTGGTGGAACTGGATGGGGACGTG
AACGGACACAAGTTCAGCGTTAGCGGAGAAGGCGAAGGCGACGCCACATACGGAAAGCTGACCC
TGAAGTTCATCTGCACCACCGGCAAGCTGCCTGTGCCTTGGCCTACACTGGTCACCACACTGAC
ATACGGCGTGCAGTGCTTCAGCAGATATCCCGACCATATGAAGCAGCACGACTTCTTCAAGAGC
GCCATGCCTGAGGGCTACGTGCAAGAGCGGACCATCTTCTTTAAGGACGACGGCAACTACAAGA
CCAGGGCCGAAGTGAAGTTCGAGGGCGACACCCTGGTCAACCGGATCGAGCTGAAGGGCATCGA
CTTCAAAGAGGACGGCAACATCCTGGGACACAAGCTCGAGTACAATTACAACTCCCACAACGTG
TACATCATGGCCGACAAGCAGAAAAACGGCATCAAAGTCAACTTCAAGATTCGGCACAACATCG
AGGACGGCTCCGTGCAGCTGGCCGATCATTATCAGCAGAACACCCCTATCGGCGACGGCCCAGT
TCTGCTGCCCGATAATCACTACCTGTCCACTCAGTCTGCCCTGAGCAAGGACCCCAACGAGAAG
AGGGATCACATGGTGCTGCTCGAGTTTGTGACCGCCGCTGGCATCACACTCGGCATGGATGAGC
TGTACAAGTGAATCTAGAAGTT
SEQ ID NO: 11 TurboID CRBN fusion protein polynucleotide sequence
ATGAAAGACAATACTGTGCCTCTGAAGCTGATCGCTCTCCTGGCTAATGGCGAGTTCCATAGTG
GCGAACAGCTGGGAGAAACCCTGGGCATGTCCAGGGCCGCTATCAACAAGCACATTCAGACTCT
GCGCGACTGGGGCGTGGACGTGTTCACCGTGCCCGGAAAGGGCTACTCTCTGCCCGAGCCTATC
CCGCTGCTGAACGCTAAACAGATTCTGGGACAGCTGGACGGCGGGAGCGTGGCAGTCCTGCCTG
TGGTCGACTCCACCAATCAGTACCTGCTGGATCGAATCGGCGAGCTGAAGAGTGGGGATGCTTG
CATTGCAGAATATCAGCAGGCAGGGAGAGGAAGCAGAGGGAGGAAATGGTTCTCTCCTTTTGGA
GCTAACCTGTACCTGAGTATGTTTTGGCGCCTGAAGCGGGGACCAGCAGCAATCGGCCTGGGCC
CGGTCATCGGAATTGTCATGGCAGAAGCGCTGCGAAAGCTGGGAGCAGACAAGGTGCGAGTCAA
ATGGCCCAATGACCTGTATCTGCAGGATAGAAAGCTGGCAGGCATCCTGGTGGAGCTGGCCGGA
ATAACAGGCGATGCTGCACAGATCGTCATTGGCGCCGGGATTAACGTGGCTATGAGGCGCGTGG
AGGAAAGCGTGGTCAATCAGGGCTGGATCACACTGCAGGAAGCAGGGATTAACCTGGACAGGAA
TACTCTGGCCGCTACGCTGATCCGAGAGCTGCGGGCAGCCCTGGAACTGTTCGAGCAGGAAGGC
CTGGCTCCATATCTGCCACGGTGGGAGAAGCTGGATAACTTCATCAATAGACCCGTGAAGCTGA
TCATTGGGGACAAAGAGATTTTCGGGATTAGCCGGGGGATTGATAAACAGGGAGCCCTGCTGCT
GGAACAGGACGGAGTTATCAAACCCTGGATGGGCGGAGAAATCAGTCTGCGGTCTGCCGAAAAG
GGTTCTGGAGCCGGCGAGGGCGACCAGCAGGATGCCGCTCATAACATGGGCAACCATCTGCCTC
TGCTGCCCGCAGAGAGCGAAGAAGAGGACGAGATGGAAGTGGAAGATCAGGACAGCAAAGAGGC
CAAGAAGCCCAACATCATCAACTTCGACACCAGCCTGCCTACCAGCCACACATATCTGGGCGCC
GACATGGAAGAGTTCCACGGCAGAACCCTGCACGACGATGACAGCTGCCAAGTGATCCCCGTGC
TGCCCCAAGTCATGATGATTCTGATCCCCGGCCAGACACTGCCCCTGCAGCTGTTTCATCCTCA
AGAGGTGTCCATGGTCCGAAACCTGATCCAGAAGGACCGGACCTTTGCCGTGCTGGCCTACAGC
AACGTGCAAGAGAGAGAGGCCCAGTTTGGCACCACCGCCGAGATCTACGCCTACAGAGAGGAAC
AGGACTTCGGCATCGAGATCGTGAAAGTGAAGGCCATCGGCCGGCAGCGGTTCAAGGTTCTGGA
ACTGAGAACCCAGAGCGACGGCATCCAGCAGGCCAAGGTTCAGATCCTGCCTGAGTGTGTGCTG
CCCAGCACAATGTCTGCCGTGCAGCTGGAAAGCCTGAACAAGTGCCAGATCTTCCCCAGCAAGC
CCGTGTCCAGAGAAGATCAGTGCAGCTACAAGTGGTGGCAGAAGTACCAGAAGCGGAAGTTCCA
CTGCGCCAACCTGACCAGCTGGCCCAGATGGCTGTACTCCCTGTACGATGCCGAGACACTGATG
GACCGGATCAAAAAGCAGCTGAGAGAGTGGGACGAGAACCTGAAGGACGACTCCCTGCCTAGCA
ACCCCATCGACTTCAGCTATAGAGTGGCCGCCTGCCTGCCTATCGACGACGTGCTGAGAATCCA
GCTGCTGAAGATCGGCAGCGCCATCCAGAGACTGAGATGCGAGCTGGACATCATGAACAAATGT
ACCAGCCTGTGCTGCAAGCAGTGCCAAGAGACAGAGATCACCACCAAGAACGAGATCTTTAGCC
TGAGCCTGTGCGGCCCTATGGCCGCCTATGTGAATCCTCACGGCTACGTGCACGAAACCCTGAC
CGTGTACAAGGCCTGCAACCTGAACCTGATCGGCAGACCTAGCACCGAGCACAGCTGGTTTCCA
GGATACGCCTGGACAGTGGCCCAGTGCAAGATCTGTGCCTCTCACATCGGCTGGAAGTTCACCG
CCACCAAAAAGGACATGAGCCCTCAGAAGTTCTGGGGCCTGACCAGAAGTGCCCTGCTGCCTAC
AATCCCCGACACCGAGGATGAGATCAGCCCCGACAAAGTGATCCTGTGCCTG
SEQ ID NO: 12 TurboID CRBN fusion protein with T2A and GFP
polynucleotide sequence
ATGAAAGACAATACTGTGCCTCTGAAGCTGATCGCTCTCCTGGCTAATGGCGAGTTCCATAGTG
GCGAACAGCTGGGAGAAACCCTGGGCATGTCCAGGGCCGCTATCAACAAGCACATTCAGACTCT
GCGCGACTGGGGCGTGGACGTGTTCACCGTGCCCGGAAAGGGCTACTCTCTGCCCGAGCCTATC
CCGCTGCTGAACGCTAAACAGATTCTGGGACAGCTGGACGGCGGGAGCGTGGCAGTCCTGCCTG
TGGTCGACTCCACCAATCAGTACCTGCTGGATCGAATCGGCGAGCTGAAGAGTGGGGATGCTTG
CATTGCAGAATATCAGCAGGCAGGGAGAGGAAGCAGAGGGAGGAAATGGTTCTCTCCTTTTGGA
GCTAACCTGTACCTGAGTATGTTTTGGCGCCTGAAGCGGGGACCAGCAGCAATCGGCCTGGGCC
CGGTCATCGGAATTGTCATGGCAGAAGCGCTGCGAAAGCTGGGAGCAGACAAGGTGCGAGTCAA
ATGGCCCAATGACCTGTATCTGCAGGATAGAAAGCTGGCAGGCATCCTGGTGGAGCTGGCCGGA
ATAACAGGCGATGCTGCACAGATCGTCATTGGCGCCGGGATTAACGTGGCTATGAGGCGCGTGG
AGGAAAGCGTGGTCAATCAGGGCTGGATCACACTGCAGGAAGCAGGGATTAACCTGGACAGGAA
TACTCTGGCCGCTACGCTGATCCGAGAGCTGCGGGCAGCCCTGGAACTGTTCGAGCAGGAAGGC
CTGGCTCCATATCTGCCACGGTGGGAGAAGCTGGATAACTTCATCAATAGACCCGTGAAGCTGA
TCATTGGGGACAAAGAGATTTTCGGGATTAGCCGGGGGATTGATAAACAGGGAGCCCTGCTGCT
GGAACAGGACGGAGTTATCAAACCCTGGATGGGCGGAGAAATCAGTCTGCGGTCTGCCGAAAAG
GGTTCTGGAGCCGGCGAGGGCGACCAGCAGGATGCCGCTCATAACATGGGCAACCATCTGCCTC
TGCTGCCCGCAGAGAGCGAAGAAGAGGACGAGATGGAAGTGGAAGATCAGGACAGCAAAGAGGC
CAAGAAGCCCAACATCATCAACTTCGACACCAGCCTGCCTACCAGCCACACATATCTGGGCGCC
GACATGGAAGAGTTCCACGGCAGAACCCTGCACGACGATGACAGCTGCCAAGTGATCCCCGTGC
TGCCCCAAGTCATGATGATTCTGATCCCCGGCCAGACACTGCCCCTGCAGCTGTTTCATCCTCA
AGAGGTGTCCATGGTCCGAAACCTGATCCAGAAGGACCGGACCTTTGCCGTGCTGGCCTACAGC
AACGTGCAAGAGAGAGAGGCCCAGTTTGGCACCACCGCCGAGATCTACGCCTACAGAGAGGAAC
AGGACTTCGGCATCGAGATCGTGAAAGTGAAGGCCATCGGCCGGCAGCGGTTCAAGGTTCTGGA
ACTGAGAACCCAGAGCGACGGCATCCAGCAGGCCAAGGTTCAGATCCTGCCTGAGTGTGTGCTG
CCCAGCACAATGTCTGCCGTGCAGCTGGAAAGCCTGAACAAGTGCCAGATCTTCCCCAGCAAGC
CCGTGTCCAGAGAAGATCAGTGCAGCTACAAGTGGTGGCAGAAGTACCAGAAGCGGAAGTTCCA
CTGCGCCAACCTGACCAGCTGGCCCAGATGGCTGTACTCCCTGTACGATGCCGAGACACTGATG
GACCGGATCAAAAAGCAGCTGAGAGAGTGGGACGAGAACCTGAAGGACGACTCCCTGCCTAGCA
ACCCCATCGACTTCAGCTATAGAGTGGCCGCCTGCCTGCCTATCGACGACGTGCTGAGAATCCA
GCTGCTGAAGATCGGCAGCGCCATCCAGAGACTGAGATGCGAGCTGGACATCATGAACAAATGT
ACCAGCCTGTGCTGCAAGCAGTGCCAAGAGACAGAGATCACCACCAAGAACGAGATCTTTAGCC
TGAGCCTGTGCGGCCCTATGGCCGCCTATGTGAATCCTCACGGCTACGTGCACGAAACCCTGAC
CGTGTACAAGGCCTGCAACCTGAACCTGATCGGCAGACCTAGCACCGAGCACAGCTGGTTTCCA
GGATACGCCTGGACAGTGGCCCAGTGCAAGATCTGTGCCTCTCACATCGGCTGGAAGTTCACCG
CCACCAAAAAGGACATGAGCCCTCAGAAGTTCTGGGGCCTGACCAGAAGTGCCCTGCTGCCTAC
AATCCCCGACACCGAGGATGAGATCAGCCCCGACAAAGTGATCCTGTGCCTGGAAGGCAGGGGT
TCTCTGCTGACCTGCGGAGATGTCGAAGAAAATCCCGGACCAATGGTGTCCAAGGGCGAAGAAC
TGTTTACCGGCGTGGTGCCCATCCTGGTGGAACTGGATGGGGACGTGAACGGACACAAGTTCAG
CGTTAGCGGAGAAGGCGAAGGCGACGCCACATACGGAAAGCTGACCCTGAAGTTCATCTGCACC
ACCGGCAAGCTGCCTGTGCCTTGGCCTACACTGGTCACCACACTGACATACGGCGTGCAGTGCT
TCAGCAGATATCCCGACCATATGAAGCAGCACGACTTCTTCAAGAGCGCCATGCCTGAGGGCTA
CGTGCAAGAGCGGACCATCTTCTTTAAGGACGACGGCAACTACAAGACCAGGGCCGAAGTGAAG
TTCGAGGGCGACACCCTGGTCAACCGGATCGAGCTGAAGGGCATCGACTTCAAAGAGGACGGCA
ACATCCTGGGACACAAGCTCGAGTACAATTACAACTCCCACAACGTGTACATCATGGCCGACAA
GCAGAAAAACGGCATCAAAGTCAACTTCAAGATTCGGCACAACATCGAGGACGGCTCCGTGCAG
CTGGCCGATCATTATCAGCAGAACACCCCTATCGGCGACGGCCCAGTTCTGCTGCCCGATAATC
ACTACCTGTCCACTCAGTCTGCCCTGAGCAAGGACCCCAACGAGAAGAGGGATCACATGGTGCT
GCTCGAGTTTGTGACCGCCGCTGGCATCACACTCGGCATGGATGAGCTGTACAAGTGA
SEQ ID NO: 13 pcLV-CMV-MCS-T2A-eGFP-IRES-Puro GOI insert
TCGCTAGCGCCGCCACCATGAAAGACAATACTGTGCCTCTGAAGCTGATCGCTCTCCTGGCTAA
TGGCGAGTTCCATAGTGGCGAACAGCTGGGAGAAACCCTGGGCATGTCCAGGGCCGCTATCAAC
AAGCACATTCAGACTCTGCGCGACTGGGGCGTGGACGTGTTCACCGTGCCCGGAAAGGGCTACT
CTCTGCCCGAGCCTATCCCGCTGCTGAACGCTAAACAGATTCTGGGACAGCTGGACGGCGGGAG
CGTGGCAGTCCTGCCTGTGGTCGACTCCACCAATCAGTACCTGCTGGATCGAATCGGCGAGCTG
AAGAGTGGGGATGCTTGCATTGCAGAATATCAGCAGGCAGGGAGAGGAAGCAGAGGGAGGAAAT
GGTTCTCTCCTTTTGGAGCTAACCTGTACCTGAGTATGTTTTGGCGCCTGAAGCGGGGACCAGC
AGCAATCGGCCTGGGCCCGGTCATCGGAATTGTCATGGCAGAAGCGCTGCGAAAGCTGGGAGCA
GACAAGGTGCGAGTCAAATGGCCCAATGACCTGTATCTGCAGGATAGAAAGCTGGCAGGCATCC
TGGTGGAGCTGGCCGGAATAACAGGCGATGCTGCACAGATCGTCATTGGCGCCGGGATTAACGT
GGCTATGAGGCGCGTGGAGGAAAGCGTGGTCAATCAGGGCTGGATCACACTGCAGGAAGCAGGG
ATTAACCTGGACAGGAATACTCTGGCCGCTACGCTGATCCGAGAGCTGCGGGCAGCCCTGGAAC
TGTTCGAGCAGGAAGGCCTGGCTCCATATCTGCCACGGTGGGAGAAGCTGGATAACTTCATCAA
TAGACCCGTGAAGCTGATCATTGGGGACAAAGAGATTTTCGGGATTAGCCGGGGGATTGATAAA
CAGGGAGCCCTGCTGCTGGAACAGGACGGAGTTATCAAACCCTGGATGGGCGGAGAAATCAGTC
TGCGGTCTGCCGAAAAGGGTTCTGGAGCCGGCGAGGGCGACCAGCAGGATGCCGCTCATAACAT
GGGCAACCATCTGCCTCTGCTGCCCGCAGAGAGCGAAGAAGAGGACGAGATGGAAGTGGAAGAT
CAGGACAGCAAAGAGGCCAAGAAGCCCAACATCATCAACTTCGACACCAGCCTGCCTACCAGCC
ACACATATCTGGGCGCCGACATGGAAGAGTTCCACGGCAGAACCCTGCACGACGATGACAGCTG
CCAAGTGATCCCCGTGCTGCCCCAAGTCATGATGATTCTGATCCCCGGCCAGACACTGCCCCTG
CAGCTGTTTCATCCTCAAGAGGTGTCCATGGTCCGAAACCTGATCCAGAAGGACCGGACCTTTG
CCGTGCTGGCCTACAGCAACGTGCAAGAGAGAGAGGCCCAGTTTGGCACCACCGCCGAGATCTA
CGCCTACAGAGAGGAACAGGACTTCGGCATCGAGATCGTGAAAGTGAAGGCCATCGGCCGGCAG
CGGTTCAAGGTTCTGGAACTGAGAACCCAGAGCGACGGCATCCAGCAGGCCAAGGTTCAGATCC
TGCCTGAGTGTGTGCTGCCCAGCACAATGTCTGCCGTGCAGCTGGAAAGCCTGAACAAGTGCCA
GATCTTCCCCAGCAAGCCCGTGTCCAGAGAAGATCAGTGCAGCTACAAGTGGTGGCAGAAGTAC
CAGAAGCGGAAGTTCCACTGCGCCAACCTGACCAGCTGGCCCAGATGGCTGTACTCCCTGTACG
ATGCCGAGACACTGATGGACCGGATCAAAAAGCAGCTGAGAGAGTGGGACGAGAACCTGAAGGA
CGACTCCCTGCCTAGCAACCCCATCGACTTCAGCTATAGAGTGGCCGCCTGCCTGCCTATCGAC
GACGTGCTGAGAATCCAGCTGCTGAAGATCGGCAGCGCCATCCAGAGACTGAGATGCGAGCTGG
ACATCATGAACAAATGTACCAGCCTGTGCTGCAAGCAGTGCCAAGAGACAGAGATCACCACCAA
GAACGAGATCTTTAGCCTGAGCCTGTGCGGCCCTATGGCCGCCTATGTGAATCCTCACGGCTAC
GTGCACGAAACCCTGACCGTGTACAAGGCCTGCAACCTGAACCTGATCGGCAGACCTAGCACCG
AGCACAGCTGGTTTCCAGGATACGCCTGGACAGTGGCCCAGTGCAAGATCTGTGCCTCTCACAT
CGGCTGGAAGTTCACCGCCACCAAAAAGGACATGAGCCCTCAGAAGTTCTGGGGCCTGACCAGA
AGTGCCCTGCTGCCTACAATCCCCGACACCGAGGATGAGATCAGCCCCGACAAAGTGATCCTGT
GCCTGGAAGGCAGGGGTTCTCTGCTGACCTGCGGAGATGTCGAAGAAAATCCCGGACCAATGGT
GTCCAAGGGCGAAGAACTGTTTACCGGCGTGGTGCCCATCCTGGTGGAACTGGATGGGGACGTG
AACGGACACAAGTTCAGCGTTAGCGGAGAAGGCGAAGGCGACGCCACATACGGAAAGCTGACCC
TGAAGTTCATCTGCACCACCGGCAAGCTGCCTGTGCCTTGGCCTACACTGGTCACCACACTGAC
ATACGGCGTGCAGTGCTTCAGCAGATATCCCGACCATATGAAGCAGCACGACTTCTTCAAGAGC
GCCATGCCTGAGGGCTACGTGCAAGAGCGGACCATCTTCTTTAAGGACGACGGCAACTACAAGA
CCAGGGCCGAAGTGAAGTTCGAGGGCGACACCCTGGTCAACCGGATCGAGCTGAAGGGCATCGA
CTTCAAAGAGGACGGCAACATCCTGGGACACAAGCTCGAGTACAATTACAACTCCCACAACGTG
TACATCATGGCCGACAAGCAGAAAAACGGCATCAAAGTCAACTTCAAGATTCGGCACAACATCG
AGGACGGCTCCGTGCAGCTGGCCGATCATTATCAGCAGAACACCCCTATCGGCGACGGCCCAGT
TCTGCTGCCCGATAATCACTACCTGTCCACTCAGTCTGCCCTGAGCAAGGACCCCAACGAGAAG
AGGGATCACATGGTGCTGCTCGAGTTTGTGACCGCCGCTGGCATCACACTCGGCATGGATGAGC
TGTACAAGTGAATCTAGAAGTT
SEQ ID NO: 14 E. coli BirA
>NP_418404.1 birA [organism = Escherichia coli str. K-12 substr.
MG1655] [GeneID = 948469]
MKDNTVPLKLIALLANGEFHSGEQLGETLGMSRAAINKHIQTLRDWGVDVFTVPGKGYSLPEPI
QLLNAKQILGQLDGGSVAVLPVIDSTNQYLLDRIGELKSGDACIAEYQQAGRGRRGRKWFSPFG
ANLYLSMFWRLEQGPAAAIGLSLVIGIVMAEVLRKLGADKVRVKWPNDLYLQDRKLAGILVELT
GKTGDAAQIVIGAGINMAMRRVEESVVNQGWITLQEAGINLDRNTLAAMLIRELRAALELFEQE
GLAPYLSRWEKLDNFINRPVKLIIGDKEIFGISRGIDKQGALLLEQDGIIKPWMGGEISLRSAE
K
SEQ ID NO: 15 BioID2 amino acid
FKNLIWLKEVDSTQERLKEWNVSYGTALVADRQTKGRGGLGRKWLSQEGGLYFSFLLNPKEFEN
LLQLPLVLGLSVSEALEEITEIPFSLKWPNDVYFQEKKVSGVLCELSKDKLIVGIGINVNQREI
PEEIKDRATTLYEITGKDWDRKEVLLKVLKRISENLKKFKEKSFKEFKGKIESKMLYLGEEVKL
LGEGKITGKLVGLSEKGGALILTEEGIKEILSGEFSLRRS
SEQ ID NO: 16 BioID2 polynucleotide
TTCAAGAACCTGATCTGGCTGAAGGAGGTGGACAGCACCCAGGAGAGACTGAAGGAGTGGAACG
TGAGCTACGGCACCGCCCTGGTGGCCGACAGACAGACCAAGGGCAGAGGCGGCCTGGGCAGAAA
GTGGCTGAGCCAGGAGGGCGGCCTGTACTTCAGCTTCCTGCTGAACCCCAAGGAGTTCGAGAAC
CTGCTGCAGCTGCCCCTGGTGCTGGGCCTGAGCGTGAGCGAGGCCCTGGAGGAGATCACCGAGA
TCCCCTTCAGCCTGAAGTGGCCCAACGACGTGTACTTCCAGGAGAAGAAGGTGAGCGGCGTGCT
GTGCGAGCTGAGCAAGGACAAGCTGATCGTGGGCATCGGCATCAACGTGAACCAGAGAGAGATC
CCCGAGGAGATCAAGGACAGAGCCACCACCCTGTACGAGATCACCGGCAAGGACTGGGACAGAA
AGGAGGTGCTGCTGAAGGTGCTGAAGAGAATCAGCGAGAACCTGAAGAAGTTCAAGGAGAAGAG
CTTCAAGGAGTTCAAGGGCAAGATCGAGAGCAAGATGCTGTACCTGGGCGAGGAGGTGAAGCTG
CTGGGCGAGGGCAAGATCACCGGCAAGCTGGTGGGCCTGAGCGAGAAGGGCGGCGCCCTGATCC
TGACCGAGGAGGGCATCAAGGAGATCCTGAGCGGCGAGTTCAGCCTGAGAAGAAGC
SEQ ID NO: 17 BASU
GKLSESEIRFGLKTEVMGQHLIYHDVLSSTQKTAHELANNNAPEGTLVVADKQTAGRGGMSRVW
HSQEGNGVWMSLILRPDIPLQKTPQLTLLAAVAVVQGIEEAAGIQTDIKWPNDILINGKKTVGI
LTEMQAEEDRVRSVIIGIGINVNQQPNDFPDELKDIATSLSQAAGEKIDRAGVIQHILLCFEKR
YRDYMTHGFTPIKLLWESYALGIGTNMRARTLNGTFYGKALGIDDEGVLLLETNEGIKKIYSAD
ISLR
SEQ ID NO: 18 miniTurbo amino acid
IPLLNAKQILGQLDGGSVAVLPVVDSTNQYLLDRIGELKSGDACIAEYQQAGRGSRGRKWFSPF
GANLYLSMFWRLKRGPAAIGLGPVIGIVMAEALRKLGADKVRVKWPNDLYLQDRKLAGILVELA
GITGDAAQIVIGAGINVAMRRVEESVVNQGWITLQEAGINLDRNTLAAMLIRELRAALELFEQE
GLAPYLSRWEKLDNFINRPVKLIIGDKEIFGISRGIDKQGALLLEQDGVIKPWMGGEISLRSAE
K
SEQ ID NO: 19 miniTurbo polynucleotide
ATCCCGCTGCTGAACGCTAAACAGATTCTGGGACAGCTGGACGGCGGGAGCGTGGCAGTCCTGC
CTGTGGTCGACTCCACCAATCAGTACCTGCTGGATCGAATCGGCGAGCTGAAGAGTGGGGATGC
TTGCATTGCAGAATATCAGCAGGCAGGGAGAGGAAGCAGAGGGAGGAAATGGTTCTCTCCTTTT
GGAGCTAACCTGTACCTGAGTATGTTTTGGCGCCTGAAGCGGGGACCAGCAGCAATCGGCCTGG
GCCCGGTCATCGGAATTGTCATGGCAGAAGCGCTGCGAAAGCTGGGAGCAGACAAGGTGCGAGT
CAAATGGCCCAATGACCTGTATCTGCAGGATAGAAAGCTGGCAGGCATCCTGGTGGAGCTGGCC
GGAATAACAGGCGATGCTGCACAGATCGTCATTGGCGCCGGGATTAACGTGGCTATGAGGCGCG
TGGAGGAAAGCGTGGTCAATCAGGGCTGGATCACACTGCAGGAAGCAGGGATTAACCTGGACAG
GAATACTCTGGCCGCTATGCTGATCCGAGAGCTGCGGGCAGCCCTGGAACTGTTCGAGCAGGAA
GGCCTGGCTCCATATCTGTCACGGTGGGAGAAGCTGGATAACTTCATCAATAGACCCGTGAAGC
TGATCATTGGGGACAAAGAGATTTTCGGGATTAGCCGGGGGATTGATAAACAGGGAGCCCTGCT
GCTGGAACAGGACGGAGTTATCAAACCCTGGATGGGCGGAGAAATCAGTCTGCGGTCTGCCGAA
AAG
SEQ ID NO: 20 turboID amino acid
KDNTVPLKLIALLANGEFHSGEQLGETLGMSRAAINKHIQTLRDWGVDVFTVPGKGYSLPEPIP
LLNAKQILGQLDGGSVAVLPVVDSTNQYLLDRIGELKSGDACIAEYQQAGRGSRGRKWFSPFGA
NLYLSMFWRLKRGPAAIGLGPVIGIVMAEALRKLGADKVRVKWPNDLYLQDRKLAGILVELAGI
TGDAAQIVIGAGINVAMRRVEESVVNQGWITLQEAGINLDRNTLAATLIRELRAALELFEQEGL
APYLPRWEKLDNFINRPVKLIIGDKEIFGISRGIDKQGALLLEQDGVIKPWMGGEISLRSAEK
SEQ ID NO: 21 turboID polynucleotide
AAAGACAATACTGTGCCTCTGAAGCTGATCGCTCTCCTGGCTAATGGCGAGTTCCATAGTGGCG
AACAGCTGGGAGAAACCCTGGGCATGTCCAGGGCCGCTATCAACAAGCACATTCAGACTCTGCG
CGACTGGGGCGTGGACGTGTTCACCGTGCCCGGAAAGGGCTACTCTCTGCCCGAGCCTATCCCG
CTGCTGAACGCTAAACAGATTCTGGGACAGCTGGACGGCGGGAGCGTGGCAGTCCTGCCTGTGG
TCGACTCCACCAATCAGTACCTGCTGGATCGAATCGGCGAGCTGAAGAGTGGGGATGCTTGCAT
TGCAGAATATCAGCAGGCAGGGAGAGGAAGCAGAGGGAGGAAATGGTTCTCTCCTTTTGGAGCT
AACCTGTACCTGAGTATGTTTTGGCGCCTGAAGCGGGGACCAGCAGCAATCGGCCTGGGCCCGG
TCATCGGAATTGTCATGGCAGAAGCGCTGCGAAAGCTGGGAGCAGACAAGGTGCGAGTCAAATG
GCCCAATGACCTGTATCTGCAGGATAGAAAGCTGGCAGGCATCCTGGTGGAGCTGGCCGGAATA
ACAGGCGATGCTGCACAGATCGTCATTGGCGCCGGGATTAACGTGGCTATGAGGCGCGTGGAGG
AAAGCGTGGTCAATCAGGGCTGGATCACACTGCAGGAAGCAGGGATTAACCTGGACAGGAATAC
TCTGGCCGCTACGCTGATCCGAGAGCTGCGGGCAGCCCTGGAACTGTTCGAGCAGGAAGGCCTG
GCTCCATATCTGCCACGGTGGGAGAAGCTGGATAACTTCATCAATAGACCCGTGAAGCTGATCA
TTGGGGACAAAGAGATTTTCGGGATTAGCCGGGGGATTGATAAACAGGGAGCCCTGCTGCTGGA
ACAGGACGGAGTTATCAAACCCTGGATGGGCGGAGAAATCAGTCTGCGGTCTGCCGAAAAG
SEQ ID NO: 22 AirID amino acid
MKDNTVPLTLISILADGEFHSGEQLGEQLGMSRAAINKHIKTLRDWGVDVERVQGKGYCLPEPI
QLLDEEKIRQQLDEGSVTVLPVIDSTNQYLLDRLDELTSGDVCIAEYQQAGRGSRGRKWFSPFG
ANLYLSMYWRLEQGPAAAMGLSLVIGIVMAETLQKLGADGVRVKWPNDLYLNDRKLAGILVEMT
GKTGDAAHIVIGAGINLSMREPETDEVDQSWINLQEAGITIDRNQLAARLIKDLRSALRQFEQQ
GLAPFLSRWEALDNFINRPVKLIIGDREIHGIARGINEQGALLLEQDGVIKPWIGGEISLRSA
SEQ ID NO: 23 AVVA amino acid
MSEQWSRKREILALLSSGHFVSGEELATQLGISRTAVSKHIAALEEYGVDIYSVKGKGYKLANP
ISLIDESKLKSAINNRCFYFDEIPSTNGEMLKHAEELKSGDICVAEYQSAGRGRRGRTWVSPYG
CHLYFSLYWRFPQGMAQAMGLSLVVACSLVKVLKSFGVDGVGVKWPNDIYLNHKKLAGVLIEMS
GQADSECHLVIGIGINMAMSEQQGKKIDQPWSDLSSLTSMPDKTELLIALQKQLKQDLELFERE
GLKAFQPRWQEADLFYGKQIKLLMGENQVEGICRGIDEQGAVLLETDDGIQAFIGGEISLRAA
SEQ ID NO: 24 AVVA polynucleotide
ATGTCTGAGCAGTGGTCTAGGAAGAGAGAGATCCTTGCTCTCCTCAGCTCTGGACATTTCGTGT
CTGGTGAGGAACTTGCTACTCAGCTCGGAATCTCTAGAACCGCTGTGAGCAAACATATCGCTGC
CCTTGAAGAGTACGGCGTGGACATCTATAGCGTGAAAGGTAAGGGATACAAGCTCGCGAACCCG
ATCTCTCTTATCGACGAGTCTAAGCTCAAGAGCGCCATCAACAACCGTTGCTTCTACTTCGACG
AGATCCCGTCTACCAACGGCTTCATGCTTAAACACGCTGAGGAACTCAAGTCCGGGGATATCTG
TGTTGCTGAGTACCAGTCTGCTGGAAGAGGACGTAGAGGAAGAACTTGGGTTTCACCTTACGGA
TGCCACCTCTACTTCAGCCTTTATTGGAGGTTCCCGCAAGGTATGGCTCAGGCTATGGGACTTT
CTCTCGTTGTTGCTTGCAGCCTCGTTAAGGTGCTCAAGTCTTTCGGAGTTGATGGTGTGGGAGT
GAAGTGGCCTAACGACATCTACCTCAACCATAAGAAACTCGCCGGTGTGCTCATCGAGATGTCT
GGACAAGCTGATTCTGAGTGCCATCTCGTTATCGGCATCGGGATCAACATGGCTATGTCTGAAC
AGCAGGGGAAGAAGATCGATCAGCCTTGGTCTGACCTCTCCTCTCTCACTTCTATGCCTGACAA
GACCGAGCTGCTTATCGCTCTCCAAAAGCAGCTTAAGCAGGACCTCGAGCTTTTCGAGAGAGAG
GGACTTAAGGCTTTCCAGCCTAGATGGCAAGAGGCTGATCTCTTCTACGGGAAGCAGATCAAGC
TTCTCATGGGAGAGAATCAGGTCGAGGGAATCTGCAGAGGAATTGATGAGCAGGGTGCTGTTCT
CCTCGAGACTGATGATGGAATCCAGGCTTTTATCGGCGGGGAGATCTCTTTGAGAGCTGCT
SEQ ID NO: 25 AHLA amino acid
MRPFPLLRLLSDGEFHSGQALAEALGVSRASIWNALRNAEALGVDVHAVRGRGYRLSEPLDWLD
EAIVARHLGEKASFFDLHVLDSVDSTNTALMERALQGAPHGTCVAAERQTAGRGRRGRAWHAVL
GGSLTFSLLWRFNLGLGSLSGLSLAVGLAVVRALNKLGVHGARLKWPNDVLTDYRKLAGILIEL
QGDMLGPAAAVIGIGLNVRLSEAARNAVDQAVVDLHSLCGAPADRNTLLADLLRELAAMLTAFE
QDGFAPLRAEWEAHHAYQDKAVRLLLPDGAGVQGVARGVDEDGALLLE
SEQ ID NO: 26 AHLA polynucleotide
ATGAGGCCTTTTCCGCTTCTCAGACTCTTGTCTGATGGGGAGTTCCATTCTGGACAAGCTCTTG
CTGAAGCTCTCGGAGTGTCTAGAGCTTCTATCTGGAACGCTCTCAGAAACGCTGAGGCTCTTGG
AGTTGATGTGCATGCTGTTAGAGGACGTGGGTACAGACTTTCTGAGCCTCTTGATTGGCTCGAC
GAGGCTATCGTTGCTAGACATCTTGGAGAGAAGGCCAGCTTCTTCGATCTCCATGTTCTCGACT
CTGTGGACTCTACTAACACGGCTCTCATGGAAAGGGCTCTCCAAGGTGCTCCTCATGGAACTTG
TGTTGCTGCTGAGAGACAGACTGCTGGAAGAGGAAGAAGAGGTAGAGCTTGGCATGCTGTGCTT
GGAGGATCTCTTACCTTCTCTCTTCTCTGGCGTTTCAACCTCGGACTCGGATCTCTTTCTGGAC
TCTCTCTTGCTGTTGGACTCGCTGTTGTTAGGGCTCTTAACAAGCTCGGAGTGCATGGTGCTAG
ACTCAAGTGGCCTAACGATGTGCTCACCGATTACAGAAAGCTCGCTGGAATCCTCATCGAGCTT
CAGGGTGATATGCTTGGACCTGCTGCTGCTGTTATCGGAATCGGACTTAACGTGAGACTCTCTG
AGGCTGCTAGGAACGCTGTTGATCAGGCTGTTGTGGATCTCCATTCTCTTTGTGGTGCTCCGGC
TGATAGAAATACCCTTCTTGCTGATCTCCTCCGTGAGCTTGCTGCTATGCTTACTGCTTTCGAG
CAGGATGGATTCGCTCCTCTTAGAGCTGAATGGGAAGCTCATCACGCTTACCAGGATAAGGCTG
TGAGACTTCTTTTGCCTGATGGTGCTGGTGTTCAGGGTGTTGCTAGAGGTGTTGATGAGGATGG
TGCTTTGCTCCTCGAGACTCAATCTGGGGAGAGAAGATTCCACAGCGGAGAGATTTCTCTTAGG
CCTGCTGCT
SEQ ID NO: 27 GFVA amino acid
MKDNTVPLTLISILADGEFHSGEQLGEQLGMSRAAINKHIKTLRDWGVDVFRVQGKGYCLPEPI
QLLDEEKIRQQLDEGSVTVLPVIDSTNQYLLDRLDELTSGDVCIAEYQQAGRGRRGRKWFSPFG
ANLYLSMYWRLEQGPAAAMGLSLVIGIVMAETLQKLGADGVRVKWPNDLYLNDRKLAGILVEMT
GKTGDAAHIVIGAGINLSMREPETDEVDQSWINLQEAGITIDRNQLAARLIKDLRSALRQFEQQ
GLAPFLSRWEALDNFINRPVKLIIGDREIHGIARGINEQGALLLEQDGVIKPWIGGEISLRSA
SEQ ID NO: 28 GFVA polynucleotide
ATGAAGGACAACACCGTTCCGCTCACGCTTATCTCTATCCTTGCTGATGGTGAGTTCCACTCTG
GTGAACAACTTGGAGAGCAGCTCGGAATGTCTAGGGCTGCTATTAACAAGCACATCAAGACCCT
CCGTGACTGGGGAGTTGATGTGTTCAGAGTTCAAGGTAAGGGGTACTGCCTTCCTGAGCCTATC
CAACTTCTCGACGAAGAGAAGATCAGGCAGCAGCTTGATGAGGGATCTGTTACTGTTCTCCCGG
TGATCGATTCGACCAACCAGTACCTTCTCGATAGGCTCGATGAGCTTACCTCTGGTGATGTGTG
TATCGCTGAGTACCAACAGGCTGGAAGAGGACGTAGAGGTAGGAAGTGGTTTTCTCCGTTCGGA
GCTAACCTCTACCTCAGCATGTATTGGAGACTTGAGCAAGGACCTGCTGCTGCTATGGGACTTT
CTCTCGTTATCGGAATCGTGATGGCTGAGACTCTCCAAAAGCTTGGAGCTGACGGTGTTAGAGT
GAAGTGGCCTAACGACCTTTACCTCAACGATAGGAAGCTCGCTGGAATCCTCGTTGAGATGACT
GGAAAGACTGGTGACGCTGCTCATATCGTGATTGGAGCTGGAATCAACCTCTCTATGCGTGAGC
CTGAGACTGATGAGGTTGACCAGTCTTGGATCAACCTCCAAGAGGCTGGTATCACCATCGATAG
AAACCAGCTTGCTGCCAGGCTCATCAAGGATCTTAGATCTGCTCTCAGGCAGTTCGAGCAACAA
GGACTTGCTCCATTCCTCAGCAGATGGGAAGCTCTCGACAACTTCATCAACAGGCCAGTGAAGC
TCATCATCGGTGATAGAGAGATCCACGGAATCGCTAGGGGAATCAACGAACAAGGGGCTCTTTT
GCTTGAGCAGGACGGTGTGATTAAGCCTTGGATTGGAGGTGAGATCAGCCTCAGATCTGCT
SEQ ID NO: 29 All amino acid
MKDKTRPLKLIAILADGQFHSGEELATQLGISRAAINKHIKTLREWGVDVESVQGKGYCLANPI
QLLDETKIKQQLKNRVTVLPVIDSTNQYLLDRLDELKSGDVCVAEYQSAGRGRRGRKWFSPFGS
NLYFSMYWRLEQGMAAAMGLSLVVGIVMAEVLKKLGADGVRVKWPNDLYLNDRKLAGILVEMTG
KTGDAAHIVIGIGINLSMSEPETNEVDQSWANLSNVGITIDRNQLVASLAKDLKSALRQFEQQG
LAAFLSRWQALDNFINRPVKLLIGDKEIHGIARGINEQGALLLEQDGGIKAYIGGEISLRSA
SEQ ID NO: 30 All polynucleotide
ATGAAGGACAAGACCAGACCGCTCAAGCTTATCGCTATCCTTGCTGATGGACAGTTCCACTCTG
GTGAGGAACTTGCTACTCAGCTCGGAATTTCTAGGGCCGCTATCAACAAGCACATCAAGACTCT
CCGTGAGTGGGGAGTTGATGTGTTCTCTGTTCAAGGTAAGGGGTACTGCCTCGCTAACCCTATC
CAACTTCTCGACGAGACTAAGATCAAGCAGCAGCTCAAGAACAGGGTGACAGTTCTCCCTGTGA
TCGACTCTACTAACCAGTACCTCCTCGATAGGCTCGACGAGCTTAAGTCTGGTGATGTTTGTGT
GGCCGAGTACCAGTCTGCTGGAAGAGGACGTAGAGGTAGGAAGTGGTTTAGCCCGTTCGGAAGC
AACCTCTACTTCAGCATGTATTGGAGGCTCGAGCAAGGTATGGCTGCTGCTATGGGACTTTCTC
TCGTTGTGGGAATCGTGATGGCTGAGGTGCTCAAGAAGCTTGGAGCTGACGGGGTTAGAGTTAA
GTGGCCTAACGATCTCTACCTCAACGACAGAAAGCTCGCTGGAATCCTCGTTGAGATGACTGGA
AAGACTGGTGACGCTGCTCATATCGTGATCGGAATCGGTATCAACCTCAGCATGTCTGAGCCTG
AGACTAACGAGGTTGACCAGTCTTGGGCTAACCTCTCTAACGTGGGAATCACCATCGATAGGAA
CCAGCTCGTTGCTTCTCTCGCTAAGGATCTCAAGTCTGCTCTCAGACAATTCGAGCAGCAGGGA
CTTGCTGCTTTCTTGTCTAGATGGCAGGCTCTCGACAACTTCATCAACAGACCTGTGAAGCTCC
TCATCGGGGACAAAGAGATTCACGGAATCGCTAGGGGAATCAACGAACAAGGGGCTCTTTTGCT
TGAGCAGGACGGTGGAATCAAGGCTTACATCGGAGGTGAGATCAGCCTCAGATCTGCT
SEQ ID NO: 31 (VHL)
>sp|P40337|VHL_HUMAN von Hippel-Lindau disease tumor suppressor
OS = Homo sapiens OX = 9606 GN = VHL PE = 1 SV = 2
MPRRAENWDEAEVGAEEAGVEEYGPEEDGGEESGAEESGPEESGPEELGAEEEMEAGRPRPVLR
SVNSREPSQVIFCNRSPRVVLPVWLNEDGEPQPYPTLPPGTGRRIHSYRGHLWLFRDAGTHDGL
LVNQTELFVPSLNVDGQPIFANITLPVYTLKERCLQVVRSLVKPENYRRLDIVRSLYEDLEDHP
NVQKDLERLTQERIAHQRMGD
SEQ ID NO: 32 (NAIP; BIRC1)
>sp|Q13075|BIRC1_HUMAN Baculoviral IAP repeat-containing protein 1
OS = Homo sapiens OX = 9606 GN = NAIP PE = 1 SV = 3
MATQQKASDERISQFDHNLLPELSALLGLDAVQLAKELEEEEQKERAKMQKGYNSQMRSEAKRL
KTFVTYEPYSSWIPQEMAAAGFYFTGVKSGIQCFCCSLILFGAGLTRLPIEDHKRFHPDCGFLL
NKDVGNIAKYDIRVKNLKSRLRGGKMRYQEEEARLASFRNWPFYVQGISPCVLSEAGFVFTGKQ
DTVQCFSCGGCLGNWEEGDDPWKEHAKWFPKCEFLRSKKSSEEITQYIQSYKGFVDITGEHFVN
SWVQRELPMASAYCNDSIFAYEELRLDSFKDWPRESAVGVAALAKAGLFYTGIKDIVQCFSCGG
CLEKWQEGDDPLDDHTRCFPNCPFLQNMKSSAEVTPDLQSRGELCELLETTSESNLEDSIAVGP
IVPEMAQGEAQWFQEAKNLNEQLRAAYTSASFRHMSLLDISSDLATDHLLGCDLSIASKHISKP
VQEPLVLPEVFGNLNSVMCVEGEAGSGKTVLLKKIAFLWASGCCPLLNRFQLVFYLSLSSTRPD
EGLASIICDQLLEKEGSVTEMCVRNIIQQLKNQVLFLLDDYKEICSIPQVIGKLIQKNHLSRTC
LLIAVRTNRARDIRRYLETILEIKAFPFYNTVCILRKLFSHNMTRLRKEMVYFGKNQSLQKIQK
TPLFVAAICAHWFQYPFDPSEDDVAVEKSYMERLSLRNKATAEILKATVSSCGELALKGFFSCC
FEFNDDDLAEAGVDEDEDLTMCLMSKFTAQRLRPFYRFLSPAFQEFLAGMRLIELLDSDRQEHQ
DLGLYHLKQINSPMMTVSAYNNFLNYVSSLPSTKAGPKIVSHLLHLVDNKESLENISENDDYLK
HQPEISLQMQLLRGLWQICPQAYFSMVSEHLLVLALKTAYQSNTVAACSPFVLQFLQGRTLTLG
ALNLQYFFDHPESLSLLRSIHFPIRGNKTSPRAHFSVLETCEDKSQVPTIDQDYASAFEPMNEW
ERNLAEKEDNVKSYMDMQRRASPDLSTGYWKLSPKQYKIPCLEVDVNDIDVVGQDMLEILMTVF
SASQRIELHLNHSRGFIESIRPALELSKASVTKCSISKLELSAAEQELLLTLPSLESLEVSGTI
QSQDQIFPNLDKFLCLKELSVDLEGNINVFSVIPEEFPNFHHMEKLLIQISAEYDPSKLVKLIQ
NSPNLHVFHLKCNFFSDFGSLMTMLVSCKKLTEIKFSDSFFQAVPFVASLPNFISLKILNLEGQ
QFPDEETSEKFAYILGSLSNLEELILPTGDGIYRVAKLIIQQCQQLHCLRVLSFFKTLNDDSVV
EIAKVAISGGFQKLENLKLSINHKITEEGYRNFFQALDNMPNLQELDISRHFTECIKAQATTVK
SLSQCVLRLPRLIRLNMLSWLLDADDIALLNVMKERHPQSKYLTILQKWILPFSPIIQK
SEQ ID NO: 33 cIAP1 (BIRC2)
>sp|Q13490|BIRC2_HUMAN Baculoviral IAP repeat-containing protein 2
OS = Homo sapiens OX = 9606 GN = BIRC2 PE = 1 SV = 2
MHKTASQRLFPGPSYQNIKSIMEDSTILSDWTNSNKQKMKYDESCELYRMSTYSTFPAGVPVSE
RSLARAGFYYTGVNDKVKCFCCGLMLDNWKLGDSPIQKHKQLYPSCSFIQNLVSASLGSTSKNT
SPMRNSFAHSLSPTLEHSSLESGSYSSLSPNPLNSRAVEDISSSRTNPYSYAMSTEEARFLTYH
MWPLTFLSPSELARAGFYYIGPGDRVACFACGGKLSNWEPKDDAMSEHRRHFPNCPFLENSLET
LRFSISNLSMQTHAARMRTFMYWPSSVPVQPEQLASAGFYYVGRNDDVKCFCCDGGLRCWESGD
DPWVEHAKWFPRCEFLIRMKGQEFVDEIQGRYPHLLEQLLSTSDTTGEENADPPIIHFGPGESS
SEDAVMMNTPVVKSALEMGENRDLVKQTVQSKILTTGENYKTVNDIVSALLNAEDEKREEEKEK
QAEEMASDDLSLIRKNRMALFQQLTCVLPILDNLLKANVINKQEHDIIKQKTQIPLQARELIDT
ILVKGNAAANIFKNCLKEIDSTLYKNLFVDKNMKYIPTEDVSGLSLEEQLRRLQEERTCKVCMD
KEVSVVFIPCGHLVVCQECAPSLRKCPICRGIIKGTVRTFLS
SEQ ID NO: 34 cIAP2 (BIRC3)
>sp|Q13489|BIRC3_HUMAN Baculoviral IAP repeat-containing protein 3
OS = Homo sapiens OX = 9606 GN = BIRC3 PE = 1 SV = 2
MNIVENSIFLSNLMKSANTFELKYDLSCELYRMSTYSTFPAGVPVSERSLARAGFYYTGVNDKV
KCFCCGLMLDNWKRGDSPTEKHKKLYPSCRFVQSLNSVNNLEATSQPTFPSSVINSTHSLLPGT
ENSGYFRGSYSNSPSNPVNSRANQDESALMRSSYHCAMNNENARLLTFQTWPLTELSPTDLAKA
GFYYIGPGDRVACFACGGKLSNWEPKDNAMSEHLRHFPKCPFIENQLQDTSRYTVSNLSMQTHA
ARFKTFFNWPSSVLVNPEQLASAGFYYVGNSDDVKCFCCDGGLRCWESGDDPWVQHAKWFPRCE
YLIRIKGQEFIRQVQASYPHLLEQLLSTSDSPGDENAESSIIHFEPGEDHSEDAIMMNTPVINA
AVEMGFSRSLVKQTVQRKILATGENYRLVNDLVLDLLNAEDEIREEERERATEEKESNDLLLIR
KNRMALFQHLTCVIPILDSLLTAGIINEQEHDVIKQKTQTSLQARELIDTILVKGNIAATVERN
SLQEAEAVLYEHLFVQQDIKYIPTEDVSDLPVEEQLRRLQEERTCKVCMDKEVSIVFIPCGHLV
VCKDCAPSLRKCPICRSTIKGTVRTFLS
SEQ ID NO: 35 (XIAP; BIRC4)
>sp|P98170|XIAP_HUMAN E3 ubiquitin-protein ligase XIAP OS = Homo
sapiens OX = 9606 GN = XIAP PE = 1 SV = 2
MTFNSFEGSKTCVPADINKEEEFVEEFNRLKTFANFPSGSPVSASTLARAGFLYTGEGDTVRCF
SCHAAVDRWQYGDSAVGRHRKVSPNCRFINGFYLENSATQSTNSGIQNGQYKVENYLGSRDHFA
LDRPSETHADYLLRTGQVVDISDTIYPRNPAMYSEEARLKSFQNWPDYAHLTPRELASAGLYYT
GIGDQVQCFCCGGKLKNWEPCDRAWSEHRRHFPNCFFVLGRNLNIRSESDAVSSDRNFPNSTNL
PRNPSMADYEARIFTFGTWIYSVNKEQLARAGFYALGEGDKVKCFHCGGGLTDWKPSEDPWEQH
AKWYPGCKYLLEQKGQEYINNIHLTHSLEECLVRTTEKTPSLTRRIDDTIFQNPMVQEAIRMGF
SFKDIKKIMEEKIQISGSNYKSLEVLVADLVNAQKDSMQDESSQTSLQKEISTEEQLRRLQEEK
LCKICMDRNIAIVFVPCGHLVTCKQCAEAVDKCPMCYTVITFKQKIEMS
SEQ ID NO: 36 (Survivin; BIRC5),
>sp|O15392|BIRC5_HUMAN Baculoviral IAP repeat-containing protein 5
OS = Homo sapiens OX = 9606 GN = BIRC5 PE = 1 SV = 3
MGAPTLPPAWQPFLKDHRISTFKNWPFLEGCACTPERMAEAGFIHCPTENEPDLAQCFFCFKEL
EGWEPDDDPIEEHKKHSSGCAFLSVKKQFEELTLGEFLKLDRERAKNKIAKETNNKKKEFEETA
KKVRRAIEQLAAMD
SEQ ID NO: 37 (BRUCE; BIRC6)
>sp|Q9NR09|BIRC6_HUMAN Baculoviral IAP repeat-containing protein 6
OS = Homo sapiens OX = 9606 GN = BIRC6 PE = 1 SV = 2
MVTGGGAAPPGTVTEPLPSVIVLSAGRKMAAAAAAASGPGCSSAAGAGAAGVSEWLVLRDGCMH
CDADGLHSLSYHPALNAILAVTSRGTIKVIDGTSGATLQASALSAKPGGQVKCQYISAVDKVIF
VDDYAVGCRKDLNGILLLDTALQTPVSKQDDVVQLELPVTEAQQLLSACLEKVDISSTEGYDLE
ITQLKDGLKNTSHETAANHKVAKWATVTFHLPHHVLKSIASAIVNELKKINQNVAALPVASSVM
DRLSYLLPSARPELGVGPGRSVDRSLMYSEANRRETFTSWPHVGYRWAQPDPMAQAGFYHQPAS
SGDDRAMCFTCSVCLVCWEPTDEPWSEHERHSPNCPFVKGEHTQNVPLSVTLATSPAQFPCTDG
TDRISCFGSGSCPHFLAAATKRGKICIWDVSKLMKVHLKFEINAYDPAIVQQLILSGDPSSGVD
SRRPTLAWLEDSSSCSDIPKLEGDSDDLLEDSDSEEHSRSDSVTGHTSQKEAMEVSLDITALSI
LQQPEKLQWEIVANVLEDTVKDLEELGANPCLTNSKSEKTKEKHQEQHNIPFPCLLAGGLLTYK
SPATSPISSNSHRSLDGLSRTQGESISEQGSTDNESCINSELNSPLVRRTLPVLLLYSIKESDE
KAGKIFSQMNNIMSKSLHDDGFTVPQIIEMELDSQEQLLLQDPPVTYIQQFADAAANLTSPDSE
KWNSVFPKPGTLVQCLRLPKFAEEENLCIDSITPCADGIHLLVGLRTCPVESLSAINQVEALNN
LNKLNSALCNRRKGELESNLAVVNGANISVIQHESPADVQTPLIIQPEQRNVSGGYLVLYKMNY
ATRIVTLEEEPIKIQHIKDPQDTITSLILLPPDILDNREDDCEEPIEDMQLTSKNGFEREKTSD
ISTLGHLVITTQGGYVKILDLSNFEILAKVEPPKKEGTEEQDTFVSVIYCSGTDRLCACTKGGE
LHFLQIGGTCDDIDEADILVDGSLSKGIEPSSEGSKPLSNPSSPGISGVDLLVDQPFTLEILTS
LVELTRFETLTPRESATVPPCWVEVQQEQQQRRHPQHLHQQHHGDAAQHTRTWKLQTDSNSWDE
HVFELVLPKACMVGHVDFKFVLNSNITNIPQIQVTLLKNKAPGLGKVNALNIEVEQNGKPSLVD
LNEEMQHMDVEESQCLRLCPFLEDHKEDILCGPVWLASGLDLSGHAGMLTLTSPKLVKGMAGGK
YRSFLIHVKAVNERGTEEICNGGMRPVVRLPSLKHQSNKGYSLASLLAKVAAGKEKSSNVKNEN
TSGTRKSENLRGCDLLQEVSVTIRRFKKTSISKERVQRCAMLQFSEFHEKLVNTLCRKTDDGQI
TEHAQSLVLDTLCWLAGVHSNGPGSSKEGNENLLSKTRKELSDIVRVCFFEAGRSIAHKCARFL
ALCISNGKCDPCQPAFGPVLLKALLDNMSFLPAATTGGSVYWYFVLLNYVKDEDLAGCSTACAS
LLTAVSRQLQDRLTPMEALLQTRYGLYSSPFDPVLFDLEMSGSSCKNVYNSSIGVQSDEIDLSD
VLSGNGKVSSCTAAEGSFTSLTGLLEVEPLHFTCVSTSDGTRIERDDAMSSFGVTPAVGGLSSG
TVGEASTALSSAAQVALQSLSHAMASAEQQLQVLQEKQQQLLKLQQQKAKLEAKLHQTTAAAAA
AASAVGPVHNSVPSNPVAAPGFFIHPSDVIPPTPKTTPLFMTPPLTPPNEAVSVVINAELAQLF
PGSVIDPPAVNLAAHNKNSNKSRMNPLGSGLALAISHASHFLQPPPHQSIIIERMHSGARRFVT
LDFGRPILLTDVLIPTCGDLASLSIDIWTLGEEVDGRRLVVATDISTHSLILHDLIPPPVCREM
KITVIGRYGSTNARAKIPLGFYYGHTYILPWESELKLMHDPLKGEGESANQPEIDQHLAMMVAL
QEDIQCRYNLACHRLETLLQSIDLPPLNSANNAQYFLRKPDKAVEEDSRVFSAYQDCIQLQLQL
NLAHNAVQRLKVALGASRKMLSETSNPEDLIQTSSTEQLRTIIRYLLDTLLSLLHASNGHSVPA
VLQSTFHAQACEELFKHLCISGTPKIRLHTGLLLVQLCGGERWWGQFLSNVLQELYNSEQLLIF
PQDRVFMLLSCIGQRSLSNSGVLESLLNLLDNLLSPLQPQLPMHRRTEGVLDIPMISWVVMLVS
RLLDYVATVEDEAAAAKKPLNGNQWSFINNNLHTQSLNRSSKGSSSLDRLYSRKIRKQLVHHKQ
QLNLLKAKQKALVEQMEKEKIQSNKGSSYKLLVEQAKLKQATSKHFKDLIRLRRTAEWSRSNLD
TEVTTAKESPEIEPLPFTLAHERCISVVQKLVLFLLSMDFTCHADLLLFVCKVLARIANATRPT
IHLCEIVNEPQLERLLLLLVGTDENRGDISWGGAWAQYSLTCMLQDILAGELLAPVAAEAMEEG
TVGDDVGATAGDSDDSLQQSSVQLLETIDEPLTHDITGAPPLSSLEKDKEIDLELLQDLMEVDI
DPLDIDLEKDPLAAKVFKPISSTWYDYWGADYGTYNYNPYIGGLGIPVAKPPANTEKNGSQTVS
VSVSQALDARLEVGLEQQAELMLKMMSTLEADSILQALTNTSPTLSQSPTGTDDSLLGGLQAAN
QTSQLIIQLSSVPMLNVCFNKLFSMLQVHHVQLESLLQLWLTLSLNSSSTGNKENGADIFLYNA
NRIPVISLNQASITSFLTVLAWYPNTLLRTWCLVLHSLTLMTNMQLNSGSSSAIGTQESTAHLL
VSDPNLIHVLVKFLSGTSPHGTNQHSPQVGPTATQAMQEFLTRLQVHLSSTCPQIFSEFLLKLI
HILSTERGAFQTGQGPLDAQVKLLEFTLEQNFEVVSVSTISAVIESVTFLVHHYITCSDKVMSR
SGSDSSVGARACFGGLFANLIRPGDAKAVCGEMTRDQLMFDLLKLVNILVQLPLSGNREYSARV
SVTTNTTDSVSDEEKVSGGKDGNGSSTSVQGSPAYVADLVLANQQIMSQILSALGLCNSSAMAM
IIGASGLHLTKHENFHGGLDAISVGDGLFTILTTLSKKASTVHMMLQPILTYMACGYMGRQGSL
ATCQLSEPLLWFILRVLDTSDALKAFHDMGGVQLICNNMVTSTRAIVNTARSMVSTIMKFLDSG
PNKAVDSTLKTRILASEPDNAEGIHNFAPLGTITSSSPTAQPAEVLLQATPPHRRARSAAWSYI
FLPEEAWCDLTIHLPAAVLLKEIHIQPHLASLATCPSSVSVEVSADGVNMLPLSTPVVTSGLTY
IKIQLVKAEVASAVCLRLHRPRDASTLGLSQIKLLGLTAFGTTSSATVNNPFLPSEDQVSKTSI
GWLRLLHHCLTHISDLEGMMASAAAPTANLLQTCAALLMSPYCGMHSPNIEVVLVKIGLQSTRI
GLKLIDILLRNCAASGSDPTDLNSPLLFGRLNGLSSDSTIDILYQLGTTQDPGTKDRIQALLKW
VSDSARVAAMKRSGRMNYMCPNSSTVEYGLLMPSPSHLHCVAAILWHSYELLVEYDLPALLDQE
LFELLFNWSMSLPCNMVLKKAVDSLLCSMCHVHPNYFSLLMGWMGITPPPVQCHHRLSMTDDSK
KQDLSSSLTDDSKNAQAPLALTESHLATLASSSQSPEAIKQLLDSGLPSLLVRSLASFCESHIS
SSESIAQSIDISQDKLRRHHVPQQCNKMPITADLVAPILRFLTEVGNSHIMKDWLGGSEVNPLW
TALLFLLCHSGSTSGSHNLGAQQTSARSASLSSAATTGLTTQQRTAIENATVAFFLQCISCHPN
NQKLMAQVLCELFQTSPQRGNLPTSGNISGFIRRLFLQLMLEDEKVTMFLQSPCPLYKGRINAT
SHVIQHPMYGAGHKFRTLHLPVSTTLSDVLDRVSDTPSITAKLISEQKDDKEKKNHEEKEKVKA
ENGFQDNYSVVVASGLKSQSKRAVSATPPRPPSRRGRTIPDKIGSTSGAEAANKIITVPVFHLF
HKLLAGQPLPAEMTLAQLLTLLYDRKLPQGYRSIDLTVKLGSRVITDPSLSKTDSYKRLHPEKD
HGDLLASCPEDEALTPGDECMDGILDESLLETCPIQSPLQVFAGMGGLALIAERLPMLYPEVIQ
QVSAPVVTSTTQEKPKDSDQFEWVTIEQSGELVYEAPETVAAEPPPIKSAVQTMSPIPAHSLAA
FGLFLRLPGYAEVLLKERKHAQCLLRLVLGVTDDGEGSHILQSPSANVLPTLPFHVLRSLESTT
PLTTDDGVLLRRMALEIGALHLILVCLSALSHHSPRVPNSSVNQTEPQVSSSHNPTSTEEQQLY
WAKGTGFGTGSTASGWDVEQALTKQRLEEEHVTCLLQVLASYINPVSSAVNGEAQSSHETRGQN
SNALPSVLLELLSQSCLIPAMSSYLRNDSVLDMARHVPLYRALLELLRAIASCAAMVPLLLPLS
TENGEEEEEQSECQTSVGTLLAKMKTCVDTYTNRLRSKRENVKTGVKPDASDQEPEGLTLLVPD
IQKTAEIVYAATTSLRQANQEKKLGEYSKKAAMKPKPLSVLKSLEEKYVAVMKKLQFDTFEMVS
EDEDGKLGFKVNYHYMSQVKNANDANSAARARRLAQEAVTLSTSLPLSSSSSVFVRCDEERLDI
MKVLITGPADTPYANGCFEFDVYFPQDYPSSPPLVNLETTGGHSVRENPNLYNDGKVCLSILNT
WHGRPEEKWNPQTSSFLQVLVSVQSLILVAEPYFNEPGYERSRGTPSGTQSSREYDGNIRQATV
KWAMLEQIRNPSPCFKEVIHKHFYLKRVEIMAQCEEWIADIQQYSSDKRVGRTMSHHAAALKRH
TAQLREELLKLPCPEGLDPDTDDAPEVCRATTGAEETLMHDQVKPSSSKELPSDFQL
SEQ ID NO: 38 (ML-IAP; BIRC7)
>sp|Q96CA5|BIRC7_HUMAN Baculoviral IAP repeat-containing protein 7
OS = Homo sapiens OX = 9606 GN = BIRC7 PE = 1 SV = 2
MGPKDSAKCLHRGPQPSHWAAGDGPTQERCGPRSLGSPVLGLDTCRAWDHVDGQILGQLRPLTE
EEEEEGAGATLSRGPAFPGMGSEELRLASFYDWPLTAEVPPELLAAAGFFHTGHQDKVRCFFCY
GGLQSWKRGDDPWTEHAKWFPSCQFLLRSKGRDFVHSVQETHSQLLGSWDPWEEPEDAAPVAPS
VPASGYPELPTPRREVQSESAQEPGGVSPAEAQRAWWVLEPPGARDVEAQLRRLQEERTCKVCL
DRAVSIVFVPCGHLVCAECAPGLQLCPICRAPVRSRVRTFLS
SEQ ID NO: 39 (ILP2; BIRC8)
>sp|Q96P09|BIRC8_HUMAN Baculoviral IAP repeat-containing protein 8
OS = Homo sapiens OX = 9606 GN = BIRC8 PE = 1 SV = 2
MTGYEARLITFGTWMYSVNKEQLARAGFYAIGQEDKVQCFHCGGGLANWKPKEDPWEQHAKWYP
GCKYLLEEKGHEYINNIHLTRSLEGALVQTTKKTPSLTKRISDTIFPNPMLQEAIRMGFDFKDV
KKIMEERIQTSGSNYKTLEVLVADLVSAQKDTTENELNQTSLQREISPEEPLRRLQEEKLCKIC
MDRHIAVVFIPCGHLVTCKQCAEAVDRCPMCSAVIDEKQRVEMS
SEQ ID NO: 40 (KEAP1)
>sp|Q14145|KEAP1_HUMAN Kelch-like ECH-associated protein 1
OS = Homo sapiens OX = 9606 GN = KEAP1 PE = 1 SV = 2
MQPDPRPSGAGACCRFLPLQSQCPEGAGDAVMYASTECKAEVTPSQHGNRTFSYTLEDHTKQAF
GIMNELRLSQQLCDVTLQVKYQDAPAAQFMAHKVVLASSSPVFKAMFTNGLREQGMEVVSIEGI
HPKVMERLIEFAYTASISMGEKCVLHVMNGAVMYQIDSVVRACSDFLVQQLDPSNAIGIANFAE
QIGCVELHQRAREYIYMHFGEVAKQEEFFNLSHCQLVTLISRDDLNVRCESEVFHACINWVKYD
CEQRRFYVQALLRAVRCHSLTPNFLQMQLQKCEILQSDSRCKDYLVKIFEELTLHKPTQVMPCR
APKVGRLIYTAGGYFRQSLSYLEAYNPSDGTWLRLADLQVPRSGLAGCVVGGLLYAVGGRNNSP
DGNTDSSALDCYNPMTNQWSPCAPMSVPRNRIGVGVIDGHIYAVGGSHGCIHHNSVERYEPERD
EWHLVAPMLTRRIGVGVAVLNRLLYAVGGFDGTNRLNSAECYYPERNEWRMITAMNTIRSGAGV
CVLHNCIYAAGGYDGQDQLNSVERYDVETETWTFVAPMKHRRSALGITVHQGRIYVLGGYDGHT
FLDSVECYDPDTDTWSEVTRMTSGRSGVGVAVTMEPCRKQIDQQNCTC
SEQ ID NO: 41 (DCAF15)
>sp|Q66K64|DCA15_HUMAN_DDB1- and CUL4-associated factor 15
OS = Homo sapiens OX = 9606 GN = DCAF15 PE = 1 SV = 1
MAPSSKSERNSGAGSGGGGPGGAGGKRAAGRRREHVLKQLERVKISGQLSPRLFRKLPPRVCVS
LKNIVDEDFLYAGHIFLGFSKCGRYVLSYTSSSGDDDFSFYIYHLYWWEFNVHSKLKLVRQVRL
FQDEEIYSDLYLTVCEWPSDASKVIVFGENTRSANGMLMNMMMMSDENHRDIYVSTVAVPPPGR
CAACQDASRAHPGDPNAQCLRHGFMLHTKYQVVYPFPTFQPAFQLKKDQVVLLNTSYSLVACAV
SVHSAGDRSFCQILYDHSTCPLAPASPPEPQSPELPPALPSFCPEAAPARSSGSPEPSPAIAKA
KEFVADIFRRAKEAKGGVPEEARPALCPGPSGSRCRAHSEPLALCGETAPRDSPPASEAPASEP
GYVNYTKLYYVLESGEGTEPEDELEDDKISLPFVVTDLRGRNLRPMRERTAVQGQYLTVEQLTL
DFEYVINEVIRHDATWGHQFCSFSDYDIVILEVCPETNQVLINIGLLLLAFPSPTEEGQLRPKT
YHTSLKVAWDLNTGIFETVSVGDLTEVKGQTSGSVWSSYRKSCVDMVMKWLVPESSGRYVNRMT
NEALHKGCSLKVLADSERYTWIVL
SEQ ID NO: 42 (RNF4)
>sp|P78317|RNF4_HUMAN E3 ubiquitin-protein ligase RNF4 OS = Homo
sapiens OX = 9606 GN = RNF4 PE = 1 SV = 1
MSTRKRRGGAINSRQAQKRTREATSTPEISLEAEPIELVETAGDEIVDLTCESLEPVVVDLTHN
DSVVIVDERRRPRRNARRLPQDHADSCVVSSDDEELSRDRDVYVTTHTPRNARDEGATGLRPSG
TVSCPICMDGYSEIVQNGRLIVSTECGHVFCSQCLRDSLKNANTCPTCRKKINHKRYHPIYI
SEQ ID NO: 43 (RNF4)
>sp|P78317-2|RNF4_HUMAN Isoform 2 of E3 ubiquitin-protein ligase
RNF4 OS = Homo sapiens OX = 9606 GN = RNF4
MSTRKRRGGAINSRQAQKRTREATSTPEISLEAEPIELVETAGDEIVDLTCESLEPVVVDLTHN
DSVVIVDGPQVLSVVPSAWTDTQRSCRMDVSSFPQNAAMSSVASASVIP
SEQ ID NO: 44 (RNF114)
>sp|Q9Y508|RN114_HUMAN E3 ubiquitin-protein ligase RNF114
OS = Homo sapiens OX = 9606 GN = RNF114 PE = 1 SV = 1
MAAQQRDCGGAAQLAGPAAEADPLGRFTCPVCLEVYEKPVQVPCGHVFCSACLQECLKPKKPVC
GVCRSALAPGVRAVELERQIESTETSCHGCRKNFFLSKIRSHVATCSKYQNYIMEGVKATIKDA
SLQPRNVPNRYTFPCPYCPEKNFDQEGLVEHCKLFHSTDTKSVVCPICASMPWGDPNYRSANFR
EHIQRRHRFSYDTFVDYDVDEEDMMNQVLQRSIIDQ
SEQ ID NO: 45 (RNF114)
>sp|Q9Y508-2|RN114_HUMAN Isoform 2 of E3 ubiquitin-protein
ligase RNF114 OS = Homo sapiens OX = 9606 GN = RNF114
MAAQQRDCGGAAQLAGPAAEADPLGRFTCPVCLEVYEKPVQVPCGHVFCSACLQECLKPKKPVC
GVCRSALAPGVRAVELERQIESTETSCHGCRKNFFLSKIRSHVATCSKYQNYIMEGVKATIKDA
SLQPRNVPNRYTFPCPYCPEKNFDQEGLVEHCKLFHSTDTKSVVSEQSPCLLSVSCYRASITY
SEQ ID NO: 46 (DCAF16)
>sp|Q9NXF7|DCA16_HUMAN DDB1- and CUL4-associated factor 16
OS = Homo sapiens OX = 9606 GN = DCAF16 PE = 1 SV = 1
MGPRNPSPDHLSESESEEEENISYLNESSGEEWDSSEEEDSMVPNLSPLESLAWQVKCLLKYST
TWKPLNPNSWLYHAKLLDPSTPVHILREIGLRLSHCSHCVPKLEPIPEWPPLASCGVPPFQKPL
TSPSRLSRDHATLNGALQFATKQLSRTLSRATPIPEYLKQIPNSCVSGCCCGWLTKTVKETTRT
EPINTTYSYTDFQKAVNKLLTASL
SEQ ID NO: 47 (AHR)
>sp|P35869|AHR_HUMAN Aryl hydrocarbon receptor OS = Homo sapiens
OX = 9606 GN = AHR PE = 1 SV = 2
MNSSSANITYASRKRRKPVQKTVKPIPAEGIKSNPSKRHRDRLNTELDRLASLLPFPQDVINKL
DKLSVLRLSVSYLRAKSFFDVALKSSPTERNGGQDNCRAANFREGLNLQEGEFLLQALNGFVLV
VTTDALVFYASSTIQDYLGFQQSDVIHQSVYELIHTEDRAEFQRQLHWALNPSQCTESGQGIEE
ATGLPQTVVCYNPDQIPPENSPLMERCFICRLRCLLDNSSGFLAMNFQGKLKYLHGQKKKGKDG
SILPPQLALFAIATPLQPPSILEIRTKNFIFRTKHKLDFTPIGCDAKGRIVLGYTEAELCTRGS
GYQFIHAADMLYCAESHIRMIKTGESGMIVFRLLTKNNRWTWVQSNARLLYKNGRPDYIIVTQR
PLTDEEGTEHLRKRNTKLPFMFTTGEAVLYEATNPFPAIMDPLPLRTKNGTSGKDSATTSTLSK
DSLNPSSLLAAMMQQDESIYLYPASSTSSTAPFENNFFNESMNECRNWQDNTAPMGNDTILKHE
QIDQPQDVNSFAGGHPGLFQDSKNSDLYSIMKNLGIDFEDIRHMQNEKFERNDESGEVDERDID
LTDEILTYVQDSLSKSPFIPSDYQQQQSLALNSSCMVQEHLHLEQQQQHHQKQVVVEPQQQLCQ
KMKHMQVNGMFENWNSNQFVPFNCPQQDPQQYNVFTDLHGISQEFPYKSEMDSMPYTQNFISCN
QPVLPQHSKCTELDYPMGSFEPSPYPTTSSLEDFVTCLQLPENQKHGLNPQSAIITPQTCYAGA
VSMYQCQPEPQHTHVGQMQYNPVLPGQQAFLNKFQNGVLNETYPAELNNINNTQTTTHLQPLHH
PSEARPFPDLTSSGFL
SEQ ID NO: 48 (MDM2)
>sp|Q00987|MDM2_HUMAN E3 ubiquitin-protein ligase Mdm2 OS = Homo
sapiens OX = 9606 GN = MDM2 PE = 1 SV = 1
MCNTNMSVPTDGAVTTSQIPASEQETLVRPKPLLLKLLKSVGAQKDTYTMKEVLFYLGQYIMTK
RLYDEKQQHIVYCSNDLLGDLFGVPSFSVKEHRKIYTMIYRNLVVVNQQESSDSGTSVSENRCH
LEGGSDQKDLVQELQEEKPSSSHLVSRPSTSSRRRAISETEENSDELSGERQRKRHKSDSISLS
FDESLALCVIREICCERSSSSESTGTPSNPDLDAGVSEHSGDWLDQDSVSDQFSVEFEVESLDS
EDYSLSEEGQELSDEDDEVYQVTVYQAGESDTDSFEEDPEISLADYWKCTSCNEMNPPLPSHCN
RCWALRENWLPEDKGKDKGEISEKAKLENSTQAEEGFDVPDCKKTIVNDSRESCVEENDDKITQ
ASQSQESEDYSQPSTSSSIIYSSQEDVKEFEREETQDKEESVESSLPLNAIEPCVICQGRPKNG
CIVHGKTGHLMACFTCAKKLKKRNKPCPVCRQPIQMIVLTYFP
SEQ ID NO: 49 (UBR2)
>sp|Q8IWV8|UBR2_HUMAN E3 ubiquitin-protein ligase UBR2 OS = Homo
sapiens OX = 9606 GN = UBR2 PE = 1 SV = 1
MASELEPEVQAIDRSLLECSAEEIAGKWLQATDLTREVYQHLAHYVPKIYCRGPNPFPQKEDML
AQHVLLGPMEWYLCGEDPAFGFPKLEQANKPSHLCGRVFKVGEPTYSCRDCAVDPTCVLCMECF
LGSIHRDHRYRMTTSGGGGFCDCGDTEAWKEGPYCQKHELNTSEIEEEEDPLVHLSEDVIARTY
NIFAITFRYAVEILTWEKESELPADLEMVEKSDTYYCMLFNDEVHTYEQVIYTLQKAVNCTQKE
AIGFATTVDRDGRRSVRYGDFQYCEQAKSVIVRNTSRQTKPLKVQVMHSSIVAHQNFGLKLLSW
LGSIIGYSDGLRRILCQVGLQEGPDGENSSLVDRLMLSDSKLWKGARSVYHQLFMSSLLMDLKY
KKLFAVRFAKNYQQLQRDEMEDDHERAVSVTALSVQFFTAPTLARMLITEENLMSIIIKTEMDH
LRHRDAQGRFQFERYTALQAFKFRRVQSLILDLKYVLISKPTEWSDELRQKFLEGFDAFLELLK
CMQGMDPITRQVGQHIEMEPEWEAAFTLQMKLTHVISMMQDWCASDEKVLIEAYKKCLAVLMQC
HGGYTDGEQPITLSICGHSVETIRYCVSQEKVSIHLPVSRLLAGLHVLLSKSEVAYKFPELLPL
SELSPPMLIEHPLRCLVLCAQVHAGMWRRNGFSLVNQIYYYHNVKCRREMFDKDVVMLQTGVSM
MDPNHFLMIMLSRFELYQIFSTPDYGKRFSSEITHKDVVQQNNTLIEEMLYLIIMLVGERFSPG
VGQVNATDEIKREIIHQLSIKPMAHSELVKSLPEDENKETGMESVIEAVAHFKKPGLTGRGMYE
LKPECAKEFNLYFYHFSRAEQSKAEEAQRKLKRQNREDTALPPPVLPPFCPLFASLVNILQSDV
MLCIMGTILQWAVEHNGYAWSESMLQRVLHLIGMALQEEKQHLENVTEEHVVTFTFTQKISKPG
EAPKNSPSILAMLETLQNAPYLEVHKDMIRWILKTFNAVKKMRESSPTSPVAETEGTIMEESSR
DKDKAERKRKAEIARLRREKIMAQMSEMQRHFIDENKELFQQTLELDASTSAVLDHSPVASDMT
LTALGPAQTQVPEQRQFVTCILCQEEQEVKVESRAMVLAAFVQRSTVLSKNRSKFIQDPEKYDP
LFMHPDLSCGTHTSSCGHIMHAHCWQRYFDSVQAKEQRRQQRLRLHTSYDVENGEFLCPLCECL
SNTVIPLLLPPRNIENNRLNFSDQPNLTQWIRTISQQIKALQFLRKEESTPNNASTKNSENVDE
LQLPEGFRPDERPKIPYSESIKEMLTTFGTATYKVGLKVHPNEEDPRVPIMCWGSCAYTIQSIE
RILSDEDKPLFGPLPCRLDDCLRSLTRFAAAHWTVASVSVVQGHFCKLFASLVPNDSHEELPCI
LDIDMFHLLVGLVLAFPALQCQDESGISLGTGDLHIFHLVTMAHIIQILLTSCTEENGMDQENP
PCEEESAVLALYKTLHQYTGSALKEIPSGWHLWRSVRAGIMPFLKCSALFFHYLNGVPSPPDIQ
VPGTSHFEHLCSYLSLPNNLICLFQENSEIMNSLIESWCRNSEVKRYLEGERDAIRYPRESNKL
INLPEDYSSLINQASNFSCPKSGGDKSRAPTLCLVCGSLLCSQSYCCQTELEGEDVGACTAHTY
SCGSGVGIFLRVRECQVLFLAGKTKGCFYSPPYLDDYGETDQGLRRGNPLHLCKERFKKIQKLW
HQHSVTEEIGHAQEANQTLVGIDWQHL
SEQ ID NO: 50 (SPOP)
>sp|O43791|SPOP_HUMAN Speckle-type POZ protein OS = Homo sapiens
OX = 9606 GN = SPOP PE = 1 SV = 1
MSRVPSPPPPAEMSSGPVAESWCYTQIKVVKFSYMWTINNFSFCREEMGEVIKSSTFSSGANDK
LKWCLRVNPKGLDEESKDYLSLYLLLVSCPKSEVRAKFKESILNAKGEETKAMESQRAYRFVQG
KDWGFKKFIRRDFLLDEANGLLPDDKLTLFCEVSVVQDSVNISGQNTMNMVKVPECRLADELGG
LWENSRFTDCCLCVAGQEFQAHKAILAARSPVFSAMFEHEMEESKKNRVEINDVEPEVEKEMMC
FIYTGKAPNLDKMADDLLAAADKYALERLKVMCEDALCSNLSVENAAEILILADLHSADQLKTQ
AVDFINYHASDVLETSGWKSMVVSHPHLVAEAYRSLASAQCPFLGPPRKRLKQS
SEQ ID NO: 51 (KLHL3)
>sp|Q9UH77|KLHL3_HUMAN Kelch-like protein 3 OS = Homo sapiens
OX = 9606 GN = KLHL3 PE = 1 SV = 2
MEGESVKLSSQTLIQAGDDEKNQRTITVNPAHMGKAFKVMNELRSKQLLCDVMIVAEDVEIEAH
RVVLAACSPYFCAMFTGDMSESKAKKIEIKDVDGQTLSKLIDYIYTAEIEVTEENVQVLLPAAS
LLQLMDVRQNCCDFLQSQLHPTNCLGIRAFADVHTCTDLLQQANAYAEQHFPEVMLGEEFLSLS
LDQVCSLISSDKLTVSSEEKVFEAVISWINYEKETRLEHMAKLMEHVRLPLLPRDYLVQTVEEE
ALIKNNNTCKDFLIEAMKYHLLPLDQRLLIKNPRTKPRTPVSLPKVMIVVGGQAPKAIRSVECY
DFEEDRWDQIAELPSRRCRAGVVFMAGHVYAVGGFNGSLRVRTVDVYDGVKDQWTSIASMQERR
STLGAAVLNDLLYAVGGFDGSTGLASVEAYSYKTNEWFFVAPMNTRRSSVGVGVVEGKLYAVGG
YDGASRQCLSTVEQYNPATNEWIYVADMSTRRSGAGVGVLSGQLYATGGHDGPLVRKSVEVYDP
GTNTWKQVADMNMCRRNAGVCAVNGLLYVVGGDDGSCNLASVEYYNPVTDKWTLLPTNMSTGRS
YAGVAVIHKSL
SEQ ID NO: 52 (KLHL12)
>sp|Q53G59|KLH12_HUMAN Kelch-like protein 12 OS = Homo sapiens
OX = 9606 GN = KLHL12 PE = 1 SV = 2
MGGIMAPKDIMTNTHAKSILNSMNSLRKSNTLCDVTLRVEQKDFPAHRIVLAACSDYFCAMFTS
ELSEKGKPYVDIQGLTASTMEILLDFVYTETVHVTVENVQELLPAACLLQLKGVKQACCEFLES
QLDPSNCLGIRDFAETHNCVDLMQAAEVFSQKHFPEVVQHEEFILLSQGEVEKLIKCDEIQVDS
EEPVFEAVINWVKHAKKEREESLPNLLQYVRMPLLTPRYITDVIDAEPFIRCSLQCRDLVDEAK
KFHLRPELRSQMQGPRTRARLGANEVLLVVGGFGSQQSPIDVVEKYDPKTQEWSFLPSITRKRR
YVASVSLHDRIYVIGGYDGRSRLSSVECLDYTADEDGVWYSVAPMNVRRGLAGATTLGDMIYVS
GGFDGSRRHTSMERYDPNIDQWSMLGDMQTAREGAGLVVASGVIYCLGGYDGLNILNSVEKYDP
HTGHWTNVTPMATKRSGAGVALLNDHIYVVGGFDGTAHLSSVEAYNIRTDSWTTVTSMTTPRCY
VGATVLRGRLYAIAGYDGNSLLSSIECYDPIIDSWEVVTSMGTQRCDAGVCVLREK
SEQ ID NO: 53 (KLHL20)
>sp|Q9Y2M5|KLH20_HUMAN Kelch-like protein 20 OS = Homo sapiens
OX = 9606 GN = KLHL20 PE = 1 SV = 4
MEGKPMRRCTNIRPGETGMDVTSRCTLGDPNKLPEGVPQPARMPYISDKHPRQTLEVINLLRKH
RELCDVVLVVGAKKIYAHRVILSACSPYFRAMFTGELAESRQTEVVIRDIDERAMELLIDFAYT
SQITVEEGNVQTLLPAACLLQLAEIQEACCEFLKRQLDPSNCLGIRAFADTHSCRELLRIADKF
TQHNFQEVMESEEFMLLPANQLIDIISSDELNVRSEEQVFNAVMAWVKYSIQERRPQLPQVLQH
VRLPLLSPKFLVGTVGSDPLIKSDEECRDLVDEAKNYLLLPQERPLMQGPRTRPRKPIRCGEVL
FAVGGWCSGDAISSVERYDPQTNEWRMVASMSKRRCGVGVSVLDDLLYAVGGHDGSSYLNSVER
YDPKTNQWSSDVAPTSTCRTSVGVAVLGGFLYAVGGQDGVSCLNIVERYDPKENKWTRVASMST
RRLGVAVAVLGGFLYAVGGSDGTSPLNTVERYNPQENRWHTIAPMGTRRKHLGCAVYQDMIYAV
GGRDDTTELSSAERYNPRTNQWSPVVAMTSRRSGVGLAVVNGQLMAVGGFDGTTYLKTIEVEDP
DANTWRLYGGMNYRRLGGGVGVIKMTHCESHIW
SEQ ID NO: 54 (KLHDC2)
>sp|Q9Y2U9|KLDC2_HUMAN Kelch domain-containing protein 2 OS = Homo
sapiens OX = 9606 GN = KLHDC2 PE = 1 SV = 1
MADGNEDLRADDLPGPAFESYESMELACPAERSGHVAVSDGRHMFVWGGYKSNQVRGLYDFYLP
REELWIYNMETGRWKKINTEGDVPPSMSGSCAVCVDRVLYLFGGHHSRGNTNKFYMLDSRSTDR
VLQWERIDCQGIPPSSKDKLGVWVYKNKLIFFGGYGYLPEDKVLGTFEFDETSFWNSSHPRGWN
DHVHILDTETFTWSQPITTGKAPSPRAAHACATVGNRGFVFGGRYRDARMNDLHYLNLDTWEWN
ELIPQGICPVGRSWHSLTPVSSDHLFLFGGFTTDKQPLSDAWTYCISKNEWIQFNHPYTEKPRL
WHTACASDEGEVIVEGGCANNLLVHHRAAHSNEILIFSVQPKSLVRLSLEAVICFKEMLANSWN
CLPKHLLHSVNQRFGSNNTSGS
SEQ ID NO: 55 (SPSB1)
>sp|Q96BD6|SPSB1_HUMAN SPRY domain-containing SOCS box protein 1
OS = Homo sapiens OX = 9606 GN = SPSB1 PE = 1 SV = 1
MGQKVTGGIKTVDMRDPTYRPLKQELQGLDYCKPTRLDLLLDMPPVSYDVQLLHSWNNNDRSLN
VFVKEDDKLIFHRHPVAQSTDAIRGKVGYTRGLHVWQITWAMRQRGTHAVVGVATADAPLHSVG
YTTLVGNNHESWGWDLGRNRLYHDGKNQPSKTYPAFLEPDETFIVPDSFLVALDMDDGTLSFIV
DGQYMGVAFRGLKGKKLYPVVSAVWGHCEIRMRYLNGLDPEPLPLMDLCRRSVRLALGRERLGE
IHTLPLPASLKAYLLYQ
SEQ ID NO: 56 (SPSB2)
>sp|Q99619|SPSB2_HUMAN SPRY domain-containing SOCS box protein 2
OS = Homo sapiens OX = 9606 GN = SPSB2 PE = 1 SV = 1
MGQTALAGGSSSTPTPQALYPDLSCPEGLEELLSAPPPDLGAQRRHGWNPKDCSENIEVKEGGL
YFERRPVAQSTDGARGKRGYSRGLHAWEISWPLEQRGTHAVVGVATALAPLQTDHYAALLGSNS
ESWGWDIGRGKLYHQSKGPGAPQYPAGTQGEQLEVPERLLVVLDMEEGTLGYAIGGTYLGPAFR
GLKGRTLYPAVSAVWGQCQVRIRYLGERRAEPHSLLHLSRLCVRHNLGDTRLGQVSALPLPPAM
KRYLLYQ
SEQ ID NO: 57 (SPSB4)
>sp|Q96A44|SPSB4_HUMAN SPRY domain-containing SOCS box protein 4
OS = Homo sapiens OX = 9606 GN = SPSB4 PE = 1 SV = 1
MGQKLSGSLKSVEVREPALRPAKRELRGAEPGRPARLDQLLDMPAAGLAVQLRHAWNPEDRSLN
VFVKDDDRLTFHRHPVAQSTDGIRGKVGHARGLHAWQINWPARQRGTHAVVGVATARAPLHSVG
YTALVGSDAESWGWDLGRSRLYHDGKNQPGVAYPAFLGPDEAFALPDSLLVVLDMDEGTLSFIV
DGQYLGVAFRGLKGKKLYPVVSAVWGHCEVTMRYINGLDPEPLPLMDLCRRSIRSALGRQRLQD
ISSLPLPQSLKNYLQYQ
SEQ ID NO: 58 (SOCS2)
>sp|O14508|SOCS2_HUMAN Suppressor of cytokine signaling 2
OS = Homo sapiens OX = 9606 GN = SOCS2 PE = 1 SV = 1
MTLRCLEPSGNGGEGTRSQWGTAGSAEEPSPQAARLAKALRELGQTGWYWGSMTVNEAKEKLKE
APEGTFLIRDSSHSDYLLTISVKTSAGPTNLRIEYQDGKFRLDSIICVKSKLKQFDSVVHLIDY
YVQMCKDKRTGPEAPRNGTVHLYLTKPLYTSAPSLQHLCRLTINKCTGAIWGLPLPTRLKDYLE
EYKFQV
SEQ ID NO: 59 (SOCS6)
>sp|O14544|SOCS6_HUMAN Suppressor of cytokine signaling 6
OS = Homo sapiens OX = 9606 GN = SOCS6 PE = 1 SV = 2
MKKISLKTLRKSFNLNKSKEETDFMVVQQPSLASDFGKDDSLFGSCYGKDMASCDINGEDEKGG
KNRSKSESLMGTLKRRLSAKQKSKGKAGTPSGSSADEDTESSSSAPIVEKDVRAQRPIRSTSLR
SHHYSPAPWPLRPTNSEETCIKMEVRVKALVHSSSPSPALNGVRKDFHDLQSETTCQEQANSLK
SSASHNGDLHLHLDEHVPVVIGLMPQDYIQYTVPLDEGMYPLEGSRSYCLDSSSPMEVSAVPPQ
VGGRAFPEDESQVDQDLVVAPEIFVDQSVNGLLIGTTGVMLQSPRAGHDDVPPLSPLLPPMQNN
QIQRNFSGLIGTEAHVAESMRCHLNFDPNSAPGVARVYDSVQSSGPMVVTSLTEELKKLAKQGW
YWGPITRWEAEGKLANVPDGSFLVRDSSDDRYLLSLSFRSHGKTLHTRIEHSNGRESFYEQPDV
EGHTSIVDLIEHSIRDSENGAFCYSRSRLPGSATYPVRLTNPVSRFMQVRSLQYLCRFVIRQYT
RIDLIQKLPLPNKMKDYLQEKHY
SEQ ID NO: 60 (FBXO4)
>sp|Q9UKT5|FBX4_HUMAN F-box only protein 4 OS = Homo sapiens
OX = 9606 GN = FBXO4 PE = 1 SV = 2
MAGSEPRSGTNSPPPPESDWGRLEAAILSGWKTFWQSVSKERVARTTSREEVDEAASTLTRLPI
DVQLYILSFLSPHDLCQLGSTNHYWNETVRDPILWRYFLLRDLPSWSSVDWKSLPDLEILKKPI
SEVTDGAFFDYMAVYRMCCPYTRRASKSSRPMYGAVTSFLHSLIIQNEPRFAMFGPGLEELNTS
LVLSLMSSEELCPTAGLPQRQIDGIGSGVNFQLNNQHKFNILILYSTTRKERDRAREEHTSAVN
KMFSRHNEGDDQQGSRYSVIPQIQKVCEVVDGFIYVANAEAHKRHEWQDEFSHIMAMTDPAFGS
SGRPLLVLSCISQGDVKRMPCFYLAHELHLNLLNHPWLVQDTEAETLTGELNGIEWILEEVESK
RAR
SEQ ID NO: 61 (FBXO31)
>sp|Q5XUX0|FBX3_HUMAN F-box only protein 31 OS = Homo sapiens
OX = 9606 GN = FBX031 PE = 1 SV = 2
MAVCARLCGVGPSRGCRRRQQRRGPAETAAADSEPDTDPEEERIEASAGVGGGLCAGPSPPPPR
CSLLELPPELLVEIFASLPGTDLPSLAQVCTKFRRILHTDTIWRRRCREEYGVCENLRKLEITG
VSCRDVYAKLLHRYRHILGLWQPDIGPYGGLLNVVVDGLFIIGWMYLPPHDPHVDDPMRFKPLF
RIHLMERKAATVECMYGHKGPHHGHIQIVKKDEFSTKCNQTDHHRMSGGRQEEFRTWLREEWGR
TLEDIFHEHMQELILMKFIYTSQYDNCLTYRRIYLPPSRPDDLIKPGLFKGTYGSHGLEIVMLS
FHGRRARGTKITGDPNIPAGQQTVEIDLRHRIQLPDLENQRNFNELSRIVLEVRERVRQEQQEG
GHEAGEGRGRQGPRESQPSPAQPRAEAPSKGPDGTPGEDGGEPGDAVAAAEQPAQCGQGQPFVL
PVGVSSRNEDYPRTCRMCFYGTGLIAGHGFTSPERTPGVFILFDEDRFGFVWLELKSFSLYSRV
QATFRNADAPSPQAFDEMLKNIQSLTS
SEQ ID NO: 62 (BTRC)
>sp|Q9Y297|FBW1A_HUMAN F-box/WD repeat-containing protein 1A
OS = Homo sapiens OX = 9606 GN = BTRC PE = 1 SV = 1
MDPAEAVLQEKALKEMCSMPRSLWLGCSSLADSMPSLRCLYNPGTGALTAFQNSSEREDCNNGE
PPRKIIPEKNSLRQTYNSCARLCLNQETVCLASTAMKTENCVAKTKLANGTSSMIVPKQRKLSA
SYEKEKELCVKYFEQWSESDQVEFVEHLISQMCHYQHGHINSYLKPMLQRDFITALPARGLDHI
AENILSYLDAKSLCAAELVCKEWYRVTSDGMLWKKLIERMVRTDSLWRGLAERRGWGQYLFKNK
PPDGNAPPNSFYRALYPKIIQDIETIESNWRCGRHSLQRIHCRSETSKGVYCLQYDDQKIVSGL
RDNTIKIWDKNTLECKRILTGHTGSVLCLQYDERVIITGSSDSTVRVWDVNTGEMLNTLIHHCE
AVLHLRFNNGMMVTCSKDRSIAVWDMASPTDITLRRVLVGHRAAVNVVDEDDKYIVSASGDRTI
KVWNTSTCEFVRTLNGHKRGIACLQYRDRLVVSGSSDNTIRLWDIECGACLRVLEGHEELVRCI
RFDNKRIVSGAYDGKIKVWDLVAALDPRAPAGTLCLRTLVEHSGRVERLQFDEFQIVSSSHDDT
ILIWDFLNDPAAQAEPPRSPSRTYTYISR
SEQ ID NO: 63 (FBW7)
>sp|Q969H0|FBXW7_HUMAN F-box/WD repeat-containing protein 7
OS = Homo sapiens OX = 9606 GN = FBXW7 PE = 1 SV = 1
MNQELLSVGSKRRRTGGSLRGNPSSSQVDEEQMNRVVEEEQQQQLRQQEEEHTARNGEVVGVEP
RPGGQNDSQQGQLEENNNRFISVDEDSSGNQEEQEEDEEHAGEQDEEDEEEEEMDQESDDEDQS
DDSSREDEHTHTNSVINSSSIVDLPVHQLSSPFYTKTTKMKRKLDHGSEVRSFSLGKKPCKVSE
YTSTTGLVPCSATPTTFGDLRAANGQGQQRRRITSVQPPTGLQEWLKMFQSWSGPEKLLALDEL
IDSCEPTQVKHMMQVIEPQFQRDFISLLPKELALYVLSFLEPKDLLQAAQTCRYWRILAEDNLL
WREKCKEEGIDEPLHIKRRKVIKPGFIHSPWKSAYIRQHRIDTNWRRGELKSPKVLKGHDDHVI
TCLQFCGNRIVSGSDDNTLKVWSAVTGKCLRTLVGHTGGVWSSQMRDNIIISGSTDRTLKVWNA
ETGECIHTLYGHTSTVRCMHLHEKRVVSGSRDATLRVWDIETGQCLHVLMGHVAAVRCVQYDGR
RVVSGAYDFMVKVWDPETETCLHTLQGHTNRVYSLQFDGIHVVSGSLDTSIRVWDVETGNCIHT
LTGHQSLTSGMELKDNILVSGNADSTVKIWDIKTGQCLQTLQGPNKHQSAVTCLQFNKNFVITS
SDDGTVKLWDLKTGEFIRNLVTLESGGSGGVVWRIRASNTKLVCAVGSRNGTEETKLLVLDEDV
DMK
SEQ ID NO: 64 (CDC20)
>sp|Q12834|CDC20_HUMAN Cell division cycle protein 20 homolog
OS = Homo sapiens OX = 9606 GN = CDC20 PE = 1 SV = 2
MAQFAFESDLHSLLQLDAPIPNAPPARWQRKAKEAAGPAPSPMRAANRSHSAGRTPGRTPGKSS
SKVQTTPSKPGGDRYIPHRSAAQMEVASFLLSKENQPENSQTPTKKEHQKAWALNLNGFDVEEA
KILRLSGKPQNAPEGYQNRLKVLYSQKATPGSSRKTCRYIPSLPDRILDAPEIRNDYYLNLVDW
SSGNVLAVALDNSVYLWSASSGDILQLLQMEQPGEYISSVAWIKEGNYLAVGTSSAEVQLWDVQ
QQKRLRNMTSHSARVGSLSWNSYILSSGSRSGHIHHHDVRVAEHHVATLSGHSQEVCGLRWAPD
GRHLASGGNDNLVNVWPSAPGEGGWVPLQTFTQHQGAVKAVAWCPWQSNVLATGGGTSDRHIRI
WNVCSGACLSAVDAHSQVCSILWSPHYKELISGHGFAQNQLVIWKYPTMAKVAELKGHTSRVLS
LTMSPDGATVASAAADETLRLWRCFELDPARRREREKASAAKSSLIHQGIR
SEQ ID NO: 65 (ITCH)
>sp|Q96J02|ITCH_HUMAN E3 ubiquitin-protein ligase Itchy homolog
OS = Homo sapiens OX = 9606 GN = ITCH PE = 1 SV = 2
MSDSGSQLGSMGSLTMKSQLQITVISAKLKENKKNWFGPSPYVEVTVDGQSKKTEKCNNTNSPK
WKQPLTVIVTPVSKLHFRVWSHQTLKSDVLLGTAALDIYETLKSNNMKLEEVVVTLQLGGDKEP
TETIGDLSICLDGLQLESEVVINGETTCSENGVSLCLPRLECNSAISAHCNLCLPGLSDSPISA
SRVAGFTGASQNDDGSRSKDETRVSINGSDDPEDAGAGENRRVSGNNSPSLSNGGFKPSRPPRP
SRPPPPTPRRPASVNGSPSATSESDGSSTGSLPPTNTNTNTSEGATSGLIIPLTISGGSGPRPL
NPVTQAPLPPGWEQRVDQHGRVYYVDHVEKRTTWDRPEPLPPGWERRVDNMGRIYYVDHFTRTT
TWQRPTLESVRNYEQWQLQRSQLQGAMQQFNQRFIYGNQDLFATSQSKEFDPLGPLPPGWEKRT
DSNGRVYFVNHNTRITQWEDPRSQGQLNEKPLPEGWEMRFTVDGIPYFVDHNRRTTTYIDPRTG
KSALDNGPQIAYVRDFKAKVQYFRFWCQQLAMPQHIKITVTRKTLFEDSFQQIMSFSPQDLRRR
LWVIFPGEEGLDYGGVAREWFFLLSHEVLNPMYCLFEYAGKDNYCLQINPASYINPDHLKYFRE
IGRFIAMALFHGKFIDTGFSLPFYKRILNKPVGLKDLESIDPEFYNSLIWVKENNIEECDLEMY
FSVDKEILGEIKSHDLKPNGGNILVTEENKEEYIRMVAEWRLSRGVEEQTQAFFEGFNEILPQQ
YLQYFDAKELEVLLCGMQEIDLNDWQRHAIYRHYARTSKQIMWFWQFVKEIDNEKRMRLLQFVT
GTCRLPVGGFADLMGSNGPQKFCIEKVGKENWLPRSHTCFNRLDLPPYKSYEQLKEKLLFAIEE
TEGFGQE
SEQ ID NO: 66 (PML)
>sp|P29590|PML_HUMAN Protein PML OS = Homo sapiens OX = 9606 GN = PML
PE = 1 SV = 3
MEPAPARSPRPQQDPARPQEPTMPPPETPSEGRQPSPSPSPTERAPASEEEFQFLRCQQCQAEA
KCPKLLPCLHTLCSGCLEASGMQCPICQAPWPLGADTPALDNVFFESLQRRLSVYRQIVDAQAV
CTRCKESADFWCFECEQLLCAKCFEAHQWELKHEARPLAELRNQSVREFLDGTRKTNNIFCSNP
NHRTPTLTSIYCRGCSKPLCCSCALLDSSHSELKCDISAEIQQRQEELDAMTQALQEQDSAFGA
VHAQMHAAVGQLGRARAETEELIRERVRQVVAHVRAQERELLEAVDARYQRDYEEMASRLGRLD
AVLQRIRTGSALVQRMKCYASDQEVLDMHGFLRQALCRLRQEEPQSLQAAVRIDGEDEFKVRLQ
DLSSCITQGKDAAVSKKASPEAASTPRDPIDVDLPEEAERVKAQVQALGLAEAQPMAVVQSVPG
AHPVPVYAFSIKGPSYGEDVSNTTTAQKRKCSQTQCPRKVIKMESEEGKEARLARSSPEQPRPS
TSKAVSPPHLDGPPSPRSPVIGSEVFLPNSNHVASGAGEAEERVVVISSSEDSDAENSSSRELD
DSSSESSDLQLEGPSTLRVLDENLADPQAEDRPLVFFDLKIDNETQKISQLAAVNRESKERVVI
QPEAFFSIYSKAVSLEVGLQHFLSFLSSMRRPILACYKLWGPGLPNFFRALEDINRLWEFQEAI
SGFLAALPLIRERVPGASSFKLKNLAQTYLARNMSERSAMAAVLAMRDLCRLLEVSPGPQLAQH
VYPFSSLQCFASLQPLVQAAVLPRAEARLLALHNVSFMELLSAHRRDRQGGLKKYSRYLSLQTT
TLPPAQPAFNLQALGTYFEGLLEGPALARAEGVSTPLAGRGLAERASQQS
SEQ ID NO: 67 (TRIM21)
>sp|P19474|RO52_HUMAN E3 ubiquitin-protein ligase TRIM21 OS = Homo
sapiens OX = 9606 GN = TRIM21 PE = 1 SV = 1
MASAARLTMMWEEVTCPICLDPFVEPVSIECGHSFCQECISQVGKGGGSVCPVCRQRFLLKNLR
PNRQLANMVNNLKEISQEAREGTQGERCAVHGERLHLFCEKDGKALCWVCAQSRKHRDHAMVPL
EEAAQEYQEKLQVALGELRRKQELAEKLEVEIAIKRADWKKTVETQKSRIHAEFVQQKNELVEE
EQRQLQELEKDEREQLRILGEKEAKLAQQSQALQELISELDRRCHSSALELLQEVIIVLERSES
WNLKDLDITSPELRSVCHVPGLKKMLRTCAVHITLDPDTANPWLILSEDRRQVRLGDTQQSIPG
NEERFDSYPMVLGAQHFHSGKHYWEVDVTGKEAWDLGVCRDSVRRKGHFLLSSKSGFWTIWLWN
KQKYEAGTYPQTPLHLQVPPCQVGIFLDYEAGMVSFYNITDHGSLIYSFSECAFTGPLRPFFSP
GFNDGGKNTAPLTLCPLNIGSQGSTDY
SEQ ID NO: 68 (TRIM24)
>sp|O15164|TIF1A_HUMAN Transcription intermediary factor 1-alpha
OS = Homo sapiens OX = 9606 GN = TRIM24 PE = 1 SV = 3
MEVAVEKAVAAAAAASAAASGGPSAAPSGENEAESRQGPDSERGGEAARLNLLDTCAVCHQNIQ
SRAPKLLPCLHSFCQRCLPAPQRYLMLPAPMLGSAETPPPVPAPGSPVSGSSPFATQVGVIRCP
VCSQECAERHIIDNFFVKDTTEVPSSTVEKSNQVCTSCEDNAEANGFCVECVEWLCKTCIRAHQ
RVKFTKDHTVRQKEEVSPEAVGVTSQRPVFCPFHKKEQLKLYCETCDKLTCRDCQLLEHKEHRY
QFIEEAFQNQKVIIDTLITKLMEKTKYIKFTGNQIQNRIIEVNQNQKQVEQDIKVAIFTLMVEI
NKKGKALLHQLESLAKDHRMKLMQQQQEVAGLSKQLEHVMHFSKWAVSSGSSTALLYSKRLITY
RLRHLLRARCDASPVTNNTIQFHCDPSFWAQNIINLGSLVIEDKESQPQMPKQNPVVEQNSQPP
SGLSSNQLSKFPTQISLAQLRLQHMQQQVMAQRQQVQRRPAPVGLPNPRMQGPIQQPSISHQQP
PPRLINFQNHSPKPNGPVLPPHPQQLRYPPNQNIPRQAIKPNPLQMAFLAQQAIKQWQISSGQG
TPSTTNSTSSTPSSPTITSAAGYDGKAFGSPMIDLSSPVGGSYNLPSLPDIDCSSTIMLDNIVR
KDTNIDHGQPRPPSNRTVQSPNSSVPSPGLAGPVTMTSVHPPIRSPSASSVGSRGSSGSSSKPA
GADSTHKVPVVMLEPIRIKQENSGPPENYDEPVVIVKQESDEESRPQNANYPRSILTSLLLNSS
QSSTSEETVLRSDAPDSTGDQPGLHQDNSSNGKSEWLDPSQKSPLHVGETRKEDDPNEDWCAVC
QNGGELLCCEKCPKVFHLSCHVPTLTNFPSGEWICTFCRDLSKPEVEYDCDAPSHNSEKKKTEG
LVKLTPIDKRKCERLLLFLYCHEMSLAFQDPVPLTVPDYYKIIKNPMDLSTIKKRLQEDYSMYS
KPEDFVADERLIFQNCAEFNEPDSEVANAGIKLENYFEELLKNLYPEKRFPKPEFRNESEDNKF
SDDSDDDFVQPRKKRLKSIEERQLLK
SEQ ID NO: 69 (TRIM33)
>sp|Q9UPN9|TRI33_HUMAN E3 ubiquitin-protein ligase TRIM33
OS = Homo sapiens OX = 9606 GN = TRIM33 PE = 1 SV = 3
MAENKGGGEAESGGGGSGSAPVTAGAAGPAAQEAEPPLTAVLVEEEEEEGGRAGAEGGAAGPDD
GGVAAASSGSAQAASSPAASVGTGVAGGAVSTPAPAPASAPAPGPSAGPPPGPPASLLDTCAVC
QQSLQSRREAEPKLLPCLHSFCLRCLPEPERQLSVPIPGGSNGDIQQVGVIRCPVCRQECRQID
LVDNYFVKDTSEAPSSSDEKSEQVCTSCEDNASAVGFCVECGEWLCKTCIEAHQRVKFTKDHLI
RKKEDVSESVGASGQRPVFCPVHKQEQLKLFCETCDRLTCRDCQLLEHKEHRYQFLEEAFQNQK
GAIENLLAKLLEKKNYVHFAATQVQNRIKEVNETNKRVEQEIKVAIFTLINEINKKGKSLLQQL
ENVTKERQMKLLQQQNDITGLSRQVKHVMNFTNWAIASGSSTALLYSKRLITFQLRHILKARCD
PVPAANGAIRFHCDPTFWAKNVVNLGNLVIESKPAPGYTPNVVVGQVPPGTNHISKTPGQINLA
QLRLQHMQQQVYAQKHQQLQQMRMQQPPAPVPTTTTTTQQHPRQAAPQMLQQQPPRLISVQTMQ
RGNMNCGAFQAHQMRLAQNAARIPGIPRHSGPQYSMMQPHLQRQHSNPGHAGPFPVVSVHNTTI
NPTSPTTATMANANRGPTSPSVTAIELIPSVINPENLPSLPDIPPIQLEDAGSSSLDNLLSRYI
SGSHLPPQPTSTMNPSPGPSALSPGSSGLSNSHTPVRPPSTSSTGSRGSCGSSGRTAEKTSLSF
KSDQVKVKQEPGTEDEICSFSGGVKQEKTEDGRRSACMLSSPESSLTPPLSTNLHLESELDALA
SLENHVKIEPADMNESCKQSGLSSLVNGKSPIRSLMHRSARIGGDGNNKDDDPNEDWCAVCQNG
GDLLCCEKCPKVFHLTCHVPTLLSFPSGDWICTFCRDIGKPEVEYDCDNLQHSKKGKTAQGLSP
VDQRKCERLLLYLYCHELSIEFQEPVPASIPNYYKIIKKPMDLSTVKKKLQKKHSQHYQIPDDF
VADVRLIFKNCERFNEMMKVVQVYADTQEINLKADSEVAQAGKAVALYFEDKLTEIYSDRTFAP
LPEFEQEEDDGEVTEDSDEDFIQPRRKRLKSDERPVHIK
SEQ ID NO: 70 (GID4)
>sp|Q8IVV7|GID4 HUMAN Glucose-induced degradation protein 4
homolog OS = Homo sapiens OX = 9606 GN = GID4 PE = 1 SV = 1
MCARGQVGRGTQLRTGRPCSQVPGSRWRPERLLRRQRAGGRPSRPHPARARPGLSLPATLLGSR
AAAAVPLPLPPALAPGDPAMPVRTECPPPAGASAASAASLIPPPPINTQQPGVATSLLYSGSKE
RGHQKSKGNSYDVEVVLQHVDTGNSYLCGYLKIKGLTEEYPTLTTFFEGEIISKKHPFLTRKWD
ADEDVDRKHWGKFLAFYQYAKSFNSDDFDYEELKNGDYVEMRWKEQFLVPDHTIKDISGASFAG
FYYICFQKSAASIEGYYYHRSSEWYQSLNLTHVPEHSAPIYEFR
SEQ ID NO: 71 (DCAF11)
>sp|Q8TEB1|DCA11_HUMAN DDB1- and CUL4-associated factor 11
OS = Homo sapiens OX = 9606 GN = DCAF11 PE = 1 SV = 1
MGSRNSSSAGSGSGDPSEGLPRRGAGLRRSEEEEEEDEDVDLAQVLAYLLRRGQVRLVQGGGAA
NLQFIQALLDSEEENDRAWDGRLGDRYNPPVDATPDTRELEFNEIKTQVELATGQLGLRRAAQK
HSFPRMLHQRERGLCHRGSFSLGEQSRVISHFLPNDLGFTDSYSQKAFCGIYSKDGQIFMSACQ
DQTIRLYDCRYGRFRKFKSIKARDVGWSVLDVAFTPDGNHFLYSSWSDYIHICNIYGEGDTHTA
LDLRPDERRFAVESIAVSSDGREVLGGANDGCLYVFDREQNRRTLQIESHEDDVNAVAFADISS
QILFSGGDDAICKVWDRRTMREDDPKPVGALAGHQDGITFIDSKGDARYLISNSKDQTIKLWDI
RRFSSREGMEASRQAATQQNWDYRWQQVPKKAWRKLKLPGDSSLMTYRGHGVLHTLIRCRESPI
HSTGQQFIYSGCSTGKVVVYDLLSGHIVKKLTNHKACVRDVSWHPFEEKIVSSSWDGNLRLWQY
RQAEYFQDDMPESEECASAPAPVPQSSTPESSPQ

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A system for detecting modulator-dependent proximity-based interactions between an E3 ligase and a target protein, the system comprising:
a) cell(s) expressing one or more fusion proteins, each fusion protein comprising an E3 ligase substrate receptor and a proximity labeling enzyme; and
b) an E3 ligase binding modulator.

2. The system of claim 1, further comprising:
c) second cell(s) expressing one or more fusion protein, each fusion protein comprising a mutant of the E3 ligase substrate receptor that is unable to bind the modulator at a canonical binding site.

3. (canceled)

4. A method for detecting modulator-dependent interaction(s) between an E3 ligase and one or more target(s), validating a predicted modulator-dependent interaction between an E3 ligase and target(s), or identifying E3 ligase(s) that interact with target(s) in a modulator-dependent manner or not the method comprising:
I)
a) providing i) first cell(s) expressing a fusion protein comprising an E3 ligase substrate receptor and a proximity labeling enzyme; and ii) an E3 ligase binding modulator;
b) incubating the first cell(s) and modulator under conditions effective for the proximity labeling enzyme to label protein(s) in the proximity of the fusion protein; and
c) detecting the presence and/or amount of labeled protein(s);
II) (1)
a) providing i) second cell(s) expressing the fusion protein; and ii) a negative control for the modulator;
b) incubating the second cell(s) and negative control under conditions effective for the proximity labeling enzyme to label protein(s) in the proximity of the fusion protein; and
c) detecting the presence and/or amount of labeled protein(s); and
d) optionally providing i) third cell(s) expressing a second fusion protein comprising a proximity labeling enzyme and an E3 ligase substrate receptor that is unable to bind the modulator at a canonical binding site; and ii) the modulator, and incubating the third cell(s) and modulator under conditions effective for the proximity labeling enzyme to label protein(s) in the proximity of the fusion protein; or
(2)
a) providing i) second cell(s) expressing a fusion comprising a proximity labeling enzyme and an E3 ligase substrate receptor that is unable to bind the modulator at a canonical binding site; and ii) a modulator;
b) incubating the second cell(s) and modulator under conditions effective for the proximity labeling enzyme to label protein(s) in the proximity of the fusion protein; and
c) detecting the presence and/or amount of labeled protein(s);
III) comparing the presence and/or amount of the protein(s) detected in step I to the presence and/or amount of those detected in step II; and
IV)
(1) determining, based on the comparing in step III, whether the protein(s) are target(s) that interact with the E3 ligase in a modulator-dependent manner;
2) validating the predicted modulator-dependent interaction between the E3 ligase and target(s) or not based on the comparing of step III; or
3) identifying E3 ligase(s) that interact with target(s) in a modulator-dependent manner not based on the comparing of step III.

5.-12. (canceled)

13. A method for identifying non-canonical E3 ligase substrate receptor binding sites, the method comprising:
I)
a) providing i) first cell(s) expressing the target(s) and a fusion protein comprising an E3 ligase substrate receptor and a proximity labeling enzyme; and ii) an E3 ligase binding modulator, wherein the E3 ligase substrate receptor is unable to bind the modulator at a canonical binding site;
b) incubating the first cell(s) and modulator under conditions effective for the proximity labeling enzyme to label protein(s) in the proximity of the fusion protein(s); and
c) detecting the presence and/or amount of labeled protein(s);
II)
a) providing i) second cell(s) expressing the target(s) and a fusion protein comprising a proximity labeling enzyme and an E3 ligase substrate receptor that is unable to bind the modulator at a canonical binding site; and ii) a negative control for the modulator;
b) incubating the second cell(s) and negative control under conditions effective for the proximity labeling enzyme to label protein(s) in the proximity of the fusion protein(s); and
c) detecting the presence and/or amount of labeled protein(s);
III) comparing the presence and/or amount of labeled target(s), from step I to those in step II; and
IV) identifying non-canonical E3 binding sites that interact with a modulator and/or target based on the comparing of step III.

14. The method of claim 4, wherein the negative control for the modulator is DMSO.

15. The method of claim 4, wherein conditions effective for the proximity labeling enzyme to label protein(s) in the proximity of the fusion protein comprise incubating in a composition comprising a substrate for the proximity labeling enzyme.

16. The method of claim 15, wherein the substrate for the proximity labeling enzyme is biotin.

17. The method of claim 4, wherein incubation is carried out in the presence of a 26S proteasome inhibitor.

18. The method of claim 17, wherein the 26S proteasome inhibitor is selected from the group consisting of bortezomib, ixazomib, carfilzomib, MG-132, MG-115, oprozomib, marizomib, MLN9708, and combinations thereof.

19. The method of claim 4, wherein detecting the presence and/or amount of labeled protein(s) comprises quantitative mass spectrometry and/or Western Blot analysis.

20. The method of claim 4, wherein the target is identified as having a modulator-dependent interaction with an E3 ligase, or vice-versa, when the amount of the target protein that is labeled after incubation with the modulator is greater than the amount of the target protein that is labeled after incubation under the same conditions with a negative control for the modulator, and/or wherein the target is identified as having a modulator-dependent interaction with an E3 ligase when the amount of the target protein that is labeled after incubation with a modulator is greater than the amount of the target protein that is labeled after incubation under the same conditions except where the E3 ligase is a mutant that is unable to bind the modulator at a canonical binding site.

21. (canceled)

22. The method of claim 20, wherein the log2 fold change of the target protein when incubated with the modulator versus the control or mutant is at least 0.5, at least 1, at least 1.5, at least 2, or at least 3.

23. The method of claim 4, wherein the E3 ligase substrate receptor is selected from the group consisting of CRBN (SEQ ID NO: 4), VHL (SEQ ID NO: 31), BIRC1 (SEQ ID NO: 32), BIRC2 (SEQ ID NO: 33), BIRC3 (SEQ ID NO: 34), BIRC4 (SEQ ID NO: 35), BIRC5 (SEQ ID NO: 36), BIRC6 (SEQ ID NO: 37), BIRC7 (SEQ ID NO: 38), BIRC8 (SEQ ID NO: 39), KEAP1 (SEQ ID NO: 40), DCAF15 (SEQ ID NO: 41), RNF4 (SEQ ID NO: 42) RNF4 isoform 2 (SEQ ID NO: 43), RNF114 (SEQ ID NO: 44), RNF114 isoform 2 (SEQ ID NO: 45), DCAF16 (SEQ ID NO: 46) AHR (SEQ ID NO: 47), MDM2 (SEQ ID NO: 48), UBR2 (SEQ ID NO: 49), SPOP (SEQ ID NO: 50), KLHL3 (SEQ ID NO: 51), KLHL12 (SEQ ID NO: 52), KLHL20 (SEQ ID NO: 53), KLHDC2 (SEQ ID NO: 54), SPSB1 (SEQ ID NO: 55), SPSB2 (SEQ ID NO: 56), SBSB4 (SEQ ID NO: 57), SOCS2 (SEQ ID NO: 58), SOCS6 (SEQ ID NO: 59), FBXO4 (SEQ ID NO: 60), FBXO31 (SEQ ID NO: 61), BTRC (SEQ ID NO: 62), FBW7 (SEQ ID NO: 63), CDC20 (SEQ ID NO: 64), ITCH (SEQ ID NO: 65), PML (SEQ ID NO: 66), TRIM21 (SEQ ID NO: 67), TRIM24 (SEQ ID NO: 68), TRIM33 (SEQ ID NO: 69), GID4 (SEQ ID NO: 70), and DCAF11 (SEQ ID NO: 71), and an enzymatically active portion or variant of any one of the foregoing E3 ligase substrate receptors.

24. The system of method of claim 4, wherein the E3 ligase substrate receptor and/or the E3 ligase substrate receptor that does not bind the modulator at a canonical binding site has an amino acid sequence of at least 95% identity to CRBN (SEQ ID NO: 4).

25. (canceled)

26. The method of claim 24, wherein the E3 ligase that does not bind the modulator at canonical binding site comprises mutations Y384A and W386A.

27. The method of claim 4, wherein the proximity labeling enzyme is a promiscuous biotinylation enzyme.

28. The method of claim 27, wherein the promiscuous biotinylation enzyme is selected from the group consisting of SEQ ID NO: 15, SEQ ID NO: 17, SEQ ID NO: 18, SEQ ID NO: 20, SEQ ID NO: 22, SEQ ID NO: 23, SEQ ID NO: 25, SEQ ID NO: 27, SEQ ID NO: 29, SEQ ID NO: 23 with the mutation corresponding to R118S of SEQ ID NO: 14, SEQ ID NO: 23 with the mutation corresponding to R118G of SEQ ID NO: 14, SEQ ID NO: 25 with the mutation corresponding to R118S of SEQ ID NO: 14, SEQ ID NO: 25 with the mutation corresponding to R118G of SEQ ID NO: 14, SEQ ID NO: 27 with the mutation corresponding to R118S of SEQ ID NO: 14, SEQ ID NO: 27 with the mutation corresponding to R118G of SEQ ID NO: 14, SEQ ID NO: 29 with the mutation corresponding to R118S of SEQ ID NO: 14, and SEQ ID NO: 29 with the mutation corresponding to R118G of SEQ ID NO: 14.

29. (canceled)

30. (canceled)

31. The method of claim 4, wherein a fusion protein comprises SEQ ID NO: 1.

32. (canceled)

33. The method of claim 4, wherein a fusion protein comprises SEQ ID NO: 2.

34. The method of claim 4, wherein the modulator is a compound selected from those in Table 4 and Table 5.

35.-42. (canceled)


SEQUENCE LISTING (71 sequences)

SEQ ID NO: 1 - protein, 764 aa, Artificial
  TurboID CRBN fusion protein
MKDNTVPLKLIALLANGEFHSGEQLGETLGMSRAAINKHIQTLRDWGVDVFTVPGKGYSLPEPIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAATLIRELRAALELFEQEGLAPYLPRWEKLDNFINRPVKLIIGDKEIFGISRGIDKQGALLLEQDGVIKPWMGGEISLRSAEKGSGAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 2 - protein, 1021 aa, Artificial
  TurboID CRBN fusion protein with T2A and GFP
MKDNTVPLKLIALLANGEFHSGEQLGETLGMSRAAINKHIQTLRDWGVDVFTVPGKGYSLPEPIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAATLIRELRAALELFEQEGLAPYLPRWEKLDNFINRPVKLIIGDKEIFGISRGIDKQGALLLEQDGVIKPWMGGEISLRSAEKGSGAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCLEGRGSLLTCGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 3 - protein, 441 aa, Homo sapiens
MAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 4 - protein, 442 aa, Homo sapiens [sequence omitted]

SEQ ID NO: 5 - protein, 379 aa, Homo sapiens
MEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 6 - protein, 398 aa, Homo sapiens
MAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFRYAWTVAQCKICASHIGWKFTATKKDMSPQKFWGLTRSALLPTIPDTEDEISPDKVILCL

SEQ ID NO: 7 - protein, 278 aa, Homo sapiens
MRLQHLLKMIFRIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 8 - protein, 278 aa, Homo sapiens; identical to SEQ ID NO: 7

SEQ ID NO: 9 - protein, 284 aa, Homo sapiens
MAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIVYFPLL

SEQ ID NO: 10 - DNA, 3094 bp, Artificial
  TurboID CRBN vector GOI region
tcgctagcgccgccaccatgaaagacaatactgtgcctctgaagctgatcgctctcctggctaatggcgagttccatagtggcgaacagctgggagaaaccctgggcatgtccagggccgctatcaacaagcacattcagactctgcgcgactggggcgtggacgtgttcaccgtgcccggaaagggctactctctgcccgagcctatcccgctgctgaacgctaaacagattctgggacagctggacggcgggagcgtggcagtcctgcctgtggtcgactccaccaatcagtacctgctggatcgaatcggcgagctgaagagtggggatgcttgcattgcagaatatcagcaggcagggagaggaagcagagggaggaaatggttctctccttttggagctaacctgtacctgagtatgttttggcgcctgaagcggggaccagcagcaatcggcctgggcccggtcatcggaattgtcatggcagaagcgctgcgaaagctgggagcagacaaggtgcgagtcaaatggcccaatgacctgtatctgcaggatagaaagctggcaggcatcctggtggagctggccggaataacaggcgatgctgcacagatcgtcattggcgccgggattaacgtggctatgaggcgcgtggaggaaagcgtggtcaatcagggctggatcacactgcaggaagcagggattaacctggacaggaatactctggccgctacgctgatccgagagctgcgggcagccctggaactgttcgagcaggaaggcctggctccatatctgccacggtgggagaagctggataacttcatcaatagacccgtgaagctgatcattggggacaaagagattttcgggattagccgggggattgataaacagggagccctgctgctggaacaggacggagttatcaaaccctggatgggcggagaaatcagtctgcggtctgccgaaaagggttctggagccggcgagggcgaccagcaggatgccgctcataacatgggcaaccatctgcctctgctgcccgcagagagcgaagaagaggacgagatggaagtggaagatcaggacagcaaagaggccaagaagcccaacatcatcaacttcgacaccagcctgcctaccagccacacatatctgggcgccgacatggaagagttccacggcagaaccctgcacgacgatgacagctgccaagtgatccccgtgctgccccaagtcatgatgattctgatccccggccagacactgcccctgcagctgtttcatcctcaagaggtgtccatggtccgaaacctgatccagaaggaccggacctttgccgtgctggcctacagcaacgtgcaagagagagaggcccagtttggcaccaccgccgagatctacgcctacagagaggaacaggacttcggcatcgagatcgtgaaagtgaaggccatcggccggcagcggttcaaggttctggaactgagaacccagagcgacggcatccagcaggccaaggttcagatcctgcctgagtgtgtgctgcccagcacaatgtctgccgtgcagctggaaagcctgaacaagtgccagatcttccccagcaagcccgtgtccagagaagatcagtgcagctacaagtggtggcagaagtaccagaagcggaagttccactgcgccaacctgaccagctggcccagatggctgtactccctgtacgatgccgagacactgatggaccggatcaaaaagcagctgagagagtgggacgagaacctgaaggacgactccctgcctagcaaccccatcgacttcagctatagagtggccgcctgcctgcctatcgacgacgtgctgagaatccagctgctgaagatcggcagcgccatccagagactgagatgcgagctggacatcatgaacaaatgtaccagcctgtgctgcaagcagtgccaagagacagagatcaccaccaagaacgagatctttagcctgagcctgtgcggccctatggccgcctatgtgaatcctcacggctacgtgcacgaaaccctgaccgtgtacaaggcctgcaacctgaacctgatcggcagacctagcaccgagcacagctggtttccaggatacgcctggacagtggcccagtgcaagatctgtgcctctcacatcggctggaagttcaccgccaccaaaaaggacatgagccctcagaagttctggggcctgaccagaagtgccctgctgcctacaatccccgacaccgaggatgagatcagccccgacaaagtgatcctgtgcctggaaggcaggggttctctgctgacctgcggagatgtcgaagaaaatcccggaccaatggtgtccaagggcgaagaactgtttaccggcgtggtgcccatcctggtggaactggatggggacgtgaacggacacaagttcagcgttagcggagaaggcgaaggcgacgccacatacggaaagctgaccctgaagttcatctgcaccaccggcaagctgcctgtgccttggcctacactggtcaccacactgacatacggcgtgcagtgcttcagcagatatcccgaccatatgaagcagcacgacttcttcaagagcgccatgcctgagggctacgtgcaagagcggaccatcttctttaaggacgacggcaactacaagaccagggccgaagtgaagttcgagggcgacaccctggtcaaccggatcgagctgaagggcatcgacttcaaagaggacggcaacatcctgggacacaagctcgagtacaattacaactcccacaacgtgtacatcatggccgacaagcagaaaaacggcatcaaagtcaacttcaagattcggcacaacatcgaggacggctccgtgcagctggccgatcattatcagcagaacacccctatcggcgacggcccagttctgctgcccgataatcactacctgtccactcagtctgccctgagcaaggaccccaacgagaagagggatcacatggtgctgctcgagtttgtgaccgccgctggcatcacactcggcatggatgagctgtacaagtgaatctagaagtt

SEQ ID NO: 11 - DNA, 2292 bp, Artificial
  TurboID CRBN fusion protein polynucleotide sequence
atgaaagacaatactgtgcctctgaagctgatcgctctcctggctaatggcgagttccatagtggcgaacagctgggagaaaccctgggcatgtccagggccgctatcaacaagcacattcagactctgcgcgactggggcgtggacgtgttcaccgtgcccggaaagggctactctctgcccgagcctatcccgctgctgaacgctaaacagattctgggacagctggacggcgggagcgtggcagtcctgcctgtggtcgactccaccaatcagtacctgctggatcgaatcggcgagctgaagagtggggatgcttgcattgcagaatatcagcaggcagggagaggaagcagagggaggaaatggttctctccttttggagctaacctgtacctgagtatgttttggcgcctgaagcggggaccagcagcaatcggcctgggcccggtcatcggaattgtcatggcagaagcgctgcgaaagctgggagcagacaaggtgcgagtcaaatggcccaatgacctgtatctgcaggatagaaagctggcaggcatcctggtggagctggccggaataacaggcgatgctgcacagatcgtcattggcgccgggattaacgtggctatgaggcgcgtggaggaaagcgtggtcaatcagggctggatcacactgcaggaagcagggattaacctggacaggaatactctggccgctacgctgatccgagagctgcgggcagccctggaactgttcgagcaggaaggcctggctccatatctgccacggtgggagaagctggataacttcatcaatagacccgtgaagctgatcattggggacaaagagattttcgggattagccgggggattgataaacagggagccctgctgctggaacaggacggagttatcaaaccctggatgggcggagaaatcagtctgcggtctgccgaaaagggttctggagccggcgagggcgaccagcaggatgccgctcataacatgggcaaccatctgcctctgctgcccgcagagagcgaagaagaggacgagatggaagtggaagatcaggacagcaaagaggccaagaagcccaacatcatcaacttcgacaccagcctgcctaccagccacacatatctgggcgccgacatggaagagttccacggcagaaccctgcacgacgatgacagctgccaagtgatccccgtgctgccccaagtcatgatgattctgatccccggccagacactgcccctgcagctgtttcatcctcaagaggtgtccatggtccgaaacctgatccagaaggaccggacctttgccgtgctggcctacagcaacgtgcaagagagagaggcccagtttggcaccaccgccgagatctacgcctacagagaggaacaggacttcggcatcgagatcgtgaaagtgaaggccatcggccggcagcggttcaaggttctggaactgagaacccagagcgacggcatccagcaggccaaggttcagatcctgcctgagtgtgtgctgcccagcacaatgtctgccgtgcagctggaaagcctgaacaagtgccagatcttccccagcaagcccgtgtccagagaagatcagtgcagctacaagtggtggcagaagtaccagaagcggaagttccactgcgccaacctgaccagctggcccagatggctgtactccctgtacgatgccgagacactgatggaccggatcaaaaagcagctgagagagtgggacgagaacctgaaggacgactccctgcctagcaaccccatcgacttcagctatagagtggccgcctgcctgcctatcgacgacgtgctgagaatccagctgctgaagatcggcagcgccatccagagactgagatgcgagctggacatcatgaacaaatgtaccagcctgtgctgcaagcagtgccaagagacagagatcaccaccaagaacgagatctttagcctgagcctgtgcggccctatggccgcctatgtgaatcctcacggctacgtgcacgaaaccctgaccgtgtacaaggcctgcaacctgaacctgatcggcagacctagcaccgagcacagctggtttccaggatacgcctggacagtggcccagtgcaagatctgtgcctctcacatcggctggaagttcaccgccaccaaaaaggacatgagccctcagaagttctggggcctgaccagaagtgccctgctgcctacaatccccgacaccgaggatgagatcagccccgacaaagtgatcctgtgcctg

SEQ ID NO: 12 - DNA, 3066 bp, Artificial
  TurboID CRBN fusion protein with T2A and GFP polynucleotide sequence
atgaaagacaatactgtgcctctgaagctgatcgctctcctggctaatggcgagttccatagtggcgaacagctgggagaaaccctgggcatgtccagggccgctatcaacaagcacattcagactctgcgcgactggggcgtggacgtgttcaccgtgcccggaaagggctactctctgcccgagcctatcccgctgctgaacgctaaacagattctgggacagctggacggcgggagcgtggcagtcctgcctgtggtcgactccaccaatcagtacctgctggatcgaatcggcgagctgaagagtggggatgcttgcattgcagaatatcagcaggcagggagaggaagcagagggaggaaatggttctctccttttggagctaacctgtacctgagtatgttttggcgcctgaagcggggaccagcagcaatcggcctgggcccggtcatcggaattgtcatggcagaagcgctgcgaaagctgggagcagacaaggtgcgagtcaaatggcccaatgacctgtatctgcaggatagaaagctggcaggcatcctggtggagctggccggaataacaggcgatgctgcacagatcgtcattggcgccgggattaacgtggctatgaggcgcgtggaggaaagcgtggtcaatcagggctggatcacactgcaggaagcagggattaacctggacaggaatactctggccgctacgctgatccgagagctgcgggcagccctggaactgttcgagcaggaaggcctggctccatatctgccacggtgggagaagctggataacttcatcaatagacccgtgaagctgatcattggggacaaagagattttcgggattagccgggggattgataaacagggagccctgctgctggaacaggacggagttatcaaaccctggatgggcggagaaatcagtctgcggtctgccgaaaagggttctggagccggcgagggcgaccagcaggatgccgctcataacatgggcaaccatctgcctctgctgcccgcagagagcgaagaagaggacgagatggaagtggaagatcaggacagcaaagaggccaagaagcccaacatcatcaacttcgacaccagcctgcctaccagccacacatatctgggcgccgacatggaagagttccacggcagaaccctgcacgacgatgacagctgccaagtgatccccgtgctgccccaagtcatgatgattctgatccccggccagacactgcccctgcagctgtttcatcctcaagaggtgtccatggtccgaaacctgatccagaaggaccggacctttgccgtgctggcctacagcaacgtgcaagagagagaggcccagtttggcaccaccgccgagatctacgcctacagagaggaacaggacttcggcatcgagatcgtgaaagtgaaggccatcggccggcagcggttcaaggttctggaactgagaacccagagcgacggcatccagcaggccaaggttcagatcctgcctgagtgtgtgctgcccagcacaatgtctgccgtgcagctggaaagcctgaacaagtgccagatcttccccagcaagcccgtgtccagagaagatcagtgcagctacaagtggtggcagaagtaccagaagcggaagttccactgcgccaacctgaccagctggcccagatggctgtactccctgtacgatgccgagacactgatggaccggatcaaaaagcagctgagagagtgggacgagaacctgaaggacgactccctgcctagcaaccccatcgacttcagctatagagtggccgcctgcctgcctatcgacgacgtgctgagaatccagctgctgaagatcggcagcgccatccagagactgagatgcgagctggacatcatgaacaaatgtaccagcctgtgctgcaagcagtgccaagagacagagatcaccaccaagaacgagatctttagcctgagcctgtgcggccctatggccgcctatgtgaatcctcacggctacgtgcacgaaaccctgaccgtgtacaaggcctgcaacctgaacctgatcggcagacctagcaccgagcacagctggtttccaggatacgcctggacagtggcccagtgcaagatctgtgcctctcacatcggctggaagttcaccgccaccaaaaaggacatgagccctcagaagttctggggcctgaccagaagtgccctgctgcctacaatccccgacaccgaggatgagatcagccccgacaaagtgatcctgtgcctggaaggcaggggttctctgctgacctgcggagatgtcgaagaaaatcccggaccaatggtgtccaagggcgaagaactgtttaccggcgtggtgcccatcctggtggaactggatggggacgtgaacggacacaagttcagcgttagcggagaaggcgaaggcgacgccacatacggaaagctgaccctgaagttcatctgcaccaccggcaagctgcctgtgccttggcctacactggtcaccacactgacatacggcgtgcagtgcttcagcagatatcccgaccatatgaagcagcacgacttcttcaagagcgccatgcctgagggctacgtgcaagagcggaccatcttctttaaggacgacggcaactacaagaccagggccgaagtgaagttcgagggcgacaccctggtcaaccggatcgagctgaagggcatcgacttcaaagaggacggcaacatcctgggacacaagctcgagtacaattacaactcccacaacgtgtacatcatggccgacaagcagaaaaacggcatcaaagtcaacttcaagattcggcacaacatcgaggacggctccgtgcagctggccgatcattatcagcagaacacccctatcggcgacggcccagttctgctgcccgataatcactacctgtccactcagtctgccctgagcaaggaccccaacgagaagagggatcacatggtgctgctcgagtttgtgaccgccgctggcatcacactcggcatggatgagctgtacaagtga

SEQ ID NO: 13 - DNA, 3094 bp, Artificial; identical to SEQ ID NO: 10
  pcLV-CMV-MCS-T2A-eGFP-IRES-Puro GOI insert

SEQ ID NO: 14 - protein, 321 aa, Escherichia coli
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEK

SEQ ID NO: 15 - protein, 232 aa, Artificial
  BioID2 amino acid
FKNLIWLKEVDSTQERLKEWNVSYGTALVADRQTKGRGGLGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKSFKEFKGKIESKMLYLGEEVKLLGEGKITGKLVGLSEKGGALILTEEGIKEILSGEFSLRRS

SEQ ID NO: 16 - DNA, 696 bp, Artificial
  BioID2 polynucleotide
ttcaagaacctgatctggctgaaggaggtggacagcacccaggagagactgaaggagtggaacgtgagctacggcaccgccctggtggccgacagacagaccaagggcagaggcggcctgggcagaaagtggctgagccaggagggcggcctgtacttcagcttcctgctgaaccccaaggagttcgagaacctgctgcagctgcccctggtgctgggcctgagcgtgagcgaggccctggaggagatcaccgagatccccttcagcctgaagtggcccaacgacgtgtacttccaggagaagaaggtgagcggcgtgctgtgcgagctgagcaaggacaagctgatcgtgggcatcggcatcaacgtgaaccagagagagatccccgaggagatcaaggacagagccaccaccctgtacgagatcaccggcaaggactgggacagaaaggaggtgctgctgaaggtgctgaagagaatcagcgagaacctgaagaagttcaaggagaagagcttcaaggagttcaagggcaagatcgagagcaagatgctgtacctgggcgaggaggtgaagctgctgggcgagggcaagatcaccggcaagctggtgggcctgagcgagaagggcggcgccctgatcctgaccgaggagggcatcaaggagatcctgagcggcgagttcagcctgagaagaagc

SEQ ID NO: 17 - protein, 260 aa, Artificial
  BASU
GKLSESEIRFGLKTEVMGQHLIYHDVLSSTQKTAHELANNNAPEGTLVVADKQTAGRGGMSRVWHSQEGNGVWMSLILRPDIPLQKTPQLTLLAAVAVVQGIEEAAGIQTDIKWPNDILINGKKTVGILTEMQAEEDRVRSVIIGIGINVNQQPNDFPDELKDIATSLSQAAGEKIDRAGVIQHILLCFEKRYRDYMTHGFTPIKLLWESYALGIGTNMRARTLNGTFYGKALGIDDEGVLLLETNEGIKKIYSADISLR

SEQ ID NO: 18 - protein, 257 aa, Artificial
  miniTurbo amino acid
IPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAAMLIRELRAALELFEQEGLAPYLSRWEKLDNFINRPVKLIIGDKEIFGISRGIDKQGALLLEQDGVIKPWMGGEISLRSAEK

SEQ ID NO: 19 - DNA, 771 bp, Artificial
  miniTurbo polynucleotide
atcccgctgctgaacgctaaacagattctgggacagctggacggcgggagcgtggcagtcctgcctgtggtcgactccaccaatcagtacctgctggatcgaatcggcgagctgaagagtggggatgcttgcattgcagaatatcagcaggcagggagaggaagcagagggaggaaatggttctctccttttggagctaacctgtacctgagtatgttttggcgcctgaagcggggaccagcagcaatcggcctgggcccggtcatcggaattgtcatggcagaagcgctgcgaaagctgggagcagacaaggtgcgagtcaaatggcccaatgacctgtatctgcaggatagaaagctggcaggcatcctggtggagctggccggaataacaggcgatgctgcacagatcgtcattggcgccgggattaacgtggctatgaggcgcgtggaggaaagcgtggtcaatcagggctggatcacactgcaggaagcagggattaacctggacaggaatactctggccgctatgctgatccgagagctgcgggcagccctggaactgttcgagcaggaaggcctggctccatatctgtcacggtgggagaagctggataacttcatcaatagacccgtgaagctgatcattggggacaaagagattttcgggattagccgggggattgataaacagggagccctgctgctggaacaggacggagttatcaaaccctggatgggcggagaaatcagtctgcggtctgccgaaaag

SEQ ID NO: 20 - protein, 319 aa, Artificial
  20 turboID amino acid
KDNTVPLKLIALLANGEFHSGEQLGETLGMSRAAINKHIQTLRDWGVDVFTVPGKGYSLPEPIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAATLIRELRAALELFEQEGLAPYLPRWEKLDNFINRPVKLIIGDKEIFGISRGIDKQGALLLEQDGVIKPWMGGEISLRSAEK

SEQ ID NO: 21 - DNA, 957 bp, Artificial
  turboID polynucleotide
aaagacaatactgtgcctctgaagctgatcgctctcctggctaatggcgagttccatagtggcgaacagctgggagaaaccctgggcatgtccagggccgctatcaacaagcacattcagactctgcgcgactggggcgtggacgtgttcaccgtgcccggaaagggctactctctgcccgagcctatcccgctgctgaacgctaaacagattctgggacagctggacggcgggagcgtggcagtcctgcctgtggtcgactccaccaatcagtacctgctggatcgaatcggcgagctgaagagtggggatgcttgcattgcagaatatcagcaggcagggagaggaagcagagggaggaaatggttctctccttttggagctaacctgtacctgagtatgttttggcgcctgaagcggggaccagcagcaatcggcctgggcccggtcatcggaattgtcatggcagaagcgctgcgaaagctgggagcagacaaggtgcgagtcaaatggcccaatgacctgtatctgcaggatagaaagctggcaggcatcctggtggagctggccggaataacaggcgatgctgcacagatcgtcattggcgccgggattaacgtggctatgaggcgcgtggaggaaagcgtggtcaatcagggctggatcacactgcaggaagcagggattaacctggacaggaatactctggccgctacgctgatccgagagctgcgggcagccctggaactgttcgagcaggaaggcctggctccatatctgccacggtgggagaagctggataacttcatcaatagacccgtgaagctgatcattggggacaaagagattttcgggattagccgggggattgataaacagggagccctgctgctggaacaggacggagttatcaaaccctggatgggcggagaaatcagtctgcggtctgccgaaaag

SEQ ID NO: 22 - protein, 319 aa, Artificial
  AirID amino acid
MKDNTVPLTLISILADGEFHSGEQLGEQLGMSRAAINKHIKTLRDWGVDVFRVQGKGYCLPEPIQLLDEEKIRQQLDEGSVTVLPVIDSTNQYLLDRLDELTSGDVCIAEYQQAGRGSRGRKWFSPFGANLYLSMYWRLEQGPAAAMGLSLVIGIVMAETLQKLGADGVRVKWPNDLYLNDRKLAGILVEMTGKTGDAAHIVIGAGINLSMREPETDEVDQSWINLQEAGITIDRNQLAARLIKDLRSALRQFEQQGLAPFLSRWEALDNFINRPVKLIIGDREIHGIARGINEQGALLLEQDGVIKPWIGGEISLRSA

SEQ ID NO: 23 - protein, 319 aa, Artificial
  AVVA amino acid
MSEQWSRKREILALLSSGHFVSGEELATQLGISRTAVSKHIAALEEYGVDIYSVKGKGYKLANPISLIDESKLKSAINNRCFYFDEIPSTNGFMLKHAEELKSGDICVAEYQSAGRGRRGRTWVSPYGCHLYFSLYWRFPQGMAQAMGLSLVVACSLVKVLKSFGVDGVGVKWPNDIYLNHKKLAGVLIEMSGQADSECHLVIGIGINMAMSEQQGKKIDQPWSDLSSLTSMPDKTELLIALQKQLKQDLELFEREGLKAFQPRWQEADLFYGKQIKLLMGENQVEGICRGIDEQGAVLLETDDGIQAFIGGEISLRAA

SEQ ID NO: 24 - DNA, 957 bp, Artificial
  AVVA polynucleotide
atgtctgagcagtggtctaggaagagagagatccttgctctcctcagctctggacatttcgtgtctggtgaggaacttgctactcagctcggaatctctagaaccgctgtgagcaaacatatcgctgcccttgaagagtacggcgtggacatctatagcgtgaaaggtaagggatacaagctcgcgaacccgatctctcttatcgacgagtctaagctcaagagcgccatcaacaaccgttgcttctacttcgacgagatcccgtctaccaacggcttcatgcttaaacacgctgaggaactcaagtccggggatatctgtgttgctgagtaccagtctgctggaagaggacgtagaggaagaacttgggtttcaccttacggatgccacctctacttcagcctttattggaggttcccgcaaggtatggctcaggctatgggactttctctcgttgttgcttgcagcctcgttaaggtgctcaagtctttcggagttgatggtgtgggagtgaagtggcctaacgacatctacctcaaccataagaaactcgccggtgtgctcatcgagatgtctggacaagctgattctgagtgccatctcgttatcggcatcgggatcaacatggctatgtctgaacagcaggggaagaagatcgatcagccttggtctgacctctcctctctcacttctatgcctgacaagaccgagctgcttatcgctctccaaaagcagcttaagcaggacctcgagcttttcgagagagagggacttaaggctttccagcctagatggcaagaggctgatctcttctacgggaagcagatcaagcttctcatgggagagaatcaggtcgagggaatctgcagaggaattgatgagcagggtgctgttctcctcgagactgatgatggaatccaggcttttatcggcggggagatctctttgagagctgct

SEQ ID NO: 25 - protein, 304 aa, Artificial
  AHLA amino acid
MRPFPLLRLLSDGEFHSGQALAEALGVSRASIWNALRNAEALGVDVHAVRGRGYRLSEPLDWLDEAIVARHLGEKASFFDLHVLDSVDSTNTALMERALQGAPHGTCVAAERQTAGRGRRGRAWHAVLGGSLTFSLLWRFNLGLGSLSGLSLAVGLAVVRALNKLGVHGARLKWPNDVLTDYRKLAGILIELQGDMLGPAAAVIGIGLNVRLSEAARNAVDQAVVDLHSLCGAPADRNTLLADLLRELAAMLTAFEQDGFAPLRAEWEAHHAYQDKAVRLLLPDGAGVQGVARGVDEDGALLLE

SEQ ID NO: 26 - DNA, 969 bp, Artificial
  AHLA polynucleotide
atgaggccttttccgcttctcagactcttgtctgatggggagttccattctggacaagctcttgctgaagctctcggagtgtctagagcttctatctggaacgctctcagaaacgctgaggctcttggagttgatgtgcatgctgttagaggacgtgggtacagactttctgagcctcttgattggctcgacgaggctatcgttgctagacatcttggagagaaggccagcttcttcgatctccatgttctcgactctgtggactctactaacacggctctcatggaaagggctctccaaggtgctcctcatggaacttgtgttgctgctgagagacagactgctggaagaggaagaagaggtagagcttggcatgctgtgcttggaggatctcttaccttctctcttctctggcgtttcaacctcggactcggatctctttctggactctctcttgctgttggactcgctgttgttagggctcttaacaagctcggagtgcatggtgctagactcaagtggcctaacgatgtgctcaccgattacagaaagctcgctggaatcctcatcgagcttcagggtgatatgcttggacctgctgctgctgttatcggaatcggacttaacgtgagactctctgaggctgctaggaacgctgttgatcaggctgttgtggatctccattctctttgtggtgctccggctgatagaaatacccttcttgctgatctcctccgtgagcttgctgctatgcttactgctttcgagcaggatggattcgctcctcttagagctgaatgggaagctcatcacgcttaccaggataaggctgtgagacttcttttgcctgatggtgctggtgttcagggtgttgctagaggtgttgatgaggatggtgctttgctcctcgagactcaatctggggagagaagattccacagcggagagatttctcttaggcctgctgct

SEQ ID NO: 27 - protein, 319 aa, Artificial
  GFVA amino acid
MKDNTVPLTLISILADGEFHSGEQLGEQLGMSRAAINKHIKTLRDWGVDVFRVQGKGYCLPEPIQLLDEEKIRQQLDEGSVTVLPVIDSTNQYLLDRLDELTSGDVCIAEYQQAGRGRRGRKWFSPFGANLYLSMYWRLEQGPAAAMGLSLVIGIVMAETLQKLGADGVRVKWPNDLYLNDRKLAGILVEMTGKTGDAAHIVIGAGINLSMREPETDEVDQSWINLQEAGITIDRNQLAARLIKDLRSALRQFEQQGLAPFLSRWEALDNFINRPVKLIIGDREIHGIARGINEQGALLLEQDGVIKPWIGGEISLRSA

SEQ ID NO: 28 - DNA, 957 bp, Artificial
  GFVA polynucleotide
atgaaggacaacaccgttccgctcacgcttatctctatccttgctgatggtgagttccactctggtgaacaacttggagagcagctcggaatgtctagggctgctattaacaagcacatcaagaccctccgtgactggggagttgatgtgttcagagttcaaggtaaggggtactgccttcctgagcctatccaacttctcgacgaagagaagatcaggcagcagcttgatgagggatctgttactgttctcccggtgatcgattcgaccaaccagtaccttctcgataggctcgatgagcttacctctggtgatgtgtgtatcgctgagtaccaacaggctggaagaggacgtagaggtaggaagtggttttctccgttcggagctaacctctacctcagcatgtattggagacttgagcaaggacctgctgctgctatgggactttctctcgttatcggaatcgtgatggctgagactctccaaaagcttggagctgacggtgttagagtgaagtggcctaacgacctttacctcaacgataggaagctcgctggaatcctcgttgagatgactggaaagactggtgacgctgctcatatcgtgattggagctggaatcaacctctctatgcgtgagcctgagactgatgaggttgaccagtcttggatcaacctccaagaggctggtatcaccatcgatagaaaccagcttgctgccaggctcatcaaggatcttagatctgctctcaggcagttcgagcaacaaggacttgctccattcctcagcagatgggaagctctcgacaacttcatcaacaggccagtgaagctcatcatcggtgatagagagatccacggaatcgctaggggaatcaacgaacaaggggctcttttgcttgagcaggacggtgtgattaagccttggattggaggtgagatcagcctcagatctgct

SEQ ID NO: 29 - protein, 318 aa, Artificial
  All amino acid
MKDKTRPLKLIAILADGQFHSGEELATQLGISRAAINKHIKTLREWGVDVFSVQGKGYCLANPIQLLDETKIKQQLKNRVTVLPVIDSTNQYLLDRLDELKSGDVCVAEYQSAGRGRRGRKWFSPFGSNLYFSMYWRLEQGMAAAMGLSLVVGIVMAEVLKKLGADGVRVKWPNDLYLNDRKLAGILVEMTGKTGDAAHIVIGIGINLSMSEPETNEVDQSWANLSNVGITIDRNQLVASLAKDLKSALRQFEQQGLAAFLSRWQALDNFINRPVKLLIGDKEIHGIARGINEQGALLLEQDGGIKAYIGGEISLRSA

SEQ ID NO: 30 - DNA, 954 bp, Artificial
  All polynucleotide
atgaaggacaagaccagaccgctcaagcttatcgctatccttgctgatggacagttccactctggtgaggaacttgctactcagctcggaatttctagggccgctatcaacaagcacatcaagactctccgtgagtggggagttgatgtgttctctgttcaaggtaaggggtactgcctcgctaaccctatccaacttctcgacgagactaagatcaagcagcagctcaagaacagggtgacagttctccctgtgatcgactctactaaccagtacctcctcgataggctcgacgagcttaagtctggtgatgtttgtgtggccgagtaccagtctgctggaagaggacgtagaggtaggaagtggtttagcccgttcggaagcaacctctacttcagcatgtattggaggctcgagcaaggtatggctgctgctatgggactttctctcgttgtgggaatcgtgatggctgaggtgctcaagaagcttggagctgacggggttagagttaagtggcctaacgatctctacctcaacgacagaaagctcgctggaatcctcgttgagatgactggaaagactggtgacgctgctcatatcgtgatcggaatcggtatcaacctcagcatgtctgagcctgagactaacgaggttgaccagtcttgggctaacctctctaacgtgggaatcaccatcgataggaaccagctcgttgcttctctcgctaaggatctcaagtctgctctcagacaattcgagcagcagggacttgctgctttcttgtctagatggcaggctctcgacaacttcatcaacagacctgtgaagctcctcatcggggacaaagagattcacggaatcgctaggggaatcaacgaacaaggggctcttttgcttgagcaggacggtggaatcaaggcttacatcggaggtgagatcagcctcagatctgct

SEQ ID NO: 31 - protein, 213 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 32 - protein, 1403 aa, Homo sapiens
MATQQKASDERISQFDHNLLPELSALLGLDAVQLAKELEEEEQKERAKMQKGYNSQMRSEAKRLKTFVTYEPYSSWIPQEMAAAGFYFTGVKSGIQCFCCSLILFGAGLTRLPIEDHKRFHPDCGFLLNKDVGNIAKYDIRVKNLKSRLRGGKMRYQEEEARLASFRNWPFYVQGISPCVLSEAGFVFTGKQDTVQCFSCGGCLGNWEEGDDPWKEHAKWFPKCEFLRSKKSSEEITQYIQSYKGFVDITGEHFVNSWVQRELPMASAYCNDSIFAYEELRLDSFKDWPRESAVGVAALAKAGLFYTGIKDIVQCFSCGGCLEKWQEGDDPLDDHTRCFPNCPFLQNMKSSAEVTPDLQSRGELCELLETTSESNLEDSIAVGPIVPEMAQGEAQWFQEAKNLNEQLRAAYTSASFRHMSLLDISSDLATDHLLGCDLSIASKHISKPVQEPLVLPEVFGNLNSVMCVEGEAGSGKTVLLKKIAFLWASGCCPLLNRFQLVFYLSLSSTRPDEGLASIICDQLLEKEGSVTEMCVRNIIQQLKNQVLFLLDDYKEICSIPQVIGKLIQKNHLSRTCLLIAVRTNRARDIRRYLETILEIKAFPFYNTVCILRKLFSHNMTRLRKFMVYFGKNQSLQKIQKTPLFVAAICAHWFQYPFDPSFDDVAVFKSYMERLSLRNKATAEILKATVSSCGELALKGFFSCCFEFNDDDLAEAGVDEDEDLTMCLMSKFTAQRLRPFYRFLSPAFQEFLAGMRLIELLDSDRQEHQDLGLYHLKQINSPMMTVSAYNNFLNYVSSLPSTKAGPKIVSHLLHLVDNKESLENISENDDYLKHQPEISLQMQLLRGLWQICPQAYFSMVSEHLLVLALKTAYQSNTVAACSPFVLQFLQGRTLTLGALNLQYFFDHPESLSLLRSIHFPIRGNKTSPRAHFSVLETCFDKSQVPTIDQDYASAFEPMNEWERNLAEKEDNVKSYMDMQRRASPDLSTGYWKLSPKQYKIPCLEVDVNDIDVVGQDMLEILMTVFSASQRIELHLNHSRGFIESIRPALELSKASVTKCSISKLELSAAEQELLLTLPSLESLEVSGTIQSQDQIFPNLDKFLCLKELSVDLEGNINVFSVIPEEFPNFHHMEKLLIQISAEYDPSKLVKLIQNSPNLHVFHLKCNFFSDFGSLMTMLVSCKKLTEIKFSDSFFQAVPFVASLPNFISLKILNLEGQQFPDEETSEKFAYILGSLSNLEELILPTGDGIYRVAKLIIQQCQQLHCLRVLSFFKTLNDDSVVEIAKVAISGGFQKLENLKLSINHKITEEGYRNFFQALDNMPNLQELDISRHFTECIKAQATTVKSLSQCVLRLPRLIRLNMLSWLLDADDIALLNVMKERHPQSKYLTILQKWILPFSPIIQK

SEQ ID NO: 33 - protein, 618 aa, Homo sapiens
MHKTASQRLFPGPSYQNIKSIMEDSTILSDWTNSNKQKMKYDFSCELYRMSTYSTFPAGVPVSERSLARAGFYYTGVNDKVKCFCCGLMLDNWKLGDSPIQKHKQLYPSCSFIQNLVSASLGSTSKNTSPMRNSFAHSLSPTLEHSSLFSGSYSSLSPNPLNSRAVEDISSSRTNPYSYAMSTEEARFLTYHMWPLTFLSPSELARAGFYYIGPGDRVACFACGGKLSNWEPKDDAMSEHRRHFPNCPFLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSTSDTTGEENADPPIIHFGPGESSSEDAVMMNTPVVKSALEMGFNRDLVKQTVQSKILTTGENYKTVNDIVSALLNAEDEKREEEKEKQAEEMASDDLSLIRKNRMALFQQLTCVLPILDNLLKANVINKQEHDIIKQKTQIPLQARELIDTILVKGNAAANIFKNCLKEIDSTLYKNLFVDKNMKYIPTEDVSGLSLEEQLRRLQEERTCKVCMDKEVSVVFIPCGHLVVCQECAPSLRKCPICRGIIKGTVRTFLS

SEQ ID NO: 34 - protein, 604 aa, Homo sapiens
MNIVENSIFLSNLMKSANTFELKYDLSCELYRMSTYSTFPAGVPVSERSLARAGFYYTGVNDKVKCFCCGLMLDNWKRGDSPTEKHKKLYPSCRFVQSLNSVNNLEATSQPTFPSSVTNSTHSLLPGTENSGYFRGSYSNSPSNPVNSRANQDFSALMRSSYHCAMNNENARLLTFQTWPLTFLSPTDLAKAGFYYIGPGDRVACFACGGKLSNWEPKDNAMSEHLRHFPKCPFIENQLQDTSRYTVSNLSMQTHAARFKTFFNWPSSVLVNPEQLASAGFYYVGNSDDVKCFCCDGGLRCWESGDDPWVQHAKWFPRCEYLIRIKGQEFIRQVQASYPHLLEQLLSTSDSPGDENAESSIIHFEPGEDHSEDAIMMNTPVINAAVEMGFSRSLVKQTVQRKILATGENYRLVNDLVLDLLNAEDEIREEERERATEEKESNDLLLIRKNRMALFQHLTCVIPILDSLLTAGIINEQEHDVIKQKTQTSLQARELIDTILVKGNIAATVFRNSLQEAEAVLYEHLFVQQDIKYIPTEDVSDLPVEEQLRRLQEERTCKVCMDKEVSIVFIPCGHLVVCKDCAPSLRKCPICRSTIKGTVRTFLS

SEQ ID NO: 35 - protein, 497 aa, Homo sapiens
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 36 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 37 - protein, 4857 aa, Homo sapiens
MVTGGGAAPPGTVTEPLPSVIVLSAGRKMAAAAAAASGPGCSSAAGAGAAGVSEWLVLRDGCMHCDADGLHSLSYHPALNAILAVTSRGTIKVIDGTSGATLQASALSAKPGGQVKCQYISAVDKVIFVDDYAVGCRKDLNGILLLDTALQTPVSKQDDVVQLELPVTEAQQLLSACLEKVDISSTEGYDLFITQLKDGLKNTSHETAANHKVAKWATVTFHLPHHVLKSIASAIVNELKKINQNVAALPVASSVMDRLSYLLPSARPELGVGPGRSVDRSLMYSEANRRETFTSWPHVGYRWAQPDPMAQAGFYHQPASSGDDRAMCFTCSVCLVCWEPTDEPWSEHERHSPNCPFVKGEHTQNVPLSVTLATSPAQFPCTDGTDRISCFGSGSCPHFLAAATKRGKICIWDVSKLMKVHLKFEINAYDPAIVQQLILSGDPSSGVDSRRPTLAWLEDSSSCSDIPKLEGDSDDLLEDSDSEEHSRSDSVTGHTSQKEAMEVSLDITALSILQQPEKLQWEIVANVLEDTVKDLEELGANPCLTNSKSEKTKEKHQEQHNIPFPCLLAGGLLTYKSPATSPISSNSHRSLDGLSRTQGESISEQGSTDNESCTNSELNSPLVRRTLPVLLLYSIKESDEKAGKIFSQMNNIMSKSLHDDGFTVPQIIEMELDSQEQLLLQDPPVTYIQQFADAAANLTSPDSEKWNSVFPKPGTLVQCLRLPKFAEEENLCIDSITPCADGIHLLVGLRTCPVESLSAINQVEALNNLNKLNSALCNRRKGELESNLAVVNGANISVIQHESPADVQTPLIIQPEQRNVSGGYLVLYKMNYATRIVTLEEEPIKIQHIKDPQDTITSLILLPPDILDNREDDCEEPIEDMQLTSKNGFEREKTSDISTLGHLVITTQGGYVKILDLSNFEILAKVEPPKKEGTEEQDTFVSVIYCSGTDRLCACTKGGELHFLQIGGTCDDIDEADILVDGSLSKGIEPSSEGSKPLSNPSSPGISGVDLLVDQPFTLEILTSLVELTRFETLTPRFSATVPPCWVEVQQEQQQRRHPQHLHQQHHGDAAQHTRTWKLQTDSNSWDEHVFELVLPKACMVGHVDFKFVLNSNITNIPQIQVTLLKNKAPGLGKVNALNIEVEQNGKPSLVDLNEEMQHMDVEESQCLRLCPFLEDHKEDILCGPVWLASGLDLSGHAGMLTLTSPKLVKGMAGGKYRSFLIHVKAVNERGTEEICNGGMRPVVRLPSLKHQSNKGYSLASLLAKVAAGKEKSSNVKNENTSGTRKSENLRGCDLLQEVSVTIRRFKKTSISKERVQRCAMLQFSEFHEKLVNTLCRKTDDGQITEHAQSLVLDTLCWLAGVHSNGPGSSKEGNENLLSKTRKFLSDIVRVCFFEAGRSIAHKCARFLALCISNGKCDPCQPAFGPVLLKALLDNMSFLPAATTGGSVYWYFVLLNYVKDEDLAGCSTACASLLTAVSRQLQDRLTPMEALLQTRYGLYSSPFDPVLFDLEMSGSSCKNVYNSSIGVQSDEIDLSDVLSGNGKVSSCTAAEGSFTSLTGLLEVEPLHFTCVSTSDGTRIERDDAMSSFGVTPAVGGLSSGTVGEASTALSSAAQVALQSLSHAMASAEQQLQVLQEKQQQLLKLQQQKAKLEAKLHQTTAAAAAAASAVGPVHNSVPSNPVAAPGFFIHPSDVIPPTPKTTPLFMTPPLTPPNEAVSVVINAELAQLFPGSVIDPPAVNLAAHNKNSNKSRMNPLGSGLALAISHASHFLQPPPHQSIIIERMHSGARRFVTLDFGRPILLTDVLIPTCGDLASLSIDIWTLGEEVDGRRLVVATDISTHSLILHDLIPPPVCRFMKITVIGRYGSTNARAKIPLGFYYGHTYILPWESELKLMHDPLKGEGESANQPEIDQHLAMMVALQEDIQCRYNLACHRLETLLQSIDLPPLNSANNAQYFLRKPDKAVEEDSRVFSAYQDCIQLQLQLNLAHNAVQRLKVALGASRKMLSETSNPEDLIQTSSTEQLRTIIRYLLDTLLSLLHASNGHSVPAVLQSTFHAQACEELFKHLCISGTPKIRLHTGLLLVQLCGGERWWGQFLSNVLQELYNSEQLLIFPQDRVFMLLSCIGQRSLSNSGVLESLLNLLDNLLSPLQPQLPMHRRTEGVLDIPMISWVVMLVSRLLDYVATVEDEAAAAKKPLNGNQWSFINNNLHTQSLNRSSKGSSSLDRLYSRKIRKQLVHHKQQLNLLKAKQKALVEQMEKEKIQSNKGSSYKLLVEQAKLKQATSKHFKDLIRLRRTAEWSRSNLDTEVTTAKESPEIEPLPFTLAHERCISVVQKLVLFLLSMDFTCHADLLLFVCKVLARIANATRPTIHLCEIVNEPQLERLLLLLVGTDFNRGDISWGGAWAQYSLTCMLQDILAGELLAPVAAEAMEEGTVGDDVGATAGDSDDSLQQSSVQLLETIDEPLTHDITGAPPLSSLEKDKEIDLELLQDLMEVDIDPLDIDLEKDPLAAKVFKPISSTWYDYWGADYGTYNYNPYIGGLGIPVAKPPANTEKNGSQTVSVSVSQALDARLEVGLEQQAELMLKMMSTLEADSILQALTNTSPTLSQSPTGTDDSLLGGLQAANQTSQLIIQLSSVPMLNVCFNKLFSMLQVHHVQLESLLQLWLTLSLNSSSTGNKENGADIFLYNANRIPVISLNQASITSFLTVLAWYPNTLLRTWCLVLHSLTLMTNMQLNSGSSSAIGTQESTAHLLVSDPNLIHVLVKFLSGTSPHGTNQHSPQVGPTATQAMQEFLTRLQVHLSSTCPQIFSEFLLKLIHILSTERGAFQTGQGPLDAQVKLLEFTLEQNFEVVSVSTISAVIESVTFLVHHYITCSDKVMSRSGSDSSVGARACFGGLFANLIRPGDAKAVCGEMTRDQLMFDLLKLVNILVQLPLSGNREYSARVSVTTNTTDSVSDEEKVSGGKDGNGSSTSVQGSPAYVADLVLANQQIMSQILSALGLCNSSAMAMIIGASGLHLTKHENFHGGLDAISVGDGLFTILTTLSKKASTVHMMLQPILTYMACGYMGRQGSLATCQLSEPLLWFILRVLDTSDALKAFHDMGGVQLICNNMVTSTRAIVNTARSMVSTIMKFLDSGPNKAVDSTLKTRILASEPDNAEGIHNFAPLGTITSSSPTAQPAEVLLQATPPHRRARSAAWSYIFLPEEAWCDLTIHLPAAVLLKEIHIQPHLASLATCPSSVSVEVSADGVNMLPLSTPVVTSGLTYIKIQLVKAEVASAVCLRLHRPRDASTLGLSQIKLLGLTAFGTTSSATVNNPFLPSEDQVSKTSIGWLRLLHHCLTHISDLEGMMASAAAPTANLLQTCAALLMSPYCGMHSPNIEVVLVKIGLQSTRIGLKLIDILLRNCAASGSDPTDLNSPLLFGRLNGLSSDSTIDILYQLGTTQDPGTKDRIQALLKWVSDSARVAAMKRSGRMNYMCPNSSTVEYGLLMPSPSHLHCVAAILWHSYELLVEYDLPALLDQELFELLFNWSMSLPCNMVLKKAVDSLLCSMCHVHPNYFSLLMGWMGITPPPVQCHHRLSMTDDSKKQDLSSSLTDDSKNAQAPLALTESHLATLASSSQSPEAIKQLLDSGLPSLLVRSLASFCFSHISSSESIAQSIDISQDKLRRHHVPQQCNKMPITADLVAPILRFLTEVGNSHIMKDWLGGSEVNPLWTALLFLLCHSGSTSGSHNLGAQQTSARSASLSSAATTGLTTQQRTAIENATVAFFLQCISCHPNNQKLMAQVLCELFQTSPQRGNLPTSGNISGFIRRLFLQLMLEDEKVTMFLQSPCPLYKGRINATSHVIQHPMYGAGHKFRTLHLPVSTTLSDVLDRVSDTPSITAKLISEQKDDKEKKNHEEKEKVKAENGFQDNYSVVVASGLKSQSKRAVSATPPRPPSRRGRTIPDKIGSTSGAEAANKIITVPVFHLFHKLLAGQPLPAEMTLAQLLTLLYDRKLPQGYRSIDLTVKLGSRVITDPSLSKTDSYKRLHPEKDHGDLLASCPEDEALTPGDECMDGILDESLLETCPIQSPLQVFAGMGGLALIAERLPMLYPEVIQQVSAPVVTSTTQEKPKDSDQFEWVTIEQSGELVYEAPETVAAEPPPIKSAVQTMSPIPAHSLAAFGLFLRLPGYAEVLLKERKHAQCLLRLVLGVTDDGEGSHILQSPSANVLPTLPFHVLRSLFSTTPLTTDDGVLLRRMALEIGALHLILVCLSALSHHSPRVPNSSVNQTEPQVSSSHNPTSTEEQQLYWAKGTGFGTGSTASGWDVEQALTKQRLEEEHVTCLLQVLASYINPVSSAVNGEAQSSHETRGQNSNALPSVLLELLSQSCLIPAMSSYLRNDSVLDMARHVPLYRALLELLRAIASCAAMVPLLLPLSTENGEEEEEQSECQTSVGTLLAKMKTCVDTYTNRLRSKRENVKTGVKPDASDQEPEGLTLLVPDIQKTAEIVYAATTSLRQANQEKKLGEYSKKAAMKPKPLSVLKSLEEKYVAVMKKLQFDTFEMVSEDEDGKLGFKVNYHYMSQVKNANDANSAARARRLAQEAVTLSTSLPLSSSSSVFVRCDEERLDIMKVLITGPADTPYANGCFEFDVYFPQDYPSSPPLVNLETTGGHSVRFNPNLYNDGKVCLSILNTWHGRPEEKWNPQTSSFLQVLVSVQSLILVAEPYFNEPGYERSRGTPSGTQSSREYDGNIRQATVKWAMLEQIRNPSPCFKEVIHKHFYLKRVEIMAQCEEWIADIQQYSSDKRVGRTMSHHAAALKRHTAQLREELLKLPCPEGLDPDTDDAPEVCRATTGAEETLMHDQVKPSSSKELPSDFQL

SEQ ID NO: 38 - protein, 298 aa, Homo sapiens
MGPKDSAKCLHRGPQPSHWAAGDGPTQERCGPRSLGSPVLGLDTCRAWDHVDGQILGQLRPLTEEEEEEGAGATLSRGPAFPGMGSEELRLASFYDWPLTAEVPPELLAAAGFFHTGHQDKVRCFFCYGGLQSWKRGDDPWTEHAKWFPSCQFLLRSKGRDFVHSVQETHSQLLGSWDPWEEPEDAAPVAPSVPASGYPELPTPRREVQSESAQEPGGVSPAEAQRAWWVLEPPGARDVEAQLRRLQEERTCKVCLDRAVSIVFVPCGHLVCAECAPGLQLCPICRAPVRSRVRTFLS

SEQ ID NO: 39 - protein, 236 aa, Homo sapiens
MTGYEARLITFGTWMYSVNKEQLARAGFYAIGQEDKVQCFHCGGGLANWKPKEDPWEQHAKWYPGCKYLLEEKGHEYINNIHLTRSLEGALVQTTKKTPSLTKRISDTIFPNPMLQEAIRMGFDFKDVKKIMEERIQTSGSNYKTLEVLVADLVSAQKDTTENELNQTSLQREISPEEPLRRLQEEKLCKICMDRHIAVVFIPCGHLVTCKQCAEAVDRCPMCSAVIDFKQRVFMS

SEQ ID NO: 40 - protein, 624 aa, Homo sapiens
MQPDPRPSGAGACCRFLPLQSQCPEGAGDAVMYASTECKAEVTPSQHGNRTFSYTLEDHTKQAFGIMNELRLSQQLCDVTLQVKYQDAPAAQFMAHKVVLASSSPVFKAMFTNGLREQGMEVVSIEGIHPKVMERLIEFAYTASISMGEKCVLHVMNGAVMYQIDSVVRACSDFLVQQLDPSNAIGIANFAEQIGCVELHQRAREYIYMHFGEVAKQEEFFNLSHCQLVTLISRDDLNVRCESEVFHACINWVKYDCEQRRFYVQALLRAVRCHSLTPNFLQMQLQKCEILQSDSRCKDYLVKIFEELTLHKPTQVMPCRAPKVGRLIYTAGGYFRQSLSYLEAYNPSDGTWLRLADLQVPRSGLAGCVVGGLLYAVGGRNNSPDGNTDSSALDCYNPMTNQWSPCAPMSVPRNRIGVGVIDGHIYAVGGSHGCIHHNSVERYEPERDEWHLVAPMLTRRIGVGVAVLNRLLYAVGGFDGTNRLNSAECYYPERNEWRMITAMNTIRSGAGVCVLHNCIYAAGGYDGQDQLNSVERYDVETETWTFVAPMKHRRSALGITVHQGRIYVLGGYDGHTFLDSVECYDPDTDTWSEVTRMTSGRSGVGVAVTMEPCRKQIDQQNCTC

SEQ ID NO: 41 - protein, 600 aa, Homo sapiens
MAPSSKSERNSGAGSGGGGPGGAGGKRAAGRRREHVLKQLERVKISGQLSPRLFRKLPPRVCVSLKNIVDEDFLYAGHIFLGFSKCGRYVLSYTSSSGDDDFSFYIYHLYWWEFNVHSKLKLVRQVRLFQDEEIYSDLYLTVCEWPSDASKVIVFGFNTRSANGMLMNMMMMSDENHRDIYVSTVAVPPPGRCAACQDASRAHPGDPNAQCLRHGFMLHTKYQVVYPFPTFQPAFQLKKDQVVLLNTSYSLVACAVSVHSAGDRSFCQILYDHSTCPLAPASPPEPQSPELPPALPSFCPEAAPARSSGSPEPSPAIAKAKEFVADIFRRAKEAKGGVPEEARPALCPGPSGSRCRAHSEPLALCGETAPRDSPPASEAPASEPGYVNYTKLYYVLESGEGTEPEDELEDDKISLPFVVTDLRGRNLRPMRERTAVQGQYLTVEQLTLDFEYVINEVIRHDATWGHQFCSFSDYDIVILEVCPETNQVLINIGLLLLAFPSPTEEGQLRPKTYHTSLKVAWDLNTGIFETVSVGDLTEVKGQTSGSVWSSYRKSCVDMVMKWLVPESSGRYVNRMTNEALHKGCSLKVLADSERYTWIVL

SEQ ID NO: 42 - protein, 190 aa, Homo sapiens
MSTRKRRGGAINSRQAQKRTREATSTPEISLEAEPIELVETAGDEIVDLTCESLEPVVVDLTHNDSVVIVDERRRPRRNARRLPQDHADSCVVSSDDEELSRDRDVYVTTHTPRNARDEGATGLRPSGTVSCPICMDGYSEIVQNGRLIVSTECGHVFCSQCLRDSLKNANTCPTCRKKINHKRYHPIYI

SEQ ID NO: 43 - protein, 113 aa, Homo sapiens
MSTRKRRGGAINSRQAQKRTREATSTPEISLEAEPIELVETAGDEIVDLTCESLEPVVVDLTHNDSVVIVDGPQVLSVVPSAWTDTQRSCRMDVSSFPQNAAMSSVASASVIP

SEQ ID NO: 44 - protein, 228 aa, Homo sapiens
MAAQQRDCGGAAQLAGPAAEADPLGRFTCPVCLEVYEKPVQVPCGHVFCSACLQECLKPKKPVCGVCRSALAPGVRAVELERQIESTETSCHGCRKNFFLSKIRSHVATCSKYQNYIMEGVKATIKDASLQPRNVPNRYTFPCPYCPEKNFDQEGLVEHCKLFHSTDTKSVVCPICASMPWGDPNYRSANFREHIQRRHRFSYDTFVDYDVDEEDMMNQVLQRSIIDQ

SEQ ID NO: 45 - protein, 191 aa, Homo sapiens
MAAQQRDCGGAAQLAGPAAEADPLGRFTCPVCLEVYEKPVQVPCGHVFCSACLQECLKPKKPVCGVCRSALAPGVRAVELERQIESTETSCHGCRKNFFLSKIRSHVATCSKYQNYIMEGVKATIKDASLQPRNVPNRYTFPCPYCPEKNFDQEGLVEHCKLFHSTDTKSVVSEQSPCLLSVSCYRASITY

SEQ ID NO: 46 - protein, 216 aa, Homo sapiens
MGPRNPSPDHLSESESEEEENISYLNESSGEEWDSSEEEDSMVPNLSPLESLAWQVKCLLKYSTTWKPLNPNSWLYHAKLLDPSTPVHILREIGLRLSHCSHCVPKLEPIPEWPPLASCGVPPFQKPLTSPSRLSRDHATLNGALQFATKQLSRTLSRATPIPEYLKQIPNSCVSGCCCGWLTKTVKETTRTEPINTTYSYTDFQKAVNKLLTASL

SEQ ID NO: 47 - protein, 848 aa, Homo sapiens
MNSSSANITYASRKRRKPVQKTVKPIPAEGIKSNPSKRHRDRLNTELDRLASLLPFPQDVINKLDKLSVLRLSVSYLRAKSFFDVALKSSPTERNGGQDNCRAANFREGLNLQEGEFLLQALNGFVLVVTTDALVFYASSTIQDYLGFQQSDVIHQSVYELIHTEDRAEFQRQLHWALNPSQCTESGQGIEEATGLPQTVVCYNPDQIPPENSPLMERCFICRLRCLLDNSSGFLAMNFQGKLKYLHGQKKKGKDGSILPPQLALFAIATPLQPPSILEIRTKNFIFRTKHKLDFTPIGCDAKGRIVLGYTEAELCTRGSGYQFIHAADMLYCAESHIRMIKTGESGMIVFRLLTKNNRWTWVQSNARLLYKNGRPDYIIVTQRPLTDEEGTEHLRKRNTKLPFMFTTGEAVLYEATNPFPAIMDPLPLRTKNGTSGKDSATTSTLSKDSLNPSSLLAAMMQQDESIYLYPASSTSSTAPFENNFFNESMNECRNWQDNTAPMGNDTILKHEQIDQPQDVNSFAGGHPGLFQDSKNSDLYSIMKNLGIDFEDIRHMQNEKFFRNDFSGEVDFRDIDLTDEILTYVQDSLSKSPFIPSDYQQQQSLALNSSCMVQEHLHLEQQQQHHQKQVVVEPQQQLCQKMKHMQVNGMFENWNSNQFVPFNCPQQDPQQYNVFTDLHGISQEFPYKSEMDSMPYTQNFISCNQPVLPQHSKCTELDYPMGSFEPSPYPTTSSLEDFVTCLQLPENQKHGLNPQSAIITPQTCYAGAVSMYQCQPEPQHTHVGQMQYNPVLPGQQAFLNKFQNGVLNETYPAELNNINNTQTTTHLQPLHHPSEARPFPDLTSSGFL

SEQ ID NO: 48 - protein, 491 aa, Homo sapiens
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 49 - protein, 1755 aa, Homo sapiens
MASELEPEVQAIDRSLLECSAEEIAGKWLQATDLTREVYQHLAHYVPKIYCRGPNPFPQKEDMLAQHVLLGPMEWYLCGEDPAFGFPKLEQANKPSHLCGRVFKVGEPTYSCRDCAVDPTCVLCMECFLGSIHRDHRYRMTTSGGGGFCDCGDTEAWKEGPYCQKHELNTSEIEEEEDPLVHLSEDVIARTYNIFAITFRYAVEILTWEKESELPADLEMVEKSDTYYCMLFNDEVHTYEQVIYTLQKAVNCTQKEAIGFATTVDRDGRRSVRYGDFQYCEQAKSVIVRNTSRQTKPLKVQVMHSSIVAHQNFGLKLLSWLGSIIGYSDGLRRILCQVGLQEGPDGENSSLVDRLMLSDSKLWKGARSVYHQLFMSSLLMDLKYKKLFAVRFAKNYQQLQRDFMEDDHERAVSVTALSVQFFTAPTLARMLITEENLMSIIIKTFMDHLRHRDAQGRFQFERYTALQAFKFRRVQSLILDLKYVLISKPTEWSDELRQKFLEGFDAFLELLKCMQGMDPITRQVGQHIEMEPEWEAAFTLQMKLTHVISMMQDWCASDEKVLIEAYKKCLAVLMQCHGGYTDGEQPITLSICGHSVETIRYCVSQEKVSIHLPVSRLLAGLHVLLSKSEVAYKFPELLPLSELSPPMLIEHPLRCLVLCAQVHAGMWRRNGFSLVNQIYYYHNVKCRREMFDKDVVMLQTGVSMMDPNHFLMIMLSRFELYQIFSTPDYGKRFSSEITHKDVVQQNNTLIEEMLYLIIMLVGERFSPGVGQVNATDEIKREIIHQLSIKPMAHSELVKSLPEDENKETGMESVIEAVAHFKKPGLTGRGMYELKPECAKEFNLYFYHFSRAEQSKAEEAQRKLKRQNREDTALPPPVLPPFCPLFASLVNILQSDVMLCIMGTILQWAVEHNGYAWSESMLQRVLHLIGMALQEEKQHLENVTEEHVVTFTFTQKISKPGEAPKNSPSILAMLETLQNAPYLEVHKDMIRWILKTFNAVKKMRESSPTSPVAETEGTIMEESSRDKDKAERKRKAEIARLRREKIMAQMSEMQRHFIDENKELFQQTLELDASTSAVLDHSPVASDMTLTALGPAQTQVPEQRQFVTCILCQEEQEVKVESRAMVLAAFVQRSTVLSKNRSKFIQDPEKYDPLFMHPDLSCGTHTSSCGHIMHAHCWQRYFDSVQAKEQRRQQRLRLHTSYDVENGEFLCPLCECLSNTVIPLLLPPRNIFNNRLNFSDQPNLTQWIRTISQQIKALQFLRKEESTPNNASTKNSENVDELQLPEGFRPDFRPKIPYSESIKEMLTTFGTATYKVGLKVHPNEEDPRVPIMCWGSCAYTIQSIERILSDEDKPLFGPLPCRLDDCLRSLTRFAAAHWTVASVSVVQGHFCKLFASLVPNDSHEELPCILDIDMFHLLVGLVLAFPALQCQDFSGISLGTGDLHIFHLVTMAHIIQILLTSCTEENGMDQENPPCEEESAVLALYKTLHQYTGSALKEIPSGWHLWRSVRAGIMPFLKCSALFFHYLNGVPSPPDIQVPGTSHFEHLCSYLSLPNNLICLFQENSEIMNSLIESWCRNSEVKRYLEGERDAIRYPRESNKLINLPEDYSSLINQASNFSCPKSGGDKSRAPTLCLVCGSLLCSQSYCCQTELEGEDVGACTAHTYSCGSGVGIFLRVRECQVLFLAGKTKGCFYSPPYLDDYGETDQGLRRGNPLHLCKERFKKIQKLWHQHSVTEEIGHAQEANQTLVGIDWQHL

SEQ ID NO: 50 - protein, 374 aa, Homo sapiens
MSRVPSPPPPAEMSSGPVAESWCYTQIKVVKFSYMWTINNFSFCREEMGEVIKSSTFSSGANDKLKWCLRVNPKGLDEESKDYLSLYLLLVSCPKSEVRAKFKFSILNAKGEETKAMESQRAYRFVQGKDWGFKKFIRRDFLLDEANGLLPDDKLTLFCEVSVVQDSVNISGQNTMNMVKVPECRLADELGGLWENSRFTDCCLCVAGQEFQAHKAILAARSPVFSAMFEHEMEESKKNRVEINDVEPEVFKEMMCFIYTGKAPNLDKMADDLLAAADKYALERLKVMCEDALCSNLSVENAAEILILADLHSADQLKTQAVDFINYHASDVLETSGWKSMVVSHPHLVAEAYRSLASAQCPFLGPPRKRLKQS

SEQ ID NO: 51 - protein, 587 aa, Homo sapiens
MEGESVKLSSQTLIQAGDDEKNQRTITVNPAHMGKAFKVMNELRSKQLLCDVMIVAEDVEIEAHRVVLAACSPYFCAMFTGDMSESKAKKIEIKDVDGQTLSKLIDYIYTAEIEVTEENVQVLLPAASLLQLMDVRQNCCDFLQSQLHPTNCLGIRAFADVHTCTDLLQQANAYAEQHFPEVMLGEEFLSLSLDQVCSLISSDKLTVSSEEKVFEAVISWINYEKETRLEHMAKLMEHVRLPLLPRDYLVQTVEEEALIKNNNTCKDFLIEAMKYHLLPLDQRLLIKNPRTKPRTPVSLPKVMIVVGGQAPKAIRSVECYDFEEDRWDQIAELPSRRCRAGVVFMAGHVYAVGGFNGSLRVRTVDVYDGVKDQWTSIASMQERRSTLGAAVLNDLLYAVGGFDGSTGLASVEAYSYKTNEWFFVAPMNTRRSSVGVGVVEGKLYAVGGYDGASRQCLSTVEQYNPATNEWIYVADMSTRRSGAGVGVLSGQLYATGGHDGPLVRKSVEVYDPGTNTWKQVADMNMCRRNAGVCAVNGLLYVVGGDDGSCNLASVEYYNPVTDKWTLLPTNMSTGRSYAGVAVIHKSL

SEQ ID NO: 52 - protein, 568 aa, Homo sapiens
MGGIMAPKDIMTNTHAKSILNSMNSLRKSNTLCDVTLRVEQKDFPAHRIVLAACSDYFCAMFTSELSEKGKPYVDIQGLTASTMEILLDFVYTETVHVTVENVQELLPAACLLQLKGVKQACCEFLESQLDPSNCLGIRDFAETHNCVDLMQAAEVFSQKHFPEVVQHEEFILLSQGEVEKLIKCDEIQVDSEEPVFEAVINWVKHAKKEREESLPNLLQYVRMPLLTPRYITDVIDAEPFIRCSLQCRDLVDEAKKFHLRPELRSQMQGPRTRARLGANEVLLVVGGFGSQQSPIDVVEKYDPKTQEWSFLPSITRKRRYVASVSLHDRIYVIGGYDGRSRLSSVECLDYTADEDGVWYSVAPMNVRRGLAGATTLGDMIYVSGGFDGSRRHTSMERYDPNIDQWSMLGDMQTAREGAGLVVASGVIYCLGGYDGLNILNSVEKYDPHTGHWTNVTPMATKRSGAGVALLNDHIYVVGGFDGTAHLSSVEAYNIRTDSWTTVTSMTTPRCYVGATVLRGRLYAIAGYDGNSLLSSIECYDPIIDSWEVVTSMGTQRCDAGVCVLREK

SEQ ID NO: 53 - protein, 609 aa, Homo sapiens
MEGKPMRRCTNIRPGETGMDVTSRCTLGDPNKLPEGVPQPARMPYISDKHPRQTLEVINLLRKHRELCDVVLVVGAKKIYAHRVILSACSPYFRAMFTGELAESRQTEVVIRDIDERAMELLIDFAYTSQITVEEGNVQTLLPAACLLQLAEIQEACCEFLKRQLDPSNCLGIRAFADTHSCRELLRIADKFTQHNFQEVMESEEFMLLPANQLIDIISSDELNVRSEEQVFNAVMAWVKYSIQERRPQLPQVLQHVRLPLLSPKFLVGTVGSDPLIKSDEECRDLVDEAKNYLLLPQERPLMQGPRTRPRKPIRCGEVLFAVGGWCSGDAISSVERYDPQTNEWRMVASMSKRRCGVGVSVLDDLLYAVGGHDGSSYLNSVERYDPKTNQWSSDVAPTSTCRTSVGVAVLGGFLYAVGGQDGVSCLNIVERYDPKENKWTRVASMSTRRLGVAVAVLGGFLYAVGGSDGTSPLNTVERYNPQENRWHTIAPMGTRRKHLGCAVYQDMIYAVGGRDDTTELSSAERYNPRTNQWSPVVAMTSRRSGVGLAVVNGQLMAVGGFDGTTYLKTIEVFDPDANTWRLYGGMNYRRLGGGVGVIKMTHCESHIW

SEQ ID NO: 54 - protein, 406 aa, Homo sapiens
MADGNEDLRADDLPGPAFESYESMELACPAERSGHVAVSDGRHMFVWGGYKSNQVRGLYDFYLPREELWIYNMETGRWKKINTEGDVPPSMSGSCAVCVDRVLYLFGGHHSRGNTNKFYMLDSRSTDRVLQWERIDCQGIPPSSKDKLGVWVYKNKLIFFGGYGYLPEDKVLGTFEFDETSFWNSSHPRGWNDHVHILDTETFTWSQPITTGKAPSPRAAHACATVGNRGFVFGGRYRDARMNDLHYLNLDTWEWNELIPQGICPVGRSWHSLTPVSSDHLFLFGGFTTDKQPLSDAWTYCISKNEWIQFNHPYTEKPRLWHTACASDEGEVIVFGGCANNLLVHHRAAHSNEILIFSVQPKSLVRLSLEAVICFKEMLANSWNCLPKHLLHSVNQRFGSNNTSGS

SEQ ID NO: 55 - protein, 273 aa, Homo sapiens
MGQKVTGGIKTVDMRDPTYRPLKQELQGLDYCKPTRLDLLLDMPPVSYDVQLLHSWNNNDRSLNVFVKEDDKLIFHRHPVAQSTDAIRGKVGYTRGLHVWQITWAMRQRGTHAVVGVATADAPLHSVGYTTLVGNNHESWGWDLGRNRLYHDGKNQPSKTYPAFLEPDETFIVPDSFLVALDMDDGTLSFIVDGQYMGVAFRGLKGKKLYPVVSAVWGHCEIRMRYLNGLDPEPLPLMDLCRRSVRLALGRERLGEIHTLPLPASLKAYLLYQ

SEQ ID NO: 56 - protein, 263 aa, Homo sapiens
MGQTALAGGSSSTPTPQALYPDLSCPEGLEELLSAPPPDLGAQRRHGWNPKDCSENIEVKEGGLYFERRPVAQSTDGARGKRGYSRGLHAWEISWPLEQRGTHAVVGVATALAPLQTDHYAALLGSNSESWGWDIGRGKLYHQSKGPGAPQYPAGTQGEQLEVPERLLVVLDMEEGTLGYAIGGTYLGPAFRGLKGRTLYPAVSAVWGQCQVRIRYLGERRAEPHSLLHLSRLCVRHNLGDTRLGQVSALPLPPAMKRYLLYQ

SEQ ID NO: 57 - protein, 273 aa, Homo sapiens
MGQKLSGSLKSVEVREPALRPAKRELRGAEPGRPARLDQLLDMPAAGLAVQLRHAWNPEDRSLNVFVKDDDRLTFHRHPVAQSTDGIRGKVGHARGLHAWQINWPARQRGTHAVVGVATARAPLHSVGYTALVGSDAESWGWDLGRSRLYHDGKNQPGVAYPAFLGPDEAFALPDSLLVVLDMDEGTLSFIVDGQYLGVAFRGLKGKKLYPVVSAVWGHCEVTMRYINGLDPEPLPLMDLCRRSIRSALGRQRLQDISSLPLPQSLKNYLQYQ

SEQ ID NO: 58 - protein, 198 aa, Homo sapiens
MTLRCLEPSGNGGEGTRSQWGTAGSAEEPSPQAARLAKALRELGQTGWYWGSMTVNEAKEKLKEAPEGTFLIRDSSHSDYLLTISVKTSAGPTNLRIEYQDGKFRLDSIICVKSKLKQFDSVVHLIDYYVQMCKDKRTGPEAPRNGTVHLYLTKPLYTSAPSLQHLCRLTINKCTGAIWGLPLPTRLKDYLEEYKFQV

SEQ ID NO: 59 - protein, 535 aa, Homo sapiens
MKKISLKTLRKSFNLNKSKEETDFMVVQQPSLASDFGKDDSLFGSCYGKDMASCDINGEDEKGGKNRSKSESLMGTLKRRLSAKQKSKGKAGTPSGSSADEDTFSSSSAPIVFKDVRAQRPIRSTSLRSHHYSPAPWPLRPTNSEETCIKMEVRVKALVHSSSPSPALNGVRKDFHDLQSETTCQEQANSLKSSASHNGDLHLHLDEHVPVVIGLMPQDYIQYTVPLDEGMYPLEGSRSYCLDSSSPMEVSAVPPQVGGRAFPEDESQVDQDLVVAPEIFVDQSVNGLLIGTTGVMLQSPRAGHDDVPPLSPLLPPMQNNQIQRNFSGLTGTEAHVAESMRCHLNFDPNSAPGVARVYDSVQSSGPMVVTSLTEELKKLAKQGWYWGPITRWEAEGKLANVPDGSFLVRDSSDDRYLLSLSFRSHGKTLHTRIEHSNGRFSFYEQPDVEGHTSIVDLIEHSIRDSENGAFCYSRSRLPGSATYPVRLTNPVSRFMQVRSLQYLCRFVIRQYTRIDLIQKLPLPNKMKDYLQEKHY

SEQ ID NO: 60 - protein, 387 aa, Homo sapiens
MAGSEPRSGTNSPPPPFSDWGRLEAAILSGWKTFWQSVSKERVARTTSREEVDEAASTLTRLPIDVQLYILSFLSPHDLCQLGSTNHYWNETVRDPILWRYFLLRDLPSWSSVDWKSLPDLEILKKPISEVTDGAFFDYMAVYRMCCPYTRRASKSSRPMYGAVTSFLHSLIIQNEPRFAMFGPGLEELNTSLVLSLMSSEELCPTAGLPQRQIDGIGSGVNFQLNNQHKFNILILYSTTRKERDRAREEHTSAVNKMFSRHNEGDDQQGSRYSVIPQIQKVCEVVDGFIYVANAEAHKRHEWQDEFSHIMAMTDPAFGSSGRPLLVLSCISQGDVKRMPCFYLAHELHLNLLNHPWLVQDTEAETLTGFLNGIEWILEEVESKRAR

SEQ ID NO: 61 - protein, 539 aa, Homo sapiens
MAVCARLCGVGPSRGCRRRQQRRGPAETAAADSEPDTDPEEERIEASAGVGGGLCAGPSPPPPRCSLLELPPELLVEIFASLPGTDLPSLAQVCTKFRRILHTDTIWRRRCREEYGVCENLRKLEITGVSCRDVYAKLLHRYRHILGLWQPDIGPYGGLLNVVVDGLFIIGWMYLPPHDPHVDDPMRFKPLFRIHLMERKAATVECMYGHKGPHHGHIQIVKKDEFSTKCNQTDHHRMSGGRQEEFRTWLREEWGRTLEDIFHEHMQELILMKFIYTSQYDNCLTYRRIYLPPSRPDDLIKPGLFKGTYGSHGLEIVMLSFHGRRARGTKITGDPNIPAGQQTVEIDLRHRIQLPDLENQRNFNELSRIVLEVRERVRQEQQEGGHEAGEGRGRQGPRESQPSPAQPRAEAPSKGPDGTPGEDGGEPGDAVAAAEQPAQCGQGQPFVLPVGVSSRNEDYPRTCRMCFYGTGLIAGHGFTSPERTPGVFILFDEDRFGFVWLELKSFSLYSRVQATFRNADAPSPQAFDEMLKNIQSLTS

SEQ ID NO: 62 - protein, 605 aa, Homo sapiens
MDPAEAVLQEKALKFMCSMPRSLWLGCSSLADSMPSLRCLYNPGTGALTAFQNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRSETSKGVYCLQYDDQKIVSGLRDNTIKIWDKNTLECKRILTGHTGSVLCLQYDERVIITGSSDSTVRVWDVNTGEMLNTLIHHCEAVLHLRFNNGMMVTCSKDRSIAVWDMASPTDITLRRVLVGHRAAVNVVDFDDKYIVSASGDRTIKVWNTSTCEFVRTLNGHKRGIACLQYRDRLVVSGSSDNTIRLWDIECGACLRVLEGHEELVRCIRFDNKRIVSGAYDGKIKVWDLVAALDPRAPAGTLCLRTLVEHSGRVFRLQFDEFQIVSSSHDDTILIWDFLNDPAAQAEPPRSPSRTYTYISR

SEQ ID NO: 63 - protein, 707 aa, Homo sapiens
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 64 - protein, 499 aa, Homo sapiens
MAQFAFESDLHSLLQLDAPIPNAPPARWQRKAKEAAGPAPSPMRAANRSHSAGRTPGRTPGKSSSKVQTTPSKPGGDRYIPHRSAAQMEVASFLLSKENQPENSQTPTKKEHQKAWALNLNGFDVEEAKILRLSGKPQNAPEGYQNRLKVLYSQKATPGSSRKTCRYIPSLPDRILDAPEIRNDYYLNLVDWSSGNVLAVALDNSVYLWSASSGDILQLLQMEQPGEYISSVAWIKEGNYLAVGTSSAEVQLWDVQQQKRLRNMTSHSARVGSLSWNSYILSSGSRSGHIHHHDVRVAEHHVATLSGHSQEVCGLRWAPDGRHLASGGNDNLVNVWPSAPGEGGWVPLQTFTQHQGAVKAVAWCPWQSNVLATGGGTSDRHIRIWNVCSGACLSAVDAHSQVCSILWSPHYKELISGHGFAQNQLVIWKYPTMAKVAELKGHTSRVLSLTMSPDGATVASAAADETLRLWRCFELDPARRREREKASAAKSSLIHQGIR

SEQ ID NO: 65 - protein, 903 aa, Homo sapiens
MSDSGSQLGSMGSLTMKSQLQITVISAKLKENKKNWFGPSPYVEVTVDGQSKKTEKCNNTNSPKWKQPLTVIVTPVSKLHFRVWSHQTLKSDVLLGTAALDIYETLKSNNMKLEEVVVTLQLGGDKEPTETIGDLSICLDGLQLESEVVTNGETTCSENGVSLCLPRLECNSAISAHCNLCLPGLSDSPISASRVAGFTGASQNDDGSRSKDETRVSTNGSDDPEDAGAGENRRVSGNNSPSLSNGGFKPSRPPRPSRPPPPTPRRPASVNGSPSATSESDGSSTGSLPPTNTNTNTSEGATSGLIIPLTISGGSGPRPLNPVTQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECDLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYARTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 66 - protein, 882 aa, Homo sapiens
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLPEEAERVKAQVQALGLAEAQPMAVVQSVPGAHPVPVYAFSIKGPSYGEDVSNTTTAQKRKCSQTQCPRKVIKMESEEGKEARLARSSPEQPRPSTSKAVSPPHLDGPPSPRSPVIGSEVFLPNSNHVASGAGEAEERVVVISSSEDSDAENSSSRELDDSSSESSDLQLEGPSTLRVLDENLADPQAEDRPLVFFDLKIDNETQKISQLAAVNRESKFRVVIQPEAFFSIYSKAVSLEVGLQHFLSFLSSMRRPILACYKLWGPGLPNFFRALEDINRLWEFQEAISGFLAALPLIRERVPGASSFKLKNLAQTYLARNMSERSAMAAVLAMRDLCRLLEVSPGPQLAQHVYPFSSLQCFASLQPLVQAAVLPRAEARLLALHNVSFMELLSAHRRDRQGGLKKYSRYLSLQTTTLPPAQPAFNLQALGTYFEGLLEGPALARAEGVSTPLAGRGLAERASQQS

SEQ ID NO: 67 - protein, 475 aa, Homo sapiens [sequence omitted]

SEQ ID NO: 68 - protein, 1050 aa, Homo sapiens
MEVAVEKAVAAAAAASAAASGGPSAAPSGENEAESRQGPDSERGGEAARLNLLDTCAVCHQNIQSRAPKLLPCLHSFCQRCLPAPQRYLMLPAPMLGSAETPPPVPAPGSPVSGSSPFATQVGVIRCPVCSQECAERHIIDNFFVKDTTEVPSSTVEKSNQVCTSCEDNAEANGFCVECVEWLCKTCIRAHQRVKFTKDHTVRQKEEVSPEAVGVTSQRPVFCPFHKKEQLKLYCETCDKLTCRDCQLLEHKEHRYQFIEEAFQNQKVIIDTLITKLMEKTKYIKFTGNQIQNRIIEVNQNQKQVEQDIKVAIFTLMVEINKKGKALLHQLESLAKDHRMKLMQQQQEVAGLSKQLEHVMHFSKWAVSSGSSTALLYSKRLITYRLRHLLRARCDASPVTNNTIQFHCDPSFWAQNIINLGSLVIEDKESQPQMPKQNPVVEQNSQPPSGLSSNQLSKFPTQISLAQLRLQHMQQQVMAQRQQVQRRPAPVGLPNPRMQGPIQQPSISHQQPPPRLINFQNHSPKPNGPVLPPHPQQLRYPPNQNIPRQAIKPNPLQMAFLAQQAIKQWQISSGQGTPSTTNSTSSTPSSPTITSAAGYDGKAFGSPMIDLSSPVGGSYNLPSLPDIDCSSTIMLDNIVRKDTNIDHGQPRPPSNRTVQSPNSSVPSPGLAGPVTMTSVHPPIRSPSASSVGSRGSSGSSSKPAGADSTHKVPVVMLEPIRIKQENSGPPENYDFPVVIVKQESDEESRPQNANYPRSILTSLLLNSSQSSTSEETVLRSDAPDSTGDQPGLHQDNSSNGKSEWLDPSQKSPLHVGETRKEDDPNEDWCAVCQNGGELLCCEKCPKVFHLSCHVPTLTNFPSGEWICTFCRDLSKPEVEYDCDAPSHNSEKKKTEGLVKLTPIDKRKCERLLLFLYCHEMSLAFQDPVPLTVPDYYKIIKNPMDLSTIKKRLQEDYSMYSKPEDFVADFRLIFQNCAEFNEPDSEVANAGIKLENYFEELLKNLYPEKRFPKPEFRNESEDNKFSDDSDDDFVQPRKKRLKSIEERQLLK

SEQ ID NO: 69 - protein, 1127 aa, Homo sapiens
MAENKGGGEAESGGGGSGSAPVTAGAAGPAAQEAEPPLTAVLVEEEEEEGGRAGAEGGAAGPDDGGVAAASSGSAQAASSPAASVGTGVAGGAVSTPAPAPASAPAPGPSAGPPPGPPASLLDTCAVCQQSLQSRREAEPKLLPCLHSFCLRCLPEPERQLSVPIPGGSNGDIQQVGVIRCPVCRQECRQIDLVDNYFVKDTSEAPSSSDEKSEQVCTSCEDNASAVGFCVECGEWLCKTCIEAHQRVKFTKDHLIRKKEDVSESVGASGQRPVFCPVHKQEQLKLFCETCDRLTCRDCQLLEHKEHRYQFLEEAFQNQKGAIENLLAKLLEKKNYVHFAATQVQNRIKEVNETNKRVEQEIKVAIFTLINEINKKGKSLLQQLENVTKERQMKLLQQQNDITGLSRQVKHVMNFTNWAIASGSSTALLYSKRLITFQLRHILKARCDPVPAANGAIRFHCDPTFWAKNVVNLGNLVIESKPAPGYTPNVVVGQVPPGTNHISKTPGQINLAQLRLQHMQQQVYAQKHQQLQQMRMQQPPAPVPTTTTTTQQHPRQAAPQMLQQQPPRLISVQTMQRGNMNCGAFQAHQMRLAQNAARIPGIPRHSGPQYSMMQPHLQRQHSNPGHAGPFPVVSVHNTTINPTSPTTATMANANRGPTSPSVTAIELIPSVTNPENLPSLPDIPPIQLEDAGSSSLDNLLSRYISGSHLPPQPTSTMNPSPGPSALSPGSSGLSNSHTPVRPPSTSSTGSRGSCGSSGRTAEKTSLSFKSDQVKVKQEPGTEDEICSFSGGVKQEKTEDGRRSACMLSSPESSLTPPLSTNLHLESELDALASLENHVKIEPADMNESCKQSGLSSLVNGKSPIRSLMHRSARIGGDGNNKDDDPNEDWCAVCQNGGDLLCCEKCPKVFHLTCHVPTLLSFPSGDWICTFCRDIGKPEVEYDCDNLQHSKKGKTAQGLSPVDQRKCERLLLYLYCHELSIEFQEPVPASIPNYYKIIKKPMDLSTVKKKLQKKHSQHYQIPDDFVADVRLIFKNCERFNEMMKVVQVYADTQEINLKADSEVAQAGKAVALYFEDKLTEIYSDRTFAPLPEFEQEEDDGEVTEDSDEDFIQPRRKRLKSDERPVHIK

SEQ ID NO: 70 - protein, 300 aa, Homo sapiens
MCARGQVGRGTQLRTGRPCSQVPGSRWRPERLLRRQRAGGRPSRPHPARARPGLSLPATLLGSRAAAAVPLPLPPALAPGDPAMPVRTECPPPAGASAASAASLIPPPPINTQQPGVATSLLYSGSKFRGHQKSKGNSYDVEVVLQHVDTGNSYLCGYLKIKGLTEEYPTLTTFFEGEIISKKHPFLTRKWDADEDVDRKHWGKFLAFYQYAKSFNSDDFDYEELKNGDYVFMRWKEQFLVPDHTIKDISGASFAGFYYICFQKSAASIEGYYYHRSSEWYQSLNLTHVPEHSAPIYEFR

SEQ ID NO: 71 - protein, 546 aa, Homo sapiens
MGSRNSSSAGSGSGDPSEGLPRRGAGLRRSEEEEEEDEDVDLAQVLAYLLRRGQVRLVQGGGAANLQFIQALLDSEEENDRAWDGRLGDRYNPPVDATPDTRELEFNEIKTQVELATGQLGLRRAAQKHSFPRMLHQRERGLCHRGSFSLGEQSRVISHFLPNDLGFTDSYSQKAFCGIYSKDGQIFMSACQDQTIRLYDCRYGRFRKFKSIKARDVGWSVLDVAFTPDGNHFLYSSWSDYIHICNIYGEGDTHTALDLRPDERRFAVFSIAVSSDGREVLGGANDGCLYVFDREQNRRTLQIESHEDDVNAVAFADISSQILFSGGDDAICKVWDRRTMREDDPKPVGALAGHQDGITFIDSKGDARYLISNSKDQTIKLWDIRRFSSREGMEASRQAATQQNWDYRWQQVPKKAWRKLKLPGDSSLMTYRGHGVLHTLIRCRFSPIHSTGQQFIYSGCSTGKVVVYDLLSGHIVKKLTNHKACVRDVSWHPFEEKIVSSSWDGNLRLWQYRQAEYFQDDMPESEECASAPAPVPQSSTPFSSPQ